(19) United States
(12) Patent Application Publication
(10) Pub. No.: US 2024/0424113 A1
(43) Pub. Date: Dec. 26, 2024

(54) CONJUGATES COMPRISING ANTIFUNGALS AND HEAT SHOCK PROTEIN 90 (HSP90) INHIBITORS AND METHODS OF USE THEREOF

(71) Applicant: Bright Angel Therapeutics Inc., Toronto (CA)

(72) Inventors: Dominic Jaikaran, Toronto (CA); Abdelmalik Slassi, Mississauga (CA); Mee Shelley, New York, NY (US); Andrew Placzek, New York, NY (US); Kejia Ding, Shanghai (CN); Jinzhu Ge, Shanghai (CN)

(73) Assignee: Bright Angel Therapeutics Inc., Toronto, ON (CA)

(21) Appl. No.: 18/702,505

(22) PCT Filed: Oct. 13, 2022
(86) PCT No.: PCT/CA2022/051504

§ 371 Date: Apr. 18, 2024

Related U.S. Application Data
(60) Provisional application No. 63/262,722, filed on Oct. 19, 2021.

(51) Int. Cl. A61K 47/55; A61K 47/54

(52) U.S. Cl. CPC A61K 47/552; A61K 47/545

(57) ABSTRACT

The present application relates conjugate compounds of Formula (I): wherein: A is a moiety that increases fungal cell uptake and/or fungal cell permeability; B is a HSP90 inhibiting moiety; and L¹ is a linker; or pharmaceutically acceptable salts, solvates and/or prodrugs thereof, to compositions comprising these compounds or pharmaceutically acceptable salts, solvates and/or prodrugs thereof, and various uses in the treatment or prevention of fungal-related diseases, disorders or conditions.

A-L¹-B (I)

### CROSS-REFERENCE TO RELATED APPLICATIONS

The present application claims the benefit of priority of co-pending U.S. Provisional Patent Application No. 63/262,722, which was filed Oct. 19, 2021, the contents of which are incorporated herein by reference in their entirety.

### FIELD

The present application relates to conjugate compounds, to processes for their preparation, to compositions comprising them, and to their use in therapy. More particularly, it relates to conjugates comprising a moiety that increases fungal cell uptake and/or fungal cell permeability and a heat shock protein inhibitor moiety, useful in the treatment or prevention of fungal-related diseases, disorders or conditions.

### BACKGROUND

Hsp90 is an essential chaperone in all eukaryotes that regulates the form and function of diverse client proteins, many of which are key signal transducers (Leach et al., Nat Rev Microbiol 10, 693-704 (2012); Taipale et al., Nat Rev Mol Cell Biol 11, 515-28 (2010)). Hsp90 function is regulated by an interplay of ATP binding and hydrolysis, interactions with co-chaperones, and post-translational modifications.

It has been established that Hsp90 promotes antifungal drug tolerance and the evolution of drug resistance in the leading fungal pathogens of humans, including species of Candida, Cryptococcus and Aspergillus (Cowen et al., Eukaryot Cell 5, 2184-8 (2006); Cowen et al., Science 309, 2185-9 (2005); Cowen, et al., Proc Natl Acad Sci USA (2009); Singh et al., PLOS Pathog 5, e1000532 (2009); Singh-Babak et al., PLOS Pathog 8, e1002718 (2012); Serpa, R. et al., Microbiology 162, 309-317 (2016)). In Candida species, polyene resistance is contingent on robust Hsp90 function (Vincent et al., PLOS Biol 11, e1001692 (2013)). In species of Candida, Cryptococcus and Aspergillus, genetic depletion or pharmacological inhibition of Hsp90 abrogates resistance to azoles and echinocandins (Cowen et al., Science 309, 2185-9 (2005); Cowen, et al., Proc Natl Acad Sci USA (2009); Singh et al., PLOS Pathog 5, e1000532 (2009); Singh-Babak et al., PLoS Pathog 8, e1002718 (2012).; Lamoth et al., Antimicrob Agents Chemother 57, 1035-9 (2013); Lamoth et al., Antimicrob Agents Chemother 58, 1889-96 (2014); Lamoth et al., J Infect Dis 209, 473-81 (2014)). Compromise of Hsp90 function abrogates drug resistance arising from diverse mutations, including resistance that evolves in the human host (Cowen et al., Science 309, 2185-9 (2005); Cowen, et al., Proc Natl Acad Sci USA (2009); Singh et al., PLOS Pathog 5, e1000532 (2009); Singh-Babak et al., PLOS Pathog 8, e1002718 (2012); LaFayette et al., PLOS Pathog 6, e1001069 (2010)). Hsp90 regulates drug resistance via the protein phosphatase calcineurin and the terminal mitogen-activated protein kinase (MAPK) of the cell wall integrity pathway, Mkc1 (Cowen et al., Science 309, 2185-9 (2005); Cowen, et al., Proc Natl Acad Sci USA (2009); Singh et al., PLOS Pathog 5, e1000532 (2009); LaFayette et al., PLOS Pathog 6, e1001069 (2010)). Targeting regulators of Hsp90 function such as lysine deacetylases or protein kinases recapitulates many of Hsp90's own effects on fungal drug resistance (Diezmann et al., PLOS Genet 8, e1002562 (2012); Robbins et al., Cell Reports 2, 878-88 (2012)). Thus, Hsp90 serves as a global hub and an Achilles heel that can be targeted to cripple fungal pathogens (Cowen et al., Curr Opin Microbiol 16, 377-84 (2013); Lamoth et al., Crit Rev Microbiol, 1-12 (2014)). See also Hendrickson J A, Hu C, Aitken S L, Beyda N. Antifungal Resistance: a Concerning Trend for the Present and Future. Curr Infect Dis Rep. 2019 Nov. 16; 21 (12): 47.

### SUMMARY

The present application includes a conjugate compound of Formula (I) or an enantiomer thereof, or a pharmaceutically acceptable salt, solvate and/or prodrug thereof,

A-L¹-B (I)

wherein: A is a moiety that increases fungal cell uptake and/or fungal cell permeability; B is a HSP90 inhibiting moiety; and L¹ is a linker comprising at least one complimentary functional group to covalently bind with A and at least one complimentary functional group to react with B, to form the conjugate of Formula (I).

Also provided is a method of treating or preventing a fungal-related disease, disorder or condition comprising administering a therapeutically effective amount of one or more conjugate compounds of the present application to a subject in need thereof.

Also included is a method of inhibiting or preventing fungal growth comprising administering a therapeutically effective amount of one or more conjugate compounds of the present application to a subject in need thereof.

Also provided is a method of inhibiting fungal HSP90 activity comprising administering a therapeutically effective amount of one or more conjugate compounds of the present application to a subject in need thereof.

Further provided is a method of selectively inhibiting fungal HSP90 activity comprising administering a therapeutically effective amount of one or more conjugate compounds of the present application to a subject in need thereof.

Also included is a method of treating or preventing mycosis comprising administering a therapeutically effective amount of one or more conjugate compounds of the present application to a subject in need thereof.

Further included is a method of treating or preventing a fungal-related disease, disorder or condition comprising administering a therapeutically effective amount of one or more conjugate compounds of the present application in combination with another known agent useful for treatment or prevention of a fungal-related disease, disorder or condition to a subject in need thereof.

Also included is a pharmaceutical composition comprising one or more conjugate compounds of the present application, or a pharmaceutically acceptable salt, and/or solvate thereof, and a pharmaceutically acceptable carrier and/or diluent.

Further provided is an agricultural composition comprising one or more conjugate compounds of the present application, or a salt, and/or solvate thereof, and an agriculturally acceptable carrier and/or diluent.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component or effect, such as an additional or second compound, the second compound as used herein is different from the other compounds or first compound. A “third” compound is different from the other, first, and second compounds, and further enumerated or “additional” compounds are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “compound of the application” or “conjugate compound of the application” and the like as used herein refers to a conjugate compound of Formula (I), or an enantiomer thereof, or pharmaceutically acceptable salts, solvates and/or prodrugs thereof.

The term “composition of the application” or “composition of the present application” and the like as used herein refers to a composition comprising one or more conjugate compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains 6 to 20 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluorosubstituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

The term “cross-coupling” as used herein refers to chemical reactions in which two different starting materials, each of which is usually endowed with an activating group, are reacted together with the aid of a metal catalyst. The result is the loss of the two activating groups and the formation of a new covalent bond between the remaining fragments.

The term “cell” as used herein refers to a single cell or a plurality of cells and includes a cell either in a cell culture or in a subject.

The term “subject” as used herein includes all members of the animal kingdom including mammals and the plant kingdom. Thus the methods and uses of the present application are applicable to human therapy, veterinary and agricultural applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a fungal-related disease, disorder or condition, or manifesting a symptom associated with a fungal-related disease, disorder or condition.

As used herein, the term “effective amount” or “therapeutically effective amount” means an amount of a compound, or one or more compounds, of the application that is effective, at dosages and for periods of time necessary to achieve the desired result.

The expression “inhibiting HSP90” as used herein refers to inhibiting, blocking and/or disrupting HSP90 activity in a fungal cell, whether direct or indirect. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “fungal-related disease, disorder or condition” means that the disease, disorder or condition to be treated is affected by, modulated by and/or has some biological basis, either direct or indirect, that includes fungal activity. These diseases respond favourably when fungal activity associated with the disease, disorder or condition is inhibited by one or more of the compounds or compositions of the application.

The term “HSP90” as used herein refers to the heat shock protein 90.

The term “fungal cell uptake” or “fungal cell permeability” as used herein refers to the regulation or controlling exchanges of molecules between the cell and its environment, for example an increased cell permeability would mean an increased ability for a molecule to internalize into the cell.

The term “administered” as used herein means administration of a therapeutically effective amount of a compound, or one or more compounds, or a composition of the application to a cell either in cell culture or in a subject.

The term “linker moiety” as used herein refers to any molecular structure that joins two or more other molecular structures together.

The term “complementary functional group” as used herein refers to a group of atoms or a single atom that will react with another group of atoms or a single atom to form a covalent bond between the two groups or atoms.

The term “reacts with” as used herein generally means that there is a flow of electrons or a transfer of electrostatic charge resulting in the formation of a covalent bond.

The term “conjugating” as used herein means to bind two molecules together via a covalent bond.

The present description refers to a number of chemical terms and abbreviations used by those skilled in the art. Nevertheless, definitions of selected terms are provided for clarity and consistency.

The term “aq.” as used herein refers to aqueous.

The term “Me” as used herein refers to methyl.

The term “Et” as used herein refers to ethyl.

The term “Pr” as used herein refers to propyl.

The term “Bu” as used herein refers to butyl.

The term “Ac” as used herein refers to acetyl.

The term “Ph” as used herein refers to phenyl.

The term “Ts” as used herein refers to tosyl.

The term “Ms” as used herein refers to mesyl.

The term “THF” as used herein refers to tetrahydrofuran.

The term “HATU” as used herein refers to hexafluorophosphate azabenzotriazole tetramethyl uronium.

The terms “DIPEA” or “DIEA” as used herein refer to N,N-diisopropylethylamine.

The term “TEA” as used herein refers to triethylamine.

The term “DMF” as used herein refers to dimethylformamide.

The term “DCM” as used herein refers to dichloromethane.

The term “DCE” as used herein refers to dichloroethane.

The term “Xphos” as used herein refers to dicyclohexyl [2′,4′,6′-tris(propan-2-yl)[1,1′-biphenyl]-2-yl] phosphane.

The term “DMAP” as used herein refers to 4-dimethylaminopyridine.

The term “TFA” as used herein refers to trifluoroacetic acid.

The term “ACN” as used herein refers to acetonitrile.

The term “TMS” as used herein refers to trimethylsilyl.

The term “SEM” as used herein refers to trimethylsilylethoxymethyl.

The term “BOC” as used herein refers to tert-butyloxycarbonyl.

The term “DMSO” as used herein refers to dimethylsulfoxide.

The term “NIS” as used herein refers to N-iodosuccinimide.

The term “NMP” as used herein refers to N-methyl-2-pyrrolidone.

The term “DMP” as used herein refers to Dess-Martin periodinane.

The term “CDI” as used herein refers to 1,1′-carbonyldiimidazole.

The term “HBPIN” as used herein refers to pinacolborane or 4,4,5,5-tetramethyl-1,3,2-dioxaborolanee.

The term “HPLC” as used herein refers to high-performance liquid chromatography.

The term “NMR” as used herein refers to nuclear magnetic resonance.

The term “LCMS” as used herein refers to liquid chromatography-mass spectrometry.

The term “SPR” as used herein refers to surface plasmon resonance.

The term “HBS” as used herein refers to phosphate buffered saline containing detergent.

The term “HEPES” as used herein refers to (4-(2-hydroxyethyl)-1-piperazineethanesulfonic acid).

The term “MIC” as used herein refers to minimum inhibitory concentrations.

The term “YPD” as used herein refers to yeast extract peptone dextrose.

### II. Compounds and Compositions

The present application describes a novel class of conjugate compounds.

Accordingly, the application includes a conjugate compound of Formula (I) or an enantiomer thereof, or a pharmaceutically acceptable salt, solvate and/or prodrug thereof,

A-L¹-B (I)
wherein:
A is a moiety that increases fungal cell uptake and/or fungal cell permeability;
B is a HSP90 inhibiting moiety; and
L¹ is a linker comprising at least one complimentary functional group to covalently link to A and at least one complimentary functional group to covalently link to B.

In some embodiments, A is an antifungal moiety. In some embodiments, A comprises at least one of: an azole moiety, a polyamine moiety, a fatty acid ester moiety, a fatty acid amide moiety, a fatty alcohol moiety, flucytosine (5-FC) moiety and a triphenylphosphonium moiety. In some embodiments, the azole moiety is selected from an imidazole moiety, a triazole moiety, tetrazole moiety and a thiazole moiety. In some embodiments, A is any antifungal agent known in the art. For example, A is selected from fluconazole, itraconazole, clotrimazole, ketoconazole, voriconazole, posaconazole, isavuconazonium, miconazole, flucytosine, olorofim, manogepix, ibrexafungerp, caspofungin, micafungin, anidulafungin, rezafungin, amphotericin B, and VT-1161.

In some embodiments, B is a C₄₋₂₀heteroaryl moiety. In some embodiments, B comprises at least one of: a pyrazinone moiety, an indazole moiety and a pyrimidine moiety. In some embodiments, B comprises a pyrrolopyrazinone moiety, a thienopyrizanone moiety, a tetrahydroindazole moiety and/or a pyrrolopyrimidine moiety. In some embodiments, B comprises any compound known to inhibit HSP90, such as the compounds listed in Table 1 below.

TABLE 1
HSP90 Inhibitors
IUPAC name

(1-(3,4-dimethoxyphenyl)cyclopropyl)(5-methoxy-2,2-dimethyl-2H-chromen-6-
yl)methanone
(1aR,14R,15aR,E)-8-chloro-9,11-dihydroxy-14-methyl-1a,2,3,14,15,15a-hexahydro-6H-
benzo[c]oxireno[2,3-k][1]oxacyclotetradecine-6,12(7H)-dione
(1aR,2Z,4E,14R,15aR)-8-chloro-9,11-dihydroxy-14-methyl-1a,14,15,15a-tetrahydro-6H-
benzo[c]oxireno[2,3-k][1]oxacyclotetradecine-6,12(7H)-dione
(1r,4r)-4-((2-carbamoyl-5-(6,6-dimethyl-4-oxo-3-(trifluoromethyl)-4,5,6,7-tetrahydro-1H-
indazol-1-yl)phenyl)amino)cyclohexyl glycinate
(2,4-dihydroxy-5-isopropylphenyl)(2-(2-methoxyethoxy)-7,8-dihydropyrido[4,3-d]pyrimidin-
6(5H)-yl)methanone
(2,4-dihydroxy-5-isopropylphenyl)(5-((4-methylpiperazin-1-yl)methyl)isoindolin-2-
yl)methanone
(2,4-dihydroxy-6-((3-(3-methoxyphenyl)-1-methyl-1H-pyrazol-5-yl)amino)phenyl)(isoindolin-
2-yl)methanone
(2R,3R)-5,7-dihydroxy-2-(3,4,5-trihydroxyphenyl)chroman-3-yl 3,4,5-trihydroxybenzoate
(2R,3R)-5,7-dihydroxy-2-phenylchroman-3-yl 4-acetoxy-3-(3-methylbut-2-en-1-yl)benzoate
(3R,4S,5R,6R)-5-hydroxy-6-((4-hydroxy-3-(4-hydroxy-3-(3-methylbut-2-en-1-
yl)benzamido)-8-methyl-2-oxo-2H-chromen-7-yl)oxy)-3-methoxy-2,2-dimethyltetrahydro-
2H-pyran-4-yl carbamate
(3R,4S,5R,6R)-6-((4-hydroxy-3-(4-hydroxy-3-(3-methylbut-2-en-1-yl)benzamido)-8-methyl-
2-oxo-2H-chromen-7-yl)oxy)-3-methoxy-2,2,5-trimethyltetrahydro-2H-pyran-4-yl carbamate
(3R,6R,9R,12S,15S)-9-benzhydryl-3,12-diisobutyl-15-isopropyl-1-methyl-6-(thiazol-4-
ylmethyl)-1,4,7,10,13-pentaazacyclopentadecane-2,5,8,11,14-pentaone
(3S,6S,9S,12S,15S)-3-benzyl-6,12,15-triisobutyl-9-isopropyl-1,4,7,10,13-
pentaazacyclopentadecane-2,5,8,11,14-pentaone
(3S,6S,9S,12S,15S)-6-benzyl-3,9,15-triisobutyl-12-isopropyl-1-oxa-4,7,10,13-
tetraazacyclopentadecane-2,5,8,11,14-pentaone
(4,6-dihydropyrrolo[3,4-c]pyrazol-5(1H)-yl)(2,4-dihydroxy-6-((3-(3-methoxyphenyl)-1-
methyl-1H-pyrazol-5-yl)amino)phenyl)methanone
(4E,6Z,8S,9S,10E,12S,13R,14S,16R)-13-hydroxy-8,14,19-trimethoxy-4,10,12,16-
tetramethyl-3,20,22-trioxo-2-azabicyclo[16.3.1]docosa-1(21),4,6,10,18-pentaen-9-yl
carbamate
(4E,6Z,8S,9S,10E,12S,13R,14S,16R)-1⁴-(allylamino)-1²,1⁵,13-trihydroxy-8,14-dimethoxy-
4,10,12,16-tetramethyl-3-oxo-2-aza-1(1,3)-benzenacycloheptadecaphane-4,6,10-trien-9-yl
carbamate
(4E,6Z,8S,9S,10E,12S,13R,14S,16R)-19-(allylamino)-13-hydroxy-8,14-dimethoxy-
4,10,12,16-tetramethyl-3,20,22-trioxo-2-azabicyclo[16.3.1]docosa-1(21),4,6,10,18-pentaen-
9-yl carbamate
(4E,6Z,8S,9S,10E,12S,13R,14S,16R)-19-amino-13-hydroxy-8,14-dimethoxy-4,10,12,16-
tetramethyl-3,20,22-trioxo-2-azabicyclo[16.3.1]docosa-1(21),4,6,10,18-pentaen-9-yl
carbamate
(5-chloro-2,4-dihydroxyphenyl)(2-methyl-7,8-dihydropyrido[4,3-d]pyrimidin-6(5H)-
yl)methanone
(7aS,13aS)-9,10-dimethoxy-3,3-dimethyl-13,13a-dihydro-3H-pyrano[2,3-c:6,5-f′]dichromen-
7(7aH)-one
(R)-2-amino-7-(4-fluoro-2-(6-methoxypyridin-2-yl)phenyl)-4-methyl-7,8-dihydropyrido[4,3-
d]pyrimidin-5(6H)-one
(S)-1-(4-(2-(6-amino-8-((6-bromobenzo[d][1,3]dioxol-5-yl)thio)-9H-purin-9-yl)ethyl)piperidin-
1-yl)-2-hydroxypropan-1-one
1-(2,4-dihydroxy-5-isopropylphenyl)-N-ethyl-5-(4-(morpholinomethyl)phenyl)-1H-1,2,3-
triazole-4-carboxamide
1-(4-aminoquinazolin-7-yl)-2,6,6-trimethyl-1,5,6,7-tetrahydro-4H-indol-4-one
1²-amino-2⁴,2⁶-dimethyl-12-thia-4,8-diaza-1(4,6)-pyrimidina-2(1,3)-
benzenacyclododecaphane-3,9-dione
2-((4-(furan-2-yl)-6-(trifluoromethyl)pyrimidin-2-yl)thio)-N-(5-methylisoxazol-3-yl)acetamide
2-((6-(dimethylamino)benzo[d][1,3]dioxol-5-yl)thio)-1-(2-(neopentyl-ë²-azaneyl)ethyl)-1H-
imidazo[4,5-c]pyridin-4-amine
2-((6-(dimethylamino)benzo[d][1,3]dioxol-5-yl)thio)-1-(2-(neopentylamino)ethyl)-1H-
imidazo[4,5-c]pyridin-4-amine
2-(2-ethyl-3,5-dihydroxy-6-(3-methoxy-4-(2-morpholinoethoxy)benzoyl)phenyl)-N,N-bis(2-
methoxyethyl)acetamide
2-(3,4-dimethoxyphenyl)-1-(5-methoxy-2,2-dimethyl-2H-chromen-6-yl)-2-methylpropan-1-
one
2-amino-4-(2,4-dichloro-5-(2-(pyrrolidin-1-yl)ethoxy)phenyl)-N-ethylthieno[2,3-d]pyrimidine-
6-carboxamide
4-((4-amino-6-(5-chloro-1H,3H-benzo[de]isochromen-6-yl)-1,3,5-triazin-2-
yl)thio)butanamide
4-(2-amino-4-chloro-7-((4-methoxy-3,5-dimethylpyridin-2-yl)methyl)-7H-pyrrolo[2,3-
d]pyrimidin-5-yl)but-3-yn-1-yl dihydrogen phosphate
4-(2,4-dimethylphenyl)-2-(methylthio)-7H-pyrrolo[2,3-d]pyrimidine-5-carbonitrile
4-(4-(2,3-dihydrobenzo[b][1,4]dioxin-6-yl)-5-methyl-1H-pyrazol-3-yl)-6-ethylbenzene-1,3-
diol
4-(5-chloro-1H,3H-benzo[de]isochromen-6-yl)-6-(methylthio)-1,3,5-triazin-2-amine
4-(5-hydroxy-4-(1-(2-(piperidin-4-yl)ethyl)-1H-indol-5-yl)-4H-1,2,4-triazol-3-yl)-6-
isopropylbenzene-1,3-diol
4-(5-hydroxy-4-(1-methyl-1H-indol-5-yl)-4H-1,2,4-triazol-3-yl)-6-isopropylbenzene-1,3-diol
4-(5,7-dichloro-2,3-dihydrobenzofuran-4-yl)-5H-pyrrolo[3,2-d]pyrimidin-2-amine
4-(6,6-dimethyl-4-oxo-3-(trifluoromethyl)-4,5,6,7-tetrahydro-1H-indazol-1-yl)-2-(((1r,4r)-4-
hydroxycyclohexyl)amino)benzamide
4-chloro-6-(5-((2-morpholinoethyl)amino)benzo[d]isoxazol-3-yl)benzene-1,3-diol
5-(((R)-sec-butyl)amino)-N1-((1R,3r,5S)-8-(5-(cyclopropanecarbonyl)pyridin-2-yl)-8-
azabicyclo[3.2.1]octan-3-yl)-2-methylterephthalamide
5-(1-(4-(tert-butyl)benzyl)-1H-1,2,3-triazol-4-yl)benzene-1,2,3-triol
5-(2-amino-4-chloro-7-((4-methoxy-3,5-dimethylpyridin-2-yl)methyl)-7H-pyrrolo[2,3-
d]pyrimidin-5-yl)-2-methylpent-4-yn-2-ol
5-(2,4-dihydroxy-5-isopropylbenzoyl)-N-ethyl-4,5,6,7-tetrahydroisoxazolo[4,5-c]pyridine-3-
carboxamide
5-(2,4-dihydroxy-5-isopropylphenyl)-4-(1-methyl-1H-indol-5-yl)-2,4-dihydro-3H-1,2,4-triazol-
3-one
5-(2,4-dihydroxy-5-isopropylphenyl)-N-ethyl-4-(2-methyl-1,2,3,4-tetrahydroisoquinolin-6-
yl)isoxazole-3-carboxamide
5-(2,4-dihydroxy-5-isopropylphenyl)-N-ethyl-4-(4-(morpholinomethyl)phenyl)isoxazole-3-
carboxamide
5-(2,4-dihydroxy-6-(4-nitrophenoxy)phenyl)-N-(1-methylpiperidin-4-yl)isoxazole-3-
carboxamide
5-(2,5-dioxo-3-(trifluoromethyl)-2,5-dihydro-1H-pyrrol-1-yl)-N-(2-methylpyridin-4-yl)furan-2-
carboxamide
5-(5-chloro-2,4-dihydroxyphenyl)-N-ethyl-4-(4-methoxyphenyl)-1H-pyrazole-3-carboxamide
5-(5-chloro-2,4-dihydroxyphenyl)-N-ethyl-4-(4-methoxyphenyl)isoxazole-3-carboxamide
6-chloro-9-((4-methoxy-3,5-dimethylpyridin-2-yl)methyl)-9H-purin-2-amine
6-chloro-N⁴-((4-chloro-3,5-dimethylpyridin-2-yl)methyl)-N⁴-((4-(4-fluorophenyl)tetrahydro-
2H-pyran-4-yl)methyl)pyrimidine-2,4-diamine
8-(((1s,4s)-4-hydroxycyclohexyl)amino)-3-propyl-6-(3,6,6-trimethyl-4-oxo-4,5,6,7-
tetrahydro-1H-indazol-1-yl)-3,4-dihydroisoquinolin-1(2H)-one
8-(((1s,4s)-4-hydroxycyclohexyl)amino)-3-propyl-6-(3,6,6-trimethyl-4-oxo-4,5,6,7-
tetrahydro-1H-indazol-1-yl)isoquinolin-1(2H)-one
8-((6-iodobenzo[d][1,3]dioxol-5-yl)thio)-9-(3-(isopropyl-ë²-azaneyl)propyl)-9H-purin-6-amine
8-((6-iodobenzo[d][1,3]dioxol-5-yl)thio)-9-(3-(isopropylamino)propyl)-9H-purin-6-amine
8-((6-iodobenzo[d][1,3]dioxol-5-yl)thio)-9-(3-(isopropylamino)propyl)-9H-purin-6-amine
8-cyclopentyl-6-(3,6,6-trimethyl-4-oxo-4,5,6,7-tetrahydro-1H-indol-1-yl)-3,4-
dihydroisoquinolin-1(2H)-one
8-cyclopentyl-6-(3,6,6-trimethyl-4-oxo-4,5,6,7-tetrahydro-1H-indol-1-yl)-3,4-
dihydroisoquinolin-1(2H)-one
9-butyl-8-(3,4,5-trimethoxybenzyl)-4,9-dihydro-8H-purin-6-amine
methyl 2-(2-(1-benzyl-1H-imidazol-2-yl)ethyl)-3-chloro-4,6-dihydroxybenzoate
N-(4′-((1-methylpiperidin-4-yl)oxy)-[1,1′-biphenyl]-4-yl)-2-naphthamide
N-(5-methyl-6-((1-methylpiperidin-4-yl)oxy)-3-oxo-3,4-dihydronaphthalen-2-yl)-1-phenethyl-
1H-1,2,3-triazole-4-carboxamide
N-(7-(((2R,3R,4S,5R)-3,4-dihydroxy-5-methoxy-6,6-dimethyltetrahydro-2H-pyran-2-yl)oxy)-
8-methyl-2-oxo-2H-chromen-3-yl)acetamide
N-ethyl-2,4-dihydroxy-5-isopropyl-N-(pyridin-3-yl)benzamide
N-ethyl-2,4-dihydroxy-N-(1H-indol-4-yl)-5-isopropylbenzamide

It would be known to a person skilled in the art that any one of the compounds listed in Table 1 can be linked to L¹ via any functional group in the structure capable of forming a covalent bond, such as an amine, thiol, halo, hydroxyl, alkoxy, carboxyl, ester, amide and/or oxo group.

In some embodiments, L¹ comprises one or more of C (O),C(S), O, S, S (O), SO₂, NR², C₁₋₁₂alkylene, C₆₋₂₀arylene, C₃₋₁₀cycloalkylene, C₂₋₂₀heteroarylene and C₃₋₁₀heterocycloalkylene, wherein each of the alkylene, arylene, cycloalkylene, heteroarylene and heterocycloalkylene is optionally substituted with one or more R¹, and each R¹ is independently selected from C₁₋₁₂alkyl, NR²C (O) R³, NR²C(S) R³, NR²R³, NR²C(S) NR²R³, halo, C (O) NR²R³, C(S) NR²R³, SR² and OR²; and R² and R³ are independently selected from H, C₁₋₁₂alkyl and C₁₋₁₂fluoroalkyl.

In some embodiments, L¹ comprises 6 groups selected from C (O), O, S, NR², C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 5 groups selected from C (O), O, NR², C₁₋₁₂alkylene, C₆₋₂₀arylene, C₃₋₁₀cycloalkylene, C₂₋₂₀heteroarylene and C₃₋₁₀heterocycloalkylene. In some embodiments, L′ comprises 4 groups selected from C (O), O, NR², C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 3 groups selected from C (O), O, NR², C₁₋₁₂alkylene, C₆₋₂₀arylene, C₃₋₁₀cycloalkylene, C₂₋₂₀heteroarylene and C₃₋₁₀heterocycloalkylene. In some embodiments, L′ comprises 2 groups selected from C (O), O, NR², C₁₋₁₂alkylene, C₆₋₂₀arylene, C₃₋₁₀cycloalkylene, C₂₋₂₀heteroarylene and C₃₋₁₀heterocycloalkylene. Each of each of the alkylene, arylene, cycloalkylene, heteroarylene and heterocycloalkylene is optionally substituted with one or more, one to six, one to five, one to four, one to three, one or two, or one R¹. In some embodiments, L¹ comprises one or more, one to six, one to five, one to four, one to three, one or two, or one of C₁₋₆alkylene, C₆₋₁₀arylene, C₃₋₈cycloalkylene, C₂₋₁₀heteroarylene and C₃₋₈heterocycloalkylene, each of which is optionally substituted with one or more, one to six, one to five, one to four, one to three, one or two, or one R¹.

In some embodiments, L¹ is selected from C₁₋₁₂alkylene-NR²—C(O), C₁₋₆alkylene-C₆₋₁₀arylene-NR²—C(O), C₁₋₆alkylene-NR²—C(O)—C₂₋₁₀heteroarylene, C₁₋₆alkylene-C₂₋₁₀heteroarylene-NR²—C(O), C₂₋₁₀heteroarylene-NR²—C(O), C₁₋₆alkylene-C₃₋₁₀heterocycloalkylene-NR²—C(O)—C₂₋₁₀heteroarylene, C₁₋₆alkylene-C₃₋₁₀heterocycloalkylene-C₂₋₁₀heteroarylene, C₁₋₆alkylene-C₃₋₁₀heterocycloalkylene-NR²—C (O), C₁₋₆alkylene-C₃₋₁₀heterocycloalkylene-C₁₋₆alkylene-NR²—C(O), C₁₋₆alkylene-C₃₋₁₀heterocycloalkylene-C₆₋₁₀arylene-NR²—C(O), C₁₋₆alkylene-C₃₋₁₀heterocycloalkylene-C₁₋₆alkylene-C₆₋₁₀arylene-NR²—C(O), C₁₋₆alkylene-C(O)—C₃₋₁₀heterocycloalkylene-NR²—C (O), C₁₋₆alkylene-C(O)—C₃₋₁₀heterocycloalkylene-NR²—C₁₋₆alkylene, C₁₋₆alkylene-O—C(O)—C₁₋₆alkylene-C₃₋₁₀heterocycloalkylene-NR²—C(O), C₁₋₆alkylene-C₃₋₁₀heterocycloalkylene-NR²—C₁₋₆alkylene, C₁₋₆alkylene-C₃₋₁₀heterocycloalkylene-C₁₋₆alkylene-NR²—C₁₋₆alkylene, C₁₋₆alkylene-C₃₋₁₀heterocycloalkylene-C₁₋₆alkylene, C₁₋₆alkylene-NR²—C₁₋₆alkylene, O—C₁₋₁₂alkylene, C₁₋₆alkylene-C₃₋₁₀heterocycloalkylene-O—C₁₋₆alkylene-C₆₋₁₀arylene-NR²—C(O), C₁₋₆alkylene-C₃₋₁₀heterocycloalkylene-O—C₁₋₆alkylene-O—C₆₋₁₀arylene-NR²—C(O), and NR²—C₁₋₆alkylene-O—C₁₋₆alkylene-O—C₁₋₆alkylene-NR². Each L¹ defined above is optionally substituted with one or more R¹. A person skilled in the art will appreciate that each of A and B are covalently bonded to either end of L¹ as defined above.

In some embodiments, each R¹ is independently selected from C₁₋₆alkyl, NR²C (O) R³, NR²R³, F, Cl, C (O) NR²R³, and OR²; and R² and R³ are independently selected from H, C₁₋₆alkyl and C₁₋₆fluoroalkyl. In some embodiments, each R¹ is independently selected from methyl, ethyl, propyl, F, Cl, NH₂, NHC (O) C₁₋₆alkyl, C (O) NH₂ and OH. In some embodiments, each R¹ is independently selected from methyl, ethyl, F, Cl, C (O) NH₂ and OH. In some embodiments, R² and R³ are independently selected from H, methyl, ethyl, propyl, CF₃, CHF₂ and CH₂F.

In some embodiments, B is selected from:

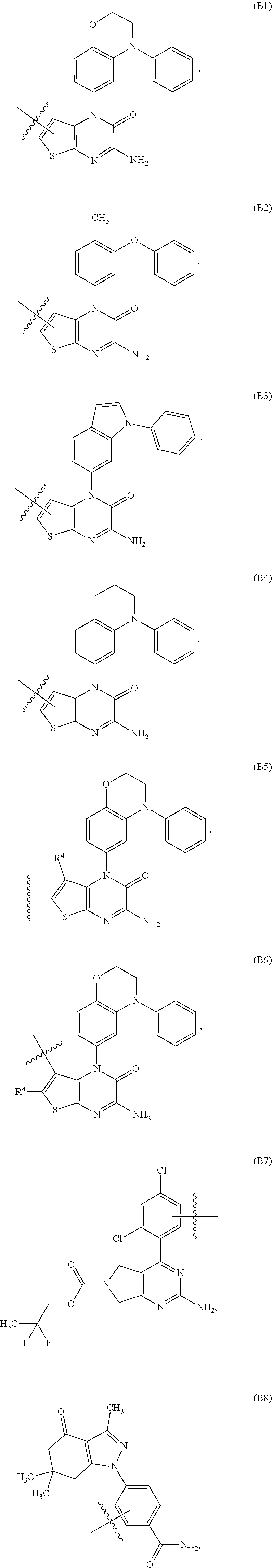

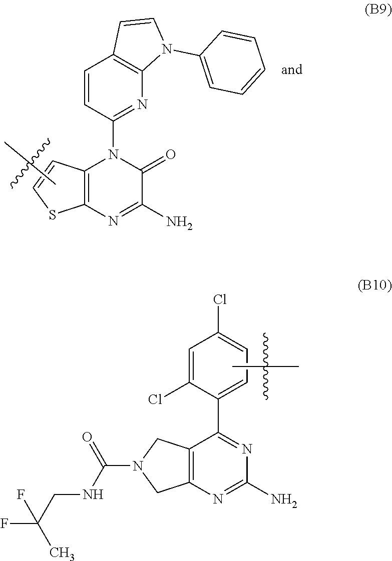

wherein R⁴ is selected from C(O)OC₁₋₆alkyl, C(S)OC₁₋₆alkyl, C(O)OH, C(O)NH₂, C(O)NHC₁₋₆alkyl, C(S)NHC₁₋₆alkyl and CH(OH)C₁₋₆alkyl, and each alkyl group is optionally fluorosubstituted; and [symbol] represents the point of attachment to the remainder of the conjugate, wherein the point of attachment is at the 6- or 7-position in B1, B2, B3, B4, and B9, or the ortho or meta position in B7, B8 and A10.

In some embodiments, B is

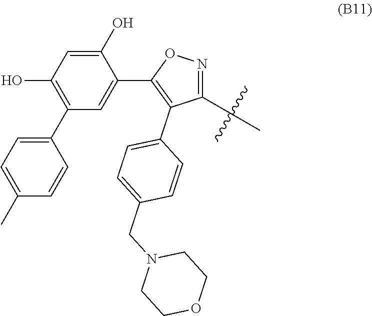 [SMILES: *c1noc(-c2cc(-c3ccc(C)cc3)c(O)cc2O)c1-c1ccc(CN2CCOCC2)cc1]

and [symbol] represents the point of attachment to the remainder of the conjugate

In some embodiments, A is selected from:

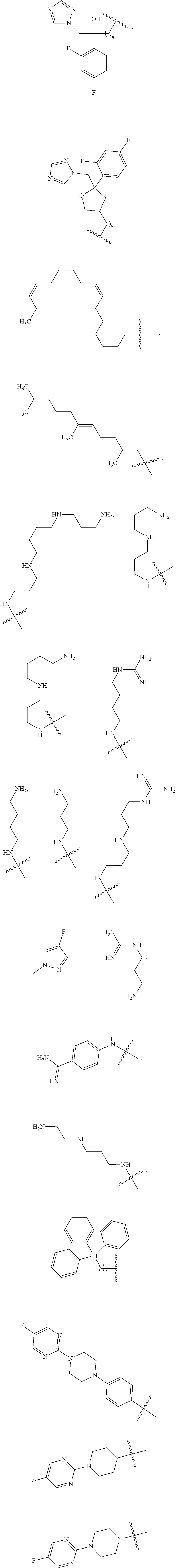

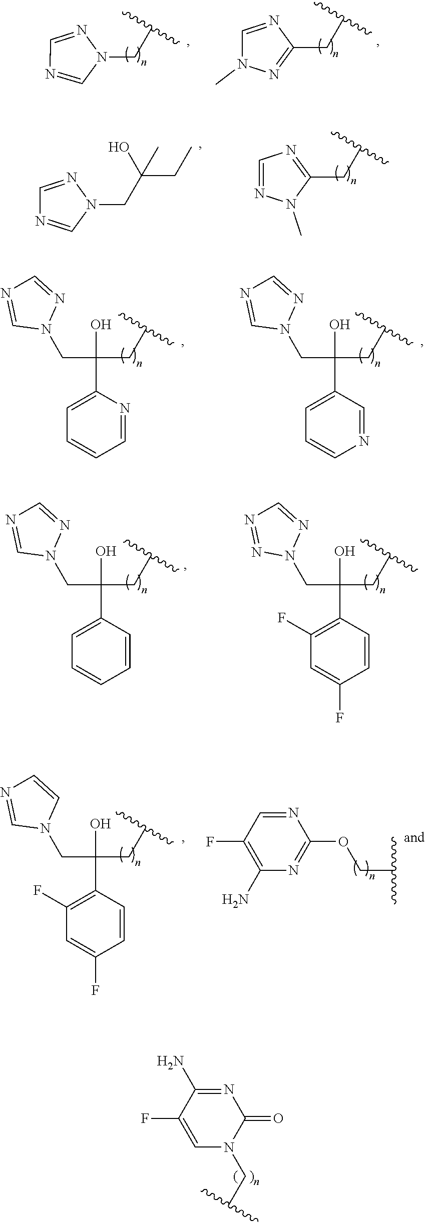

wherein each n is independently an integer from 0 to 4, and [symbol] represents the point of attachment to the remainder of the conjugate.

In some embodiments, A is

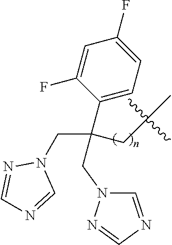

wherein each n is independently an integer from 0 to 4, and [symbol] represents the point of attachment to the remainder of the conjugate.

In some embodiments, L¹ is selected from:

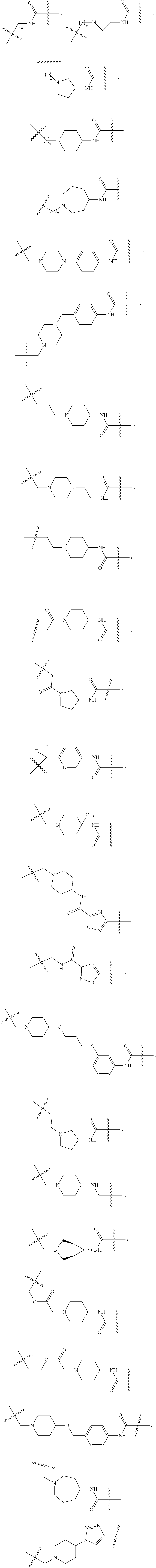

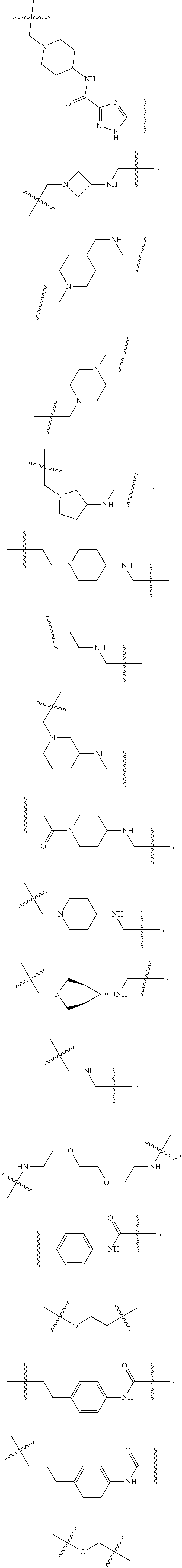

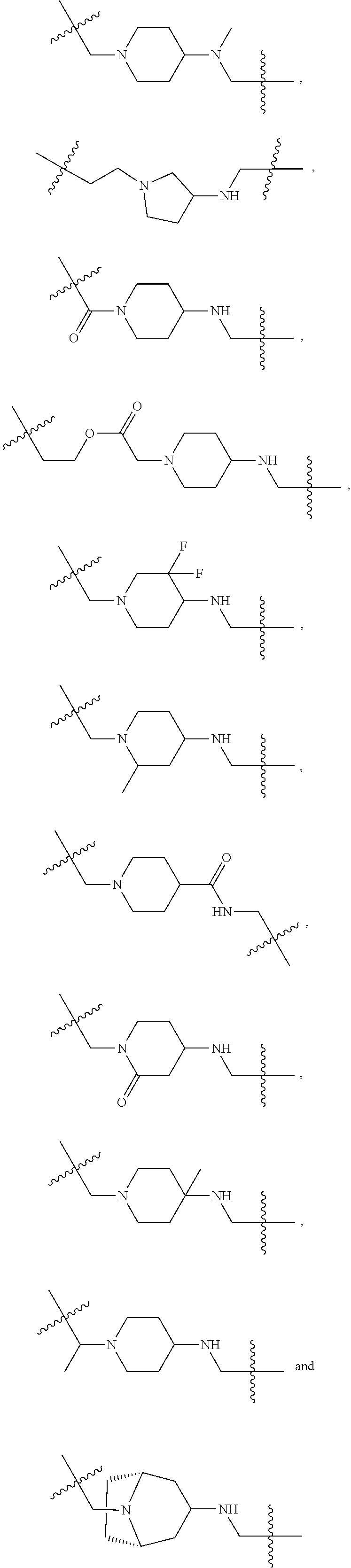

wherein each n is independently an integer from 0 to 4, and [symbol] represents the points of attachment to the remainder of the conjugate.

In some embodiments, L1 is selected from

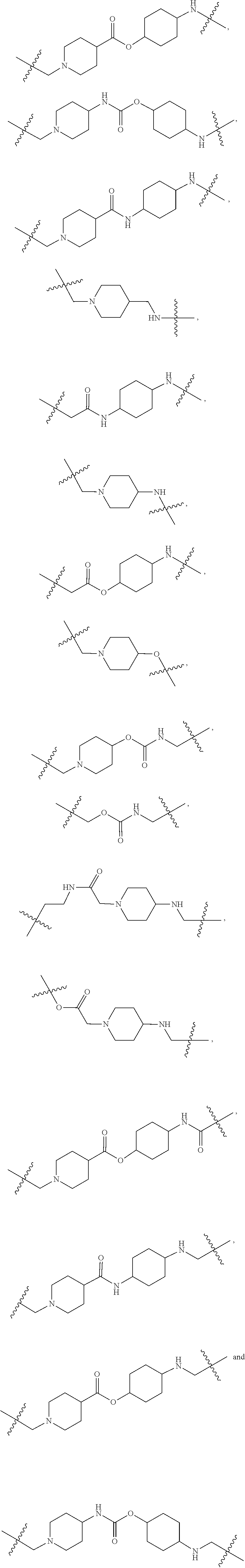

wherein [symbol] represents the points of attachment to the remainder of the conjugate.

In some embodiments, the conjugate compound of Formula (1) is selected from the compounds listed in Table 2:

TABLE 2

| Com- | | |
|---|---|---|
| pound | | |
| ID# | IUPAC Name | Structure |
| I-1 | 3-amino-N-(3-(2,4- difluorophenyl)-3-hydroxy-4- (1H-1,2,4-triazol-1-yl)butyl)-2- oxo-1-(4-phenyl-3,4-dihydro- 2H-benzo[b][1,4]oxazin-6-yl)- 1,2-dihydrothieno[2,3- b]pyrazine-6-carboxamide | 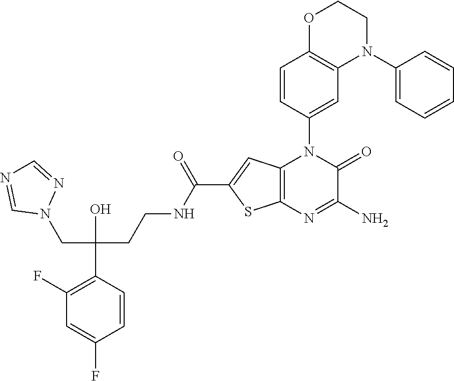 [SMILES: Nc1nc2sc(C(=O)NCCC(O)(Cn3cncn3)c3ccc(F)cc3F)cc2n(-c2ccc3c(c2)N(c2ccccc2)CCO3)c1=O] |
| I-2 | 3-amino-N-(2-(2,4- difluorophenyl)-2-hydroxy-3- (1H-1,2,4-triazol-1-yl)propyl)-2- oxo-1-(4-phenyl-3,4-dihydro- 2H-benzo[b][1,4]oxazin-6-yl)- 1,2-dihydrothieno[2,3- b]pyrazine-6-carboxamide | 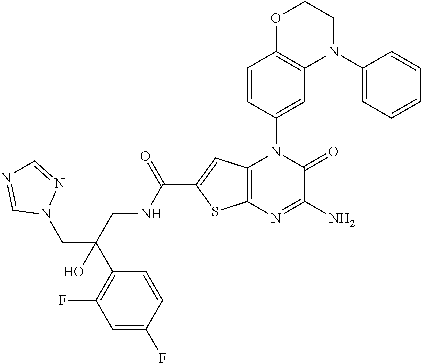 [SMILES: Nc1nc2sc(C(=O)NCC(O)(Cn3cncn3)c3ccc(F)cc3F)cc2n(-c2ccc3c(c2)N(c2ccccc2)CCO3)c1=O] |
| I-3 | 3-amino-N-(1-(2-(2,4- difluorophenyl)-2-hydroxy-3- (1H-1,2,4-triazol-1- yl)propyl)azetidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 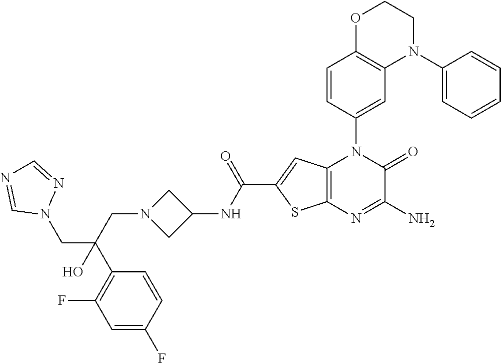 [SMILES: Nc1nc2sc(C(=O)NC3CN(CC(O)(Cn4cncn4)c4ccc(F)cc4F)C3)cc2n(-c2ccc3c(c2)N(c2ccccc2)CCO3)c1=O] |
| I-4 | 3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 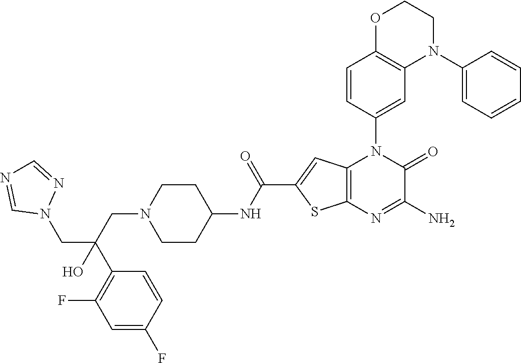 [SMILES: Nc1nc2sc(C(=O)NC3CCN(CC(O)(Cn4cncn4)c4ccc(F)cc4F)CC3)cc2n(-c2ccc3c(c2)N(c2ccccc2)CCO3)c1=O] |
| I-5 | 3-amino-N-((3R)-1-(2-(2,4- difluorophenyl)-2-hydroxy-3- (1H-1,2,4-triazol-1- yl)propyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 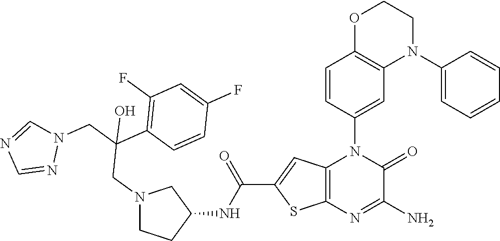 [SMILES: Nc1nc2sc(C(=O)N[C@@H]3CCN(CC(O)(Cn4cncn4)c4ccc(F)cc4F)C3)cc2n(-c2ccc3c(c2)N(c2ccccc2)CCO3)c1=O] |
| I-6 | (R)-3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 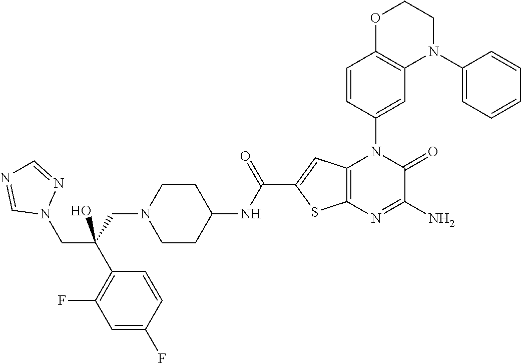 [SMILES: Nc1nc2sc(C(=O)NC3CCN(C[C@@](O)(Cn4cncn4)c4ccc(F)cc4F)CC3)cc2n(-c2ccc3c(c2)N(c2ccccc2)CCO3)c1=O] |
| I-7 | (S)-3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 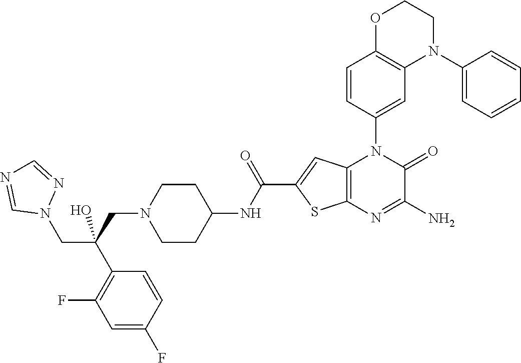 [SMILES: Nc1nc2sc(C(=O)NC3CCN(C[C@](O)(Cn4cncn4)c4ccc(F)cc4F)CC3)cc2n(-c2ccc3c(c2)N(c2ccccc2)CCO3)c1=O] |
| I-8 | 3-amino-N-(4-(4-(2-(2,4- difluorophenyl)-2-hydroxy-3- (1H-1,2,4-triazol-1- yl)propyl)piperazin-1- yl)phenyl)-2-oxo-1-(4-phenyl- 3,4-dihydro-2H- benzo[b][1,4]oxazin-6-yl)-1,2- dihydrothieno[2,3-b]pyrazine- 6-carboxamide | 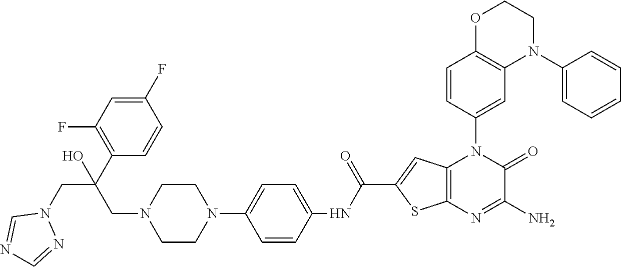 [SMILES: Nc1nc2sc(C(=O)Nc3ccc(N4CCN(CC(O)(Cn5cncn5)c5ccc(F)cc5F)CC4)cc3)cc2n(-c2ccc3c(c2)N(c2ccccc2)CCO3)c1=O] |
| I-9 | 3-amino-N-(4-((4-(2-(2,4- difluorophenyl)-2-hydroxy-3- (1H-1,2,4-triazol-1- yl)propyl)piperazin-1- yl)methyl)phenyl)-2-oxo-1-(4- phenyl-3,4-dihydro-2H- benzo[b][1,4]oxazin-6-yl)-1,2- dihydrothieno[2,3-b]pyrazine- 6-carboxamide | 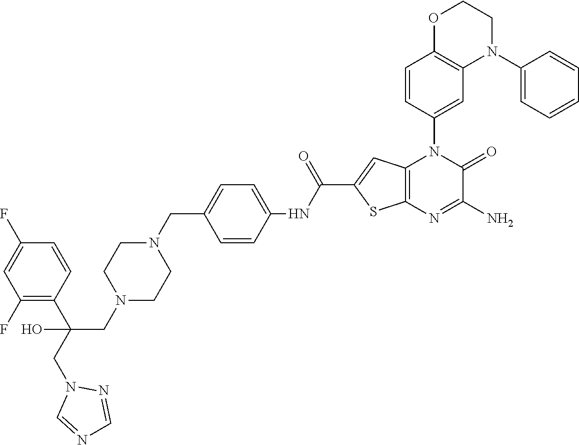 [SMILES: Nc1nc2sc(C(=O)Nc3ccc(CN4CCN(CC(O)(Cn5cncn5)c5ccc(F)cc5F)CC4)cc3)cc2n(-c2ccc3c(c2)N(c2ccccc2)CCO3)c1=O] |
| I-10 | 3-amino-N-((R)-1-((S)-2-(2,4- difluorophenyl)-2-hydroxy-3- (1H-1,2,4-triazol-1- yl)propyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 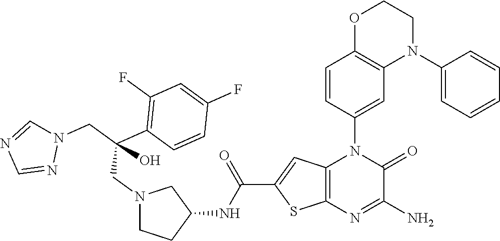 [SMILES: Nc1nc2sc(C(=O)N[C@@H]3CCN(C[C@](O)(Cn4cncn4)c4ccc(F)cc4F)C3)cc2n(-c2ccc3c(c2)N(c2ccccc2)CCO3)c1=O] |
| I-11 | 3-amino-N-((R)-1-((R)-2-(2,4- difluorophenyl)-2-hydroxy-3- (1H-1,2,4-triazol-1- yl)propyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 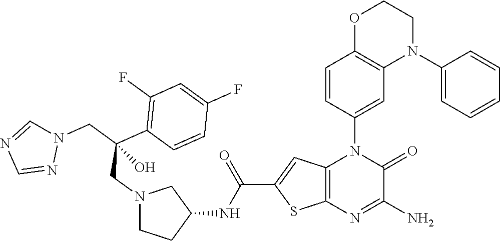 [SMILES: Nc1nc2sc(C(=O)N[C@@H]3CCN(C[C@@](O)(Cn4cncn4)c4ccc(F)cc4F)C3)cc2n(-c2ccc3c(c2)N(c2ccccc2)CCO3)c1=O] |
| I-12 | 3-amino-N-(1-(2-(2,4- difluorophenyl)-2-hydroxy-3- (1H-1,2,4-triazol-1- yl)propyl)piperidin-4-yl)-1-(4- methyl-3-phenoxyphenyl)-2- oxo-1,2-dihydrothieno[2,3- b]pyrazine-6-carboxamide | 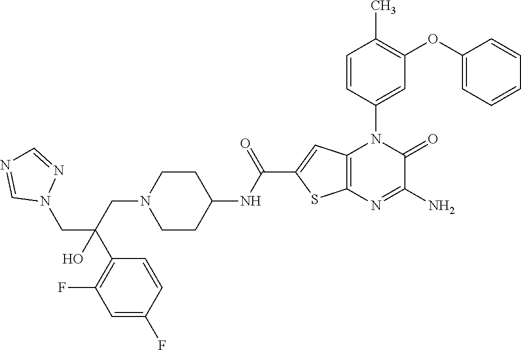 [SMILES: Cc1ccc(-n2c(=O)c(N)nc3sc(C(=O)NC4CCN(CC(O)(Cn5cncn5)c5ccc(F)cc5F)CC4)cc32)cc1Oc1ccccc1] |
| I-13 | 3-amino-N-(1-(2-(2,4- difluorophenyl)-2-hydroxy-3- (1H-1,2,4-triazol-1- yl)propyl)piperidin-4-yl)-2-oxo- 1-(1-phenyl-1H-indol-6-yl)-1,2- dihydrothieno[2,3-b]pyrazine- 6-carboxamide | 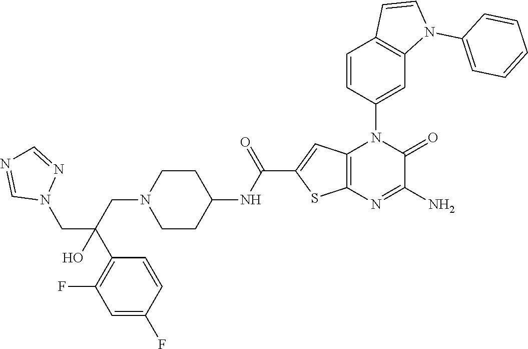 [SMILES: Nc1nc2sc(C(=O)NC3CCN(CC(O)(Cn4cncn4)c4ccc(F)cc4F)CC3)cc2n(-c2ccc3ccn(-c4ccccc4)c3c2)c1=O] |
| I-14 | 3-amino-N-((3S)-1-(2-(2,4- difluorophenyl)-2-hydroxy-3- (1H-1,2,4-triazol-1- yl)propyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 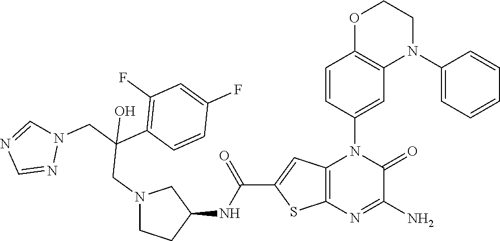 [SMILES: Nc1nc2sc(C(=O)N[C@H]3CCN(CC(O)(Cn4cncn4)c4ccc(F)cc4F)C3)cc2n(-c2ccc3c(c2)N(c2ccccc2)CCO3)c1=O] |
| I-15 | 3-amino-N-(2-(4-(2-(2,4- difluorophenyl)-2-hydroxy-3- (1H-1,2,4-triazol-1- yl)propyl)piperazin-1-yl)ethyl)- 2-oxo-1-(4-phenyl-3,4-dihydro- 2H-benzo[b][1,4]oxazin-6-yl)- 1,2-dihydrothieno[2,3- b]pyrazine-6-carboxamide | 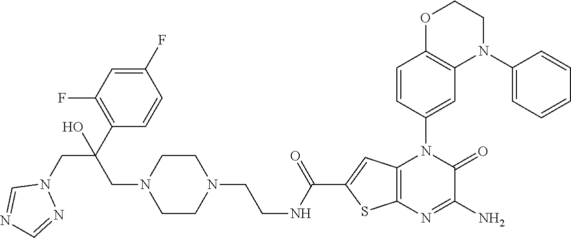 [SMILES: Nc1nc2sc(C(=O)NCCN3CCN(CC(O)(Cn4cncn4)c4ccc(F)cc4F)CC3)cc2n(-c2ccc3c(c2)N(c2ccccc2)CCO3)c1=O] |
| I-16 | 3-amino-N-(1-(2-(2,4- difluorophenyl)-2-hydroxy-3- (1H-1,2,4-triazol-1- yl)propyl)piperidin-4-yl)-2-oxo- 1-(1-phenyl-1,2,3,4- tetrahydroquinolin-7-yl)-1,2- dihydrothieno[2,3-b]pyrazine- 6-carboxamide | 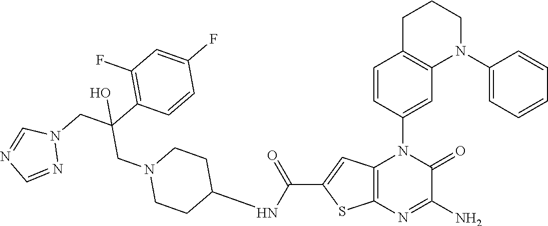 [SMILES: Nc1nc2sc(C(=O)NC3CCN(CC(O)(Cn4cncn4)c4ccc(F)cc4F)CC3)cc2n(-c2ccc3c(c2)N(c2ccccc2)CCC3)c1=O] |
| I-17 | 3-amino-N-((3S)-1-(2-(2,4- difluorophenyl)-2-hydroxy-3- (1H-1,2,4-triazol-1- yl)propyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 7-carboxamide | 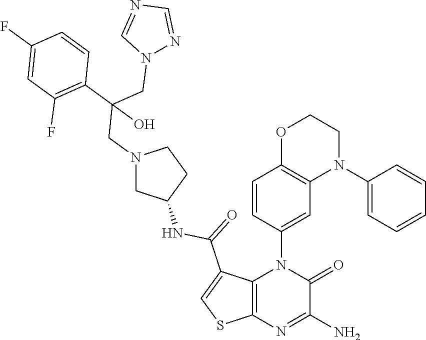 [SMILES: Nc1nc2scc(C(=O)N[C@H]3CCN(CC(O)(Cn4cncn4)c4ccc(F)cc4F)C3)c2n(-c2ccc3c(c2)N(c2ccccc2)CCO3)c1=O] |
| I-18 | 3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 7-carboxamide | 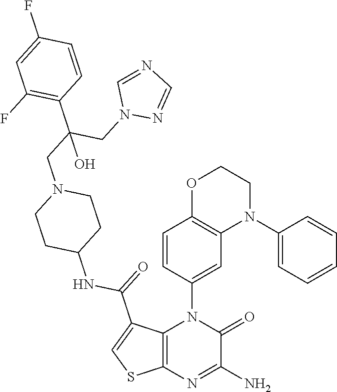 [SMILES: Nc1nc2scc(C(=O)NC3CCN(CC(O)(Cn4cncn4)c4ccc(F)cc4F)CC3)c2n(-c2ccc3c(c2)N(c2ccccc2)CCO3)c1=O] |
| I-19 | 3-amino-N-(1-(2-(2,4- difluorophenyl)-2-hydroxy-3- (1H-1,2,4-triazol-1- yl)propyl)azetidin-3-yl)-2-oxo- 1-(4-phenyl-3,4-dihydro-2H- benzo[b][1,4]oxazin-6-yl)-1,2- dihydrothieno[2,3-b]pyrazine- 7-carboxamide | 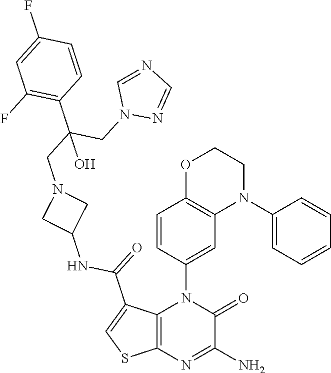 [SMILES: Nc1nc2scc(C(=O)NC3CN(CC(O)(Cn4cncn4)c4ccc(F)cc4F)C3)c2n(-c2ccc3c(c2)N(c2ccccc2)CCO3)c1=O] |
| I-20 | (R)-3-amino-N-(1-(2-(2,4- difluorophenyl)-2-hydroxy-3- (1H-1,2,4-triazol-1- yl)propyl)piperidin-4-yl)-2-oxo- 1-(1-phenyl-1,2,3,4- tetrahydroquinolin-7-yl)-1,2- dihydrothieno[2,3-b]pyrazine- 6-carboxamide | 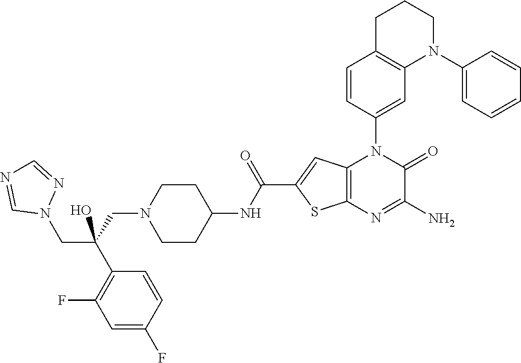 [SMILES: Nc1nc2sc(C(=O)NC3CCN(C[C@@](O)(Cn4cncn4)c4ccc(F)cc4F)CC3)cc2n(-c2ccc3c(c2)N(c2ccccc2)CCC3)c1=O] |
| I-21 | (S)-3-amino-N-(1-(2-(2,4- difluorophenyl)-2-hydroxy-3- (1H-1,2,4-triazol-1- yl)propyl)piperidin-4-yl)-2-oxo- 1-(1-phenyl-1,2,3,4- tetrahydroquinolin-7-yl)-1,2- dihydrothieno[2,3-b]pyrazine- 6-carboxamide | 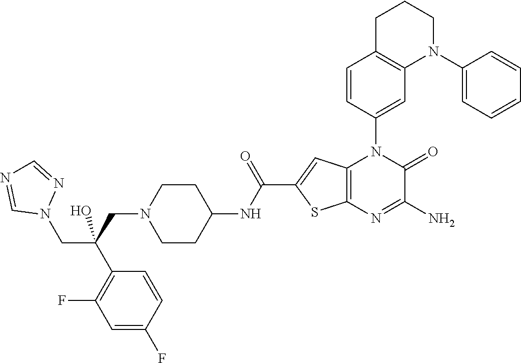 [SMILES: Nc1nc2sc(C(=O)NC3CCN(C[C@](O)(Cn4cncn4)c4ccc(F)cc4F)CC3)cc2n(-c2ccc3c(c2)N(c2ccccc2)CCC3)c1=O] |
| I-22 | (R)-3-amino-N-(1-(2-(2,4- difluorophenyl)-2-hydroxy-3- (1H-1,2,4-triazol-1- yl)propyl)piperidin-4-yl)-2-oxo- 1-(1-phenyl-1H-indol-6-yl)-1,2- dihydrothieno[2,3-b]pyrazine- 6-carboxamide | 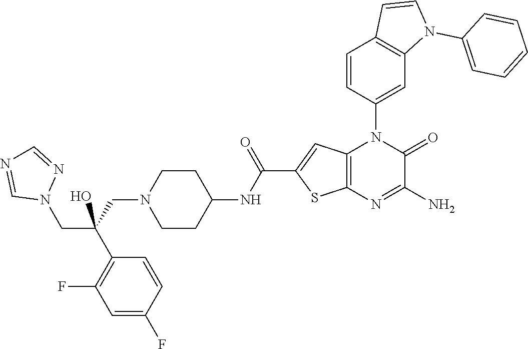 [SMILES: Nc1nc2sc(C(=O)NC3CCN(C[C@@](O)(Cn4cncn4)c4ccc(F)cc4F)CC3)cc2n(-c2ccc3ccn(-c4ccccc4)c3c2)c1=O] |
| I-23 | (S)-3-amino-N-(1-(2-(2,4- difluorophenyl)-2-hydroxy-3- (1H-1,2,4-triazol-1- yl)propyl)piperidin-4-yl)-2-oxo- 1-(1-phenyl-1H-indol-6-yl)-1,2- dihydrothieno[2,3-b]pyrazine- 6-carboxamide | 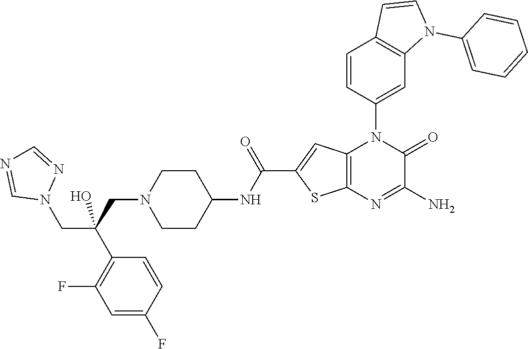 [SMILES: Nc1nc2sc(C(=O)NC3CCN(C[C@](O)(Cn4cncn4)c4ccc(F)cc4F)CC3)cc2n(-c2ccc3ccn(-c4ccccc4)c3c2)c1=O] |
| I-24 | (R)-3-amino-N-(1-(2-(2,4- difluorophenyl)-2-hydroxy-3- (1H-1,2,4-triazol-1- yl)propyl)piperidin-4-yl)-1-(4- methyl-3-phenoxyphenyl)-2- oxo-1,2-dihydrothieno[2,3- b]pyrazine-6-carboxamide | 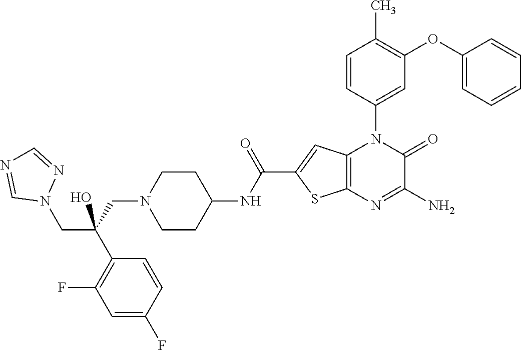 [SMILES: Cc1ccc(-n2c(=O)c(N)nc3sc(C(=O)NC4CCN(C[C@@](O)(Cn5cncn5)c5ccc(F)cc5F)CC4)cc32)cc1Oc1ccccc1] |
| I-25 | (S)-3-amino-N-(1-(2-(2,4- difluorophenyl)-2-hydroxy-3- (1H-1,2,4-triazol-1- yl)propyl)piperidin-4-yl)-1-(4- methyl-3-phenoxyphenyl)-2- oxo-1,2-dihydrothieno[2,3- b]pyrazine-6-carboxamide | 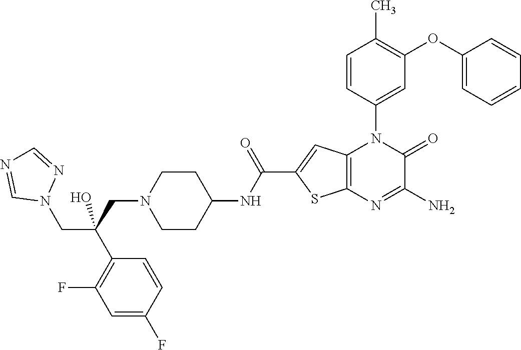 [SMILES: Cc1ccc(-n2c(=O)c(N)nc3sc(C(=O)NC4CCN(C[C@](O)(Cn5cncn5)c5ccc(F)cc5F)CC4)cc32)cc1Oc1ccccc1] |
| I-26 | N-(((3R,5R)-5-((1H-1,2,4- triazol-1-yl)methyl)-5-(2,4- difluorophenyl)tetrahydrofuran- 3-yl)methyl)-3-amino-2-oxo-1- (4-phenyl-3,4-dihydro-2H- benzo[b][1,4]oxazin-6-yl)-1,2- dihydrothieno[2,3-b]pyrazine- 6-carboxamide | 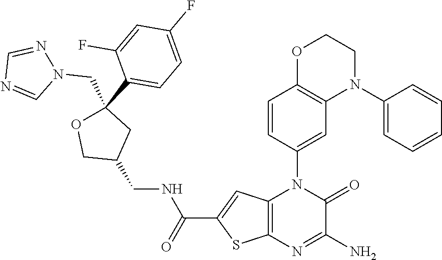 |
| I-27 | N-(((3S,5S)-5-((1H-1,2,4- triazol-1-yl)methyl)-5-(2,4- difluorophenyl)tetrahydrofuran- 3-yl)methyl)-3-amino-2-oxo-1- (4-phenyl-3,4-dihydro-2H- benzo[b][1,4]oxazin-6-yl)-1,2- dihydrothieno[2,3-b]pyrazine- 6-carboxamide | 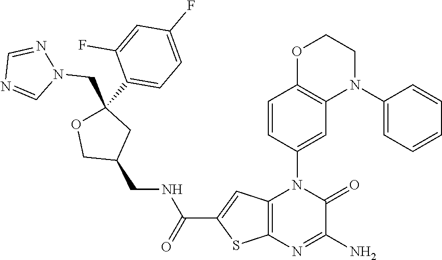 [SMILES: Nc1nc2sc(C(=O)NC[C@H]3CO[C@](Cn4cncn4)(c4ccc(F)cc4F)C3)cc2n(-c2ccc3c(c2)N(c2ccccc2)CCO3)c1=O] |
| I-28 | 3-amino-N-(1-(3-(2,4- difluorophenyl)-3-hydroxy-4- (1H-1,2,4-triazol-1- yl)butyl)piperidin-4-yl)-2-oxo-1- (4-phenyl-3,4-dihydro-2H- benzo[b][1,4]oxazin-6-yl)-1,2- dihydrothieno[2,3-b]pyrazine- 6-carboxamide | 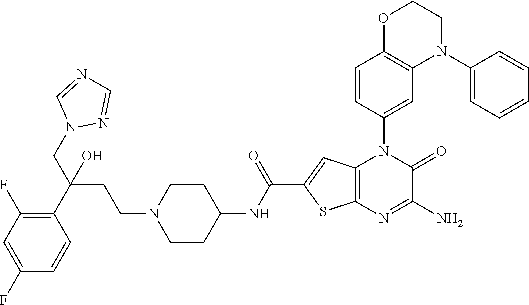 [SMILES: Nc1nc2sc(C(=O)NC3CCN(CCC(O)(Cn4cncn4)c4ccc(F)cc4F)CC3)cc2n(-c2ccc3c(c2)N(c2ccccc2)CCO3)c1=O] |
| I-29 | 3-amino-N-(1-(3-(2,4- difluorophenyl)-3-hydroxy-4- (1H-1,2,4-triazol-1- yl)butanoyl)piperidin-4-yl)-2- oxo-1-(4-phenyl-3,4-dihydro- 2H-benzo[b][1,4]oxazin-6-yl)- 1,2-dihydrothieno[2,3- b]pyrazine-6-carboxamide | 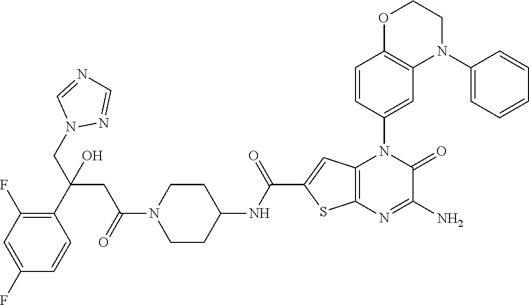 [SMILES: Nc1nc2sc(C(=O)NC3CCN(C(=O)CC(O)(Cn4cncn4)c4ccc(F)cc4F)CC3)cc2n(-c2ccc3c(c2)N(c2ccccc2)CCO3)c1=O] |
| I-30 | 3-amino-N-(6-(2-(2,4- difluorophenyl)-1,1-difluoro-2- hydroxy-3-(1H-1,2,4-triazol-1- yl)propyl)pyridin-3-yl)-2-oxo-1- (4-phenyl-3,4-dihydro-2H- benzo[b][1,4]oxazin-6-yl)-1,2- dihydrothieno[2,3-b]pyrazine- 6-carboxamide | 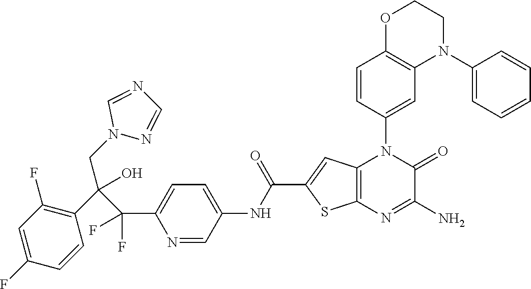 [SMILES: Nc1nc2sc(C(=O)Nc3ccc(C(F)(F)C(O)(Cn4cncn4)c4ccc(F)cc4F)nc3)cc2n(-c2ccc3c(c2)N(c2ccccc2)CCO3)c1=O] |
| I-31 | 3-amino-N-(1-(2-(2,4- difluorophenyl)-2-hydroxy-3- (1H-1,2,4-triazol-1-yl)propyl)-4- methylpiperidin-4-yl)-2-oxo-1- (4-phenyl-3,4-dihydro-2H- benzo[b][1,4]oxazin-6-yl)-1,2- dihydrothieno[2,3-b]pyrazine- 6-carboxamide | 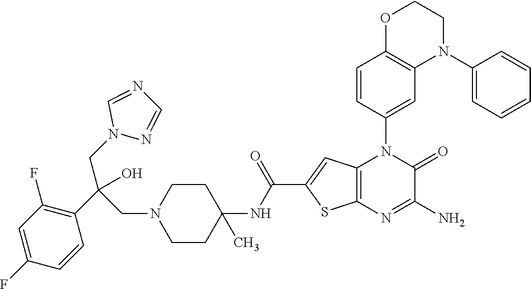 [SMILES: CC1(NC(=O)c2cc3c(nc(N)c(=O)n3-c3ccc4c(c3)N(c3ccccc3)CCO4)s2)CCN(CC(O)(Cn2cncn2)c2ccc(F)cc2F)CC1] |
| I-32 | N-(1-((5-((1H-1,2,4-triazol-1- yl)methyl)-5-(2,4- difluorophenyl)tetrahydrofuran- 3-yl)methyl)piperidin-4-yl)-3- amino-2-oxo-1-(4-phenyl-3,4- dihydro-2H- benzo[b][1,4]oxazin-6-yl)-1,2- dihydrothieno[2,3-b]pyrazine- 6-carboxamide | 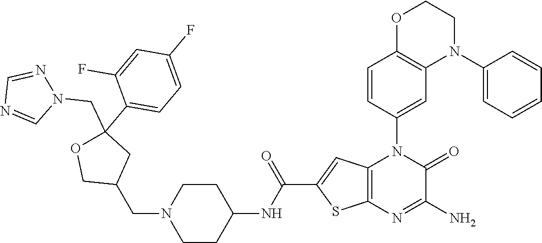 [SMILES: Nc1nc2sc(C(=O)NC3CCN(CC4COC(Cn5cncn5)(c5ccc(F)cc5F)C4)CC3)cc2n(-c2ccc3c(c2)N(c2ccccc2)CCO3)c1=O] |
| I-33 | 3-amino-N-(3-(3-((1-(2-(2,4- difluorophenyl)-2-hydroxy-3- (1H-1,2,4-triazol-1- yl)propyl)piperidin-4- yl)oxy)propoxy)phenyl)-2-oxo- 1-(4-phenyl-3,4-dihydro-2H- benzo[b][1,4]oxazin-6-yl)-1,2- dihydrothieno[2,3-b]pyrazine- 6-carboxamide | 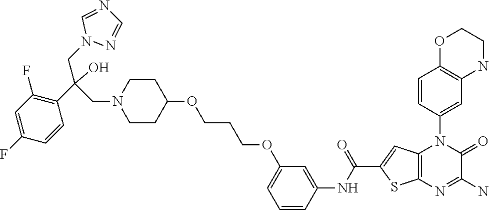 [SMILES: Nc1nc2sc(C(=O)Nc3cccc(OCCCOC4CCN(CC(O)(Cn5cncn5)c5ccc(F)cc5F)CC4)c3)cc2n(-c2ccc3c(c2)NCCO3)c1=O] |
| I-34 | (R)-3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 7-carboxamide | 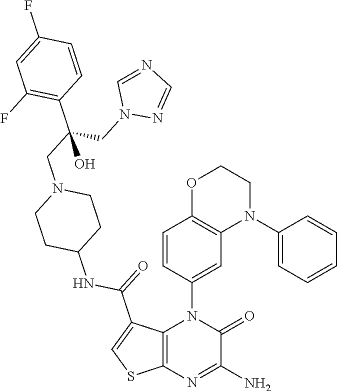 [SMILES: Nc1nc2scc(C(=O)NC3CCN(C[C@@](O)(Cn4cncn4)c4ccc(F)cc4F)CC3)c2n(-c2ccc3c(c2)N(c2ccccc2)CCO3)c1=O] |
| I-35 | (S)-3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 7-carboxamide | 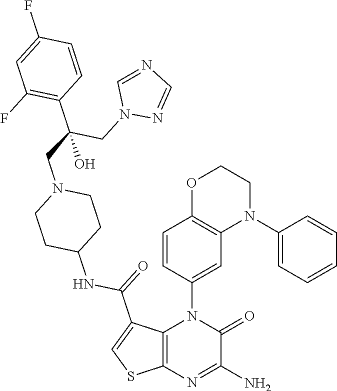 [SMILES: Nc1nc2scc(C(=O)NC3CCN(C[C@](O)(Cn4cncn4)c4ccc(F)cc4F)CC3)c2n(-c2ccc3c(c2)N(c2ccccc2)CCO3)c1=O] |
| I-36 | 3-amino-N-((3R)-1-(3-(2,4- difluorophenyl)-3-hydroxy-4- (1H-1,2,4-triazol-1- yl)butyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 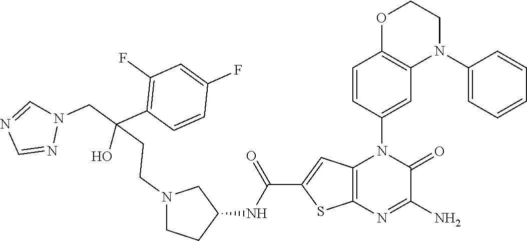 [SMILES: Nc1nc2sc(C(=O)N[C@@H]3CCN(CCC(O)(Cn4cncn4)c4ccc(F)cc4F)C3)cc2n(-c2ccc3c(c2)N(c2ccccc2)CCO3)c1=O] |
| I-37 | 3-amino-N-((3R)-1-(3-(2,4- difluorophenyl)-3-hydroxy-4- (1H-1,2,4-triazol-1- yl)butanoyl)pyrrolidin-3-yl)-2- oxo-1-(4-phenyl-3,4-dihydro- 2H-benzo[b][1,4]oxazin-6-yl)- 1,2-dihydrothieno[2,3- b]pyrazine-6-carboxamide | 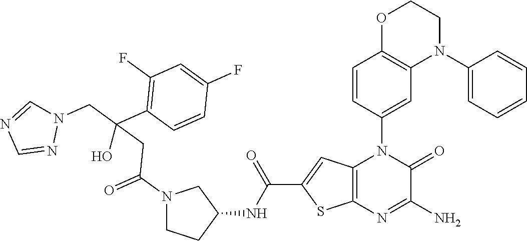 [SMILES: Nc1nc2sc(C(=O)N[C@@H]3CCN(C(=O)CC(O)(Cn4cncn4)c4ccc(F)cc4F)C3)cc2n(-c2ccc3c(c2)N(c2ccccc2)CCO3)c1=O] |
| I-38 | 3-amino-N-((1R,5S,6s)-3-(2- (2,4-difluorophenyl)-2-hydroxy- 3-(1H-1,2,4-triazol-1-yl)propyl)- 3-azabicyclo[3.1.0]hexan-6-yl)- 2-oxo-1-(4-phenyl-3,4-dihydro- 2H-benzo[b][1,4]oxazin-6-yl)- 1,2-dihydrothieno[2,3- b]pyrazine-6-carboxamide | 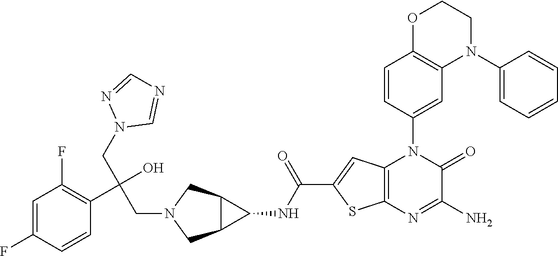 [SMILES: Nc1nc2sc(C(=O)N[C@H]3[C@@H]4CN(CC(O)(Cn5cncn5)c5ccc(F)cc5F)C[C@@H]43)cc2n(-c2ccc3c(c2)N(c2ccccc2)CCO3)c1=O] |
| I-39 | N-(1-((5-((1H-1,2,4-triazol-1- yl)methyl)-5-(2,4- difluorophenyl)tetrahydrofuran- 3-yl)methyl)azetidin-3-yl)-3- amino-2-oxo-1-(4-phenyl-3,4- dihydro-2H- benzo[b][1,4]oxazin-6-yl)-1,2- dihydrothieno[2,3-b]pyrazine- 6-carboxamide | 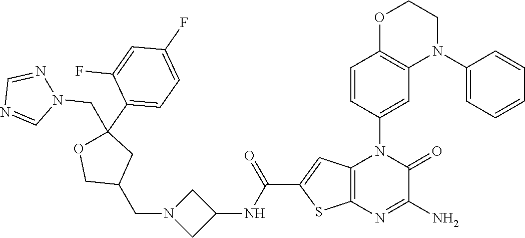 [SMILES: Nc1nc2sc(C(=O)NC3CN(CC4COC(Cn5cncn5)(c5ccc(F)cc5F)C4)C3)cc2n(-c2ccc3c(c2)N(c2ccccc2)CCO3)c1=O] |
| I-40 | N-((5-((1H-1,2,4-triazol-1- yl)methyl)-5-(2,4- difluorophenyl)tetrahydrofuran- 3-yl)methyl)-3-amino-1-(4- methyl-3-phenoxyphenyl)-2- oxo-1,2-dihydrothieno[2,3- b]pyrazine-6-carboxamide | 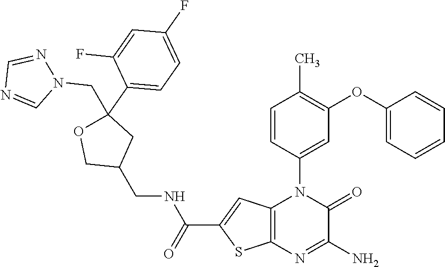 [SMILES: Cc1ccc(-n2c(=O)c(N)nc3sc(C(=O)NCC4COC(Cn5cncn5)(c5ccc(F)cc5F)C4)cc32)cc1Oc1ccccc1] |
| I-41 | N-((5-((1H-1,2,4-triazol-1- yl)methyl)-5-(2,4- difluorophenyl)tetrahydrofuran- 3-yl)methyl)-3-amino-2-oxo-1- (1-phenylindolin-6-yl)-1,2- dihydrothieno[2,3-b]pyrazine- 6-carboxamide | 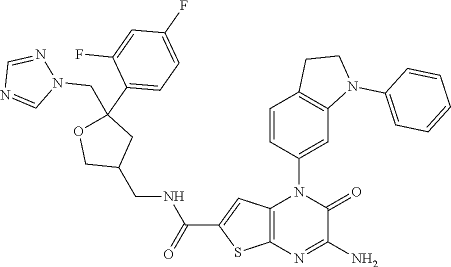 [SMILES: Nc1nc2sc(C(=O)NCC3COC(Cn4cncn4)(c4ccc(F)cc4F)C3)cc2n(-c2ccc3c(c2)N(c2ccccc2)CC3)c1=O] |
| I-42 | 2-(2,4-difluorophenyl)-2- hydroxy-3-(1H-1,2,4-triazol-1- yl)propyl 2-(4-(3-amino-2-oxo- 1-(4-phenyl-3,4-dihydro-2H- benzo[b][1,4]oxazin-6-yl)-1,2- dihydrothieno[2,3-b]pyrazine- 6-carboxamido)piperidin-1- yl)acetate | 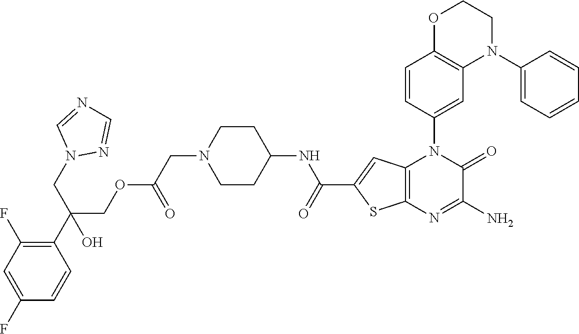 [SMILES: Nc1nc2sc(C(=O)NC3CCN(CC(=O)OCC(O)(Cn4cncn4)c4ccc(F)cc4F)CC3)cc2n(-c2ccc3c(c2)N(c2ccccc2)CCO3)c1=O] |
| I-43 | N-((5-((1H-1,2,4-triazol-1- yl)methyl)-5-(2,4- difluorophenyl)tetrahydrofuran- 3-yl)methyl)-3-amino-2-oxo-1- (1-phenyl-1,2,3,4- tetrahydroquinolin-7-yl)-1,2- dihydrothieno[2,3-b]pyrazine- 6-carboxamide | 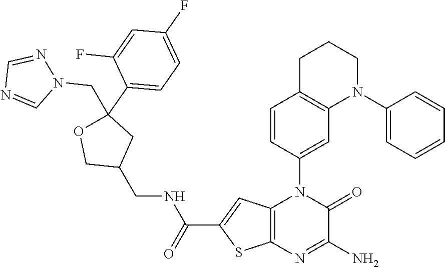 [SMILES: Nc1nc2sc(C(=O)NCC3COC(Cn4cncn4)(c4ccc(F)cc4F)C3)cc2n(-c2ccc3c(c2)N(c2ccccc2)CCC3)c1=O] |
| I-44 | N-((3R)-1-((5-((1H-1,2,4- triazol-1-yl)methyl)-5-(2,4- difluorophenyl)tetrahydrofuran- 3-yl)methyl)pyrrolidin-3-yl)-3- amino-2-oxo-1-(4-phenyl-3,4- dihydro-2H- benzo[b][1,4]oxazin-6-yl)-1,2- dihydrothieno[2,3-b]pyrazine- 6-carboxamide | 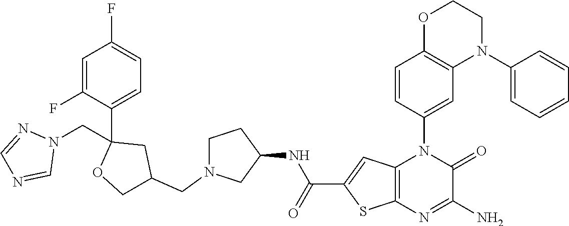 [SMILES: Nc1nc2sc(C(=O)N[C@@H]3CCN(CC4COC(Cn5cncn5)(c5ccc(F)cc5F)C4)C3)cc2n(-c2ccc3c(c2)N(c2ccccc2)CCO3)c1=O] |
| I-45 | N-((3S)-1-((5-((1H-1,2,4- triazol-1-yl)methyl)-5-(2,4- difluorophenyl)tetrahydrofuran- 3-yl)methyl)pyrrolidin-3-yl)-3- amino-2-oxo-1-(4-phenyl-3,4- dihydro-2H- benzo[b][1,4]oxazin-6-yl)-1,2- dihydrothieno[2,3-b]pyrazine- 6-carboxamide | 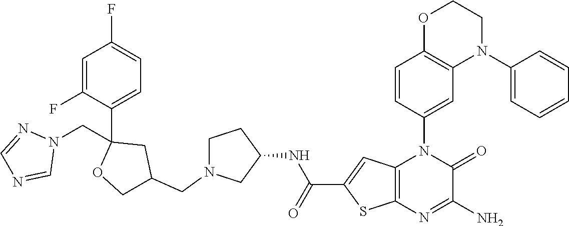 [SMILES: Nc1nc2sc(C(=O)N[C@H]3CCN(CC4COC(Cn5cncn5)(c5ccc(F)cc5F)C4)C3)cc2n(-c2ccc3c(c2)N(c2ccccc2)CCO3)c1=O] |
| I-46 | 3-amino-N-(4-(((1-(2-(2,4- difluorophenyl)-2-hydroxy-3- (1H-1,2,4-triazol-1- yl)propyl)piperidin-4- yl)oxy)methyl)phenyl)-2-oxo-1- (4-phenyl-3,4-dihydro-2H- benzo[b][1,4]oxazin-6-yl)-1,2- dihydrothieno[2,3-b]pyrazine- 6-carboxamide | 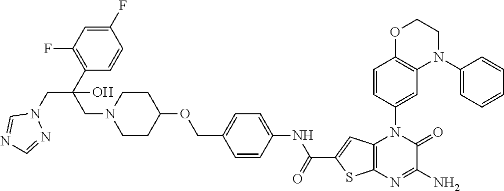 [SMILES: Nc1nc2sc(C(=O)Nc3ccc(COC4CCN(CC(O)(Cn5cncn5)c5ccc(F)cc5F)CC4)cc3)cc2n(-c2ccc3c(c2)N(c2ccccc2)CCO3)c1=O] |
| I-47 | 3-amino-6-(((1-(2-(2,4- difluorophenyl)-2-hydroxy-3- (1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 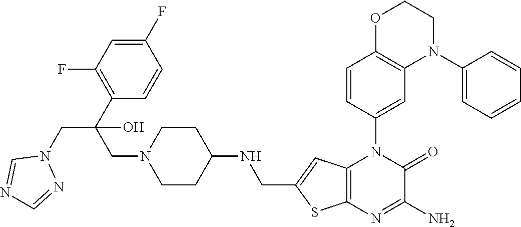 [SMILES: Nc1nc2sc(CNC3CCN(CC(O)(Cn4cncn4)c4ccc(F)cc4F)CC3)cc2n(-c2ccc3c(c2)N(c2ccccc2)CCO3)c1=O] |
| I-48 | 3-(2,4-difluorophenyl)-3- hydroxy-4-(1H-1,2,4-triazol-1- yl)butyl 2-(4-(3-amino-2-oxo-1- (4-phenyl-3,4-dihydro-2H- benzo[b][1,4]oxazin-6-yl)-1,2- dihydrothieno[2,3-b]pyrazine- 6-carboxamido)piperidin-1- yl)acetate--methane | 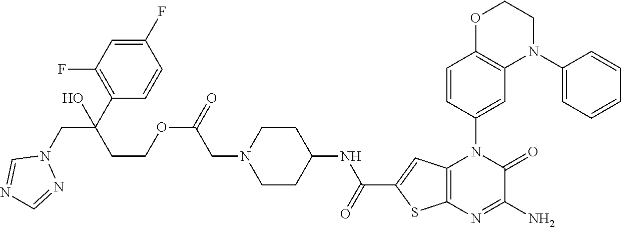 [SMILES: Nc1nc2sc(C(=O)NC3CCN(CC(=O)OCCC(O)(Cn4cncn4)c4ccc(F)cc4F)CC3)cc2n(-c2ccc3c(c2)N(c2ccccc2)CCO3)c1=O] |
| I-49 | 3-amino-N-(2-(2,4- difluorophenyl)-2-hydroxy-3- (1H-1,2,4-triazol-1-yl)propyl)-2- oxo-1-(4-phenyl-3,4-dihydro- 2H-benzo[b][1,4]oxazin-6-yl)- 1,2-dihydrothieno[2,3- b]pyrazine-7-carboxamide | 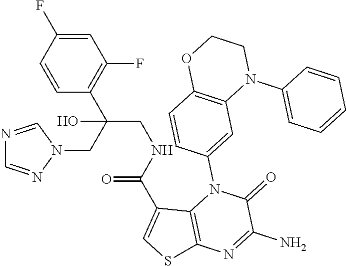 [SMILES: Nc1nc2scc(C(=O)NCC(O)(Cn3cncn3)c3ccc(F)cc3F)c2n(-c2ccc3c(c2)N(c2ccccc2)CCO3)c1=O] |
| I-50 | 3-amino-N-(1-(2-(2,4- difluorophenyl)-2-hydroxy-3- (1H-1,2,4-triazol-1- yl)propyl)azepan-4-yl)-2-oxo-1- (4-phenyl-3,4-dihydro-2H- benzo[b][1,4]oxazin-6-yl)-1,2- dihydrothieno[2,3-b]pyrazine- 6-carboxamide | 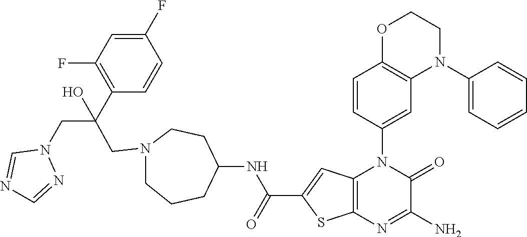 [SMILES: Nc1nc2sc(C(=O)NC3CCCN(CC(O)(Cn4cncn4)c4ccc(F)cc4F)CC3)cc2n(-c2ccc3c(c2)N(c2ccccc2)CCO3)c1=O] |
| I-51 | 3-amino-6-(1-(1-(2-(2,4- difluorophenyl)-2-hydroxy-3- (1H-1,2,4-triazol-1- yl)propyl)piperidin-4-yl)-1H- 1,2,3-triazol-4-yl)-1-(4-phenyl- 3,4-dihydro-2H- benzo[b][1,4]oxazin-6- yl)thieno[2,3-b]pyrazin-2(1H)- one | 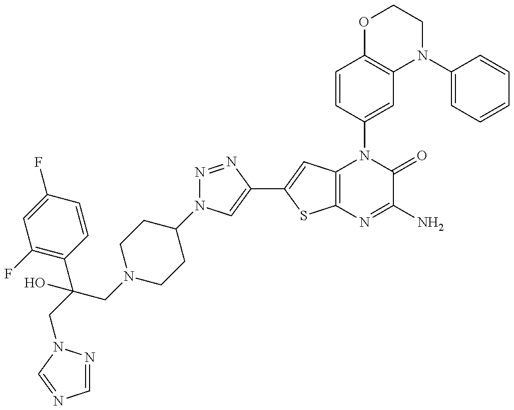 [SMILES: Nc1nc2sc(-c3cn(C4CCN(CC(O)(Cn5cncn5)c5ccc(F)cc5F)CC4)nn3)cc2n(-c2ccc3c(c2)N(c2ccccc2)CCO3)c1=O] |
| I-52 | (R)-3-amino-N-(1-(2-(2,4- difluorophenyl)-2-hydroxy-3- (1H-1,2,4-triazol-1-yl)propyl)-4- methylpiperidin-4-yl)-2-oxo-1- (4-phenyl-3,4-dihydro-2H- benzo[b][1,4]oxazin-6-yl)-1,2- dihydrothieno[2,3-b]pyrazine- 6-carboxamide | 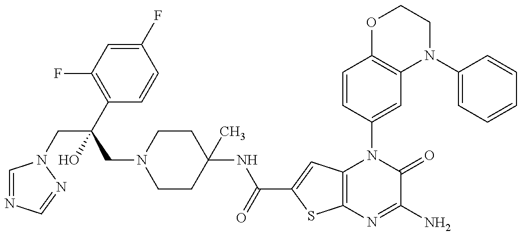 [SMILES: CC1(NC(=O)c2cc3c(nc(N)c(=O)n3-c3ccc4c(c3)N(c3ccccc3)CCO4)s2)CCN(C[C@@](O)(Cn2cncn2)c2ccc(F)cc2F)CC1] |
| I-53 | (S)-3-amino-N-(1-(2-(2,4- difluorophenyl)-2-hydroxy-3- (1H-1,2,4-triazol-1-yl)propyl)-4- methylpiperidin-4-yl)-2-oxo-1- (4-phenyl-3,4-dihydro-2H- benzo[b][1,4]oxazin-6-yl)-1,2- dihydrothieno[2,3-b]pyrazine- 6-carboxamide | 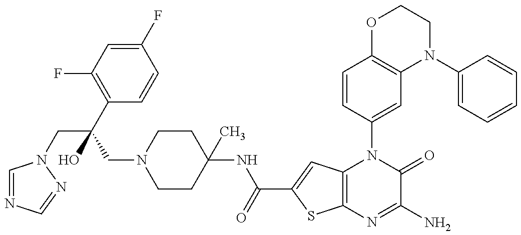 [SMILES: CC1(NC(=O)c2cc3c(nc(N)c(=O)n3-c3ccc4c(c3)N(c3ccccc3)CCO4)s2)CCN(C[C@](O)(Cn2cncn2)c2ccc(F)cc2F)CC1] |
| I-54 | 3-(3-amino-2-oxo-1-(4-phenyl- 3,4-dihydro-2H- benzo[b][1,4]oxazin-6-yl)-1,2- dihydrothieno[2,3-b]pyrazin-6- yl)-N-(4-(2-(2,4- difluorophenyl)-2-hydroxy-3- (1H-1,2,4-triazol-1- yl)propyl)piperazin-1-yl)-1,2,4- oxadiazole-5-carboxamide | 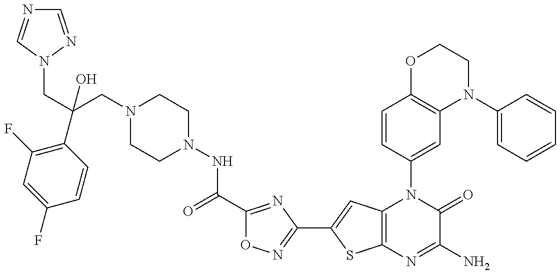 [SMILES: Nc1nc2sc(-c3noc(C(=O)NN4CCN(CC(O)(Cn5cncn5)c5ccc(F)cc5F)CC4)n3)cc2n(-c2ccc3c(c2)N(c2ccccc2)CCO3)c1=O] |
| I-55 | methyl 3-amino-6-((1-(2-(2,4- difluorophenyl)-2-hydroxy-3- (1H-1,2,4-triazol-1- yl)propyl)piperidin-4- yl)carbamoyl)-2-oxo-1-(4- phenyl-3,4-dihydro-2H- benzo[b][1,4]oxazin-6-yl)-1,2- dihydrothieno[2,3-b]pyrazine- 7-carboxylate | 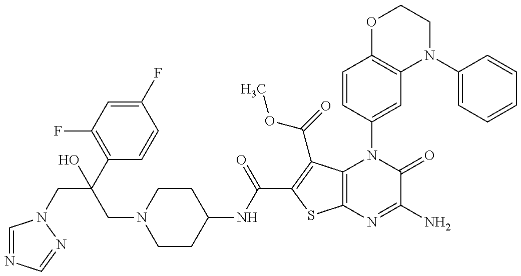 [SMILES: COC(=O)c1c(C(=O)NC2CCN(CC(O)(Cn3cncn3)c3ccc(F)cc3F)CC2)sc2nc(N)c(=O)n(-c3ccc4c(c3)N(c3ccccc3)CCO4)c12] |
| I-56 | 3-amino-N-(1-(2-(2,4- difluorophenyl)-2-hydroxy-3- (1H-1,2,4-triazol-1- yl)propyl)piperidin-4-yl)-7-(1- hydroxyethyl)-2-oxo-1-(1- phenyl-1H-indol-6-yl)-1,2- dihydrothieno[2,3-b]pyrazine- 6-carboxamide | 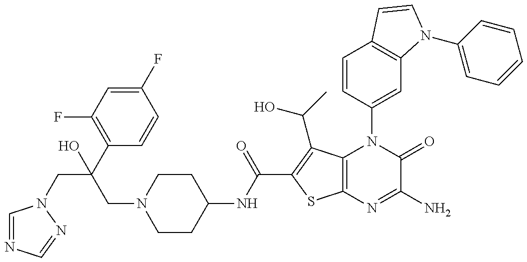 [SMILES: CC(O)c1c(C(=O)NC2CCN(CC(O)(Cn3cncn3)c3ccc(F)cc3F)CC2)sc2nc(N)c(=O)n(-c3ccc4ccn(-c5ccccc5)c4c3)c12] |
| I-57 | 5-(3-amino-2-oxo-1-(4-phenyl- 3,4-dihydro-2H- benzo[b][1,4]oxazin-6-yl)-1,2- dihydrothieno[2,3-b]pyrazin-6- yl)-N-(2-(2,4-difluorophenyl)-2- hydroxy-3-(1H-1,2,4-triazol-1- yl)propyl)-1,2,4-oxadiazole-3- carboxamide | 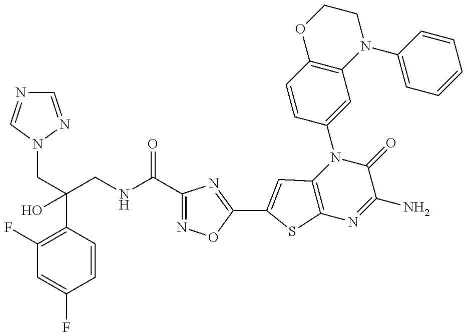 [SMILES: Nc1nc2sc(-c3nc(C(=O)NCC(O)(Cn4cncn4)c4ccc(F)cc4F)no3)cc2n(-c2ccc3c(c2)N(c2ccccc2)CCO3)c1=O] |
| I-58 | ethyl 3-amino-7-((1-(2-(2,4- difluorophenyl)-2-hydroxy-3- (1H-1,2,4-triazol-1- yl)propyl)piperidin-4- yl)carbamoyl)-2-oxo-1-(4- phenyl-3,4-dihydro-2H- benzo[b][1,4]oxazin-6-yl)-1,2- dihydrothieno[2,3-b]pyrazine- 6-carboxylate | 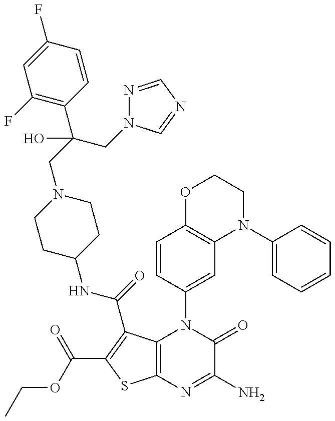 [SMILES: CCOC(=O)c1sc2nc(N)c(=O)n(-c3ccc4c(c3)N(c3ccccc3)CCO4)c2c1C(=O)NC1CCN(CC(O)(Cn2cncn2)c2ccc(F)cc2F)CC1] |
| I-59 | 3-amino-N7-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6,7-dicarboxamide | 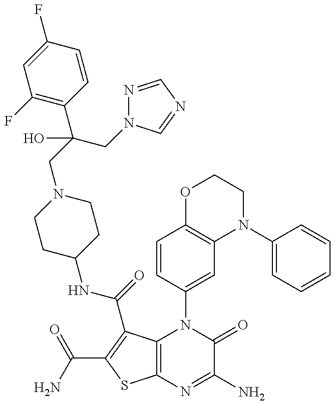 [SMILES: NC(=O)c1sc2nc(N)c(=O)n(-c3ccc4c(c3)N(c3ccccc3)CCO4)c2c1C(=O)NC1CCN(CC(O)(Cn2cncn2)c2ccc(F)cc2F)CC1] |
| I-60 | 3-amino-N7-(1-(2-(2,4- difluorophenyl)-2-hydroxy-3- (1H-1,2,4-triazol-1- yl)propyl)piperidin-4-yl)-N6- ethyl-2-oxo-1-(4-phenyl-3,4- dihydro-2H- benzo[b][1,4]oxazin-6-yl)-1,2- dihydrothieno[2,3-b]pyrazine- 6,7-dicarboxamide | 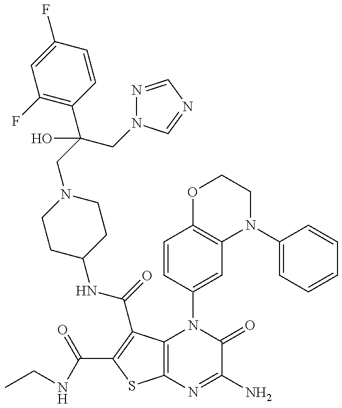 [SMILES: CCNC(=O)c1sc2nc(N)c(=O)n(-c3ccc4c(c3)N(c3ccccc3)CCO4)c2c1C(=O)NC1CCN(CC(O)(Cn2cncn2)c2ccc(F)cc2F)CC1] |
| I-61 | 3-amino-7-((1-(2-(2,4- difluorophenyl)-2-hydroxy-3- (1H-1,2,4-triazol-1- yl)propyl)piperidin-4- yl)carbamoyl)-2-oxo-1-(4- phenyl-3,4-dihydro-2H- benzo[b][1,4]oxazin-6-yl)-1,2- dihydrothieno[2,3-b]pyrazine- 6-carboxylic acid | 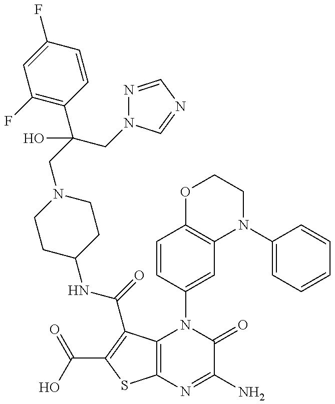 [SMILES: Nc1nc2sc(C(=O)O)c(C(=O)NC3CCN(CC(O)(Cn4cncn4)c4ccc(F)cc4F)CC3)c2n(-c2ccc3c(c2)N(c2ccccc2)CCO3)c1=O] |
| I-62 | (R)-3-amino-6-(((1-(2-(2,4- difluorophenyl)-2-hydroxy-3- (1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 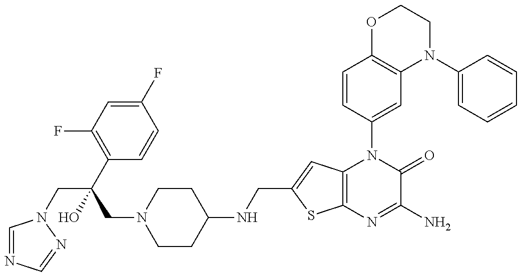 [SMILES: Nc1nc2sc(CNC3CCN(C[C@@](O)(Cn4cncn4)c4ccc(F)cc4F)CC3)cc2n(-c2ccc3c(c2)N(c2ccccc2)CCO3)c1=O] |
| I-63 | (S)-3-amino-6-(((1-(2-(2,4- difluorophenyl)-2-hydroxy-3- (1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 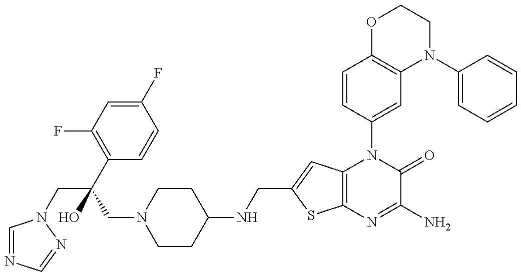 [SMILES: Nc1nc2sc(CNC3CCN(C[C@](O)(Cn4cncn4)c4ccc(F)cc4F)CC3)cc2n(-c2ccc3c(c2)N(c2ccccc2)CCO3)c1=O] |
| I-64 | 3-amino-N-(1-(4-(2,4- difluorophenyl)-4-hydroxy-5- (1H-1,2,4-triazol-1- yl)pentyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 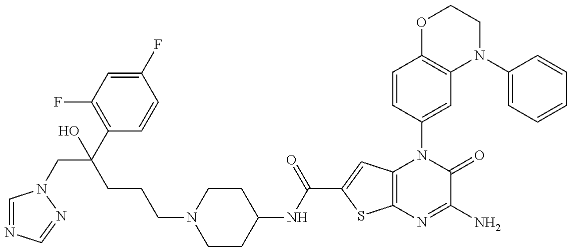 [SMILES: Nc1nc2sc(C(=O)NC3CCN(CCCC(O)(Cn4cncn4)c4ccc(F)cc4F)CC3)cc2n(-c2ccc3c(c2)N(c2ccccc2)CCO3)c1=O] |
| I-65 | 5-(3-amino-2-oxo-1-(4-phenyl- 3,4-dihydro-2H- benzo[b][1,4]oxazin-6-yl)-1,2- dihydrothieno[2,3-b]pyrazin-6- yl)-N-(1-(2-(2,4- difluorophenyl)-2-hydroxy-3- (1H-1,2,4-triazol-1- yl)propyl)piperidin-4-yl)-1H- 1,2,4-triazole-3-carboxamide | 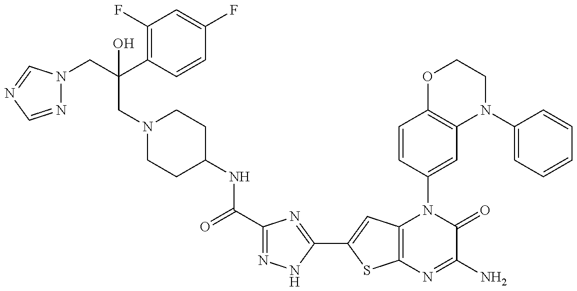 [SMILES: Nc1nc2sc(-c3nc(C(=O)NC4CCN(CC(O)(Cn5cncn5)c5ccc(F)cc5F)CC4)n[nH]3)cc2n(-c2ccc3c(c2)N(c2ccccc2)CCO3)c1=O] |
| I-66 | 3-amino-6-(((1-(2-(2,4- difluorophenyl)-2-hydroxy-3- (1H-1,2,4-triazol-1- yl)propyl)azetidin-3- yl)amino)methyl)-1-(4-phenyl- 3,4-dihydro-2H- benzo[b][1,4]oxazin-6- yl)thieno[2,3-b]pyrazin-2(1H)- one | 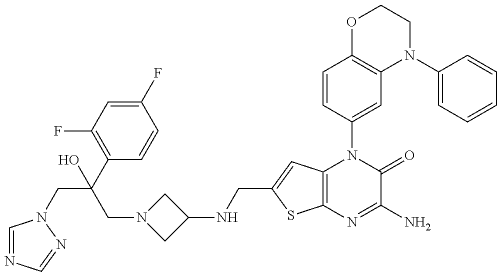 [SMILES: Nc1nc2sc(CNC3CN(CC(O)(Cn4cncn4)c4ccc(F)cc4F)C3)cc2n(-c2ccc3c(c2)N(c2ccccc2)CCO3)c1=O] |
| I-67 | 3-amino-6-((((1-(2-(2,4- difluorophenyl)-2-hydroxy-3- (1H-1,2,4-triazol-1- yl)propyl)piperidin-4- yl)methyl)amino)methyl)-1-(4- phenyl-3,4-dihydro-2H- benzo[b][1,4]oxazin-6- yl)thieno[2,3-b]pyrazin-2(1H)- one | 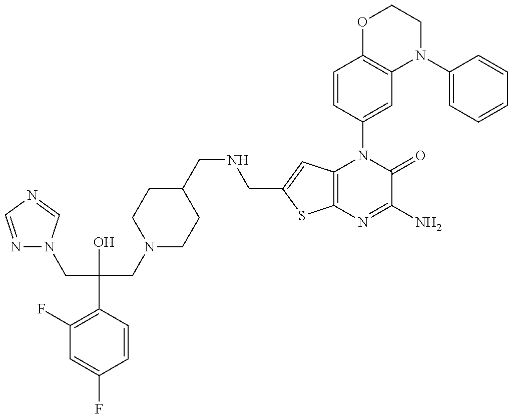 [SMILES: Nc1nc2sc(CNCC3CCN(CC(O)(Cn4cncn4)c4ccc(F)cc4F)CC3)cc2n(-c2ccc3c(c2)N(c2ccccc2)CCO3)c1=O] |
| I-68 | 3-amino-6-((4-(2-(2,4- difluorophenyl)-2-hydroxy-3- (1H-1,2,4-triazol-1- yl)propyl)piperazin-1- yl)methyl)-1-(4-phenyl-3,4- dihydro-2H- benzo[b][1,4]oxazin-6- yl)thieno[2,3-b]pyrazin-2(1H)- one | 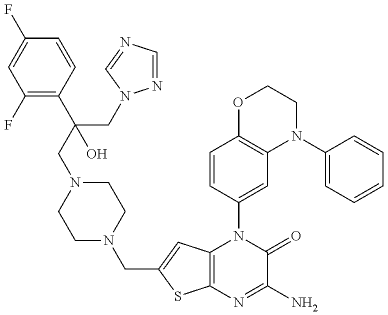 [SMILES: Nc1nc2sc(CN3CCN(CC(O)(Cn4cncn4)c4ccc(F)cc4F)CC3)cc2n(-c2ccc3c(c2)N(c2ccccc2)CCO3)c1=O] |
| I-69 | 3-amino-N-((R)-1-((R)-3-(2,4- difluorophenyl)-3-hydroxy-4- (1H-1,2,4-triazol-1- yl)butyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 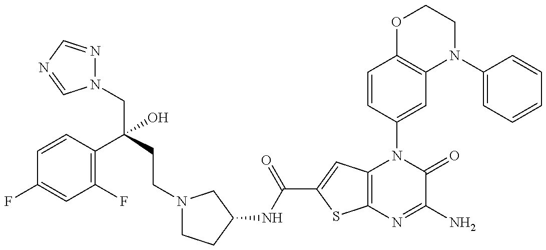 [SMILES: Nc1nc2sc(C(=O)N[C@@H]3CCN(CC[C@](O)(Cn4cncn4)c4ccc(F)cc4F)C3)cc2n(-c2ccc3c(c2)N(c2ccccc2)CCO3)c1=O] |
| I-70 | 3-amino-N-((R)-1-((S)-3-(2,4- difluorophenyl)-3-hydroxy-4- (1H-1,2,4-triazol-1- yl)butyl)pyrrolidin-3-yl)-2-oxo- 1-(4-phenyl-3,4-dihydro-2H- benzo[b][1,4]oxazin-6-yl)-1,2- dihydrothieno[2,3-b]pyrazine- 6-carboxamide | 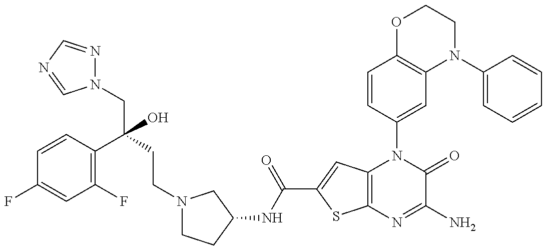 [SMILES: Nc1nc2sc(C(=O)N[C@@H]3CCN(CC[C@@](O)(Cn4cncn4)c4ccc(F)cc4F)C3)cc2n(-c2ccc3c(c2)N(c2ccccc2)CCO3)c1=O] |
| I-71 | 3-amino-6-((((3S)-1-(2-(2,4- difluorophenyl)-2-hydroxy-3- (1H-1,2,4-triazol-1- yl)propyl)pyrrolidin-3- yl)amino)methyl)-1-(4-phenyl- 3,4-dihydro-2H- benzo[b][1,4]oxazin-6- yl)thieno[2,3-b]pyrazin-2(1H)- one | 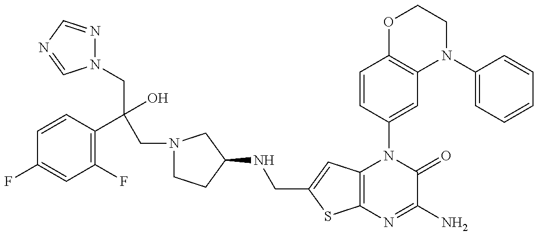 [SMILES: Nc1nc2sc(CN[C@H]3CCN(CC(O)(Cn4cncn4)c4ccc(F)cc4F)C3)cc2n(-c2ccc3c(c2)N(c2ccccc2)CCO3)c1=O] |
| I-72 | 3-amino-6-(((1-(3-(2,4- difluorophenyl)-3-hydroxy-4- (1H-1,2,4-triazol-1- yl)butyl)piperidin-4- yl)amino)methyl)-1-(4-phenyl- 3,4-dihydro-2H- benzo[b][1,4]oxazin-6- yl)thieno[2,3-b]pyrazin-2(1H)- one | 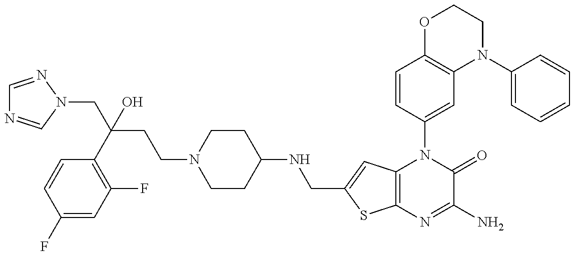 [SMILES: Nc1nc2sc(CNC3CCN(CCC(O)(Cn4cncn4)c4ccc(F)cc4F)CC3)cc2n(-c2ccc3c(c2)N(c2ccccc2)CCO3)c1=O] |
| I-73 | 3-amino-6-(((1-(4-(2,4- difluorophenyl)-4-hydroxy-5- (1H-1,2,4-triazol-1- yl)pentyl)piperidin-4- yl)amino)methyl)-1-(4-phenyl- 3,4-dihydro-2H- benzo[b][1,4]oxazin-6- yl)thieno[2,3-b]pyrazin-2(1H)- one | 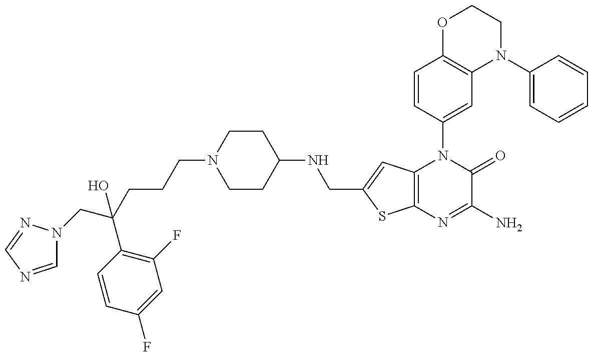 [SMILES: Nc1nc2sc(CNC3CCN(CCCC(O)(Cn4cncn4)c4ccc(F)cc4F)CC3)cc2n(-c2ccc3c(c2)N(c2ccccc2)CCO3)c1=O] |
| I-74 | 3-amino-6-(((3-(2,4- difluorophenyl)-3-hydroxy-4- (1H-1,2,4-triazol-1- yl)butyl)amino)methyl)-1-(4- phenyl-3,4-dihydro-2H- benzo[b][1,4]oxazin-6- yl)thieno[2,3-b]pyrazin-2(1H)- one | 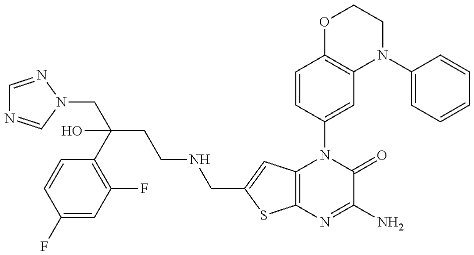 [SMILES: Nc1nc2sc(CNCCC(O)(Cn3cncn3)c3ccc(F)cc3F)cc2n(-c2ccc3c(c2)N(c2ccccc2)CCO3)c1=O] |
| I-75 | 3-amino-6-((((3R)-1-(2-(2,4- difluorophenyl)-2-hydroxy-3- (1H-1,2,4-triazol-1- yl)propyl)piperidin-3- yl)amino)methyl)-1-(4-phenyl- 3,4-dihydro-2H- benzo[b][1,4]oxazin-6- yl)thieno[2,3-b]pyrazin-2(1H)- one | 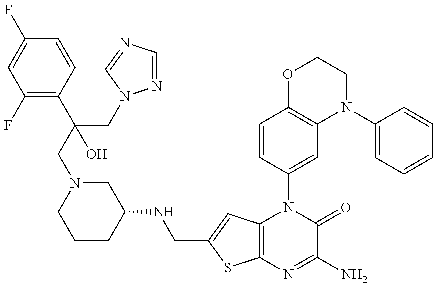 [SMILES: Nc1nc2sc(CN[C@@H]3CCCN(CC(O)(Cn4cncn4)c4ccc(F)cc4F)C3)cc2n(-c2ccc3c(c2)N(c2ccccc2)CCO3)c1=O] |
| I-76 | 3-amino-N-(1-(2-(2,4- difluorophenyl)-2-hydroxy-3- (1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 7-carboxamide | 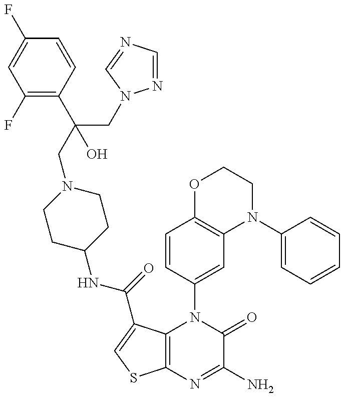 [SMILES: Nc1nc2scc(C(=O)NC3CCN(CC(O)(Cn4cncn4)c4ccc(F)cc4F)CC3)c2n(-c2ccc3c(c2)N(c2ccccc2)CCO3)c1=O] |
| I-77 | 3-amino-6-((((3R)-1-(2-(2,4- difluorophenyl)-2-hydroxy-3- (1H-1,2,4-triazol-1- yl)propyl)pyrrolidin-3- yl)amino)methyl)-1-(4-phenyl- 3,4-dihydro-2H- benzo[b][1,4]oxazin-6- yl)thieno[2,3-b]pyrazin-2(1H)- one | 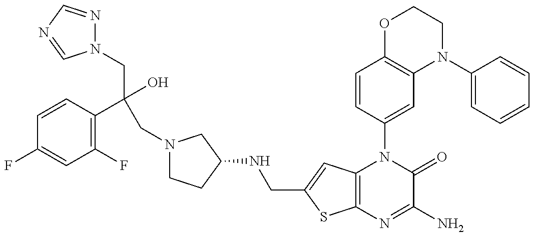 [SMILES: Nc1nc2sc(CN[C@@H]3CCN(CC(O)(Cn4cncn4)c4ccc(F)cc4F)C3)cc2n(-c2ccc3c(c2)N(c2ccccc2)CCO3)c1=O] |
| I-78 | 3-amino-6-(((1-(3-(2,4- difluorophenyl)-3-hydroxy-4- (1H-1,2,4-triazol-1- yl)butanoyl)piperidin-4- yl)amino)methyl)-1-(4-phenyl- 3,4-dihydro-2H- benzo[b][1,4]oxazin-6- yl)thieno[2,3-b]pyrazin-2(1H)- one | 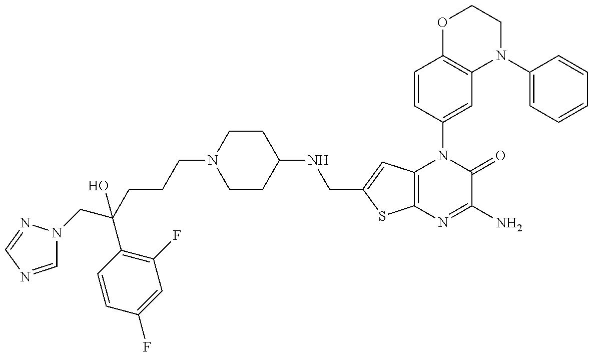 [SMILES: Nc1nc2sc(CNC3CCN(CCCC(O)(Cn4cncn4)c4ccc(F)cc4F)CC3)cc2n(-c2ccc3c(c2)N(c2ccccc2)CCO3)c1=O] |
| I-79 | 3-amino-6-((((3S)-1-(2-(2,4- difluorophenyl)-2-hydroxy-3- (1H-1,2,4-triazol-1- yl)propyl)piperidin-3- yl)amino)methyl)-1-(4-phenyl- 3,4-dihydro-2H- benzo[b][1,4]oxazin-6- yl)thieno[2,3-b]pyrazin-2(1H)- one | 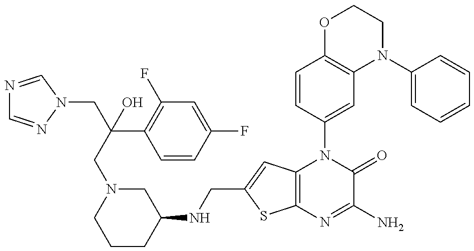 [SMILES: Nc1nc2sc(CN[C@H]3CCCN(CC(O)(Cn4cncn4)c4ccc(F)cc4F)C3)cc2n(-c2ccc3c(c2)N(c2ccccc2)CCO3)c1=O] |
| I-80 | 3-amino-6-(((1-(2-(2,4- difluorophenyl)-2-hydroxy-3- (1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 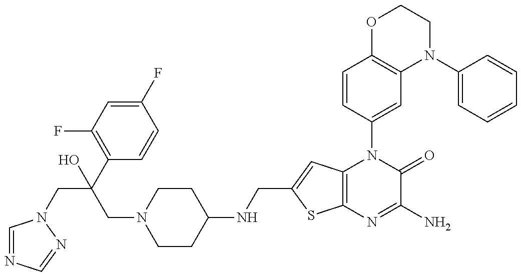 [SMILES: Nc1nc2sc(CNC3CCN(CC(O)(Cn4cncn4)c4ccc(F)cc4F)CC3)cc2n(-c2ccc3c(c2)N(c2ccccc2)CCO3)c1=O] |
| I-81 | 3-amino-6-((((1R,5S,6s)-3-(2- (2,4-difluorophenyl)-2-hydroxy- 3-(1H-1,2,4-triazol-1-yl)propyl)- 3-azabicyclo[3.1.0]hexan-6- yl)amino)methyl)-1-(4-phenyl- 3,4-dihydro-2H- benzo[b][1,4]oxazin-6- yl)thieno[2,3-b]pyrazin-2(1H)- one | 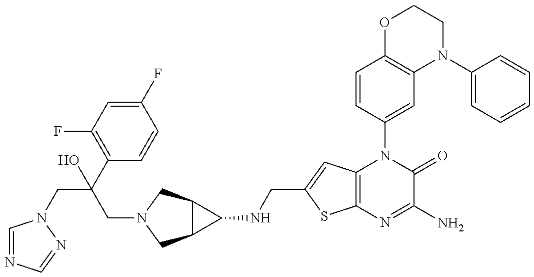 [SMILES: Nc1nc2sc(CN[C@H]3[C@@H]4CN(CC(O)(Cn5cncn5)c5ccc(F)cc5F)C[C@@H]43)cc2n(-c2ccc3c(c2)N(c2ccccc2)CCO3)c1=O] |
| I-82 | 3-amino-6-(((2-(2,4- difluorophenyl)-2-hydroxy-3- (1H-1,2,4-triazol-1- yl)propyl)amino)methyl)-1-(4- phenyl-3,4-dihydro-2H- benzo[b][1,4]oxazin-6- yl)thieno[2,3-b]pyrazin-2(1H)- one | 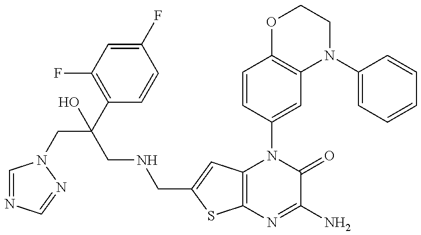 [SMILES: Nc1nc2sc(CNCC(O)(Cn3cncn3)c3ccc(F)cc3F)cc2n(-c2ccc3c(c2)N(c2ccccc2)CCO3)c1=O] |
| I-83 | N-((5-((1H-1,2,4-triazol-1- yl)methyl)-5-(2,4- difluorophenyl)tetrahydrofuran- 3-yl)methyl)-3-amino-2-oxo-1- (4-phenyl-3,4-dihydro-2H- benzo[b][1,4]oxazin-6-yl)-1,2- dihydrothieno[2,3-b]pyrazine- 6-carboxamide | 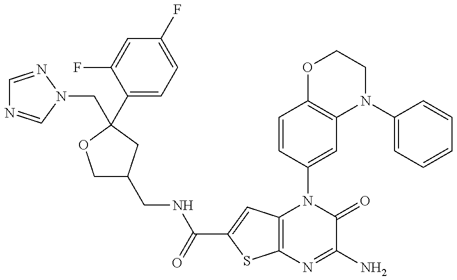 [SMILES: Nc1nc2sc(C(=O)NCC3COC(Cn4cncn4)(c4ccc(F)cc4F)C3)cc2n(-c2ccc3c(c2)N(c2ccccc2)CCO3)c1=O] |
| I-84 | 2-((2-(2-(2-((2-(2,4- difluorophenyl)-2-hydroxy-3- (1H-1,2,4-triazol-1- yl)propyl)amino)ethoxy)ethoxy) ethyl)amino)-4-(3,6,6-trimethyl- 4-oxo-4,5,6,7-tetrahydro-1H- indazol-1-yl)benzamide | 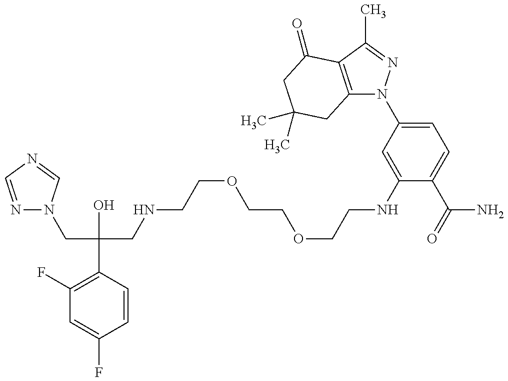 [SMILES: Cc1nn(-c2ccc(C(N)=O)c(NCCOCCOCCNCC(O)(Cn3cncn3)c3ccc(F)cc3F)c2)c2c1C(=O)CC(C)(C)C2] |
| I-85 | 3-amino-N-(4-(4-(5- fluoropyrimidin-2-yl)piperazin- 1-yl)phenyl)-2-oxo-1-(4- phenyl-3,4-dihydro-2H- benzo[b][1,4]oxazin-6-yl)-1,2- dihydrothieno[2,3-b]pyrazine- 6-carboxamide | 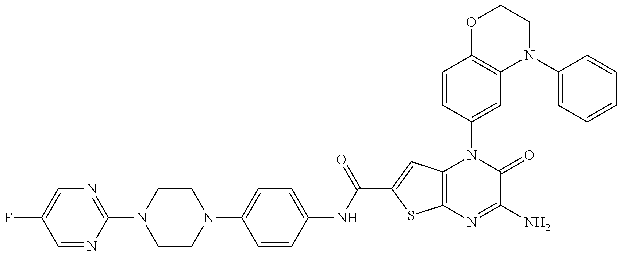 [SMILES: Nc1nc2sc(C(=O)Nc3ccc(N4CCN(c5ncc(F)cn5)CC4)cc3)cc2n(-c2ccc3c(c2)N(c2ccccc2)CCO3)c1=O] |
| I-86 | 3-amino-N-(1-((9Z,12Z,15Z)- octadeca-9,12,15- trienoyl)azetidin-3-yl)-2-oxo-1- (4-phenyl-3,4-dihydro-2H- benzo[b][1,4]oxazin-6-yl)-1,2- dihydrothieno[2,3-b]pyrazine- 6-carboxamide | 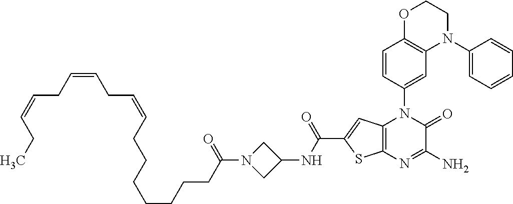 [SMILES: CC/C=C\C/C=C\C/C=C\CCCCCCCC(=O)N1CC(NC(=O)c2cc3c(nc(N)c(=O)n3-c3ccc4c(c3)N(c3ccccc3)CCO4)s2)C1] |
| I-87 | 3-amino-N-(1-(5- fluoropyrimidin-2-yl)piperidin- 4-yl)-2-oxo-1-(4-phenyl-3,4- dihydro-2H- benzo[b][1,4]oxazin-6-yl)-1,2- dihydrothieno[2,3-b]pyrazine- 6-carboxamide | 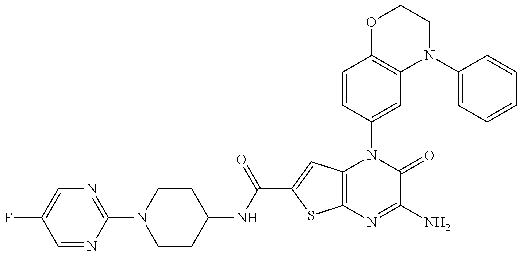 [SMILES: Nc1nc2sc(C(=O)NC3CCN(c4ncc(F)cn4)CC3)cc2n(-c2ccc3c(c2)N(c2ccccc2)CCO3)c1=O] |
| I-88 | 3-amino-N-((S)-1-(3- ((9Z,12Z,15Z)-octadeca- 9,12,15- trienamido)propyl)pyrrolidin-3- yl)-2-oxo-1-(4-phenyl-3,4- dihydro-2H- benzo[b][1,4]oxazin-6-yl)-1,2- dihydrothieno[2,3-b]pyrazine- 6-carboxamide | 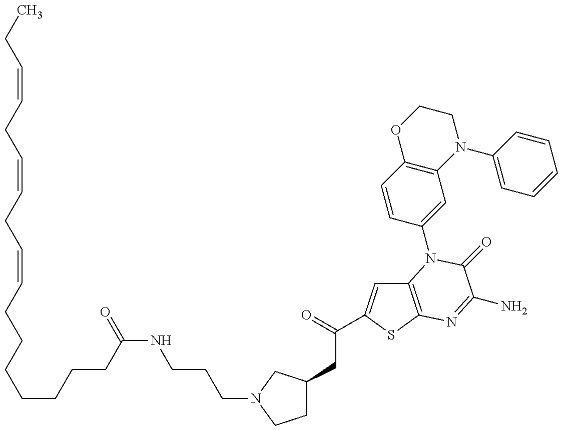 [SMILES: CC/C=C\C/C=C\C/C=C\CCCCCCCC(=O)NCCCN1CC[C@H](CC(=O)c2cc3c(nc(N)c(=O)n3-c3ccc4c(c3)N(c3ccccc3)CCO4)s2)C1] |
| I-89 | 3-amino-N-(4-((4-(5- fluoropyrimidin-2-yl)piperazin- 1-yl)methyl)phenyl)-2-oxo-1- (4-phenyl-3,4-dihydro-2H- benzo[b][1,4]oxazin-6-yl)-1,2- dihydrothieno[2,3-b]pyrazine- 6-carboxamide | 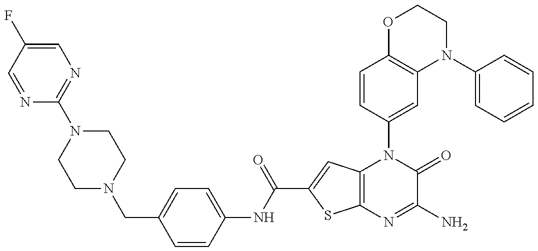 [SMILES: Nc1nc2sc(C(=O)Nc3ccc(CN4CCN(c5ncc(F)cn5)CC4)cc3)cc2n(-c2ccc3c(c2)N(c2ccccc2)CCO3)c1=O] |
| I-90 | 3-amino-2-oxo-1-(4-phenyl- 3,4-dihydro-2H- benzo[b][1,4]oxazin-6-yl)-N-(1- ((2E,6E)-3,7,11- trimethyldodeca-2,6,10-trien-1- yl)piperidin-4-yl)-1,2- dihydrothieno[2,3-b]pyrazine- 6-carboxamide | 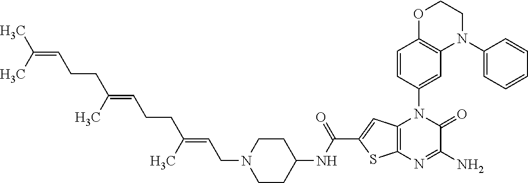 [SMILES: CC(C)=CCC/C(C)=C/CC/C(C)=C/CN1CCC(NC(=O)c2cc3c(nc(N)c(=O)n3-c3ccc4c(c3)N(c3ccccc3)CCO4)s2)CC1] |
| I-91 | 3-amino-2-oxo-1-(4-phenyl- 3,4-dihydro-2H- benzo[b][1,4]oxazin-6-yl)-N- ((R)-1-((2E,6E)-3,7,11- trimethyldodeca-2,6, 10-trien-1- yl)pyrrolidin-3-yl)-1,2- dihydrothieno[2,3-b]pyrazine- 6-carboxamide | 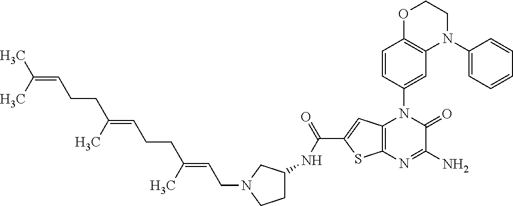 |
| I-92 | (R)-3-amino-N-(1-(4-(4-(5- fluoropyrimidin-2-yl)piperazin- 1-yl)phenethyl)pyrrolidin-3-yl)- 2-oxo-1-(3-phenyl-2,3- dihydrobenzo[d]oxazol-5-yl)- 1,2-dihydrothieno[2,3- b]pyrazine-6-carboxamide | 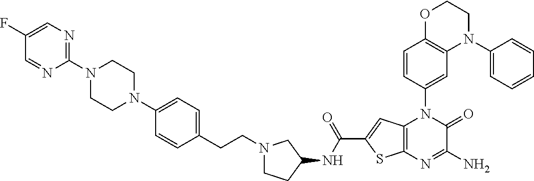 |
| I-93 | 3-amino-N-(4-(((3-((4-((3- aminopropyl)amino)butyl)amino) propyl)amino)methyl)phenyl)- 2-oxo-1-(4-phenyl-3,4- dihydro-2H- benzo[b][1,4]oxazin-6-yl)-1,2- dihydrothieno[2,3-b]pyrazine- 6-carboxamide | 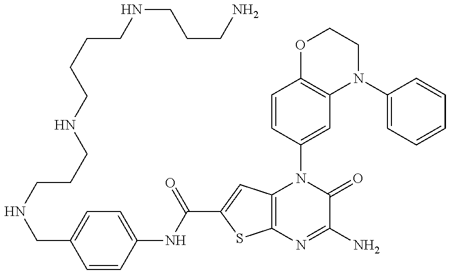 [SMILES: NCCCNCCCCNCCCNCc1ccc(NC(=O)c2cc3c(nc(N)c(=O)n3-c3ccc4c(c3)N(c3ccccc3)CCO4)s2)cc1] |
| I-94 | (R)-3-amino-N-(1-(N-(3-((3- aminopropyl)amino)propyl) carbamimidoyl)pyrrolidin-3-yl)-2- oxo-1-(4-phenyl-3,4-dihydro- 2H-benzo[b][1,4]oxazin-6-yl)- 1,2-dihydrothieno[2,3- b]pyrazine-6-carboxamide | 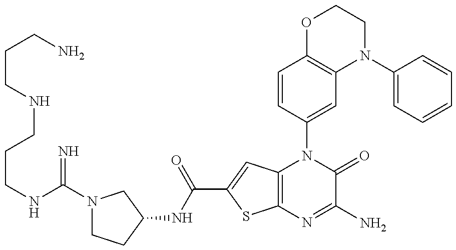 [SMILES: N=C(NCCCNCCCN)N1CC[C@@H](NC(=O)c2cc3c(nc(N)c(=O)n3-c3ccc4c(c3)N(c3ccccc3)CCO4)s2)C1] |
| I-95 | (R)-3-amino-N-(1-(4-(4-(5- fluoropyrimidin-2-yl)piperazin- 1-yl)phenethyl)pyrrolidin-3-yl)- 2-oxo-1-(1-phenyl-1H-indol-6- yl)-1,2-dihydrothieno[2,3- b]pyrazine-6-carboxamide | 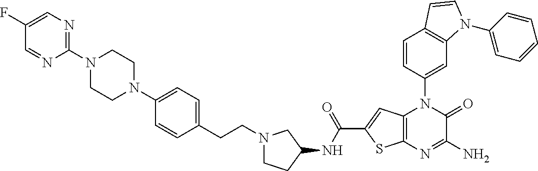 |
| I-96 | 3-amino-N-(4-(((3-((3- guanidinopropyl)amino)propyl) amino)methyl)phenyl)-2-oxo-1- (4-phenyl-3,4-dihydro-2H- benzo[b][1,4]oxazin-6-yl)-1,2- dihydrothieno[2,3-b]pyrazine- 6-carboxamide | 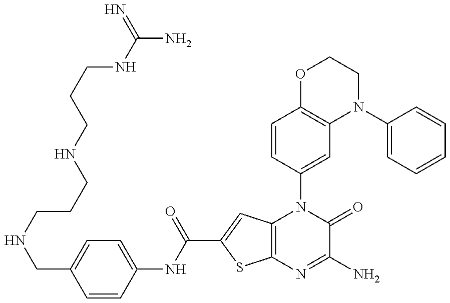 [SMILES: N=C(N)NCCCNCCCNCc1ccc(NC(=O)c2cc3c(nc(N)c(=O)n3-c3ccc4c(c3)N(c3ccccc3)CCO4)s2)cc1] |
| I-97 | 3-amino-N-(4-(((3-((3- aminopropyl)amino)propyl) amino)methyl)phenyl)-2-oxo-1- (4-phenyl-3,4-dihydro-2H- benzo[b][1,4]oxazin-6-yl)-1,2- dihydrothieno[2,3-b]pyrazine- 6-carboxamide | 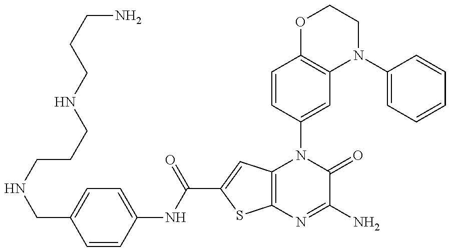 [SMILES: NCCCNCCCNCc1ccc(NC(=O)c2cc3c(nc(N)c(=O)n3-c3ccc4c(c3)N(c3ccccc3)CCO4)s2)cc1] |
| I-98 | (R)-3-amino-N-(1-(N-(3-((4- aminobutyl)amino)propyl) carbamimidoyl)pyrrolidin-3-yl)-2- oxo-1-(4-phenyl-3,4-dihydro- 2H-benzo[b][1,4]oxazin-6-yl)- 1,2-dihydrothieno[2,3- b]pyrazine-6-carboxamide | 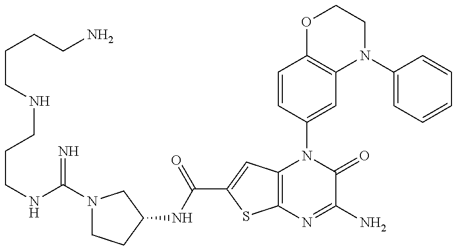 [SMILES: N=C(NCCCNCCCCN)N1CC[C@@H](NC(=O)c2cc3c(nc(N)c(=O)n3-c3ccc4c(c3)N(c3ccccc3)CCO4)s2)C1] |
| I-99 | 3-amino-N-(3-((4-((3- aminopropyl)amino)butyl)amino) propyl)-2-oxo-1-(4-phenyl- 3,4-dihydro-2H- benzo[b][1,4]oxazin-6-yl)-1,2- dihydrothieno[2,3-b]pyrazine- 6-carboxamide | 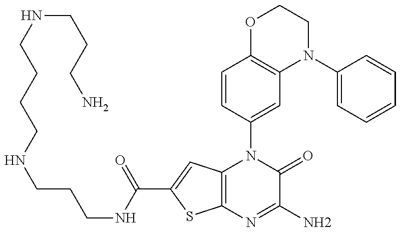 [SMILES: NCCCNCCCCNCCCNC(=O)c1cc2c(nc(N)c(=O)n2-c2ccc3c(c2)N(c2ccccc2)CCO3)s1] |
| I-100 | 3-amino-N-(3-((4- guanidinobutyl)amino)propyl)- 2-oxo-1-(4-phenyl-3,4-dihydro- 2H-benzo[b][1,4]oxazin-6-yl)- 1,2-dihydrothieno[2,3- b]pyrazine-6-carboxamide | 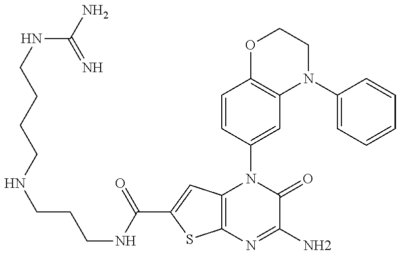 [SMILES: N=C(N)NCCCCNCCCNC(=O)c1cc2c(nc(N)c(=O)n2-c2ccc3c(c2)N(c2ccccc2)CCO3)s1] |
| I-101 | 3-amino-N-(3-((4- aminobutyl)amino)propyl)-2- oxo-1-(4-phenyl-3,4-dihydro- 2H-benzo[b][1,4]oxazin-6-yl)- 1,2-dihydrothieno[2,3- b]pyrazine-6-carboxamide | 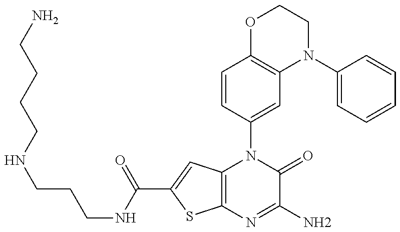 [SMILES: NCCCCNCCCNC(=O)c1cc2c(nc(N)c(=O)n2-c2ccc3c(c2)N(c2ccccc2)CCO3)s1] |
| I-102 | 3-amino-N-(3-((3- guanidinopropyl)amino)propyl)- 2-oxo-1-(4-phenyl-3,4- dihydro-2H- benzo[b][1,4]oxazin-6-yl)-1,2- dihydrothieno[2,3-b]pyrazine- 6-carboxamide | 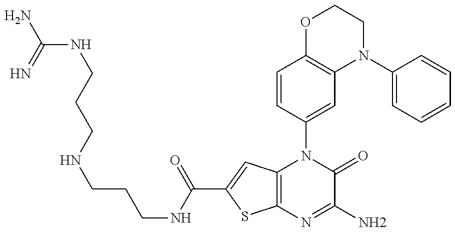 [SMILES: N=C(N)NCCCNCCCNC(=O)c1cc2c(nc(N)c(=O)n2-c2ccc3c(c2)N(c2ccccc2)CCO3)s1] |
| I-103 | 3-amino-N-(3-((3- aminopropyl)amino)propyl)-2- oxo-1-(4-phenyl-3,4-dihydro- 2H-benzo[b][1,4]oxazin-6-yl)- 1,2-dihydrothieno[2,3- b]pyrazine-6-carboxamide | 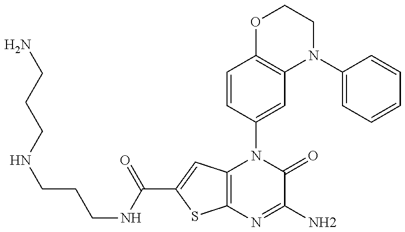 [SMILES: NCCCNCCCNC(=O)c1cc2c(nc(N)c(=O)n2-c2ccc3c(c2)N(c2ccccc2)CCO3)s1] |
| I-104 | 3-amino-N-(4-(2-(4-(5- fluoropyrimidin-2-yl)piperazin- 1-yl)ethyl)phenyl)-2-oxo-1-(4- phenyl-3,4-dihydro-2H- benzo[b][1,4]oxazin-6-yl)-1,2- dihydrothieno[2,3-b]pyrazine- 6-carboxamide | 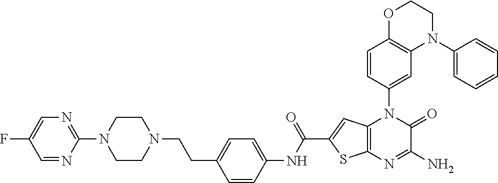 [SMILES: Nc1nc2sc(C(=O)Nc3ccc(CCN4CCN(c5ncc(F)cn5)CC4)cc3)cc2n(-c2ccc3c(c2)N(c2ccccc2)CCO3)c1=O] |
| I-105 | 3-amino-N-(4-(3-(4-(5- fluoropyrimidin-2-yl)piperazin- 1-yl)propyl)phenyl)-2-oxo-1-(4- phenyl-3,4-dihydro-2H- benzo[b][1,4]oxazin-6-yl)-1,2- dihydrothieno[2,3-b]pyrazine- 6-carboxamide | 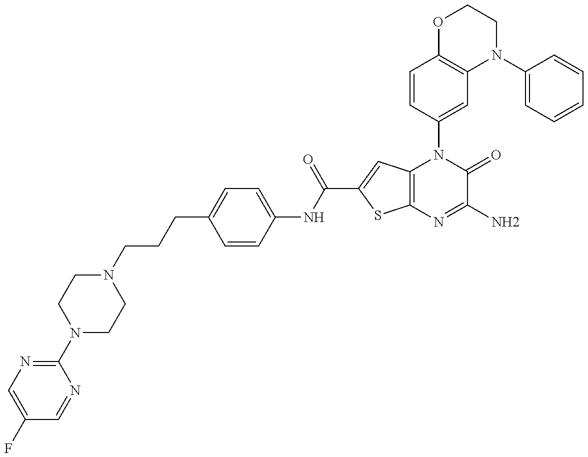 [SMILES: Nc1nc2sc(C(=O)Nc3ccc(CCCN4CCN(c5ncc(F)cn5)CC4)cc3)cc2n(-c2ccc3c(c2)N(c2ccccc2)CCO3)c1=O] |
| I-106 | 3-amino-N-(1-(3-((3- aminopropyl)amino)propyl) piperidin-4-yl)-2-oxo-1-(4- phenyl-3,4-dihydro-2H- benzo[b][1,4]oxazin-6-yl)-1,2- dihydrothieno[2,3-b]pyrazine- 6-carboxamide | 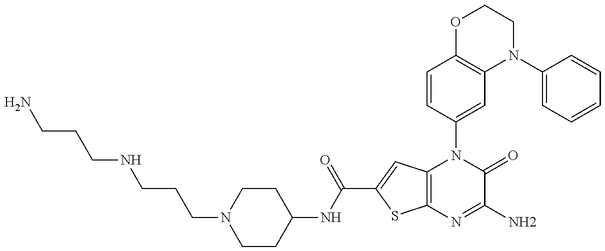 [SMILES: NCCCNCCCN1CCC(NC(=O)c2cc3c(nc(N)c(=O)n3-c3ccc4c(c3)N(c3ccccc3)CCO4)s2)CC1] |
| I-107 | 3-amino-N-(1-(2-((4- carbamimidoylphenyl)amino) ethyl)pyrrolidin-3-yl)-2-oxo-1-(4- phenyl-3,4-dihydro-2H- benzo[b][1,4]oxazin-6-yl)-1,2- dihydrothieno[2,3-b]pyrazine- 6-carboxamide | 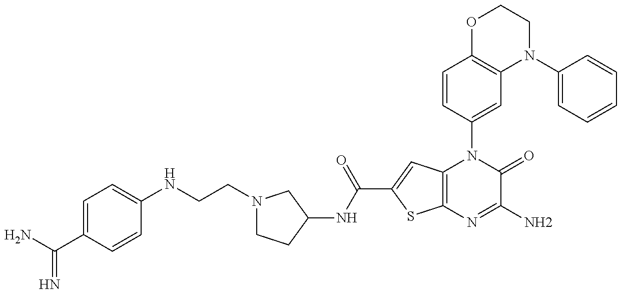 [SMILES: N=C(N)c1ccc(NCCN2CCC(NC(=O)c3cc4c(nc(N)c(=O)n4-c4ccc5c(c4)N(c4ccccc4)CCO5)s3)C2)cc1] |
| I-108 | 3-amino-N-(1-(2-((4- carbamimidoylphenyl)amino) ethyl)piperidin-4-yl)-2-oxo-1-(4- phenyl-3,4-dihydro-2H- benzo[b][1,4]oxazin-6-yl)-1,2- dihydrothieno[2,3-b]pyrazine- 6-carboxamide | 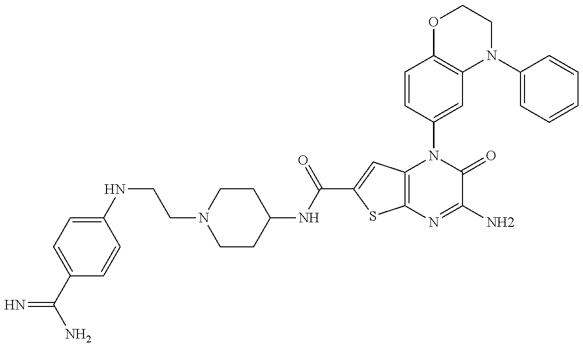 [SMILES: N=C(N)c1ccc(NCCN2CCC(NC(=O)c3cc4c(nc(N)c(=O)n4-c4ccc5c(c4)N(c4ccccc4)CCO5)s3)CC2)cc1] |
| I-109 | 3-amino-N-(1-(2-((4- carbamimidoylphenyl)amino) ethyl)piperidin-4-yl)-2-oxo-1-(1- phenyl-1,2,3,4- tetrahydroquinolin-7-yl)-1,2- dihydrothieno[2,3-b]pyrazine- 6-carboxamide | 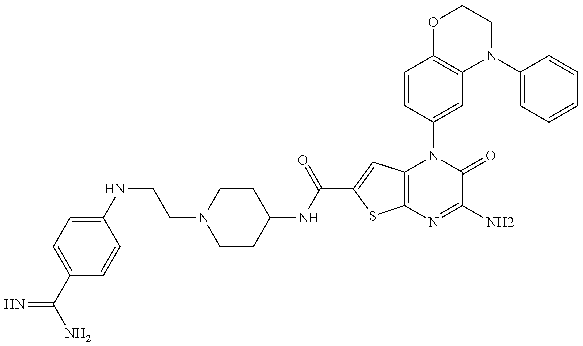 [SMILES: N=C(N)c1ccc(NCCN2CCC(NC(=O)c3cc4c(nc(N)c(=O)n4-c4ccc5c(c4)N(c4ccccc4)CCO5)s3)CC2)cc1] |
| I-110 | 3-amino-2-oxo-1-(4-phenyl- 3,4-dihydro-2H- benzo[b][1,4]oxazin-6-yl)-N-(1- (3-(triphenyl-l5- phosphaneyl)propyl)piperidin- 4-yl)-1,2-dihydrothieno[2,3- b]pyrazine-6-carboxamide | 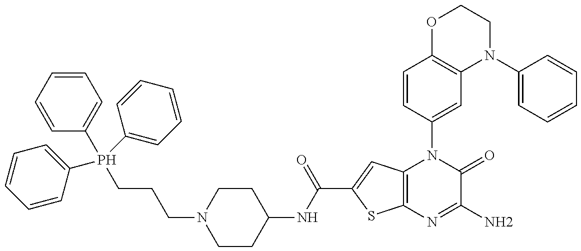 |
| I-111 | 3-amino-N-(1-(3-((3-((2- aminoethyl)amino)propyl)amino) propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 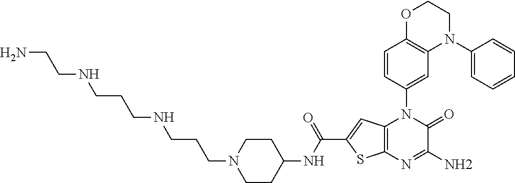 [SMILES: NCCNCCCNCCCN1CCC(NC(=O)c2cc3c(nc(N)c(=O)n3-c3ccc4c(c3)N(c3ccccc3)CCO4)s2)CC1] |
| I-112 | 3-amino-N-(1-(3-((3-((2- aminoethyl)amino)propyl)amino) propyl)piperidin-4-yl)-2-oxo- 1-(1-phenyl-1,2,3,4- tetrahydroquinolin-7-yl)-1,2- dihydrothieno[2,3-b]pyrazine- 6-carboxamide | 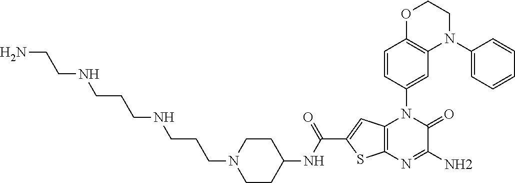 [SMILES: NCCNCCCNCCCN1CCC(NC(=O)c2cc3c(nc(N)c(=O)n3-c3ccc4c(c3)N(c3ccccc3)CCO4)s2)CC1] |
| I-113 | 2,2-difluoropropyl 2-amino-4- (2,4-dichloro-6-(2-(4-fluoro-1H- pyrazol-1-yl)ethoxy)phenyl)- 5,7-dihydro-6H-pyrrolo[3,4- d]pyrimidine-6-carboxylate | 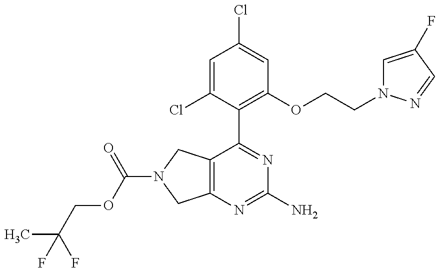 [SMILES: CC(F)(F)COC(=O)N1Cc2nc(N)nc(-c3c(Cl)cc(Cl)cc3OCCn3cc(F)cn3)c2C1] |
| I-114 | 2,2-difluoropropyl 2-amino-4- (2,4-dichloro-6-(2-(2,4- difluorophenyl)-2-hydroxy-3- (1H-1,2,4-triazol-1- yl)propoxy)phenyl)-5,7- dihydro-6H-pyrrolo[3,4- d]pyrimidine-6-carboxylate | 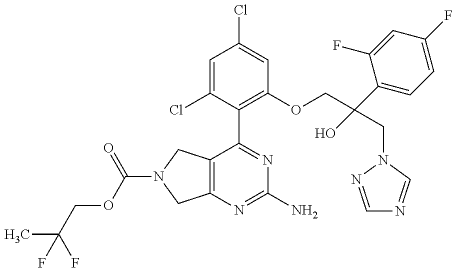 [SMILES: CC(F)(F)COC(=O)N1Cc2nc(N)nc(-c3c(Cl)cc(Cl)cc3OCC(O)(Cn3cncn3)c3ccc(F)cc3F)c2C1] |
| I-115 | 2,2-difluoropropyl 2-amino-4- (2,4-dichloro-6-(3-(2,4- difluorophenyl)-3-hydroxy-4- (1H-1,2,4-triazol-1- yl)butoxy)phenyl)-5,7-dihydro- 6H-pyrrolo[3,4-d]pyrimidine-6- carboxylate | 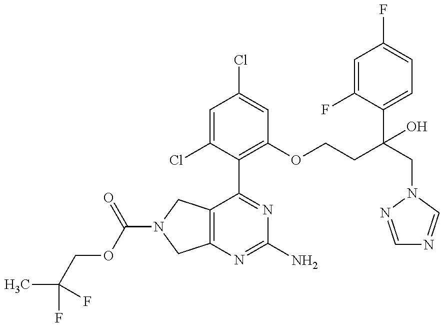 [SMILES: CC(F)(F)COC(=O)N1Cc2nc(N)nc(-c3c(Cl)cc(Cl)cc3OCCC(O)(Cn3cncn3)c3ccc(F)cc3F)c2C1] |
| I-116 | 3-amino-6-((((1R,5S)-3-(2- (2,4-difluorophenyl)-2-hydroxy- 3-(1H-1,2,4-triazol-1-yl)propyl)- 3-azabicyclo[3.2.1]octan-8- yl)amino)methyl)-1-(4-phenyl- 3,4-dihydro-2H- benzo[b][1,4]oxazin-6- yl)thieno[2,3-b]pyrazin-2(1H)- one | 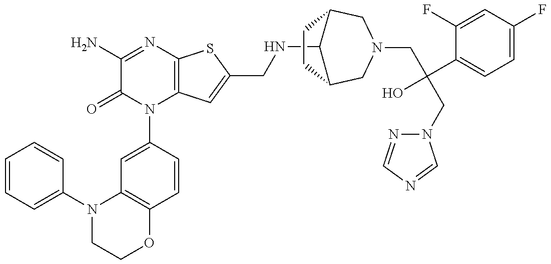 [SMILES: Nc1nc2sc(CNC3[C@@H]4CC[C@H]3CN(CC(O)(Cn3cncn3)c3ccc(F)cc3F)C4)cc2n(-c2ccc3c(c2)N(c2ccccc2)CCO3)c1=O] |
| I-117 | 3-amino-6-(((1-(2-(2,4- difluorophenyl)-2-hydroxy-3- (1H-1,2,4-triazol-1-yl)propyl)-4- methylpiperidin-4- yl)amino)methyl)-1-(4-phenyl- 3,4-dihydro-2H- benzo[b][1,4]oxazin-6- yl)thieno[2,3-b]pyrazin-2(1H)- one | 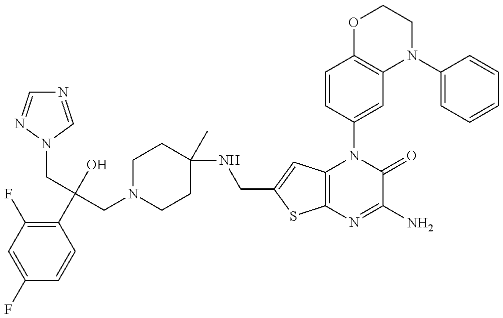 [SMILES: CC1(NCc2cc3c(nc(N)c(=O)n3-c3ccc4c(c3)N(c3ccccc3)CCO4)s2)CCN(CC(O)(Cn2cncn2)c2ccc(F)cc2F)CC1] |
| I-118 | 3-amino-6-(((1-(6-(2,4- difluorophenyl)-6-hydroxy-7- (1H-1,2,4-triazol-1- yl)heptyl)piperidin-4- yl)amino)methyl)-1-(4-phenyl- 3,4-dihydro-2H- benzo[b][1,4]oxazin-6- yl)thieno[2,3-b]pyrazin-2(1H)- one | 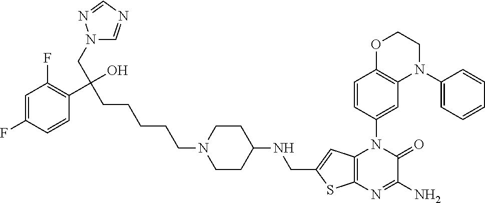 [SMILES: Nc1nc2sc(CNC3CCN(CCCCCC(O)(Cn4cncn4)c4ccc(F)cc4F)CC3)cc2n(-c2ccc3c(c2)N(c2ccccc2)CCO3)c1=O] |
| I-119 | 3-amino-6-(((1-(2-(2,4- difluorophenyl)-2-hydroxy-3- (1H-1,2,4-triazol-1- yl)propyl)piperidin-4- yl)(methyl)amino)methyl)-1-(4- phenyl-3,4-dihydro-2H- benzo[b][1,4]oxazin-6- yl)thieno[2,3-b]pyrazin-2(1H)- one | 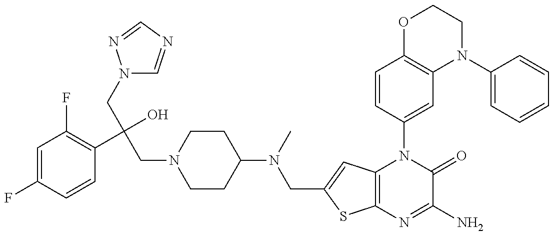 [SMILES: CN(Cc1cc2c(nc(N)c(=O)n2-c2ccc3c(c2)N(c2ccccc2)CCO3)s1)C1CCN(CC(O)(Cn2cncn2)c2ccc(F)cc2F)CC1] |
| I-120 | 3-amino-6-((((3R)-1-(3-(2,4- difluorophenyl)-3-hydroxy-4- (1H-1,2,4-triazol-1- yl)butyl)pyrrolidin-3- yl)amino)methyl)-1-(4-phenyl- 3,4-dihydro-2H- benzo[b][1,4]oxazin-6- yl)thieno[2,3-b]pyrazin-2(1H)- one | 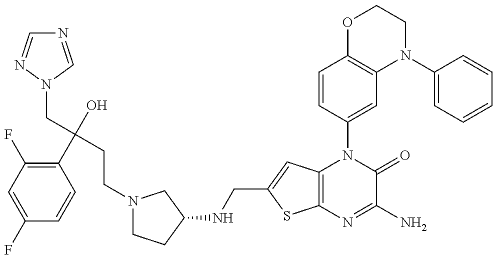 [SMILES: Nc1nc2sc(CN[C@@H]3CCN(CCC(O)(Cn4cncn4)c4ccc(F)cc4F)C3)cc2n(-c2ccc3c(c2)N(c2ccccc2)CCO3)c1=O] |
| I-121 | 3-amino-6-((((S)-1-((R)-2-(2,4- difluorophenyl)-2-hydroxy-3- (1H-1,2,4-triazol-1- yl)propyl)piperidin-3- yl)amino)methyl)-1-(4-phenyl- 3,4-dihydro-2H- benzo[b][1,4]oxazin-6- yl)thieno[2,3-b]pyrazin-2(1H)- one | 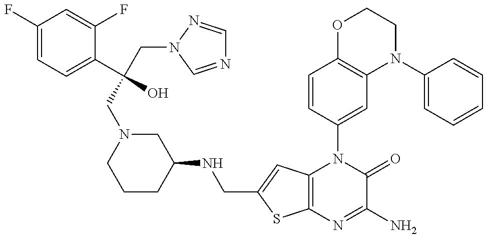 [SMILES: Nc1nc2sc(CN[C@H]3CCCN(C[C@@](O)(Cn4cncn4)c4ccc(F)cc4F)C3)cc2n(-c2ccc3c(c2)N(c2ccccc2)CCO3)c1=O] |
| I-122 | (R)-3-amino-6-((((1-(2-(2,4- difluorophenyl)-2-hydroxy-3- (1H-1,2,4-triazol-1- yl)propyl)piperidin-4- yl)methyl)amino)methyl)-1-(4- phenyl-3,4-dihydro-2H- benzo[b][1,4]oxazin-6- yl)thieno[2,3-b]pyrazin-2(1H)- one | 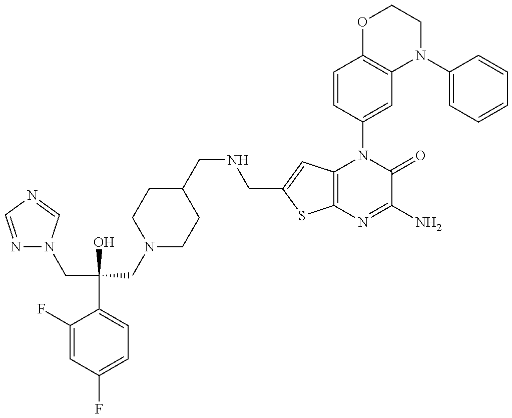 [SMILES: Nc1nc2sc(CNCC3CCN(C[C@@](O)(Cn4cncn4)c4ccc(F)cc4F)CC3)cc2n(-c2ccc3c(c2)N(c2ccccc2)CCO3)c1=O] |
| I-123 | 3-amino-6-((((R)-1-((S)-3-(2,4- difluorophenyl)-3-hydroxy-4- (1H-1,2,4-triazol-1- yl)butyl)pyrrolidin-3- yl)amino)methyl)-1-(4-phenyl- 3,4-dihydro-2H- benzo[b][1,4]oxazin-6- yl)thieno[2,3-b]pyrazin-2(1H)- one | 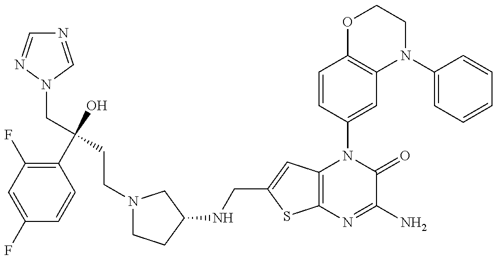 [SMILES: Nc1nc2sc(CN[C@@H]3CCN(CC[C@@](O)(Cn4cncn4)c4ccc(F)cc4F)C3)cc2n(-c2ccc3c(c2)N(c2ccccc2)CCO3)c1=O] |
| I-124 | 3-amino-6-((((R)-1-((R)-3-(2,4- difluorophenyl)-3-hydroxy-4- (1H-1,2,4-triazol-1- yl)butyl)pyrrolidin-3- yl)amino)methyl)-1-(4-phenyl- 3,4-dihydro-2H- benzo[b][1,4]oxazin-6- yl)thieno[2,3-b]pyrazin-2(1H)- one | 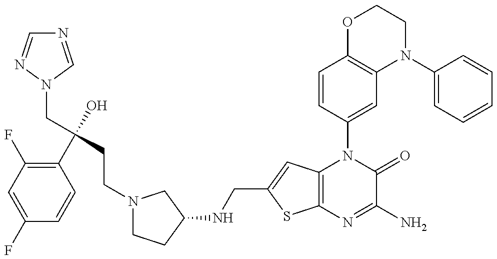 [SMILES: Nc1nc2sc(CN[C@@H]3CCN(CC[C@](O)(Cn4cncn4)c4ccc(F)cc4F)C3)cc2n(-c2ccc3c(c2)N(c2ccccc2)CCO3)c1=O] |
| I-125 | 3-amino-6-((((1R,5S,6R)-3- ((S)-2-(2,4-difluorophenyl)-2- hydroxy-3-(1H-1,2,4-triazol-1- yl)propyl)-3- azabicyclo[3.1.0]hexan-6- yl)amino)methyl)-1-(4-phenyl- 3,4-dihydro-2H- benzo[b][1,4]oxazin-6- yl)thieno[2,3-b]pyrazin-2(1H)- one | 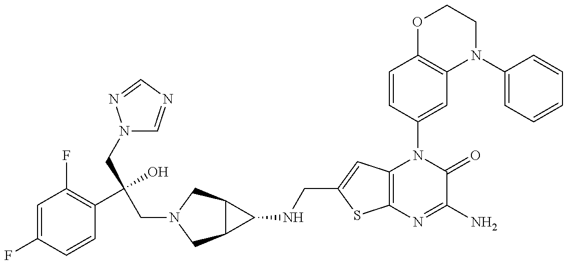 [SMILES: Nc1nc2sc(CN[C@H]3[C@@H]4CN(C[C@](O)(Cn5cncn5)c5ccc(F)cc5F)C[C@@H]43)cc2n(-c2ccc3c(c2)N(c2ccccc2)CCO3)c1=O] |
| I-126 | 3-amino-1-(4-phenyl-3,4- dihydro-2H- benzo[b][1,4]oxazin-6-yl)-6- ((piperidin-4- ylamino)methyl)thieno[2,3- b]pyrazin-2(1H)-one | 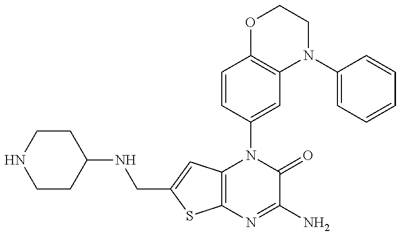 [SMILES: Nc1nc2sc(CNC3CCNCC3)cc2n(-c2ccc3c(c2)N(c2ccccc2)CCO3)c1=O] |
| I-127 | 3-amino-6-((((1R,5S,6S)-3- ((R)-2-(2,4-difluorophenyl)-2- hydroxy-3-(1H-1,2,4-triazol-1- yl)propyl)-3- azabicyclo[3.1.0]hexan-6- yl)amino)methyl)-1-(4-phenyl- 3,4-dihydro-2H- benzo[b][1,4]oxazin-6- yl)thieno[2,3-b]pyrazin-2(1H)- one | 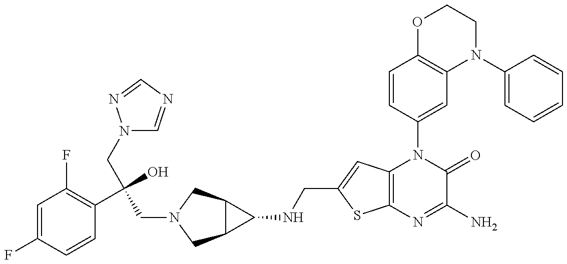 [SMILES: Nc1nc2sc(CN[C@H]3[C@@H]4CN(C[C@@](O)(Cn5cncn5)c5ccc(F)cc5F)C[C@@H]43)cc2n(-c2ccc3c(c2)N(c2ccccc2)CCO3)c1=O] |
| I-128 | (R)-3-amino-6-(((1-(3-(2,4- difluorophenyl)-3-hydroxy-4- (1H-1,2,4-triazol-1- yl)butanoyl)piperidin-4- yl)amino)methyl)-1-(4-phenyl- 3,4-dihydro-2H- benzo[b][1,4]oxazin-6- yl)thieno[2,3-b]pyrazin-2(1H)- one | 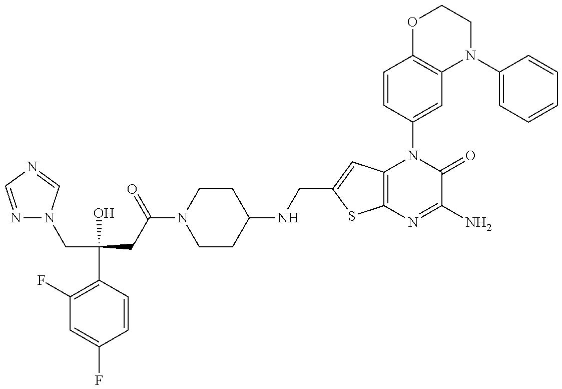 [SMILES: Nc1nc2sc(CNC3CCN(C(=O)C[C@](O)(Cn4cncn4)c4ccc(F)cc4F)CC3)cc2n(-c2ccc3c(c2)N(c2ccccc2)CCO3)c1=O] |
| I-129 | (S)-3-amino-6-(((1-(3-(2,4- difluorophenyl)-3-hydroxy-4- (1H-1,2,4-triazol-1- yl)butanoyl)piperidin-4- yl)amino)methyl)-1-(4-phenyl- 3,4-dihydro-2H- benzo[b][1,4]oxazin-6- yl)thieno[2,3-b]pyrazin-2(1H)- one | 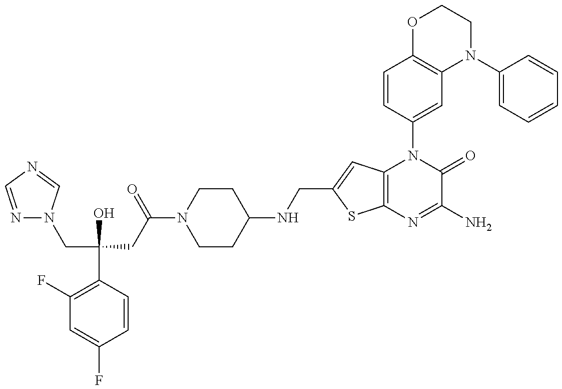 [SMILES: Nc1nc2sc(CNC3CCN(C(=O)C[C@@](O)(Cn4cncn4)c4ccc(F)cc4F)CC3)cc2n(-c2ccc3c(c2)N(c2ccccc2)CCO3)c1=O] |
| I-130 | 3-amino-6-((((S)-1-((S)-2-(2,4- difluorophenyl)-2-hydroxy-3- (1H-1,2,4-triazol-1- yl)propyl)piperidin-3- yl)amino)methyl)-1-(4-phenyl- 3,4-dihydro-2H- benzo[b][1,4]oxazin-6- yl)thieno[2,3-b]pyrazin-2(1H)- one | 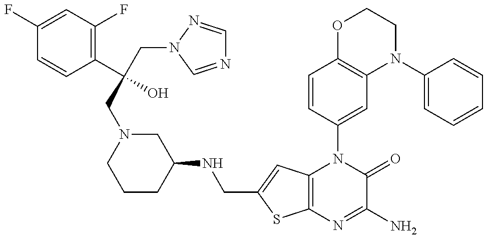 [SMILES: Nc1nc2sc(CN[C@H]3CCCN(C[C@](O)(Cn4cncn4)c4ccc(F)cc4F)C3)cc2n(-c2ccc3c(c2)N(c2ccccc2)CCO3)c1=O] |
| I-131 | (S)-3-amino-6-((((1-(2-(2,4- difluorophenyl)-2-hydroxy-3- (1H-1,2,4-triazol-1- yl)propyl)piperidin-4- yl)methyl)amino)methyl)-1-(4- phenyl-3,4-dihydro-2H- benzo[b][1,4]oxazin-6- yl)thieno[2,3-b]pyrazin-2(1H)- one | 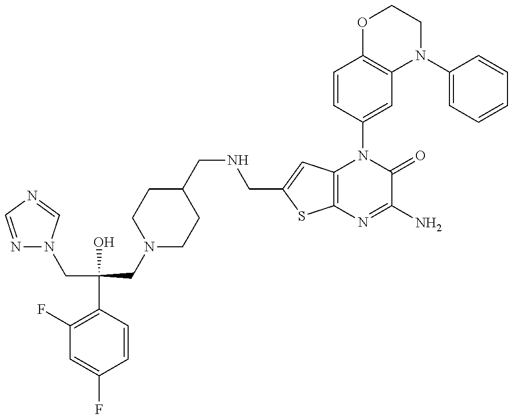 [SMILES: Nc1nc2sc(CNCC3CCN(C[C@](O)(Cn4cncn4)c4ccc(F)cc4F)CC3)cc2n(-c2ccc3c(c2)N(c2ccccc2)CCO3)c1=O] |
| I-132 | (R)-3-amino-6-(((1-(3-(2,4- difluorophenyl)-3-hydroxy-4- (1H-1,2,4-triazol-1- yl)butyl)piperidin-4- yl)amino)methyl)-1-(4-phenyl- 3,4-dihydro-2H- benzo[b][1,4]oxazin-6- yl)thieno[2,3-b]pyrazin-2(1H)- one | 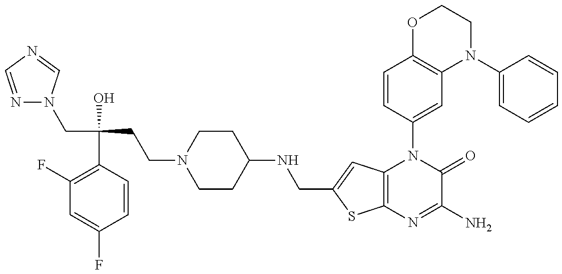 [SMILES: Nc1nc2sc(CNC3CCN(CC[C@](O)(Cn4cncn4)c4ccc(F)cc4F)CC3)cc2n(-c2ccc3c(c2)N(c2ccccc2)CCO3)c1=O] |
| I-133 | (S)-3-amino-6-(((1-(3-(2,4- difluorophenyl)-3-hydroxy-4- (1H-1,2,4-triazol-1- yl)butyl)piperidin-4- yl)amino)methyl)-1-(4-phenyl- 3,4-dihydro-2H- benzo[b][1,4]oxazin-6- yl)thieno[2,3-b]pyrazin-2(1H)- one | 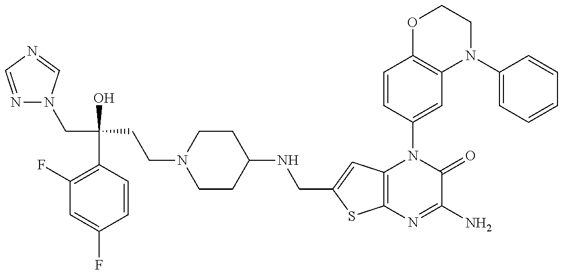 [SMILES: Nc1nc2sc(CNC3CCN(CC[C@@](O)(Cn4cncn4)c4ccc(F)cc4F)CC3)cc2n(-c2ccc3c(c2)N(c2ccccc2)CCO3)c1=O] |
| I-134 | 3-amino-6-((4-((2-(2,4- difluorophenyl)-2-hydroxy-3- (1H-1,2,4-triazol-1- yl)propyl)amino)piperidin-1- yl)methyl)-1-(4-phenyl-3,4- dihydro-2H- benzo[b][1,4]oxazin-6- yl)thieno[2,3-b]pyrazin-2(1H)- one | 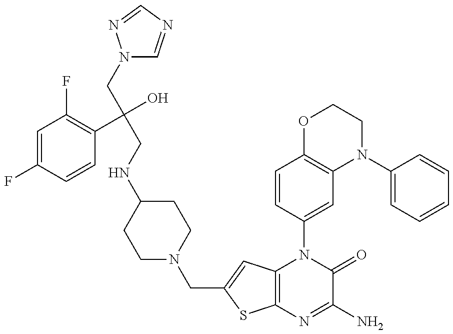 [SMILES: Nc1nc2sc(CN3CCC(NCC(O)(Cn4cncn4)c4ccc(F)cc4F)CC3)cc2n(-c2ccc3c(c2)N(c2ccccc2)CCO3)c1=O] |
| I-135 | 3-amino-6-(((1-(2-(2,4- difluorophenyl)-2-hydroxy-3- (1H-1,2,4-triazol-1- yl)propanoyl)piperidin-4- yl)amino)methyl)-1-(4-phenyl- 3,4-dihydro-2H- benzo[b][1,4]oxazin-6- yl)thieno[2,3-b]pyrazin-2(1H)- one | 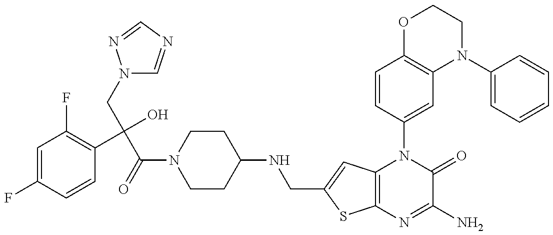 [SMILES: Nc1nc2sc(CNC3CCN(C(=O)C(O)(Cn4cncn4)c4ccc(F)cc4F)CC3)cc2n(-c2ccc3c(c2)N(c2ccccc2)CCO3)c1=O] |
| I-136 | 3-(2,4-difluorophenyl)-3- hydroxy-4-(1H-1,2,4-triazol-1- yl)butyl 2-(4-(((3-amino-2-oxo- 1-(4-phenyl-3,4-dihydro-2H- benzo[b][1,4]oxazin-6-yl)-1,2- dihydrothieno[2,3-b]pyrazin-6- yl)methyl)amino)piperidin-1- yl)acetate | 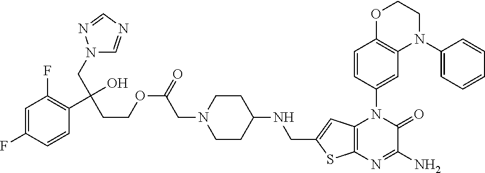 [SMILES: Nc1nc2sc(CNC3CCN(CC(=O)OCCC(O)(Cn4cncn4)c4ccc(F)cc4F)CC3)cc2n(-c2ccc3c(c2)N(c2ccccc2)CCO3)c1=O] |
| I-137 | 3-amino-6-(((1-(2-(2,4- difluorophenyl)-2-hydroxy-3- (1H-1,2,4-triazol-1-yl)propyl)- 3,3-difluoropiperidin-4- yl)amino)methyl)-1-(4-phenyl- 3,4-dihydro-2H- benzo[b][1,4]oxazin-6- yl)thieno[2,3-b]pyrazin-2(1H)- one | 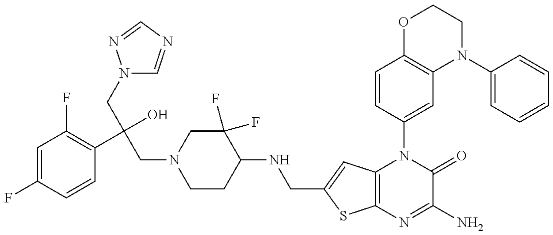 [SMILES: Nc1nc2sc(CNC3CCN(CC(O)(Cn4cncn4)c4ccc(F)cc4F)CC3(F)F)cc2n(-c2ccc3c(c2)N(c2ccccc2)CCO3)c1=O] |
| I-138 | 3-amino-6-(((1-(2-(2,4- difluorophenyl)-2-hydroxy-3- (1H-1,2,4-triazol-1-yl)propyl)-2- methylpiperidin-4- yl)amino)methyl)-1-(4-phenyl- 3,4-dihydro-2H- benzo[b][1,4]oxazin-6- yl)thieno[2,3-b]pyrazin-2(1H)- one | 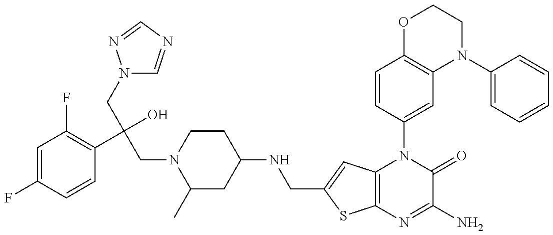 [SMILES: CC1CC(NCc2cc3c(nc(N)c(=O)n3-c3ccc4c(c3)N(c3ccccc3)CCO4)s2)CCN1CC(O)(Cn1cncn1)c1ccc(F)cc1F] |
| I-139 | 3-amino-6-(((1-(2-(2,4- difluorophenyl)-2-hydroxy-3- (1H-imidazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 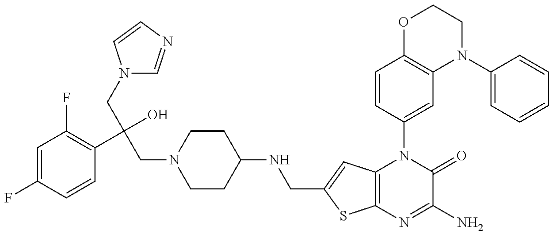 [SMILES: Nc1nc2sc(CNC3CCN(CC(O)(Cn4ccnc4)c4ccc(F)cc4F)CC3)cc2n(-c2ccc3c(c2)N(c2ccccc2)CCO3)c1=O] |
| I-140 | N-((3-amino-2-oxo-1-(4- phenyl-3,4-dihydro-2H- benzo[b][1,4]oxazin-6-yl)-1,2- dihydrothieno[2,3-b]pyrazin-6- yl)methyl)-1-(2-(2,4- difluorophenyl)-2-hydroxy-3- (1H-1,2,4-triazol-1- yl)propyl)piperidine-4- carboxamide | 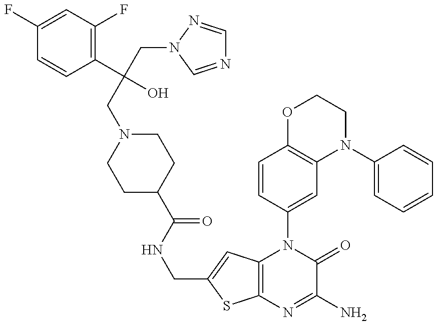 [SMILES: Nc1nc2sc(CNC(=O)C3CCN(CC(O)(Cn4cncn4)c4ccc(F)cc4F)CC3)cc2n(-c2ccc3c(c2)N(c2ccccc2)CCO3)c1=O] |
| I-141 | 3-amino-6-(((1-(2-(2,4- difluorophenyl)-2-hydroxy-3- (1H-1,2,4-triazol-1-yl)propyl)-2- oxopiperidin-4- yl)amino)methyl)-1-(4-phenyl- 3,4-dihydro-2H- benzo[b][1,4]oxazin-6- yl)thieno[2,3-b]pyrazin-2(1H)- one | 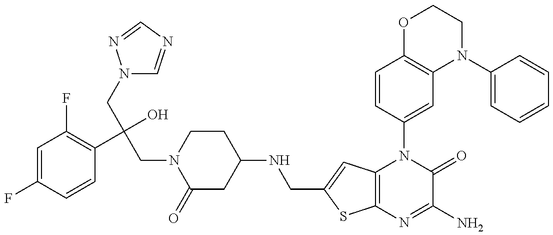 [SMILES: Nc1nc2sc(CNC3CCN(CC(O)(Cn4cncn4)c4ccc(F)cc4F)C(=O)C3)cc2n(-c2ccc3c(c2)N(c2ccccc2)CCO3)c1=O] |
| I-142 | 3-amino-N-(1-(2-hydroxy-2- (pyridin-2-yl)-3-(1H-1,2,4- triazol-1-yl)propyl)piperidin-4- yl)-2-oxo-1-(4-phenyl-3,4- dihydro-2H- benzo[b][1,4]oxazin-6-yl)-1,2- dihydrothieno[2,3-b]pyrazine- 6-carboxamide | 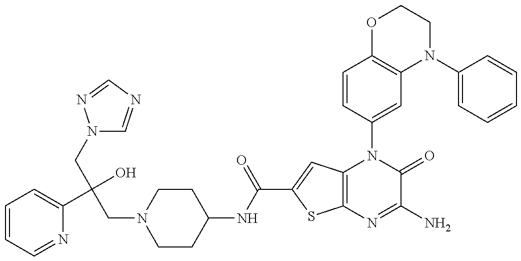 [SMILES: Nc1nc2sc(C(=O)NC3CCN(CC(O)(Cn4cncn4)c4ccccn4)CC3)cc2n(-c2ccc3c(c2)N(c2ccccc2)CCO3)c1=O] |
| I-143 | 3-amino-6-(((1-((2R,3S)-3- (2,4-difluorophenyl)-3-hydroxy- 4-(1H-1,2,4-triazol-1-yl)butan- 2-yl)piperidin-4- yl)amino)methyl)-1-(4-phenyl- 3,4-dihydro-2H- benzo[b][1,4]oxazin-6- yl)thieno[2,3-b]pyrazin-2(1H)- one | 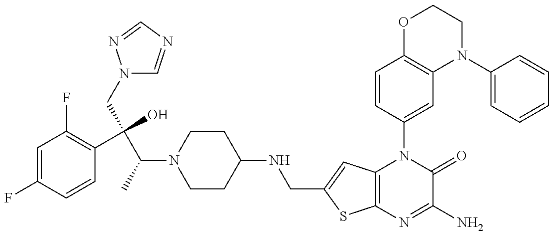 [SMILES: C[C@@H](N1CCC(NCc2cc3c(nc(N)c(=O)n3-c3ccc4c(c3)N(c3ccccc3)CCO4)s2)CC1)[C@@](O)(Cn1cncn1)c1ccc(F)cc1F] |
| I-144 | 3-amino-N-(1-(2-hydroxy-2- (pyridin-3-yl)-3-(1H-1,2,4- triazol-1-yl)propyl)piperidin-4- yl)-2-oxo-1-(4-phenyl-3,4- dihydro-2H- benzo[b][1,4]oxazin-6-yl)-1,2- dihydrothieno[2,3-b]pyrazine- 6-carboxamide | 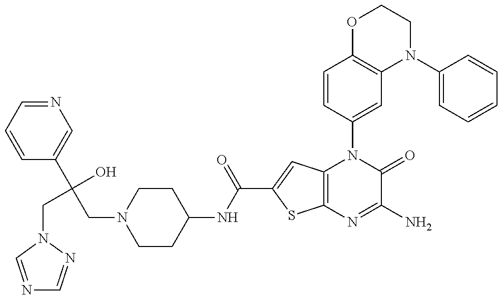 [SMILES: Nc1nc2sc(C(=O)NC3CCN(CC(O)(Cn4cncn4)c4cccnc4)CC3)cc2n(-c2ccc3c(c2)N(c2ccccc2)CCO3)c1=O] |
| I-145 | 3-amino-6-(((1-(2-hydroxy-2- phenyl-3-(1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 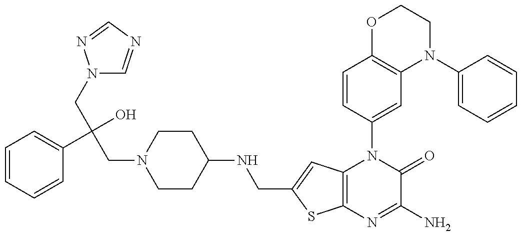 [SMILES: Nc1nc2sc(CNC3CCN(CC(O)(Cn4cncn4)c4ccccc4)CC3)cc2n(-c2ccc3c(c2)N(c2ccccc2)CCO3)c1=O] |
| I-146 | 3-amino-6-(((1-(2-hydroxy-2- (pyridin-3-yl)-3-(1H-1,2,4- triazol-1-yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 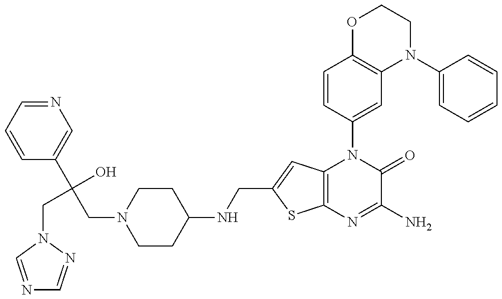 [SMILES: Nc1nc2sc(CNC3CCN(CC(O)(Cn4cncn4)c4cccnc4)CC3)cc2n(-c2ccc3c(c2)N(c2ccccc2)CCO3)c1=O] |
| I-147 | 3-amino-6-(((1-(2-hydroxy-2- (pyridin-2-yl)-3-(1H-1,2,4- triazol-1-yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 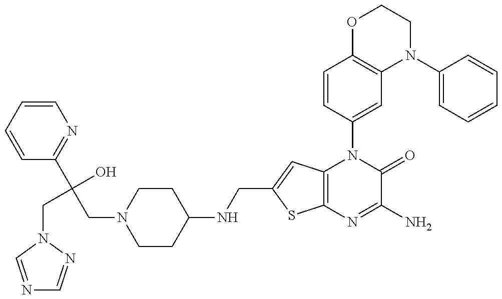 [SMILES: Nc1nc2sc(CNC3CCN(CC(O)(Cn4cncn4)c4ccccn4)CC3)cc2n(-c2ccc3c(c2)N(c2ccccc2)CCO3)c1=O] |
| I-148 | 3-amino-6-(((1-((2S,3S)-3-(2,4- difluorophenyl)-3-hydroxy-4- (1H-1,2,4-triazol-1-yl)butan-2- yl)piperidin-4- yl)amino)methyl)-1-(4-phenyl- 3,4-dihydro-2H- benzo[b][1,4]oxazin-6- yl)thieno[2,3-b]pyrazin-2(1H)- one | 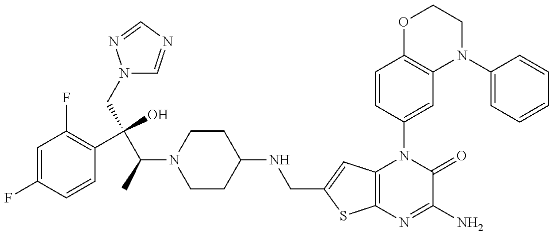 [SMILES: C[C@H](N1CCC(NCc2cc3c(nc(N)c(=O)n3-c3ccc4c(c3)N(c3ccccc3)CCO4)s2)CC1)[C@@](O)(Cn1cncn1)c1ccc(F)cc1F] |
| I-149 | 3-amino-6-(((1-((2S,3R)-3- (2,4-difluorophenyl)-3-hydroxy- 4-(1H-1,2,4-triazol-1-yl)butan- 2-yl)piperidin-4- yl)amino)methyl)-1-(4-phenyl- 3,4-dihydro-2H- benzo[b][1,4]oxazin-6- yl)thieno[2,3-b]pyrazin-2(1H)- one | 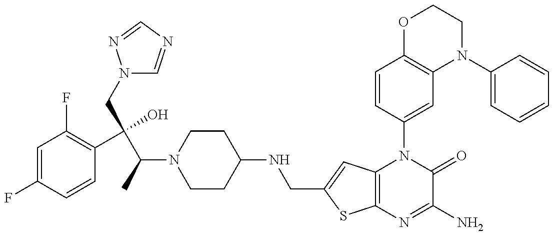 [SMILES: C[C@H](N1CCC(NCc2cc3c(nc(N)c(=O)n3-c3ccc4c(c3)N(c3ccccc3)CCO4)s2)CC1)[C@](O)(Cn1cncn1)c1ccc(F)cc1F] |
| I-150 | 3-amino-6-(((1-(2-(2,4- difluorophenyl)-2-hydroxy-3- (2H-tetrazol-2- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 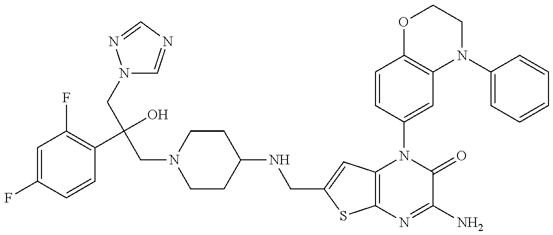 [SMILES: Nc1nc2sc(CNC3CCN(CC(O)(Cn4cncn4)c4ccc(F)cc4F)CC3)cc2n(-c2ccc3c(c2)N(c2ccccc2)CCO3)c1=O] |
| I-151 | (R)-3-amino-6-(((1-(2-(2,4- difluorophenyl)-2-hydroxy-3- (1H-imidazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 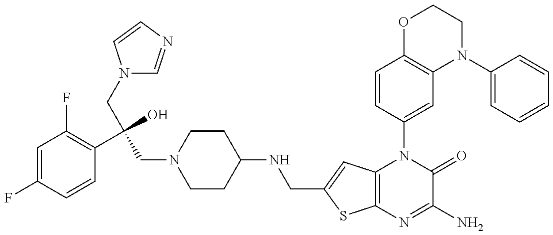 [SMILES: Nc1nc2sc(CNC3CCN(C[C@@](O)(Cn4ccnc4)c4ccc(F)cc4F)CC3)cc2n(-c2ccc3c(c2)N(c2ccccc2)CCO3)c1=O] |
| I-152 | (S)-3-amino-6-(((1-(2-(2,4- difluorophenyl)-2-hydroxy-3- (1H-imidazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 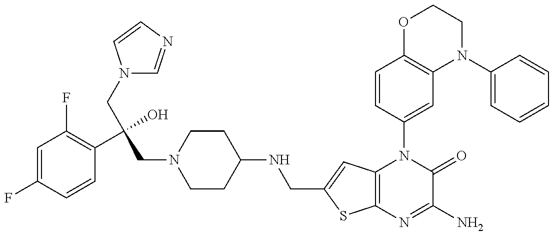 [SMILES: Nc1nc2sc(CNC3CCN(C[C@](O)(Cn4ccnc4)c4ccc(F)cc4F)CC3)cc2n(-c2ccc3c(c2)N(c2ccccc2)CCO3)c1=O] |
| I-153 | 3-amino-N-(1-(2-(2,4- difluorophenyl)-2-hydroxy-3- (1H-imidazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 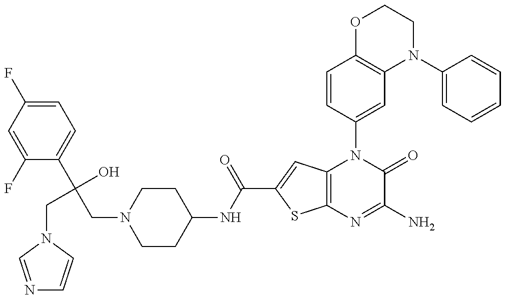 [SMILES: Nc1nc2sc(C(=O)NC3CCN(CC(O)(Cn4ccnc4)c4ccc(F)cc4F)CC3)cc2n(-c2ccc3c(c2)N(c2ccccc2)CCO3)c1=O] |
| I-154 | 3-amino-N-(1-(2-hydroxy-2- phenyl-3-(1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 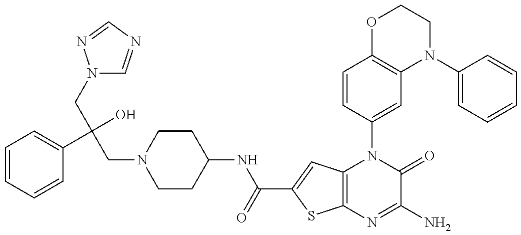 [SMILES: Nc1nc2sc(C(=O)NC3CCN(CC(O)(Cn4cncn4)c4ccccc4)CC3)cc2n(-c2ccc3c(c2)N(c2ccccc2)CCO3)c1=O] |
| I-155 | (R)-3-amino-6-(((1-(2-hydroxy- 2-phenyl-3-(1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 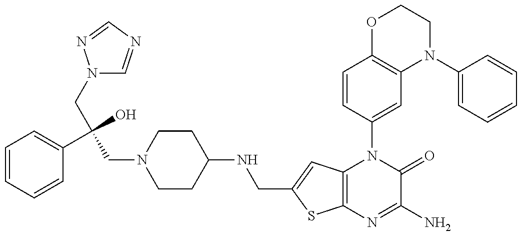 [SMILES: Nc1nc2sc(CNC3CCN(C[C@@](O)(Cn4cncn4)c4ccccc4)CC3)cc2n(-c2ccc3c(c2)N(c2ccccc2)CCO3)c1=O] |
| I-156 | (S)-3-amino-6-(((1-(2-hydroxy- 2-phenyl-3-(1H-1,2,4-triazol-1- yl)propyl)piperidin-4- yl)amino)methyl)-1-(4-phenyl- 3,4-dihydro-2H- benzo[b][1,4]oxazin-6- yl)thieno[2,3-b]pyrazin-2(1H)- one | 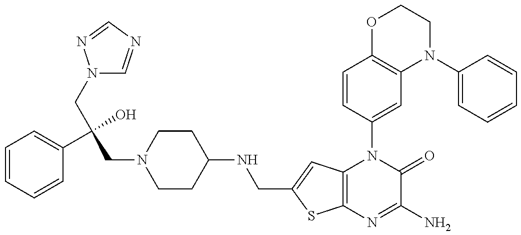 [SMILES: Nc1nc2sc(CNC3CCN(C[C@](O)(Cn4cncn4)c4ccccc4)CC3)cc2n(-c2ccc3c(c2)N(c2ccccc2)CCO3)c1=O] |
| I-157 | 2-amino-4-(2,4-dichloro-6-(3- (2,4-difluorophenyl)-3-hydroxy- 4-(1H-1,2,4-triazol-1- yl)butoxy)phenyl)-N-(2,2- difluoropropyl)-5,7-dihydro-6H- pyrrolo[3,4-d]pyrimidine-6- carboxamide | 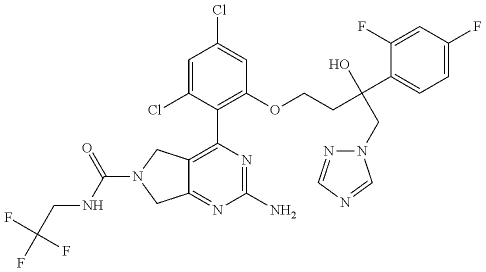 [SMILES: Nc1nc2c(c(-c3c(Cl)cc(Cl)cc3OCCC(O)(Cn3cncn3)c3ccc(F)cc3F)n1)CN(C(=O)NCC(F)(F)F)C2] |
| I-158 | 2,2-difluoropropyl 2-amino-4- (2,4-dichloro-6-((R)-3-(2,4- difluorophenyl)-3-hydroxy-4- (1H-1,2,4-triazol-1- yl)butoxy)phenyl)-5,7-dihydro- 6H-pyrrolo[3,4-d]pyrimidine-6- carboxylate | 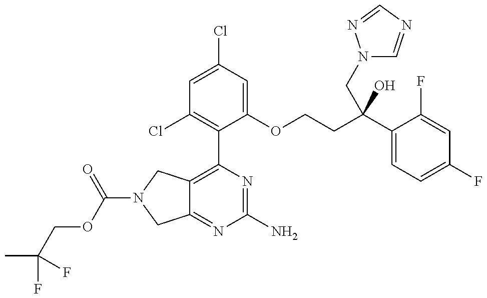 [SMILES: CC(F)(F)COC(=O)N1Cc2nc(N)nc(-c3c(Cl)cc(Cl)cc3OCC[C@](O)(Cn3cncn3)c3ccc(F)cc3F)c2C1] |
| I-159 | 2,2-difluoropropyl 2-amino-4- (2,4-dichloro-6-((S)-3-(2,4- difluorophenyl)-3-hydroxy-4- (1H-1,2,4-triazol-1- yl)butoxy)phenyl)-5,7-dihydro- 6H-pyrrolo[3,4-d]pyrimidine-6- carboxylate | 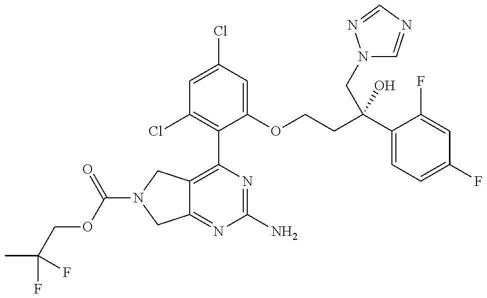 [SMILES: CC(F)(F)COC(=O)N1Cc2nc(N)nc(-c3c(Cl)cc(Cl)cc3OCC[C@@](O)(Cn3cncn3)c3ccc(F)cc3F)c2C1] |
| I-160 | 3-amino-N-(1-(2-hydroxy-3- (1H-imidazol-1-yl)-2- phenylpropyl)piperidin-4-yl)-2- oxo-1-(4-phenyl-3,4-dihydro- 2H-benzo[b][1,4]oxazin-6-yl)- 1,2-dihydrothieno[2,3- b]pyrazine-6-carboxamide | 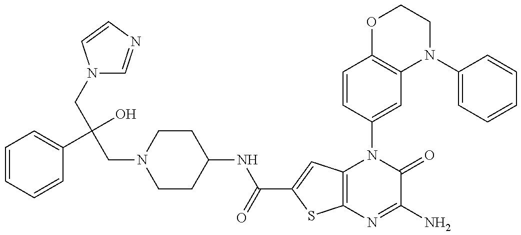 [SMILES: Nc1nc2sc(C(=O)NC3CCN(CC(O)(Cn4ccnc4)c4ccccc4)CC3)cc2n(-c2ccc3c(c2)N(c2ccccc2)CCO3)c1=O] |
| I-161 | 2-amino-4-(2,4-dichloro-6-((R)- 3-(2,4-difluorophenyl)-3- hydroxy-4-(1H-1,2,4-triazol-1- yl)butoxy)phenyl)-N-(2,2- difluoropropyl)-5,7-dihydro-6H- pyrrolo[3,4-d]pyrimidine-6- carboxamide | 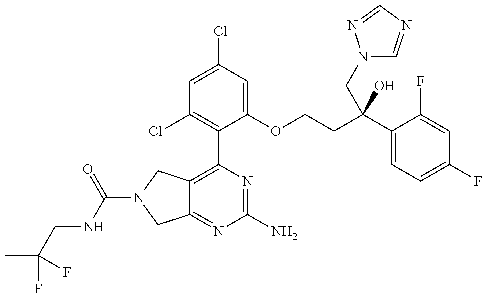 [SMILES: CC(F)(F)CNC(=O)N1Cc2nc(N)nc(-c3c(Cl)cc(Cl)cc3OCC[C@](O)(Cn3cncn3)c3ccc(F)cc3F)c2C1] |
| I-162 | 2-amino-4-(2,4-dichloro-6-((S)- 3-(2,4-difluorophenyl)-3- hydroxy-4-(1H-1,2,4-triazol-1- yl)butoxy)phenyl)-N-(2,2- difluoropropyl)-5,7-dihydro-6H- pyrrolo[3,4-d]pyrimidine-6- carboxamide | 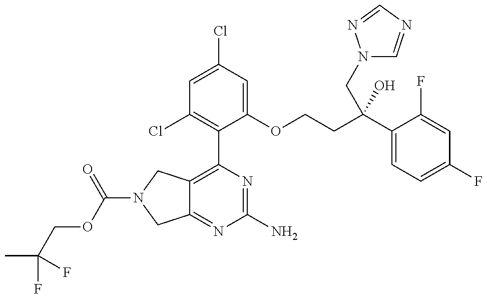 [SMILES: CC(F)(F)COC(=O)N1Cc2nc(N)nc(-c3c(Cl)cc(Cl)cc3OCC[C@@](O)(Cn3cncn3)c3ccc(F)cc3F)c2C1] |
| I-163 | N-(2-(1H-1,2,4-triazol-1- yl)ethyl)-3-amino-2-oxo-1-(4- phenyl-3,4-dihydro-2H- benzo[b][1,4]oxazin-6-yl)-1,2- dihydrothieno[2,3-b]pyrazine- 6-carboxamide | 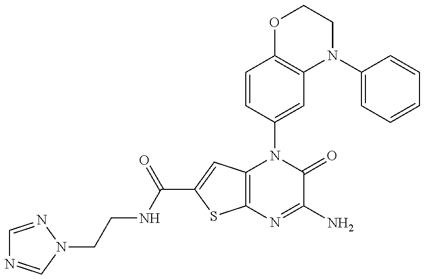 [SMILES: Nc1nc2sc(C(=O)NCCn3cncn3)cc2n(-c2ccc3c(c2)N(c2ccccc2)CCO3)c1=O] |
| I-164 | 3-amino-N-((1-methyl-1H- 1,2,4-triazol-3-yl)methyl)-2- oxo-1-(4-phenyl-3,4-dihydro- 2H-benzo[b][1,4]oxazin-6-yl)- 1,2-dihydrothieno[2,3- b]pyrazine-6-carboxamide | 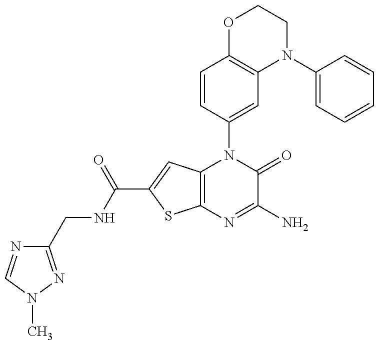 [SMILES: Cn1cnc(CNC(=O)c2cc3c(nc(N)c(=O)n3-c3ccc4c(c3)N(c3ccccc3)CCO4)s2)n1] |
| I-165 | 3-amino-N-(2-hydroxy-2- methyl-3-(1H-1,2,4-triazol-1- yl)propyl)-2-oxo-1-(4-phenyl- 3,4-dihydro-2H- benzo[b][1,4]oxazin-6-yl)-1,2- dihydrothieno[2,3-b]pyrazine- 6-carboxamide | 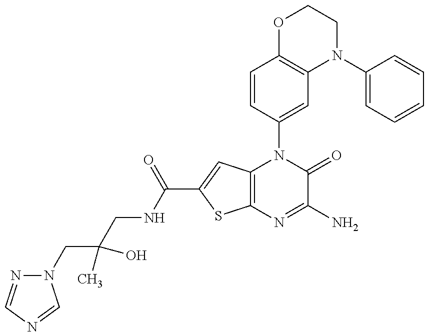 [SMILES: CC(O)(CNC(=O)c1cc2c(nc(N)c(=O)n2-c2ccc3c(c2)N(c2ccccc2)CCO3)s1)Cn1cncn1] |
| I-166 | (R)-N-(1-(2-(4H-1,2,4-triazol-4- yl)ethyl)pyrrolidin-3-yl)-3- amino-2-oxo-1-(4-phenyl-3,4- dihydro-2H- benzo[b][1,4]oxazin-6-yl)-1,2- dihydrothieno[2,3-b]pyrazine- 6-carboxamide | 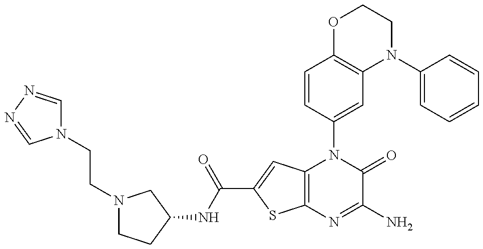 [SMILES: Nc1nc2sc(C(=O)N[C@@H]3CCN(CCn4cnnc4)C3)cc2n(-c2ccc3c(c2)N(c2ccccc2)CCO3)c1=O] |
| I-167 | (R)-3-amino-N-(1-((4-methyl- 4H-1,2,4-triazol-3- yl)methyl)pyrrolidin-3-yl)-2- oxo-1-(4-phenyl-3,4-dihydro- 2H-benzo[b][1,4]oxazin-6-yl)- 1,2-dihydrothieno[2,3- b]pyrazine-6-carboxamide | 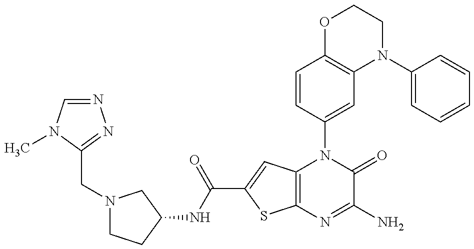 [SMILES: Cn1cnnc1CN1CC[C@@H](NC(=O)c2cc3c(nc(N)c(=O)n3-c3ccc4c(c3)N(c3ccccc3)CCO4)s2)C1] |
| I-168 | 3-amino-N-(2-(1-methyl-1H- 1,2,4-triazol-3-yl)ethyl)-2-oxo- 1-(4-phenyl-3,4-dihydro-2H- benzo[b][1,4]oxazin-6-yl)-1,2- dihydrothieno[2,3-b]pyrazine- 6-carboxamide | 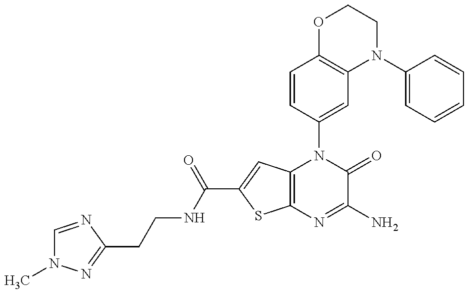 [SMILES: Cn1cnc(CCNC(=O)c2cc3c(nc(N)c(=O)n3-c3ccc4c(c3)N(c3ccccc3)CCO4)s2)n1] |
| I-169 | 3-amino-N-(2-(1-methyl-1H- 1,2,4-triazol-5-yl)ethyl)-2-oxo- 1-(4-phenyl-3,4-dihydro-2H- benzo[b][1,4]oxazin-6-yl)-1,2- dihydrothieno[2,3-b]pyrazine- 6-carboxamide | 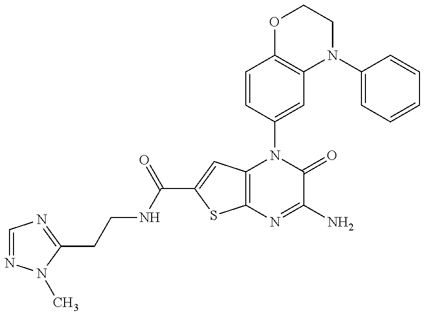 [SMILES: Cn1ncnc1CCNC(=O)c1cc2c(nc(N)c(=O)n2-c2ccc3c(c2)N(c2ccccc2)CCO3)s1] |
| I-170 | 3-amino-N-(1-(2-hydroxy-2- methyl-3-(1H-1,2,4-triazol-1- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 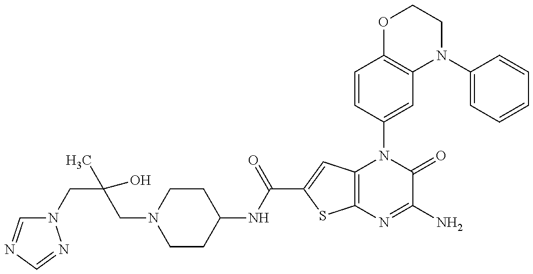 [SMILES: CC(O)(CN1CCC(NC(=O)c2cc3c(nc(N)c(=O)n3-c3ccc4c(c3)N(c3ccccc3)CCO4)s2)CC1)Cn1cncn1] |
| I-171 | 3-amino-N-(1-(3-((4-amino-5- fluoropyrimidin-2- yl)oxy)propyl)piperidin-4-yl)-2- oxo-1-(4-phenyl-3,4-dihydro- 2H-benzo[b][1,4]oxazin-6-yl)- 1,2-dihydrothieno[2,3- b]pyrazine-6-carboxamide | 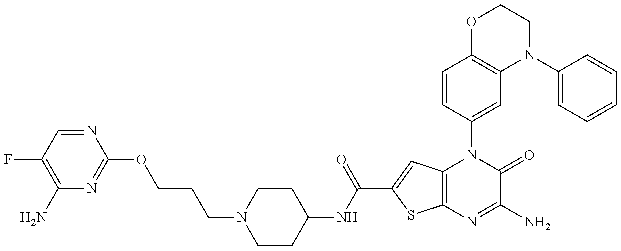 [SMILES: Nc1nc(OCCCN2CCC(NC(=O)c3cc4c(nc(N)c(=O)n4-c4ccc5c(c4)N(c4ccccc4)CCO5)s3)CC2)ncc1F] |
| I-172 | 3-amino-N-(1-(3-(4-amino-5- fluoro-2-oxopyrimidin-1(2H)- yl)propyl)piperidin-4-yl)-2-oxo- 1-(4-phenyl-3,4-dihydro-2H- benzo[b][1,4]oxazin-6-yl)-1,2- dihydrothieno[2,3-b]pyrazine- 6-carboxamide | 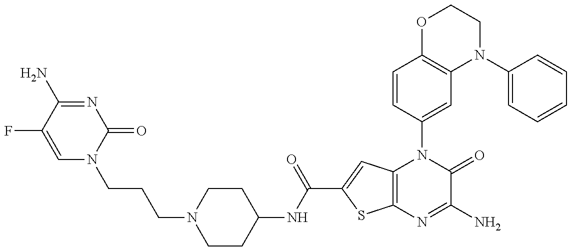 [SMILES: Nc1nc(=O)n(CCCN2CCC(NC(=O)c3cc4c(nc(N)c(=O)n4-c4ccc5c(c4)N(c4ccccc4)CCO5)s3)CC2)cc1F] |
| I-173 | 2-(2-(2,4-difluorophenyl)-2- hydroxy-3-(1H-1,2,4-triazol-1- yl)propyl)-6-(6,6-dimethyl-4- oxo-3-(trifluoromethyl)-4,5,6,7- tetrahydro-1H-pyrazolo[4,3- c]pyridin-1-yl)-8-(((1r,4r)-4- hydroxycyclohexyl)amino)-3,4- dihydroisoquinolin-1(2H)-one | 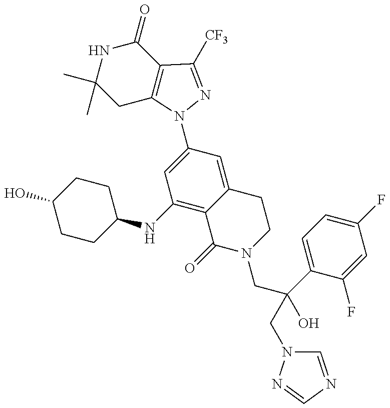 [SMILES: CC1(C)Cc2c(c(C(F)(F)F)nn2-c2cc3c(c(N[C@H]4CC[C@H](O)CC4)c2)C(=O)N(CC(O)(Cn2cncn2)c2ccc(F)cc2F)CC3)C(=O)N1] |
| I-174 | (1r,4r)-4-((2-carbamoyl-5-(3,6,6- trimethyl-4-oxo-4,5,6,7- tetrahydro-1H-indazol-1- yl)phenyl)amino)cyclohexyl 1-(2-(3,5-difluorophenyl)- 2-hydroxy-3- (1H-1,2,4-triazol-1- yl)propyl)piperidine- 4-carboxylate | 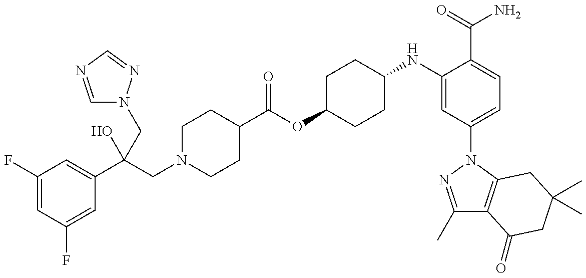 [SMILES: Cc1nn(-c2ccc(C(N)=O)c(N[C@H]3CC[C@H](OC(=O)C4CCN(CC(O)(Cn5cncn5)c5cc(F)cc(F)c5)CC4)CC3)c2)c2c1C(=O)CC(C)(C)C2] |
| I-175 | (1r,4r)-4-((2-carbamoyl-5-(3,6,6- trimethyl-4-oxo-4,5,6,7- tetrahydro-1H-indazol-1- yl)phenyl)amino)cyclohexyl (1-(2-(2,4-difluorophenyl)- 2-hydroxy-3- (1H-1,2,4-triazol-1- yl)propyl)piperidin-4- yl)carbamate | 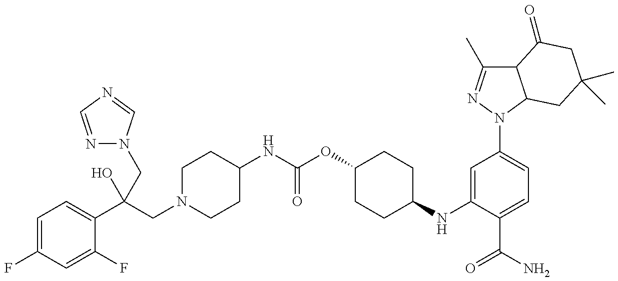 [SMILES: CC1=NN(c2ccc(C(N)=O)c(N[C@H]3CC[C@H](OC(=O)NC4CCN(CC(O)(Cn5cncn5)c5ccc(F)cc5F)CC4)CC3)c2)C2CC(C)(C)CC(=O)C12] |
| I-176 | 2-(((1-(2-(2,4-difluorophenyl)-2- hydroxy-3-(1H-1,2,4-triazol-1- yl)propyl)piperidin-4- yl)methyl)amino)-4-(3,6,6- trimethyl-4-oxo-4,5,6,7- tetrahydro-1H-indazol-1- yl)benzamide | 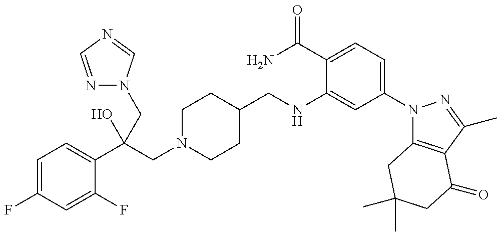 [SMILES: Cc1nn(-c2ccc(C(N)=O)c(NCC3CCN(CC(O)(Cn4cncn4)c4ccc(F)cc4F)CC3)c2)c2c1C(=O)CC(C)(C)C2] |
| I-177 | 2-(3-(2,4-difluorophenyl)-3- hydroxy-4-(1H-1,2,4-triazol-1- yl)butoxy)-4-(3,6,6-trimethyl-4- oxo-4,5,6,7-tetrahydro-1H- indazol-1-yl)benzamide | 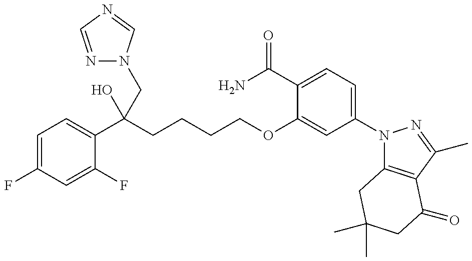 [SMILES: Cc1nn(-c2ccc(C(N)=O)c(OCCCCC(O)(Cn3cncn3)c3ccc(F)cc3F)c2)c2c1C(=O)CC(C)(C)C2] |
| I-178 | 2-((4-(2,4-difluorophenyl)-4- hydroxy-5-(1H-1,2,4-triazol-1- yl)pentyl)oxy)-4- (3,6,6-trimethyl-4- oxo-4,5,6,7-tetrahydro-1H- indazol-1-yl)benzamide | 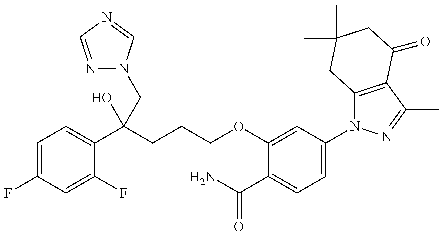 [SMILES: Cc1nn(-c2ccc(C(N)=O)c(OCCCC(O)(Cn3cncn3)c3ccc(F)cc3F)c2)c2c1C(=O)CC(C)(C)C2] |
| I-179 | (R)-2-((1-(2-(2,4-difluorophenyl)- 2-hydroxy-3-(1H-1,2,4-triazol-1- yl)propyl)piperidin-4-yl)amino)-4- (3,6,6-trimethyl-4-oxo-4,5,6,7- tetrahydro-1H-indazol-1- yl)benzamide | 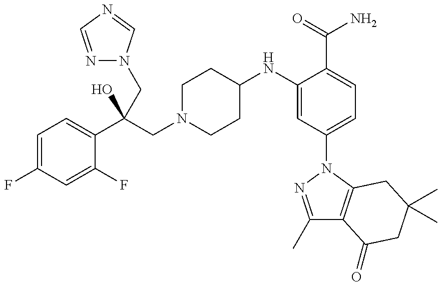 [SMILES: Cc1nn(-c2ccc(C(N)=O)c(NC3CCN(C[C@@](O)(Cn4cncn4)c4ccc(F)cc4F)CC3)c2)c2c1C(=O)CC(C)(C)C2] |
| I-180 | (S)-2-((1-(2-(2,4-difluorophenyl)- 2-hydroxy-3-(1H-1,2,4-triazol-1- yl)propyl)piperidin-4-yl)amino)-4- (3,6,6-trimethyl-4-oxo-4,5,6,7- tetrahydro-1H-indazol-1- yl)benzamide | 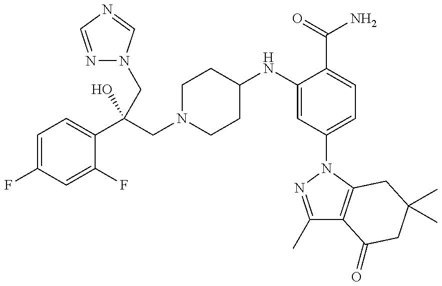 [SMILES: Cc1nn(-c2ccc(C(N)=O)c(NC3CCN(C[C@](O)(Cn4cncn4)c4ccc(F)cc4F)CC3)c2)c2c1C(=O)CC(C)(C)C2] |
| I-181 | (1r,4R)-4-((2-carbamoyl-5-(3,6,6- trimethyl-4-oxo-4,5,6,7- tetrahydro-1H-indazol-1- yl)phenyl)amino)cyclohexyl 1- ((R)-2-(2,4-difluorophenyl)-2- hydroxy-3-(1H-1,2,4-triazol-1- yl)propyl)piperidine-4-carboxylate | 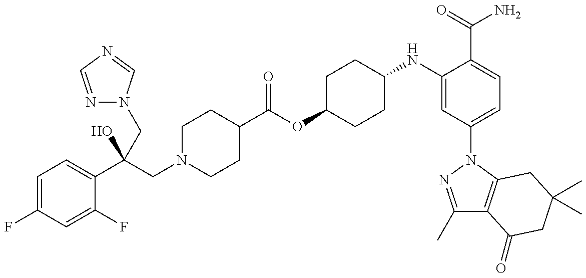 [SMILES: Cc1nn(-c2ccc(C(N)=O)c(N[C@H]3CC[C@H](OC(=O)C4CCN(C[C@@](O)(Cn5cncn5)c5ccc(F)cc5F)CC4)CC3)c2)c2c1C(=O)CC(C)(C)C2] |
| I-182 | (1r,4S)-4-((2-carbamoyl-5-(3,6,6- trimethyl-4-oxo-4,5,6,7- tetrahydro-1H-indazol-1- yl)phenyl)amino)cyclohexyl 1- ((S)-2-(2,4-difluorophenyl)-2- hydroxy-3-(1H-1,2,4-triazol-1- yl)propyl)piperidine-4-carboxylate | 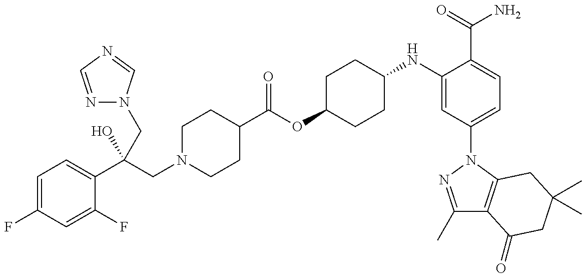 [SMILES: Cc1nn(-c2ccc(C(N)=O)c(N[C@H]3CC[C@H](OC(=O)C4CCN(C[C@](O)(Cn5cncn5)c5ccc(F)cc5F)CC4)CC3)c2)c2c1C(=O)CC(C)(C)C2] |
| I-183 | 2-(((1r,4r)-4-(3-(2,4- difluorophenyl)-3-hydroxy-4-(1H- 1,2,4-triazol-1- yl)butanamido)cyclohexyl)amino)- 4-(3,6,6-trimethyl-4-oxo-4,5,6,7- tetrahydro-1H-indazol-1- yl)benzamide | 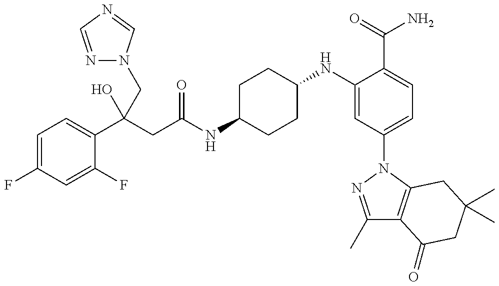 [SMILES: Cc1nn(-c2ccc(C(N)=O)c(N[C@H]3CC[C@H](NC(=O)CC(O)(Cn4cncn4)c4ccc(F)cc4F)CC3)c2)c2c1C(=O)CC(C)(C)C2] |
| I-184 | 2-((1-(2-(2,4-difluorophenyl)-2- hydroxy-3-(1H-1,2,4-triazol-1- yl)propyl)piperidin-4-yl)oxy)-4- (3,6,6-trimethyl-4-oxo-4,5,6,7- tetrahydro-1H-indazol-1- yl)benzamide | 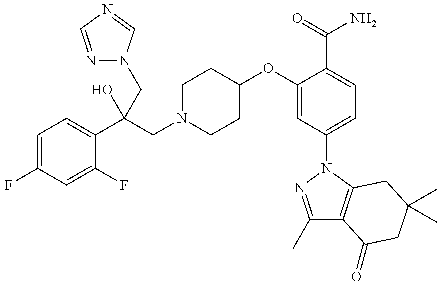 [SMILES: Cc1nn(-c2ccc(C(N)=O)c(OC3CCN(CC(O)(Cn4cncn4)c4ccc(F)cc4F)CC3)c2)c2c1C(=O)CC(C)(C)C2] |
| I-185 | N-((1r,4r)-4-((2-carbamoyl-5- (3,6,6-trimethyl-4-oxo-4,5,6,7- tetrahydro-1H-indazol-1- yl)phenyl)amino)cyclohexyl)-1-(2- (2,4-difluorophenyl)-2-hydroxy-3- (1H-1,2,4-triazol-1- yl)propyl)piperidine-4- carboxamide | 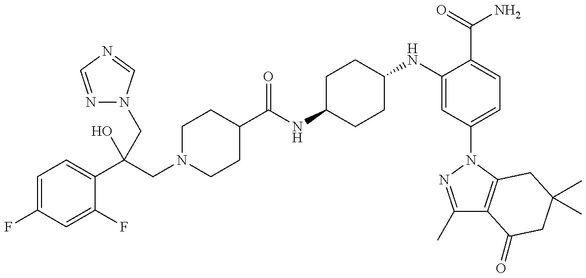 [SMILES: Cc1nn(-c2ccc(C(N)=O)c(N[C@H]3CC[C@H](NC(=O)C4CCN(CC(O)(Cn5cncn5)c5ccc(F)cc5F)CC4)CC3)c2)c2c1C(=O)CC(C)(C)C2] |
| I-186 | (1r,4r)-4-((2-carbamoyl-5-(3,6,6- trimethyl-4-oxo-4,5,6,7- tetrahydro-1H-indazol-1- yl)phenyl)amino)cyclohexyl 3- (2,4-difluorophenyl)-3-hydroxy-4- (1H-1,2,4-triazol-1-yl)butanoate | 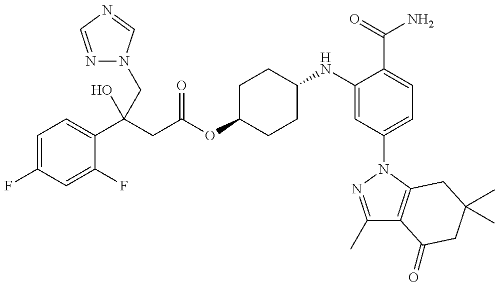 [SMILES: Cc1nn(-c2ccc(C(N)=O)c(N[C@H]3CC[C@H](OC(=O)CC(O)(Cn4cncn4)c4ccc(F)cc4F)CC3)c2)c2c1C(=O)CC(C)(C)C2] |
| I-187 | (1r,4S)-4-((2-carbamoyl-5-(3,6,6- trimethyl-4-oxo-4,5,6,7- tetrahydro-1H-indazol-1- yl)phenyl)amino)cyclohexyl (1- ((S)-2-(2,4-difluorophenyl)-2- hydroxy-3-(1H-1,2,4-triazol-1- yl)propyl)piperidin-4-yl)carbamate | 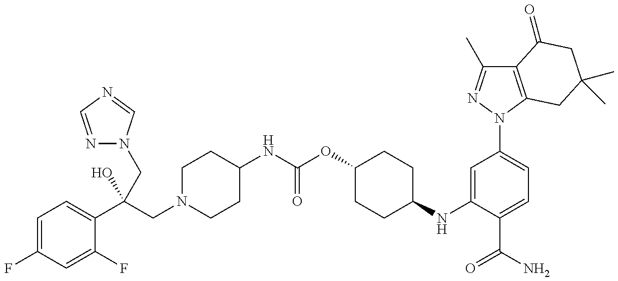 [SMILES: Cc1nn(-c2ccc(C(N)=O)c(N[C@H]3CC[C@H](OC(=O)NC4CCN(C[C@](O)(Cn5cncn5)c5ccc(F)cc5F)CC4)CC3)c2)c2c1C(=O)CC(C)(C)C2] |
| I-188 | (1r,4R)-4-((2-carbamoyl-5-(3,6,6- trimethyl-4-oxo-4,5,6,7- tetrahydro-1H-indazol-1- yl)phenyl)amino)cyclohexyl (1- ((R)-2-(2,4-difluorophenyl)-2- hydroxy-3-(1H-1,2,4-triazol-1- yl)propyl)piperidin-4-yl)carbamate | 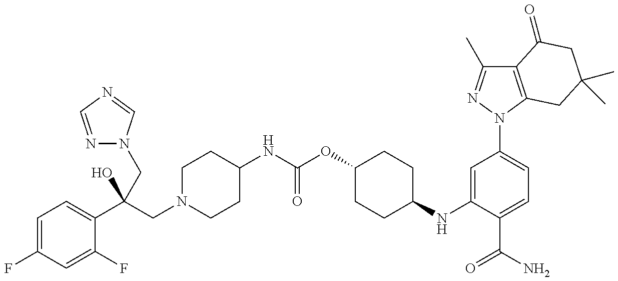 [SMILES: Cc1nn(-c2ccc(C(N)=O)c(N[C@H]3CC[C@H](OC(=O)NC4CCN(C[C@@](O)(Cn5cncn5)c5ccc(F)cc5F)CC4)CC3)c2)c2c1C(=O)CC(C)(C)C2] |
| I-189 | 3-(2,4-difluorophenyl)-3-hydroxy- 4-(1H-1,2,4-triazol-1-yl)butyl 4- (((3-amino-2-oxo-1-(4-phenyl-3,4- dihydro-2H-benzo[b][1,4]oxazin-6- yl)-1,2-dihydrothieno[2,3- b]pyrazin-6- yl)methyl)amino)piperidine-1- carboxylate | 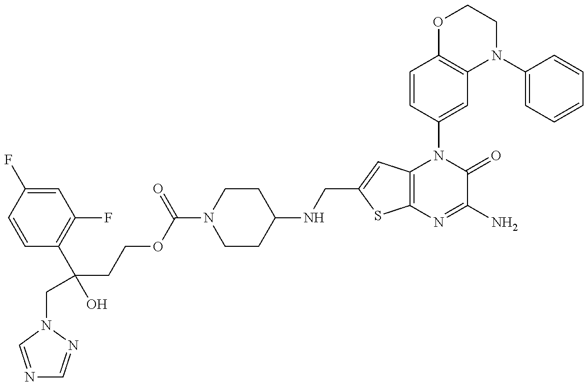 [SMILES: Nc1nc2sc(CNC3CCN(C(=O)OCCC(O)(Cn4cncn4)c4ccc(F)cc4F)CC3)cc2n(-c2ccc3c(c2)N(c2ccccc2)CCO3)c1=O] |
| I-190 | 1-(2-(3,5-difluorophenyl)-2- hydroxy-3-(1H-1,2,4-triazol-1- yl)propyl)piperidin-4-yl ((3-amino- 2-oxo-1-(4-phenyl-3,4-dihydro- 2H-benzo[b][1,4]oxazin-6-yl)-1,2- dihydrothieno[2,3-b]pyrazin-6- yl)methyl)carbamate | 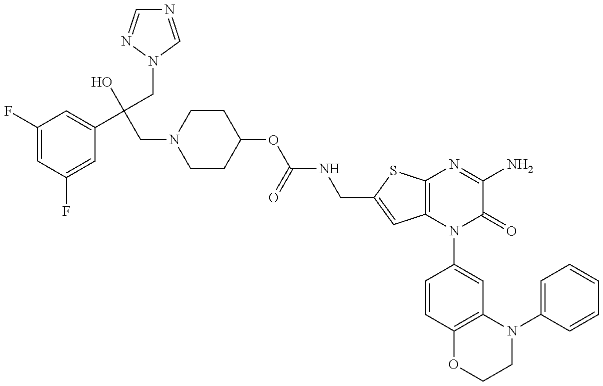 [SMILES: Nc1nc2sc(CNC(=O)OC3CCN(CC(O)(Cn4cncn4)c4cc(F)cc(F)c4)CC3)cc2n(-c2ccc3c(c2)N(c2ccccc2)CCO3)c1=O] |
| I-191 | 2-(3,5-difluorophenyl)-2-hydroxy- 3-(1H-1,2,4-triazol-1-yl)propyl ((3- amino-2-oxo-1-(4-phenyl-3,4- dihydro-2H-benzo[b][1,4]oxazin-6- yl)-1,2-dihydrothieno[2,3- b]pyrazin-6-yl)methyl)carbamate | 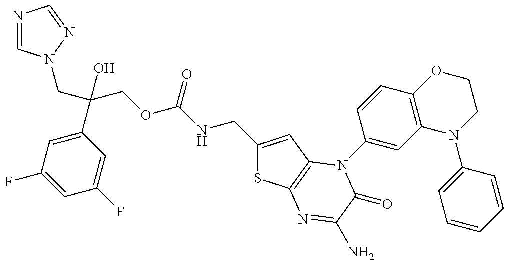 [SMILES: Nc1nc2sc(CNC(=O)OCC(O)(Cn3cncn3)c3cc(F)cc(F)c3)cc2n(-c2ccc3c(c2)N(c2ccccc2)CCO3)c1=O] |
| I-192 | 2-(4-(((3-amino-2-oxo-1-(4- phenyl-3,4-dihydro-2H- benzo[b][1,4]oxazin-6-yl)-1,2- dihydrothieno[2,3-b]pyrazin-6- yl)methyl)amino)piperidin-1-yl)-N- (3-(2,4-difluorophenyl)-3-hydroxy- 4-(1H-1,2,4-triazol-1- yl)butyl)acetamide | 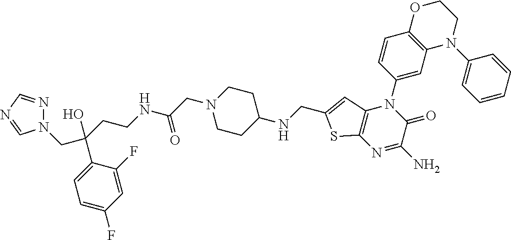 [SMILES: Nc1nc2sc(CNC3CCN(CC(=O)NCCC(O)(Cn4cncn4)c4ccc(F)cc4F)CC3)cc2n(-c2ccc3c(c2)N(c2ccccc2)CCO3)c1=O] |
| I-193 | 2-(2,4-difluorophenyl)-1,3-di(1H- 1,2,4-triazol-1-yl)propan-2-yl 2-(4- (((3-amino-2-oxo-1-(4-phenyl-3,4- dihydro-2H-benzo[b][1,4]oxazin-6- yl)-1,2-dihydrothieno[2,3- b]pyrazin-6- yl)methyl)amino)piperidin-1- yl)acetate | 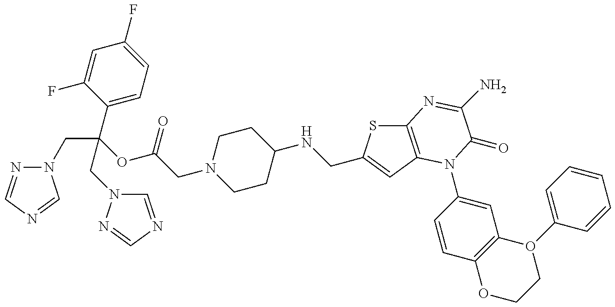 |
| I-194 | 2-(2,4-difluorophenyl)-1,3-di(1H- 1,2,4-triazol-1-yl)propan-2-yl 2-(4- (3-amino-2-oxo-1-(4-phenyl-3,4- dihydro-2H-benzo[b][1,4]oxazin-6- yl)-1,2-dihydrothieno[2,3- b]pyrazine-6- carboxamido)piperidin-1- yl)acetate | 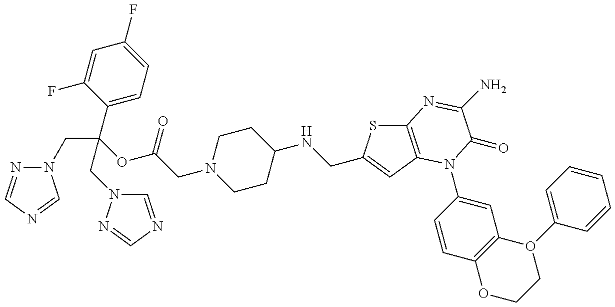 |
| I-195 | (1r,4r)-4-(3-amino-2-oxo-1-(4- phenyl-3,4-dihydro-2H- benzo[b][1,4]oxazin-6-yl)-1,2- dihydrothieno[2,3-b]pyrazine-6- carboxamido)cyclohexyl 1-(2-(2,4- difluorophenyl)-2-hydroxy-3-(1H- 1,2,4-triazol-1- yl)propyl)piperidine-4-carboxylate | 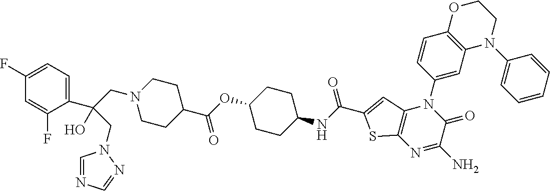 [SMILES: Nc1nc2sc(C(=O)N[C@H]3CC[C@H](OC(=O)C4CCN(CC(O)(Cn5cncn5)c5ccc(F)cc5F)CC4)CC3)cc2n(-c2ccc3c(c2)N(c2ccccc2)CCO3)c1=O] |
| I-196 | (1r,4r)-4-(((3-amino-2-oxo-1-(4- phenyl-3,4-dihydro-2H- benzo[b][1,4]oxazin-6-yl)-1,2- dihydrothieno[2,3-b]pyrazin-6- yl)methyl)amino)cyclohexyl (1-(2- (2,4-difluorophenyl)-2-hydroxy-3- (1H-1,2,4-triazol-1- yl)propyl)piperidin-4-yl)carbamate | 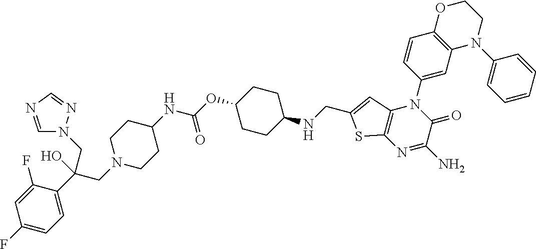 [SMILES: Nc1nc2sc(CN[C@H]3CC[C@H](OC(=O)NC4CCN(CC(O)(Cn5cncn5)c5ccc(F)cc5F)CC4)CC3)cc2n(-c2ccc3c(c2)N(c2ccccc2)CCO3)c1=O] |
| I-197 | (1r,4r)-4-(((3-amino-2-oxo-1-(4- phenyl-3,4-dihydro-2H- benzo[b][1,4]oxazin-6-yl)-1,2- dihydrothieno[2,3-b]pyrazin-6- yl)methyl)amino)cyclohexyl 1-(2- (2,4-difluorophenyl)-2-hydroxy-3- (1H-1,2,4-triazol-1- yl)propyl)piperidine-4-carboxylate | 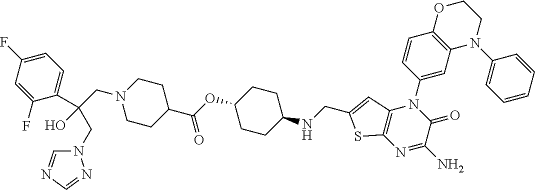 [SMILES: Nc1nc2sc(CN[C@H]3CC[C@H](OC(=O)C4CCN(CC(O)(Cn5cncn5)c5ccc(F)cc5F)CC4)CC3)cc2n(-c2ccc3c(c2)N(c2ccccc2)CCO3)c1=O] |
| I-198 | N-((1r,4r)-4-(((3-amino-2-oxo-1- (4-phenyl-3,4-dihydro-2H- benzo[b][1,4]oxazin-6-yl)-1,2- dihydrothieno[2,3-b]pyrazin-6- yl)methyl)amino)cyclohexyl)-1-(2- (2,4-difluorophenyl)-2-hydroxy-3- (1H-1,2,4-triazol-1- yl)propyl)piperidine-4- carboxamide | 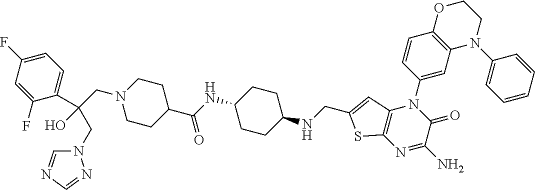 [SMILES: Nc1nc2sc(CN[C@H]3CC[C@H](NC(=O)C4CCN(CC(O)(Cn5cncn5)c5ccc(F)cc5F)CC4)CC3)cc2n(-c2ccc3c(c2)N(c2ccccc2)CCO3)c1=O] |
| I-199 | N-(1-(2-(2,4-difluorophenyl)-2- hydroxy-3-(1H-1,2,4-triazol-1- yl)propyl)piperidin-4-yl)-5-(2,4- dihydroxy-5-isopropylphenyl)-4- (4- (morpholinomethyl)phenyl) isoxazole-3-carboxamide | 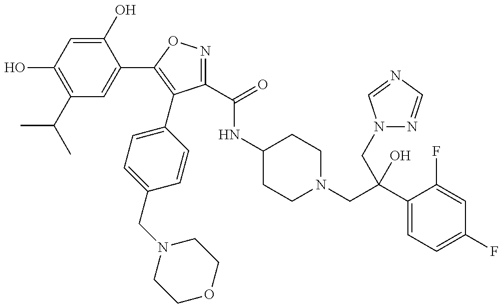 [SMILES: CC(C)c1cc(-c2onc(C(=O)NC3CCN(CC(O)(Cn4cncn4)c4ccc(F)cc4F)CC3)c2-c2ccc(CN3CCOCC3)cc2)c(O)cc1O] |
| I-200 | N-(1-(2-(2,4-difluorophenyl)-2- hydroxy-3-(1H-1,2,4-triazol-1- yl)propyl)piperidin-4-yl)-5-(4,6- dihydroxy-4′-methyl-[1,1′- biphenyl]-3-yl)-4-(4- (morpholinomethyl)phenyl) isoxazole-3-carboxamide | 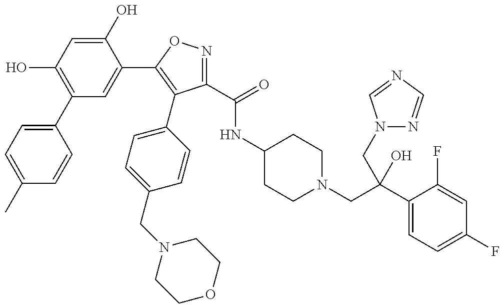 [SMILES: Cc1ccc(-c2cc(-c3onc(C(=O)NC4CCN(CC(O)(Cn5cncn5)c5ccc(F)cc5F)CC4)c3-c3ccc(CN4CCOCC4)cc3)c(O)cc2O)cc1] |

or, an enantiomer thereof (where relevant), or a pharmaceutically acceptable salt, solvate and/or prodrug thereof.

The present application further includes a pharmaceutical composition comprising one or more conjugate compounds of the present application, or a pharmaceutically acceptable salt, solvate and/or prodrug thereof, and a pharmaceutically acceptable carrier and/or diluent. In some embodiments, the pharmaceutical composition further comprising an additional therapeutic agent.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a composition in which the one or more compounds of the application (the active ingredient) is in association with an acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I)), an additional therapeutic agent, and a pharmaceutically acceptable carrier.

To be clear, in the above, the term “a compound” also includes embodiments wherein one or more compounds are referenced.

### III. Methods and Uses of the Application

The compounds of the application have been shown to be capable of inhibiting fungal activity.

Accordingly, the present application further includes a method of treating or prenventing a fungal-related disease, disorder or condition comprising administering a therapeutically effective amount of one or more conjugate compounds of the present application to a subject in need thereof.

The present application also includes a method of inhibiting or preventing fungal growth comprising administering a therapeutically effective amount of one or more conjugate compounds of the present application to a subject in need thereof.

Also provided is a method of inhibiting fungal HSP90 activity comprising administering a therapeutically effective amount of one or more conjugate compounds of the present application to a subject in need thereof. In some embodiments, the method comprises selectively inhibiting fungal HSP90 activity.

The present application further provides a method of treating a fungal-related disease, disorder or condition that is treatable by inhibiting fungal HSP90 comprising administering a therapeutically effective amount of one or more conjugate compounds of the present application in combination with another known agent useful for treatment of a fungal-related disease, disorder or condition that is treatable by inhibiting fungal HSP90 to a subject in need thereof.

The application also includes a use of one or more conjugate compounds of the application for treating or preventing a fungal-related disease, disorder or condition as well as a use of one or more conjugates compounds of the application for the preparation of a medicament for treating or preventing a fungal-related disease, disorder or condition. The application further includes one or more conjugate compounds of the application for use in treating or preventing a fungal-related disease, disorder or condition.

As the conjugate compounds of the application have been shown to be capable of inhibiting fungal activity, the conjugate compounds of the application are useful for treating or preventing a fungal-related disease, disorder or condition by inhibiting fungal activity. Therefore the conjugate compounds of the present application are useful as medicaments. Accordingly, the present application includes a conjugate compound of the application for use as a medicament.

In some embodiments, the fungal-related disease, disorder or condition comprises mycosis including superficial, subcutaneous and systemic mycosis. In some embodiments, the fungal-related disease, disorder or condition includes dermatomycosis, candidiasis, pneumocytosis, Pityriasis versicolor, aspergillosis, mucormycosis, talaromycosis, basidiobolomycosis, blastomycosis, chromoblastomycosis cryptococcosis, coccidioidomycosis, conidiobolomycosis, eumycetoma, histoplasmosis, lobomycosis, paracoccidioidomycosis, phaeohyphomycosis, scedosporisis, sporotrichosis and emmonsiosis.

Accordingly, the present application thus provides a method of treating or preventing a fungal infection or mycosis comprising administering a therapeutically effective amount of one or more conjugate compounds of the present application to a subject in need thereof. In some embodiments, the mycosis is candidiasis,

In some embodiments, fungi that cause infections include the yeasts, molds, and/or dimorphic fungi responsible for the infections specified herein.

When used in combination with other agents or therapies useful in treating fungal-related diseases, disorders or conditions, it is an embodiment that the conjugate compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

In some embodiments, the subject is a mammal. In some embodiments, the subject is a livestock, such as cattle, sheep, goat, poultry, etc. In some embodiments, the subject is human. In some embodiments, mycoses in human affect skin, mucosa such as mouth and vagina, nails, organs such as lungs and brain, eyes, nose, sinuses, bones, joints, etc.

In some embodiments, the subject is a crop, such as rice, wheat, barley, oat, rye, sugarcane and other sugar crops, maize (corn), potatoes, palm, canola, flax, safflower cassava, legume pulses such as beans, soybeans, peas, chickpeas, peanuts, lentils, lupins, mesquite, carob, tamarind, alfalfa, and clover, sunflower, rape, mustard, sorghum, millet, hemp, sugar beet, groundnuts, sweet potatoes, bananas, cotton, yarns, various nuts or other vegetables or fruits.

In the context of treating a fungal-related disease, disorder or condition, an effective amount is an amount that, for example, inhibits fungal activity, compared to the inhibition without administration of the one or more conjugate compounds. Effective amounts may vary according to factors such as the disease state, age, sex and/or weight of the subject, type of fungi. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 pg/cc to about 1000 pg/cc, or about 0.1 pg/cc to about 100 pg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

### IV. Methods of Preparing the Compounds of the Application

Compounds of the present application can be prepared by various synthetic processes. The choice of particular structural features and/or substituents may influence the selection of one process over another. The selection of a particular process to prepare a given conjugate compound of Formula (I) is within the purview of the person of skill in the art. Some starting materials for preparing compounds of the present application are available from commercial chemical sources. Other starting materials, are readily prepared from available precursors using straightforward transformations that are well known in the art.

In some embodiments, the compounds of Formula I are assembled by attaching the linker group to one of A or B following by attachment of the other of A or B. Standard chemistries known in the art can be used to assemble the compounds of Formula I, including but not limited to, nucleophilic displacements, cross-couplings, Michael reactions and/or activating group strategies. As many of the A and B groups are known, or are based on known compounds, compounds that can be used to react with the linker group are readily available either from commercial sources or using synthetic methods known in the art. For example, many such compounds have at least one functional group in their structure capable of forming a covalent bond with the linker group. Examples of such include an amine, thiol, halo, hydroxyl, alkoxy, carboxyl, ester, amide and/or oxo group.

Throughout the processes it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T.W. Green, P.G.M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R.C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

The following non-limiting examples are illustrative of the present application.

### EXAMPLES

### General Methods

All starting materials used herein were commercially available or earlier described in the literature. The ¹H and ¹³C NMR spectra were recorded either on Bruker 300, Bruker DPX400 or Varian+400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Preparation of Intermediates and Examples

Scheme III outlines the synthesis of compounds of Formula I, represented by BRI-2121, when R₁ is amine, R₂ is aryl moiety R₃ is hydrogen and R₄ group is amide.

### Synthesis of 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-6-carboxamide (I-4)

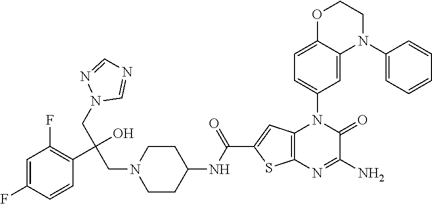 [SMILES: Nc1nc2sc(C(=O)NC3CCN(CC(O)(Cn4cncn4)c4ccc(F)cc4F)CC3)cc2n(-c2ccc3c(c2)N(c2ccccc2)CCO3)c1=O]

### Synthesis of 4-phenyl-3,4-dihydro-2H-benzo[b][1,4]oxazin-6-amine (1-4)

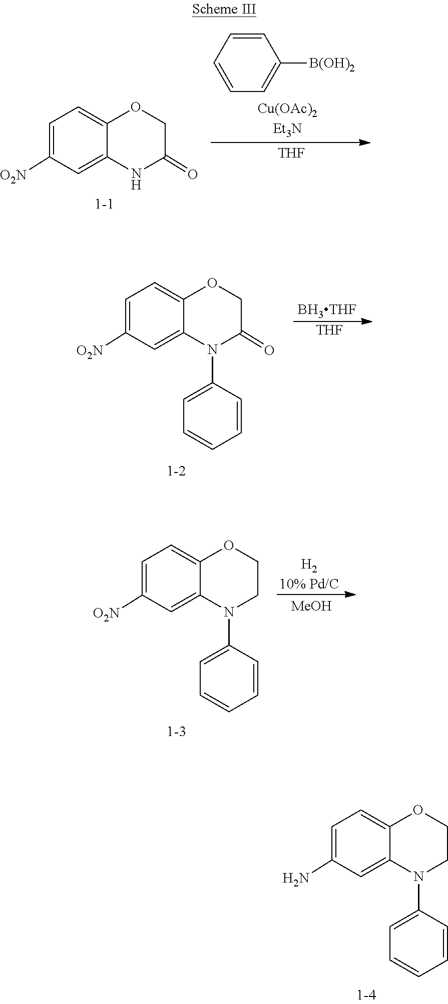

### 6-nitro-4-phenyl-2H-benzo[b][1,4]oxazin-3(4H)-one (1-2)

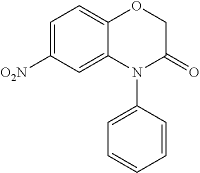 [SMILES: O=C1COc2ccc([N+](=O)[O-])cc2N1c1ccccc1]

To a solution of 6-nitro-2H-benzo[b][1,4]oxazin-3(4H)-one (50 g, 257 mmol) in THF (500 mL) was added molecular sieves (50 g), Cu(OAc)₂ (46 g, 257 mmol), Et₃N (52 g, 514 mmol) and phenylboronic acid (47 g, 385 mmol). The reaction mixture was stirred at 60° C. overnight. The reaction mixture was filtered and concentrated. The crude product was purified by column chromatography on silica gel (petroleum ether/EtOAc=10:1) to give intermediate 6-nitro-4-phenyl-2H-benzo[b][1,4]oxazin-3(4H)-one 1-2 as a red solid (36 g, 52% yield).

LCMS: Calculated Exact Mass=270.1, Found [M+H]⁺ (ESI+)=271.1.

### 6-nitro-4-phenyl-3,4-dihydro-2H-benzo[b][1,4]oxazine (1-3)

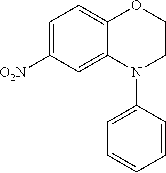 [SMILES: O=[N+]([O-])c1ccc2c(c1)N(c1ccccc1)CCO2]

To a solution of 6-nitro-4-phenyl-2H-benzo[b][1,4]oxazin-3(4H)-one 1-2 (70.0 g, 258 mmol) in THF (500 mL) was added BH₃-THF (1 M, 774 mL, 774 mmol) drop-wise at 0° C. The reaction mixture was stirred at room temperature for 30 minutes and then heated to 80° C. overnight. The reaction mixture was cooled down and MeOH (500 mL) was added. The mixture was refluxed for 1 hour and concentrated. The crude product was purified by column chromatography on silica gel (petroleum ether/EtOAc=5:1) to give 6-nitro-4-phenyl-3,4-dihydro-2H-benzo[b][1,4]oxazine 1-3 as a red solid (61 g, 92% yield).

LCMS: Calculated Exact Mass=256.1, Found [M+H]⁺ (ESI+)=257.2.

### 4-phenyl-3,4-dihydro-2H-benzo[b][1,4]oxazin-6-amine (1-4)

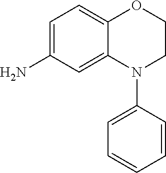 [SMILES: Nc1ccc2c(c1)N(c1ccccc1)CCO2]

To a solution of 6-nitro-4-phenyl-3,4-dihydro-2H-benzo[b][1,4]oxazine 1-3 (60.0 g, 233 mmol) in MeOH (1000 mL) was added 10% Pd/C (6.0 g, 10% wt). The reaction mixture was stirred at room temperature overnight under H₂ atmosphere. The reaction mixture was filtered and concentrated to give crude 4-phenyl-3,4-dihydro-2H-benzo[b][1,4]oxazin-6-amine 1-4 as a red solid (48 g, 80% yield).

LCMS: Calculated Exact Mass=226.1, Found [M+H]⁺ (ESI+)=227.1.

### Synthesis of 3-amino-2-oxo-1-(4-phenyl-3,4-dihydro-2H-benzo[b][1,4]oxazin-6-yl)-1,2-dihydrothieno[2,3-b]pyrazine-6-carboxylic acid (1)

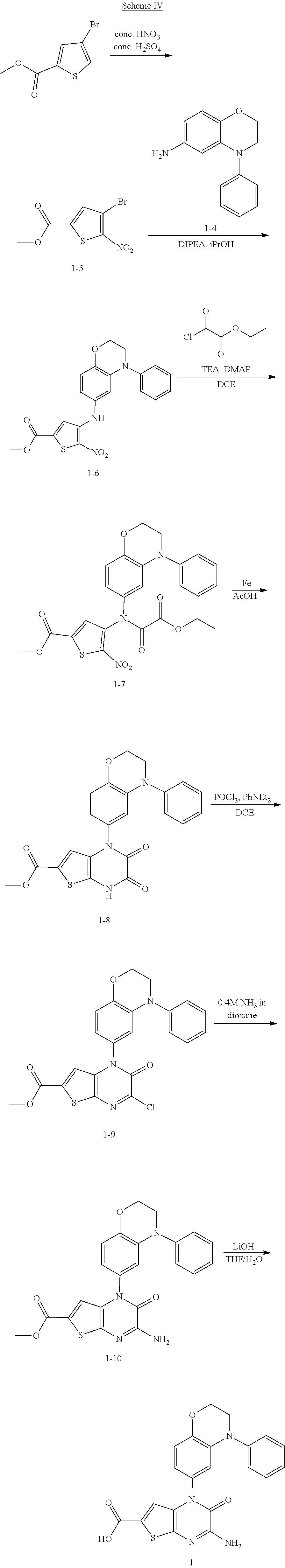

### Methyl 4-bromo-5-nitrothiophene-2-carboxylate (1-5)

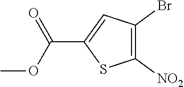

Methyl 4-bromothiophene-2-carboxylate (100 g, 452 mmol) in H₂SO₄ (250 mL) was cooled to 0° C. and fuming nitric acid (45 mL) was added drop by drop over 1 hour. The reaction was stirred for 1 hour at room temperature. The reaction mixture was poured into 2 L of ice-water, resulting in a pale yellow precipitate that was isolated by suction filtration and washed with cold water. The solid was dried by vacuum to afford methyl 4-bromo-5-nitrothiophene-2-carboxylate 1-5 as a pale yellow solid (116 g, 96% yield).

¹H NMR (400 MHz, DMSO) σ=8.00 (s, 1H), 3.91 (s, 3H).

### Methyl-5-nitro-4-((4-phenyl-3,4-dihydro-2H-benzo[b][1,4]oxazin-6-yl)amino)thiophene-2-carboxylate (1-6)

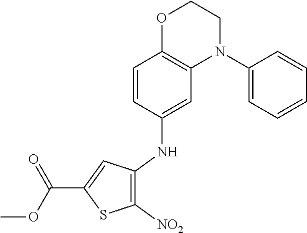 [SMILES: COC(=O)c1cc(Nc2ccc3c(c2)N(c2ccccc2)CCO3)c([N+](=O)[O-])s1]

To a solution of 4-phenyl-3,4-dihydro-2H-benzo[b][1,4]oxazin-6-amine 1-4 (50.0 g, 220 mmol) and methyl 4-bromo-5-nitrothiophene-2-carboxylate 1-5 (69.0 g, 259 mmol) in isopropyl alcohol (500 mL) was added DIPEA (57.2 g, 440 mmol). The reaction mixture was stirred at 90° C. overnight. The reaction mixture was cooled down and concentrated. The crude product was purified by column chromatography on silica gel (petroleum ether/EtOAc=4:1) to give methyl-5-nitro-4-((4-phenyl-3,4-dihydro-2H-benzo[b][1,4]oxazin-6-yl)amino)thiophene-2-carboxylate 1-6 as a red solid (62.1 g, 68% yield).

LCMS: Calculated Exact Mass=411.1, Found [M+H]⁺ (ESI+)=412.2.

### Methyl-4-(2-ethoxy-2-oxo-N-(4-phenyl-3,4-dihydro-2H-benzo[b][1,4]oxazin-6-yl)acetamido)-5-nitrothiophene-2-carboxylate (1-7)

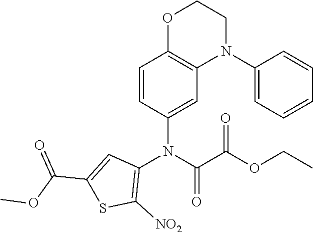 [SMILES: CCOC(=O)C(=O)N(c1ccc2c(c1)N(c1ccccc1)CCO2)c1cc(C(=O)OC)sc1[N+](=O)[O-]]

To a solution of methyl-5-nitro-4-((4-phenyl-3,4-dihydro-2H-benzo[b][1,4]oxazin-6-yl)amino)thiophene-2-carboxylate 1-6 (60.0 g, 145 mmol) and Et₃N (44.1 g, 436 mmol) in DCE (360 mL) was added DMAP (1.7 g, 14.5 mmol). Ethyl 2-chloro-2-oxoacetate (21.7 g, 160 mmol) was added to the reaction mixture drop-wise at 0° C. and stirred at 0° C. for 2 hours. The reaction mixture was quenched with H₂O (500 mL) and extracted with DCM (500 mL×2). The organic phase was washed with brine (500 mL×2), dried over anhydrous Na₂SO₄, filtered and concentrated under reduced pressure to give crude methyl-4-(2-ethoxy-2-oxo-N-(4-phenyl-3,4-dihydro-2H-benzo[b][1,4]oxazin-6-yl)acetamido)-5-nitrothiophene-2-carboxylate 1-7 as a red solid (70 g, 94% yield).

LCMS: Calculated Exact Mass=511.1, Found [M+H]⁺ (ESI+)=512.2.

### Methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate (1-8)

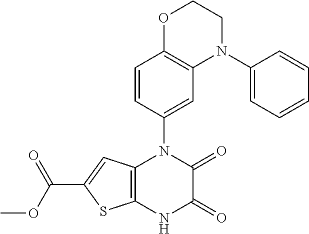 [SMILES: COC(=O)c1cc2c([nH]c(=O)c(=O)n2-c2ccc3c(c2)N(c2ccccc2)CCO3)s1]

To a solution of methyl-4-(2-ethoxy-2-oxo-N-(4-phenyl-3,4-dihydro-2H-benzo[b][1,4]oxazin-6-yl)acetamido)-5-nitrothiophene-2-carboxylate 1-7 (100.0 g, 195 mmol) in AcOH (1000 mL) was added iron powder (54.6 g, 976 mmol). The reaction mixture was stirred at 50° C. for 2 hours. The reaction mixture was filtered and concentrated. H₂O (500 mL) was added to the residue and extracted with DCM (500 mL×3). The organic phase was concentrated under reduced pressure and purified by column chromatography on silica gel (DCM/MeOH=10:1) to give methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate 1-8 as a red solid (61.9 g, 73% yield).

LCMS: Calculated Exact Mass=435.1, Found [M+H]⁺ (ESI+)=436.3.

¹H NMR (400 MHz, DMSO) σ 7.36 (t, J=7.6 Hz, 2H), 7.28 (d, J=7.8 Hz, 2H), 7.08 (t, J=7.1 Hz, 1H), 6.99 (d, J=9.0 Hz, 1H), 6.73 (s, 1H), 6.71-6.67 (m, 2H), 4.35 (t, J=4.1 Hz, 2H), 3.76 (t, J=4.0 Hz, 2H), 3.69 (s, 3H).

### Methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate (1-9)

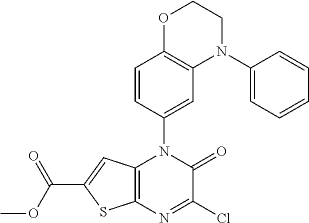 [SMILES: COC(=O)c1cc2c(nc(Cl)c(=O)n2-c2ccc3c(c2)N(c2ccccc2)CCO3)s1]

To a solution of methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate 1-8 (20.0 g, 45.8 mmol) and N,N-diethylaniline (20.6 g, 137.4 mmol) in DCE (400 mL) was added POCl₃ (21.0 g, 137.4 mmol) at 0° C. The reaction mixture was stirred at 90° C. for 4 hours. The reaction mixture was cooled down and poured in ice-water (200 mL), extracted with DCM (500 mL). The organic phase was concentrated under reduced pressure and purified by column chromatography on silica gel (DCM/MeOH=10:1) to give methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate 1-9 as a yellow solid (8.5 g, 41% yield).

LCMS: Calculated Exact Mass=453.1, Found [M+H]⁺ (ESI+)=454.1.

### Methyl-3-amino-2-oxo-1-(4-phenyl-3,4-dihydro-2H-benzo[b][1,4]oxazin-6-yl)-1,2-dihydrothieno[2,3-b]pyrazine-6-carboxylate (1-10)

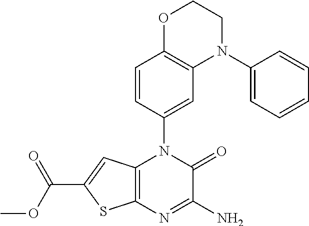 [SMILES: COC(=O)c1cc2c(nc(N)c(=O)n2-c2ccc3c(c2)N(c2ccccc2)CCO3)s1]

Methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate 1-9 (20.0 g, 44.0 mmol) was dissolved in 0.4 M dioxane solution of ammonia (500 mL) and the mixture was stirred at 110° C. overnight. The reaction mixture was cooled down and concentrated under reduced pressure and the crude product was purified by column chromatography on silica gel (DCM/MeOH=10:1) to give methyl-3-amino-2-oxo-1-(4-phenyl-3,4-dihydro-2H-benzo[b][1,4]oxazin-6-yl)-1,2-dihydrothieno[2,3-b]pyrazine-6-carboxylate 1-10 as a yellow solid (16.1 g, 84% yield).

LCMS: Calculated Exact Mass=434.1, Found [M+H]⁺ (ESI+)=435.1.

¹H NMR (400 MHz, DMSO) σ 7.61 (br, 2H), 7.35 (t, J=7.8 Hz, 2H), 7.28 (d, J=7.4 Hz, 2H), 7.09 (t, J=7.2 Hz, 1H), 7.02 (d, J=8.4 Hz, 1H), 6.86 (s, 1H), 6.84 (d, J=2.4 Hz, 1H), 6.77 (dd, J=8.4, 2.4 Hz, 1H), 4.36 (t, J=4.1 Hz, 2H), 3.81-3.70 (m, 5H).

### 3-amino-2-oxo-1-(4-phenyl-3,4-dihydro-2H-benzo[b][1,4]oxazin-6-yl)-1,2-dihydrothieno[2,3-b]pyrazine-6-carboxylic acid (1)

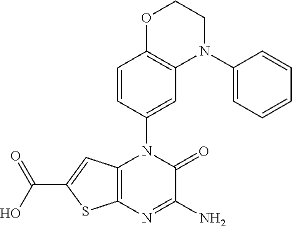 [SMILES: Nc1nc2sc(C(=O)O)cc2n(-c2ccc3c(c2)N(c2ccccc2)CCO3)c1=O]

To a solution of methyl-3-amino-2-oxo-1-(4-phenyl-3,4-dihydro-2H-benzo[b][1,4]oxazin-6-yl)-1,2-dihydrothieno[2,3-b]pyrazine-6-carboxylate 1-10 (10.0 g, 23.0 mmol) in THF (200 mL) was added a solution of LiOH (5.5 g, 229 mmol) in H₂O (200 mL). The mixture was stirred at 50° C. overnight. The reaction mixture was cooled down and the pH was adjusted to 3. The precipitate was collected by filtration and washed with H₂O (50 mL) and dried under reduced pressure to give 3-amino-2-oxo-1-(4-phenyl-3,4-dihydro-2H-benzo[b][1,4]oxazin-6-yl)-1,2-dihydrothieno[2,3-b]pyrazine-6-carboxylic acid 1 as a yellow solid (8.1 g, 84% yield).

LCMS: Calculated Exact Mass=420.1, Found [M+H]⁺ (ESI+)=421.1.

¹H NMR (400 MHz, DMSO) σ 12.96 (br, 1H), 7.56 (br, 2H), 7.38-7.34 (m, 2H), 7.30-7.27 (m, 2H), 7.09 (dd, J=10.3, 4.2 Hz, 1H), 7.03 (d, J=8.4 Hz, 1H), 6.84 (d, J=2.4 Hz, 1H), 6.82 (s, 1H), 6.78 (dd, J=8.4, 2.4 Hz, 1H), 4.36 (t, J=4.1 Hz, 2H), 3.77 (d, J=2.2 Hz, 2H).

### Synthesis of compound 1-4-3-amino-N-(1-(2-(2,4-difluorophenyl)-2-hydroxy-3-(1H-1,2,4-triazol-1-yl)propyl)86iperidine-4-yl)-2-oxo-1-(4-phenyl-3,4-dihydro-2H-benzo[b][1,4]oxazin-6-yl)-1,2-dihydrothieno[2,3-b]pyrazine-6-carboxamide (I-4)

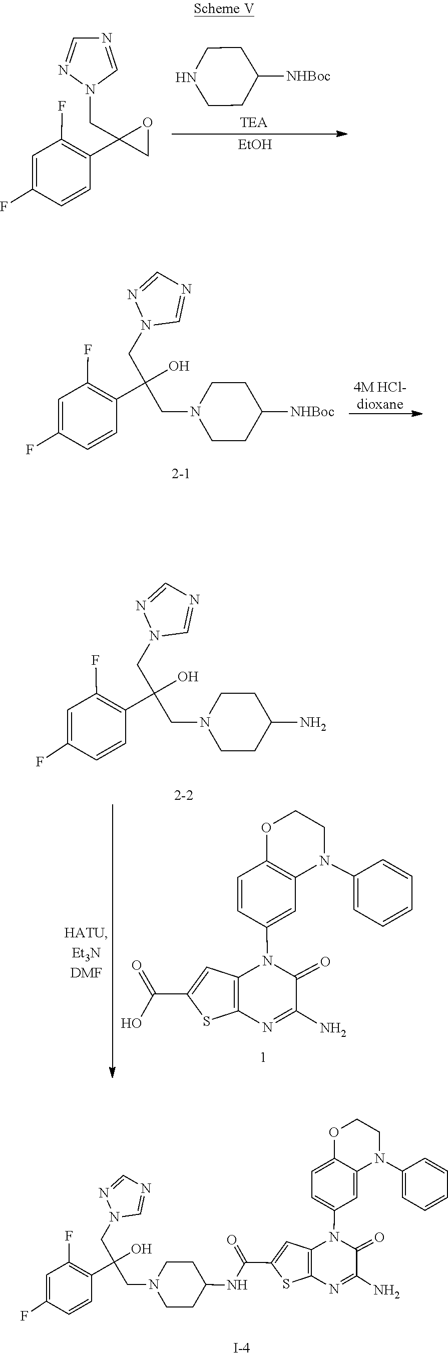

### Tert-butyl-(1-(2-(2,4-difluorophenyl)-2-hydroxy-3-(1H-1,2,4-triazol-1-yl)propyl)piperidin-4-yl) carbamate (2-1)

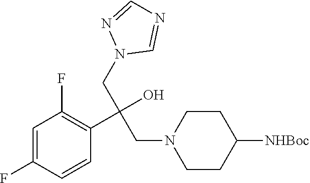 [SMILES: CC(C)(C)OC(=O)NC1CCN(CC(O)(Cn2cncn2)c2ccc(F)cc2F)CC1]

1-((2-(2,4-difluorophenyl)oxiran-2-yl)methyl)-1H-1,2,4-triazole (17.0 g, 71.4 mmol) was dissolved in EtOH (200 mL) and treated with 4-N-Boc-aminopiperidine (21.4 g, 107 mmol) and triethylamine (21.6 g, 214 mmol). The mixture was stirred at 80° C. for 6 hours. When the reaction was complete, the solvent was evaporated and the product was isolated by flash chromatography (DCM/MeOH=10/1) to give Tert-butyl-(1-(2-(2,4-difluorophenyl)-2-hydroxy-3-(1H-1,2,4-triazol-1-yl)propyl)piperidin-4-yl)carbamate 2-1 as a yellow solid (29 g, 93% yield).

LCMS: Calculated Exact Mass=437.2, Found [M+H]⁺ (ESI+)=438.2.

¹H NMR (400 MHz, DMSO) σ 8.30 (s, 1H), 7.74 (s, 1H), 7.39 (dd, J=15.9, 8.9 Hz, 1H), 7.15 (ddd, J=11.7, 9.2, 2.4 Hz, 1H), 6.95 (td, J=8.5, 2.4 Hz, 1H), 6.71 (d, J=7.6 Hz, 1H), 5.64 (s, 1H), 4.62-4.45 (m, 2H), 3.11 (d, J=7.0 Hz, 1H), 2.83 (d, J=13.9 Hz, 1H), 2.74-2.58 (m, 3H), 2.11 (t, J=11.6 Hz, 2H), 1.53-1.51 (m, 2H), 1.35 (s, 9H), 1.29-1.17 (m, 2H).

### Tert-butyl-(1-(2-(2,4-difluorophenyl)-2-hydroxy-3-(1H-1,2,4-triazol-1-yl)propyl)piperidin-4-yl)carbamate (2-2)

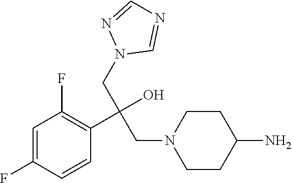 [SMILES: NC1CCN(CC(O)(Cn2cncn2)c2ccc(F)cc2F)CC1]

To a solution of tert-butyl-(1-(2-(2,4-difluorophenyl)-2-hydroxy-3-(1H-1,2,4-triazol-1-yl) propyl)piperidin-4-yl)carbamate 2-1 (29 g, 66.3 mmol) in DCM (100 mL) was added the HCl-dioxane (4 M, 83 mL, 332 mmol). The reaction mixture was stirred for 2 hours at room temperature. When the reaction was complete, the solvent was evaporated to afford crude product. The crude product was dissolved in MeOH (200 mL) and treated with sodium methanolate (10.7 g, 3.0 equiv). The mixture was stirred at room temperature for 4 hours. The solution was filtered and concentreated. The residue was dissolved in DCM (50 mL), filtered and concentrated for three times to afford tert-butyl-(1-(2-(2,4-difluorophenyl)-2-hydroxy-3-(1H-1,2,4-triazol-1-yl)propyl)piperidin-4-yl)carbamate 2-2 as a yellow oil (19 g, 85% yield).

LCMS: Calculated Exact Mass=337.2, Found [M+H]⁺ (ESI+)=338.2.

¹H NMR (400 MHz, MeOD) δ 8.34 (s, 1H), 7.74 (s, 1H), 7.50-7.44 (m 1H), 6.95-6.82 (m, 2H), 4.62 (dd, J=38.0, 14.3 Hz, 2H), 2.98 (dd, J=13.8, 1.6 Hz, 1H), 2.80-2.71 (m, 2H), 2.65-2.50 (m, 2H), 2.34 (td, J=11.8, 5.9 Hz, 1H), 2.15 (td, J=11.8, 2.6 Hz, 1H), 1.77-1.69 (m, 1H), 1.67-1.60 (m, 1H), 1.45-1.23 (m, 2H).

### 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-6-carboxamide (I-4)

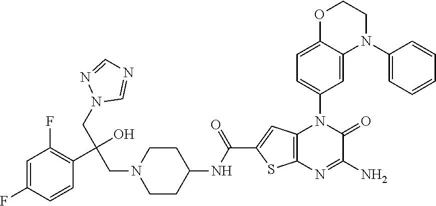 [SMILES: Nc1nc2sc(C(=O)NC3CCN(CC(O)(Cn4cncn4)c4ccc(F)cc4F)CC3)cc2n(-c2ccc3c(c2)N(c2ccccc2)CCO3)c1=O]

3-amino-2-oxo-1-(4-phenyl-3,4-dihydro-2H-benzo[b][1,4]oxazin-6-yl)-1,2-dihydrothieno[2,3-b]pyrazine-6-carboxylic acid 1 (50 mg, 0.11 mmol) was dissolved in DMF (3 mL) and treated with Et₃N (33 mg, 0.33 mmol) and HATU (54 mg, 0.14 mmol) followed by tert-butyl-(1-(2-(2,4-difluorophenyl)-2-hydroxy-3-(1H-1,2,4-triazol-1-yl)propyl)piperidin-4-yl)carbamate 2-2 (74 mg, 0.22 mmol) at 0° C. The mixture was stirred at room temperature for 3 hours. The reaction mixture was diluted with H₂O (20 mL), extracted with EtOAc (50 mL), dried over Na₂SO₄, filtered and concentrated. The crude was purified by Prep-HPLC (ACN/H₂O with 0.5% NH₃ as the mobile phase) to afford 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-6-carboxamide 1-4 as a white solid (4.3 mg, 5.3% yield).

LCMS: Calculated Exact Mass=739.3, Found [M+H]⁺ (ESI+)=740.2.

¹H NMR (400 MHz, DMSO) σ 8.27 (s, 1H), 8.16 (d, J=7.9 Hz, 1H), 7.75 (s, 1H), 7.42-7.25 (m, 6H), 7.20 (s, 1H), 7.14 (ddd, J=11.8, 9.2, 2.4 Hz, 1H), 7.08 (dt, J=12.1, 4.1 Hz, 1H), 7.03 (d, J=8.3 Hz, 1H), 6.94 (td, J=8.5, 2.2 Hz, 1H), 6.80 (d, J=2.2 Hz, 1H), 6.77 (dd, J=8.4, 2.3 Hz, 1H), 5.66 (s, 1H), 4.60-4.47 (m, 2H), 4.46-4.26 (m, 2H), 3.86-3.70 (m, 2H), 3.67-3.55 (m, 2H), 2.96-2.86 (m, 1H), 2.84-2.72 (m, 1H), 2.70-2.57 (m, 2H), 2.21-2.06 (m, 2H), 1.63-1.49 (m, 2H), 1.43-1.24 (m, 2H).

In a similar manner, using the above procedure, the following amide compounds were synthesized.

| Compound | | | Yield | |
|---|---|---|---|---|
| ID# | ¹H NMR | LCMS | % | Appearance |
| I-1 | ¹H NMR: (400 MHz, DMSO-d₆) δ 8.32 (t, J = 5.6 Hz, | Calculated | 14.9% | White |
| | 1H), 8.29 (s, 1H), 7.77 (s, 1H), 7.41 (td, J = 9.0, 6.8 Hz, | exact mass = | | solid |
| | 2H), 7.33 (d, J = 5.6 Hz, 5H), 7.16 (t, J = 10.6 Hz, 1H), | 671.7/ | | |
| | 7.11-7.06 (m, 2H), 7.03 (d, J = 8.4 Hz, 1H), 6.97 (s, | Found | | |
| | 1H), 6.80 (d, J = 2.4 Hz, 1H), 6.76 (dd, J = 8.5, 2.4 Hz, | [M + H]⁺ | | |
| | 1H), 5.92 (s, 1H), 4.51 (s, 2H), 4.36 (s, 2H), 3.84-3.73 | (ESI+) = 671.2 | | |
| | (m, 2H), 3.24 (s, 1H), 2.75 (s, 1H), 2.33-2.22 (m, 1H), | | | |
| | 1.91-1.82 (m, 1H). | | | |
| I-2 | ¹H NMR: (400 MHz, DMSO-d₆) δ 8.61 (s, 1H), 8.33 (s, | Calculated | 14.2% | White |
| | 1H), 7.72 (s, 1H), 7.33 (d, J = 14.6 Hz, 6H), 7.20-7.13 | exact mass = | | solid |
| | (m, 2H), 7.08 (ddt, J = 6.7, 4.9, 2.1 Hz, 1H), 7.03 (d, J = | 657.662 | | |
| | 8.4 Hz, 1H), 6.90 (d, J = 9.0 Hz, 1H), 6.82 (d, J = 2.4 Hz, | Found | | |
| | 1H), 6.75 (dd, J = 8.4, 2.4 Hz, 1H), 6.19 (s, 1H), 4.67 (d, | [M + H]⁺ | | |
| | J = 14.5 Hz, 1H), 4.53 (s, 1H), 4.35 (s, 2H), 3.74 (d, J = | (ESI+) = 657.0 | | |
| | 20.4 Hz, 3H), 3.63 (s, 1H). | | | |
| I-3 | ¹H NMR: (400 MHz, DMSO-d₆) δ 8.64 (s, 1H), 8.28 (s, | Calculated | 7.9% | White |
| | 1H), 7.74 (s, 1H), 7.40 (td, J = 9.1, 6.9 Hz, 2H), 7.33 (d, | exact mass = | | solid |
| | J = 4.2 Hz, 5H), 7.19 (s, 1H), 7.14 (s, 1H), 7.09 (q, J = | 712.74 | | |
| | 4.3 Hz, 1H), 7.04 (d, J = 8.4 Hz, 1H), 6.97 (td, J = 8.5, | Found | | |
| | 2.6 Hz, 1H), 6.82 (d, J = 2.4 Hz, 1H), 6.78 (dd, J = 8.4, | [M + H]⁺ | | |
| | 2.4 Hz, 1H), 5.59 (s, 1H), 4.50 (d, J = 1.6 Hz, 2H), 4.41- | (ESI+) = 712.2 | | |
| | 4.33 (m, 2H), 4.32-4.25 (m, 1H), 3.77 (q, J = 4.1 Hz, | | | |
| | 2H), 3.51 (s, 1H), 3.20 (s, 1H), 3.01 (t, J = 7.1 Hz, 1H), | | | |
| | 2.93 (dd, J = 12.8, 1.9 Hz, 1H), 2.87 (t, J = 7.1 Hz, 1H), | | | |
| | 2.75 (d, J = 13.0 Hz, 1H). | | | |
| I-5 | ¹H NMR: (400 MHz, DMSO-d₆) δ 8.34 (s, 1H), 8.26 | Calculated | 18.4% | White |
| | (d, J = 1.3 Hz, 1H), 7.73 (d, J = 1.5 Hz, 1H), 7.33 (h, J = 5.9, | exact mass = | | solid |
| | 4.9 Hz, 7H), 7.21 (d, J = 5.4 Hz, 1H), 7.16-7.06 (m, | 726.767 | | |
| | 2H), 7.03 (d, J = 8.4 Hz, 1H), 6.82 (q, J = 13.9, 10.5 Hz, | Found | | |
| | 3H), 5.62 (s, 1H), 4.59-4.49 (m, 2H), 4.36 (s, 2H), 4.21 | [M + H]⁺ | | |
| | (d, J = 7.5 Hz, 1H), 3.76 (s, 2H), 2.98 (dd, J = 16.7, 13.7 | (ESI+) = 726.2 | | |
| | Hz, 1H), 2.90-2.81 (m, 1H), 2.74 (d, J = 6.1 Hz, 1H), | | | |
| | 2.66-2.54 (m, 1H), 2.29 (s, 1H), 1.97 (s, 1H), 1.60 (s, | | | |
| | 1H) | | | |
| I-8 | ¹H NMR: (400 MHz, DMSO-d₆) δ 9.97 (s, 1H), 8.30 (s, | Calculated | 11.8% | White |
| | 1H), 7.76 (s, 1H), 7.53-7.39 (m, 5H), 7.38-7.28 (m, | exact mass = | | solid |
| | 5H), 7.17 (ddd, J = 11.9, 9.2, 2.5 Hz, 1H), 7.06 (dd, J = | 817.878 | | |
| | 7.7, 6.7 Hz, 2H), 6.97 (td, J = 8.5, 2.6 Hz, 1H), 6.90- | Found | | |
| | 6.79 (m, 4H), 5.70 (s, 1H), 4.59 (s, 2H), 4.36 (d, J = 11.6 | [M + H]⁺ | | |
| | Hz, 2H), 3.85-3.71 (m, 2H), 3.02-2.88 (m, 5H), 2.71 | (ESI+) = 817.2 | | |
| | (d, J = 13.8 Hz, 1H), 2.61-2.52 (m, 4H). | | | |
| I-9 | ¹H NMR: (400 MHz, DMSO-d₆) δ 10.12 (s, 1H), 8.28 (s, | Calculated | 3.2% | White |
| | 1H), 7.73 (s, 1H), 7.61-7.55 (m, 2H), 7.47 (s, 1H), 7.35 | exact mass = | | solid |
| | (dddd, J = 15.6, 13.2, 8.7, 6.8 Hz, 6H), 7.21-7.17 (m, | 831.904 | | |
| | 2H), 7.16-7.10 (m, 1H), 7.08-7.02 (m, 2H), 6.94 (td, | Found | | |
| | J = 8.5, 2.6 Hz, 1H), 6.86 (d, J = 2.4 Hz, 1H), 6.83 | [M + H]⁺ | | |
| | (dd, J = 8.4, 2.4 Hz, 1H), 5.61 (s, 1H), 4.54 (s, 2H), 4.37 (s, | (ESI+) = 831.2 | | |
| | 2H), 3.84-3.69 (m, 2H), 2.84 (dd, J = 13.7, 1.8 Hz, 1H), | | | |
| | 2.65 (d, J = 13.9 Hz, 1H), 2.39 (s, 4H), 2.22 (s, 4H). | | | |
| I-6 | ¹H NMR: (400 MHz, DMSO-d₆) δ 8.28 (s, 1H), 8.15 | Calculated | 7.4% | White |
| | (d, J = 7.9 Hz, 1H), 7.75 (s, 1H), 7.44-7.27 (m, 6H), 7.20 (s, | exact mass = | | solid |
| | 1H), 7.14 (ddd, J = 11.9, 9.1, 2.6 Hz, 1H), 7.08 (td, J = | 740.794 | | |
| | 5.2, 2.4 Hz, 1H), 7.03 (d, J = 8.4 Hz, 1H), 6.95 (dt, J = | Found | | |
| | 8.5, 4.3 Hz, 1H), 6.81-6.75 (m, 2H), 5.67 (s, 1H), 4.53 | [M + H]⁺ | | |
| | (s, 2H), 4.36 (d, J = 12.4 Hz, 2H), 3.77 (q, J = 4.1 Hz, | (ESI+) = 740.2 | | |
| | 2H), 3.67-3.53 (m, 1H), 2.97-2.85 (m, 1H), 2.78 | | | |
| | (d, J = 11.4 Hz, 1H), 2.63 (t, J = 14.4 Hz, 2H), 2.22-2.07 (m, | | | |
| | 2H), 1.57 (d, J = 11.9 Hz, 2H), 1.35 (s, 2H). | | | |
| I-7 | ¹H NMR: (400 MHz, DMSO-d₆) δ 8.27 (s, 1H), 8.15 | Calculated | 9.0% | White |
| | (d, J = 7.9 Hz, 1H), 7.75 (s, 1H), 7.43-7.37 (m, 1H), 7.35- | exact mass = | | solid |
| | 7.28 (m, 5H), 7.20 (s, 1H), 7.14 (ddd, J = 11.8, 9.1, 2.6 | 740.794 | | |
| | Hz, 1H), 7.10-7.05 (m, 1H), 7.03 (d, J = 8.4 Hz, 1H), | Found | | |
| | 6.94 (d, J = 2.6 Hz, 1H), 6.81-6.75 (m, 2H), 5.64 (s, | [M + H]⁺ | | |
| | 1H), 4.53 (s, 2H), 4.36 (d, J = 12.6 Hz, 2H), 3.82-3.71 | (ESI+) = 740.2 | | |
| | (m, 2H), 3.67-3.55 (m, 1H), 2.96-2.84 (m, 2H), 2.77 | | | |
| | (d, J = 11.5 Hz, 1H), 2.65 (d, J = 15.6 Hz, 2H), 2.14 | | | |
| | (dt, J = 21.4, 11.5 Hz, 3H), 1.57 (d, J = 11.8 Hz, 2H), 1.34 (s, | | | |
| | 2H). | | | |
| I-10 | ¹H NMR: (400 MHz, DMSO-d₆) δ 8.34 (s, 1H), 8.25 | Calculated | 5.3% | White |
| | (d, J = 1.6 Hz, 1H), 7.73 (d, J = 1.5 Hz, 1H), 7.39-7.28 (m, | exact mass = | | solid |
| | 6H), 7.20 (s, 1H), 7.16-7.05 (m, 2H), 7.03 (d, J = 8.4 | 726.767 | | |
| | Hz, 1H), 6.92-6.77 (m, 3H), 5.62 (s, 1H), 4.58-4.48 | Found | | |
| | (m, 2H), 4.35 (s, 2H), 4.21 (q, J = 7.0 Hz, 1H), 3.75 (s, | [M + H]⁺ | | |
| | 2H), 2.98 (ddd, J = 16.3, 13.5, 1.8 Hz, 1H), 2.86 (d, J = | (ESI+) = 726.2 | | |
| | 12.4 Hz, 1H), 2.74 (d, J = 8.2 Hz, 1H), 2.65-2.54 (m, | | | |
| | 1H), 2.29 (s, 1H), 1.97 (s, 1H), 1.59 (d, J = 6.9 Hz, 1H). | | | |
| I-11 | ¹H NMR: (400 MHz, DMSO-d₆) δ 8.34 (s, 1H), 8.26 | calculated | 4.2% | White |
| | (d, J = 1.3 Hz, 1H), 7.73 (d, J = 1.5 Hz, 1H), 7.33 (h, J = 5.9, | exact mass = | | solid |
| | 4.9 Hz, 7H), 7.21 (d, J = 5.4 Hz, 1H), 7.16-7.06 (m, | 726.767 | | |
| | 2H), 7.03 (d, J = 8.4 Hz, 1H), 6.82 (q, J = 13.9, 10.5 Hz, | Found | | |
| | 3H), 5.62 (s, 1H), 4.59-4.49 (m, 2H), 4.36 (s, 2H), 4.21 | [M + H]⁺ | | |
| | (d, J = 7.5 Hz, 1H), 3.76 (s, 2H), 2.98 (dd, J = 16.7, 13.7 | (ESI+) = 726.2 | | |
| | Hz, 1H), 2.90-2.81 (m, 1H), 2.74 (d, J = 6.1 Hz, 1H), | | | |
| | 2.66-2.54 (m, 1H), 2.29 (s, 1H), 1.97 (s, 1H), 1.60 (s, | | | |
| | 1H). | | | |
| I-12 | ¹H NMR: (400 MHz, DMSO-d₆) δ 8.28 (s, 1H), 8.08 | Calculated | 10.3% | White |
| | (d, J = 7.9 Hz, 1H), 7.76 (s, 1H), 7.57 (d, J = 8.1 Hz, 1H), 7.43- | exact mass = | | solid |
| | 7.32 (m, 4H), 7.21 (dd, J = 8.0, 2.1 Hz, 1H), 7.18- | 713.768 | | |
| | 7.08 (m, 3H), 7.04 (d, J = 8.0 Hz, 2H), 7.01-6.91 (m, | Found | | |
| | 2H), 5.68 (s, 1H), 4.54 (s, 2H), 3.64-3.53 (m, 1H), 2.91 | [M + H]⁺ | | |
| | (d, J = 13.8 Hz, 1H), 2.77 (d, J = 11.5 Hz, 1H), 2.63 (t, J = | (ESI+) = 713.2 | | |
| | 14.3 Hz, 2H), 2.31 (s, 3H), 2.14 (dt, J = 22.4, 11.5 Hz, | | | |
| | 2H), 1.55 (s, 2H), 1.42-1.19 (m, 3H). | | | |
| I-13 | ¹H NMR: (400 MHz, DMSO-d₆) δ 8.25 (s, 1H), 8.04 | Calculated | 50.2% | White |
| | (d, J = 7.9 Hz, 1H), 7.90-7.83 (m, 2H), 7.74 (s, 1H), 7.69 (d, | exact mass = | | solid |
| | J = 1.7 Hz, 1H), 7.63-7.52 (m, 4H), 7.44-7.30 (m, | 722.778 | | |
| | 3H), 7.17 (dd, J = 8.4, 1.8 Hz, 1H), 7.14-7.10 (m, 1H), | Found | | |
| | 7.08 (s, 1H), 6.91 (td, J = 8.5, 2.5 Hz, 1H), 6.87 (dd, J = | [M + H]⁺ | | |
| | 3.3, 0.8 Hz, 1H), 5.60 (s, 1H), 4.50 (s, 2H), 3.53 (dt, J = | (ESI+) = 722.2 | | |
| | 7.8, 4.1 Hz, 1H), 2.90-2.82 (m, 1H), 2.71 (d, J = 11.8 | | | |
| | Hz, 1H), 2.58 (d, J = 13.7 Hz, 2H), 2.10 (q, J = 12.4 Hz, | | | |
| | 2H), 1.49 (s, 2H), 1.24 (s, 2H). | | | |
| I-14 | ¹H NMR: (400 MHz, Methanol-d₄) δ 8.32 (d, J = 4.8 Hz, | Calculated | 4.7% | White |
| | 1H), 7.89 (s, 1H), 7.60 (s, 1H), 7.30 (t, J = 7.7 Hz, 2H), | exact mass = | | solid |
| | 7.27-7.22 (m, 2H), 7.08 (q, J = 7.2 Hz, 2H), 7.02- | 726.767 | | |
| | 6.93 (m, 3H), 6.72 (d, J = 2.3 Hz, 1H), 6.64 (dt, J = 8.7, | Found | | |
| | 2.0 Hz, 1H), 4.70 (d, J = 7.5 Hz, 2H), 4.40-4.33 (m, | [M + H]⁺ | | |
| | 2H), 4.23 (s, 1H), 3.95 (s, 1H), 3.81-3.57 (m, 4H), 3.50- | (ESI+) = 726.2 | | |
| | 3.39 (m, 1H), 3.21-2.90 (m, 2H), 2.41 (s, 1H), 1.98 | | | |
| | (s, 1H). | | | |
| I-15 | ¹H NMR: (400 MHz, DMSO-d₆) δ 8.37 (t, J = 5.7 Hz, | Calculated | 2.5% | White |
| | 1H), 8.28 (s, 1H), 7.74 (s, 1H), 7.40-7.30 (m, 6H), 7.17- | exact mass = | | solid |
| | 7.11 (m, 2H), 7.07 (tt, J = 6.5, 2.2 Hz, 1H), 7.02 (d, J = | 769.835 | | |
| | 8.4 Hz, 1H), 6.94 (td, J = 8.6, 2.6 Hz, 1H), 6.81 (d, J = | Found | | |
| | 2.4 Hz, 1H), 6.77 (dd, J = 8.4, 2.4 Hz, 1H), 5.61 (s, 1H), | [M + H]⁺ | | |
| | 4.54 (s, 2H), 4.35 (d, J = 13.8 Hz, 2H), 3.84-3.70 (m, | (ESI+) = 769.2 | | |
| | 2H), 3.22 (d, J = 7.0 Hz, 2H), 2.83 (dd, J = 13.9, 1.8 Hz, | | | |
| | 1H), 2.64 (d, J = 13.7 Hz, 1H), 2.38 (s, 4H), 2.30 (dd, J = | | | |
| | 16.6, 9.6 Hz, 6H). | | | |
| I-16 | ¹H NMR: (400 MHz, DMSO-d₆) δ 8.27 (s, 1H), 8.19 | Calculated | 47.4% | White |
| | (d, J = 8.0 Hz, 1H), 7.75 (s, 1H), 7.39 (td, J = 9.0, 6.9 Hz, 1H), | exact mass = | | solid |
| | 7.35-7.27 (m, 5H), 7.23 (d, J = 7.9 Hz, 1H), 7.19 (s, | 738.821 | | |
| | 1H), 7.17-7.11 (m, 1H), 7.07 (tt, J = 6.6, 1.9 Hz, 1H), | Found | | |
| | 6.94 (td, J = 8.5, 2.6 Hz, 1H), 6.69 (dd, J = 7.9, 2.0 Hz, | [M + H]⁺ | | |
| | 1H), 6.51 (d, J = 2.0 Hz, 1H), 5.65 (s, 1H), 4.53 (s, 2H), | (ESI+) = 738.2 | | |
| | 3.69 (s, 1H), 3.60 (d, J = 4.4 Hz, 2H), 2.90 (d, J = 12.0 | | | |
| | Hz, 3H), 2.78 (d, J = 11.2 Hz, 1H), 2.69-2.58 (m, 2H), | | | |
| | 2.15 (dt, J = 21.1, 11.8 Hz, 2H), 2.04 (d, J = 3.4 Hz, 2H), | | | |
| | 1.57 (s, 2H), 1.35 (d, J = 13.4 Hz, 2H). | | | |
| I-20 | ¹H NMR: (400 MHz, DMSO-d₆) δ 8.27 (s, 1H), 8.19 | Calculated | 37.5% | White |
| | (d, J = 8.0 Hz, 1H), 7.75 (s, 1H), 7.39 (td, J = 9.0, 6.8 Hz, 1H), | exact mass = | | solid |
| | 7.35-7.27 (m, 5H), 7.23 (d, J = 8.0 Hz, 1H), 7.19 (s, | 738.821 | | |
| | 1H), 7.14 (ddd, J = 11.8, 9.1, 2.6 Hz, 1H), 7.10-7.05 | Found | | |
| | (m, 1H), 6.94 (td, J = 8.5, 2.6 Hz, 1H), 6.69 (dd, J = 7.9, | [M + H]⁺ | | |
| | 2.1 Hz, 1H), 6.52 (d, J = 2.1 Hz, 1H), 5.65 (s, 1H), 4.53 | (ESI+) = 738.2 | | |
| | (s, 2H), 3.69 (s, 1H), 3.60 (d, J = 4.5 Hz, 2H), 2.90 (d, J = | | | |
| | 11.9 Hz, 3H), 2.78 (d, J = 10.9 Hz, 1H), 2.63 (t, J = 12.2 | | | |
| | Hz, 2H), 2.16 (dd, J = 20.3, 10.7 Hz, 2H), 2.08-1.99 | | | |
| | (m, 2H), 1.57 (s, 2H), 1.34 (d, J = 11.9 Hz, 2H). | | | |
| I-21 | ¹H NMR: (400 MHz, DMSO-d₆) δ 8.27 (s, 1H), 8.19 | Calculated | 33.2% | White |
| | (d, J = 8.0 Hz, 1H), 7.75 (s, 1H), 7.39 (td, J = 9.0, 6.8 Hz, 1H), | exact mass = | | solid |
| | 7.35-7.27 (m, 5H), 7.23 (d, J = 8.0 Hz, 1H), 7.19 (s, | 738.821 | | |
| | 1H), 7.14 (ddd, J = 11.8, 9.1, 2.6 Hz, 1H), 7.07 (tt, J = | Found | | |
| | 6.6, 1.9 Hz, 1H), 6.94 (td, J = 8.5, 2.6 Hz, 1H), 6.69 (dd, | [M + H]⁺ | | |
| | J = 7.9, 2.1 Hz, 1H), 6.51 (d, J = 2.0 Hz, 1H), 5.69 (s, | (ESI+) = 738.3 | | |
| | 1H), 4.53 (s, 2H), 3.74-3.65 (m, 1H), 3.60 (dq, J = | | | |
| | 15.1, 7.0, 5.5 Hz, 2H), 2.98-2.85 (m, 3H), 2.78 (d, J = | | | |
| | 10.9 Hz, 1H), 2.70-2.57 (m, 2H), 2.16 (dd, J = 20.8, | | | |
| | 10.9 Hz, 2H), 2.04 (dt, J = 9.6, 4.1 Hz, 2H), 1.57 (s, 2H), | | | |
| | 1.44-1.27 (m, 2H). | | | |
| I-22 | ¹H NMR: (400 MHz, DMSO-d₆) δ 8.25 (s, 1H), 8.04 | Calculated | 38.4% | White |
| | (d, J = 7.9 Hz, 1H), 7.90-7.83 (m, 2H), 7.74 (s, 1H), 7.71- | exact mass = | | solid |
| | 7.67 (m, 1H), 7.62-7.53 (m, 4H), 7.43-7.30 (m, 3H), | 722.778 | | |
| | 7.17 (dd, J = 8.4, 1.8 Hz, 1H), 7.13-7.06 (m, 2H), 6.91 | Found | | |
| | (td, J = 8.5, 2.6 Hz, 1H), 6.87 (dd, J = 3.4, 0.9 Hz, 1H), | [M + H]⁺ | | |
| | 5.60 (s, 1H), 4.50 (s, 2H), 3.60-3.48 (m, 1H), 2.91- | (ESI+) = 722.2 | | |
| | 2.83 (m, 1H), 2.71 (d, J = 11.5 Hz, 1H), 2.58 (d, J = 13.7 | | | |
| | Hz, 2H), 2.10 (q, J = 12.5 Hz, 2H), 1.49 (s, 2H), 1.24 (s, | | | |
| | 2H). | | | |
| I-23 | ¹H NMR: (400 MHz, DMSO-d₆) δ 8.25 (s, 1H), 8.04 | Calculated | 37.3% | White |
| | (d, J = 8.0 Hz, 1H), 7.90-7.84 (m, 2H), 7.74 (s, 1H), 7.71- | exact mass = | | solid |
| | 7.67 (m, 1H), 7.62-7.53 (m, 4H), 7.43-7.30 (m, 3H), | 722.778 | | |
| | 7.17 (dd, J = 8.3, 1.8 Hz, 1H), 7.14-7.05 (m, 2H), 6.91 | Found | | |
| | (td, J = 8.5, 2.6 Hz, 1H), 6.87 (dd, J = 3.3, 0.9 Hz, 1H), | [M + H]⁺ | | |
| | 5.68-5.54 (m, 1H), 4.50 (s, 2H), 3.58-3.47 (m, 1H), | (ESI+) = 722.2 | | |
| | 2.86 (d, J = 13.8 Hz, 1H), 2.71 (d, J = 11.5 Hz, 1H), 2.58 | | | |
| | (d, J = 13.7 Hz, 2H), 2.10 (q, J = 12.5 Hz, 2H), 1.49 (s, | | | |
| | 2H), 1.23 (s, 2H). | | | |
| I-24 | ¹H NMR: (400 MHz, Methanol-d₄) δ 8.33 (s, 1H), 7.75 | Calculated | 16.7% | White |
| | (s, 1H), 7.55-7.51 (m, 1H), 7.47 (td, J = 9.0, 6.6 Hz, | exact mass = | | solid |
| | 1H), 7.36-7.31 (m, 2H), 7.12 (dd, J = 8.0, 2.1 Hz, 1H), | 713.768 | | |
| | 7.10-7.06 (m, 1H), 7.05 (s, 1H), 7.03-6.99 (m, 2H), | Found | | |
| | 6.96-6.89 (m, 2H), 6.84 (td, J = 8.5, 2.6 Hz, 1H), 4.67 | [M + H]⁺ | | |
| | (d, J = 14.3 Hz, 1H), 4.60 (d, J = 14.3 Hz, 1H), 3.69 (ddd, | (ESI+) = 713.2 | | |
| | J = 11.3, 7.1, 4.2 Hz, 1H), 3.01 (dd, J = 13.8, 1.6 Hz, | | | |
| | 1H), 2.79 (dd, J = 13.0, 8.4 Hz, 2H), 2.58 (d, J = 11.9 Hz, | | | |
| | 1H), 2.45-2.36 (m, 1H), 2.22 (td, J = 11.9, 2.5 Hz, 1H), | | | |
| | 1.77 (d, J = 12.6 Hz, 1H), 1.66 (d, J = 12.7 Hz, 1H), 1.48 | | | |
| | (dqd, J = 39.5, 11.9, 3.9 Hz, 2H). | | | |
| I-25 | ¹H NMR: (400 MHz, Methanol-d₄) δ 8.32 (s, 1H), 7.75 | Calculated | 13.3% | White |
| | (s, 1H), 7.53 (dd, J = 8.0, 0.9 Hz, 1H), 7.47 (td, J = 8.9, | exact mass = | | solid |
| | 6.6 Hz, 1H), 7.36-7.29 (m, 2H), 7.12 (dd, J = 8.0, 2.1 | 713.768 | | |
| | Hz, 1H), 7.10-7.04 (m, 2H), 7.03-6.98 (m, 2H), 6.96- | Found | | |
| | 6.87 (m, 2H), 6.84 (td, J = 8.5, 2.6 Hz, 1H), 4.67 (d, J = | [M + H]⁺ | | |
| | 14.3 Hz, 1H), 4.59(d, J = 14.3 Hz, 1H), 3.73-3.62 (m, | (ESI+) = 713.2 | | |
| | 1H), 3.00 (dd, J = 13.8, 1.6 Hz, 1H), 2.78 (dd, J = 12.8, | | | |
| | 7.4 Hz, 2H), 2.57 (d, J = 11.8 Hz, 1H), 2.40 (dd, J = 12.0, | | | |
| | 2.6 Hz, 1H), 2.21 (td, J = 11.9, 2.6 Hz, 1H), 1.76 (d, J = | | | |
| | 12.5 Hz, 1H), 1.65 (d, J = 12.9 Hz, 1H), 1.58-1.48 (m, | | | |
| | 1H), 1.47-1.37 (m, 1H). | | | |
| I-27 | ¹H NMR: (400 MHz, DMSO-d₆) δ 8.48 (s, 1H), 8.30 (s, | Calculated | 25.8% | White |
| | 1H), 7.74 (s, 1H), 7.32 (d, J = 4.3 Hz, 5H), 7.29-7.20 | exact mass = | | solid |
| | (m, 3H), 7.13 (s, 1H), 7.07 (d, J = 7.4 Hz, 1H), 7.03 | 697.726 | | |
| | (d, J = 8.4 Hz, 1H), 6.95 (s, 1H), 6.81 (d, J = 2.4 Hz, 1H), 6.77 | Found | | |
| | (dd, J = 8.4, 2.4 Hz, 1H), 4.56 (d, J = 3.9 Hz, 2H), 4.36 | [M + H]⁺ | | |
| | (d, J = 13.6 Hz, 2H), 3.95 (t, J = 7.6 Hz, 1H), 3.80-3.74 | (ESI+) = 697.2 | | |
| | (m, 2H), 3.54 (t, J = 7.8 Hz, 1H), 3.21-3.06 (m, 2H), | | | |
| | 2.36-2.26 (m, 3H), 2.01-1.95 (m, 1H), 1.23 (s, 2H). | | | |
| I-28 | ¹H NMR: (400 MHz, DMSO-d₆) δ 8.24 (d, J = 12.7 Hz, | Calculated | 18.6% | White |
| | 2H), 7.74 (s, 1H), 7.46-7.38 (m, 1H), 7.35 (d, J = 4.3 | exact mass = | | solid |
| | Hz, 4H), 7.23-7.16 (m, 2H), 7.09 (dd, J = 6.5, 3.0 Hz, | 754.82 | | |
| | 1H), 7.04 (d, J = 8.4 Hz, 1H), 7.00 (dd, J = 8.4, 2.5 Hz, | Found | | |
| | 1H), 6.82 (d, J = 2.4 Hz, 1H), 6.80-6.76 (m, 1H), 4.49 | [M + H]⁺ | | |
| | (s, 2H), 4.42-4.31 (m, 2H), 3.78 (q, J = 4.1 Hz, 2H), | (ESI+) = 754.3 | | |
| | 3.68 (d, J = 6.9 Hz, 1H), 2.89 (d, J = 11.2 Hz, 1H), 2.27- | | | |
| | 2.19 (m, 2H), 2.08-1.93 (m, 3H), 1.87 (t, J = 11.7 Hz, | | | |
| | 1H), 1.72 (d, J = 13.4 Hz, 2H), 1.47 (d, J = 13.2 Hz, 2H). | | | |
| I-29 | ¹H NMR: (400 MHz, DMSO-d₆) δ 8.32 (s, 1H), 8.25 | Calculated | 20.5% | White |
| | (d, J = 7.7 Hz, 1H), 7.75 (s, 1H), 7.44-7.37 (m, 1H), 7.37- | exact mass = | | solid |
| | 7.28 (m, 5H), 7.21 (s, 1H), 7.20-7.13 (m, 1H), 7.08 (s, | 768.804 | | |
| | 1H), 7.03 (d, J = 8.4 Hz, 1H), 6.95 (t, J = 8.5 Hz, 1H), | Found | | |
| | 6.83-6.76 (m, 2H), 6.50 (d, J = 6.3 Hz, 1H), 4.62- | [M + H]⁺ | | |
| | 4.51 (m, 2H), 4.36 (d, J = 11.8 Hz, 2H), 4.24 (s, 1H), | (ESI+) = 768.2 | | |
| | 3.90 (d, J = 13.5 Hz, 2H), 3.77 (s, 2H), 3.24 (d, J = 15.8 | | | |
| | Hz, 1H), 3.07 (d, J = 13.4 Hz, 1H), 2.79 (t, J = 14.3 Hz, | | | |
| | 1H), 2.59 (d, J = 17.4 Hz, 1H), 1.83 (s, 1H), 1.73 (d, J = | | | |
| | 12.4 Hz, 1H), 1.48-1.08 (m, 2H). | | | |
| I-30 | ¹H NMR: (400 MHz, DMSO-d₆) δ 10.50 (s, 1H), 8.79 (d, | Calculated | 6.1% | White |
| | J = 2.4 Hz, 1H), 8.36 (s, 1H), 8.20 (dd, J = 8.7, 2.5 Hz, | exact mass = | | solid |
| | 1H), 7.70 (s, 1H), 7.50 (s, 1H), 7.42 (d, J = 8.7 Hz, 1H), | 770.727 | | |
| | 7.37-7.30 (m, 4H), 7.22 (td, J = 8.9, 6.7 Hz, 1H), 7.13- | Found | | |
| | 7.03 (m, 3H), 6.99 (s, 1H), 6.88 (d, J = 2.4 Hz, 1H), | [M + H]⁺ | | |
| | 6.84 (dp, J = 8.3, 2.5 Hz, 2H), 5.34 (d, J = 14.6 Hz, 1H), | (ESI+) = 770.2 | | |
| | 4.82 (d, J = 14.8 Hz, 1H), 4.45-4.30 (m, 2H), 3.78 (q, J = | | | |
| | 4.5 Hz, 2H). | | | |
| I-31 | ¹H NMR: (400 MHz, DMSO-d₆) δ 8.28 (s, 1H), 7.72 (s, | Calculated | 40.9% | White |
| | 1H), 7.51 (s, 1H), 7.42-7.36 (m, 1H), 7.35-7.29 (m, | exact mass = | | solid |
| | 5H), 7.14 (s, 1H), 7.09-7.06 (m, 1H), 7.03 (d, J = 8.4 | 754.82 | | |
| | Hz, 1H), 6.97-6.91 (m, 1H), 6.82 (d, J = 2.4 Hz, 1H), | Found | | |
| | 6.78 (dd, J = 8.5, 2.4 Hz, 1H), 5.58 (s, 1H), 4.54 (d, J = | [M + H]⁺ | | |
| | 3.3 Hz, 2H), 4.35 (d, J = 13.2 Hz, 2H), 3.83-3.70 (m, | (ESI+) = 754.2 | | |
| | 2H), 2.83 (d, J = 13.8 Hz, 1H), 2.67 (d, J = 13.7 Hz, 1H), | | | |
| | 2.45 (d, J = 11.5 Hz, 1H), 2.38-2.26 (m, 3H), 2.06 (d, J = | | | |
| | 45.8 Hz, 2H), 1.44 (s, 2H), 1.25 (s, 3H). | | | |
| I-32 | ¹H NMR: (400 MHz, DMSO-d₆) δ 8.32 (d, J = 7.1 Hz, | Calculated | 15.0% | White |
| | 1H), 8.21 (d, J = 8.0 Hz, 1H), 7.79 (d, J = 12.3 Hz, 1H), | exact mass = | | solid |
| | 7.38-7.33 (m, 4H), 7.31-7.24 (m, 2H), 7.22 (s, 1H), | 780.857 | | |
| | 7.09 (dt, J = 5.0, 3.1 Hz, 1H), 7.04 (dd, J = 8.3, 2.0 Hz, | Found | | |
| | 1H), 6.97 (td, J = 8.5, 2.5 Hz, 1H), 6.83-6.75 (m, 2H), | [M + H]⁺ | | |
| | 4.52 (d, J = 2.2 Hz, 1H), 4.38 (d, J = 12.7 Hz, 2H), 4.04- | (ESI+) = 780.2 | | |
| | 3.93 (m, 1H), 3.77 (d, J = 4.2 Hz, 2H), 3.67-3.59 (m, | | | |
| | 1H), 3.56-3.51 (m, 1H), 2.71 (td, J = 28.7, 26.6, 13.6 | | | |
| | Hz, 3H), 2.33-2.26 (m, 1H), 2.18-2.04 (m, 3H), 1.97- | | | |
| | 1.77 (m, 3H), 1.69 (d, J = 12.1 Hz, 2H), 1.47 (d, J = | | | |
| | 12.5 Hz, 2H). | | | |
| I-33 | ¹H NMR: (400 MHz, Methanol-d₄) δ 8.31 (s, 1H), 7.72 | Calculated | 8.2% | White |
| | (s, 1H), 7.71-7.65 (m, 1H), 7.44 (td, J = 9.0, 6.6 Hz, | exact mass = | | solid |
| | 1H), 7.35-7.25 (m, 7H), 7.18 (t, J = 8.1 Hz, 1H), 7.12- | 890.968 | | |
| | 7.09 (m, 1H), 7.09-7.01 (m, 2H), 6.88 (ddd, J = 11.6, | Found | | |
| | 8.9, 2.5 Hz, 1H), 6.82 (d, J = 8.8 Hz, 1H), 6.79 (d, J = 2.5 | [M + H]⁺ | | |
| | Hz, 1H), 6.74 (dd, J = 8.4, 2.4 Hz, 1H), 6.66 (ddd, J = | (ESI+) = 890.2 | | |
| | 8.2, 2.5, 1.0 Hz, 1H), 4.68-4.48 (m, 8H), 4.38 (t, J = | | | |
| | 4.3 Hz, 2H), 4.04 (t, J = 6.1 Hz, 2H), 3.77 (s, 2H), 3.59 | | | |
| | (d, J = 6.0 Hz, 2H), 2.94 (d, J = 13.8 Hz, 1H), 2.76 (d, J = | | | |
| | 13.8 Hz, 1H), 2.65-2.52 (m, 2H), 2.26 (dt, J = 20.7, | | | |
| | 10.4 Hz, 2H), 1.96 (t, J = 6.1 Hz, 2H), 1.73 (d, J = 17.1 | | | |
| | Hz, 2H), 1.56-1.43 (m, 2H). | | | |
| I-36 | ¹H NMR: (400 MHz, DMSO-d₆) δ 8.45 (d, J = 6.9 Hz, | Calculated | 17.4% | White |
| | 1H), 8.26 (d, J = 10.9 Hz, 1H), 7.75 (d, J = 6.3 Hz, 1H), | exact mass = | | solid |
| | 7.46-7.38 (m, 1H), 7.37-7.29 (m, 5H), 7.25 (d, J = | 740.794 | | |
| | 3.6 Hz, 1H), 7.18 (ddd, J = 11.8, 9.0, 2.6 Hz, 1H), 7.08 | Found | | |
| | (q, J = 4.2 Hz, 1H), 7.03 (dd, J = 8.4, 2.6 Hz, 1H), 6.98 | [M + H]⁺ | | |
| | (td, J = 8.5, 2.6 Hz, 1H), 6.82 (d, J = 2.3 Hz, 1H), 6.79 | (ESI+) = 740.2 | | |
| | (dt, J = 8.4, 2.1 Hz, 1H), 6.72 (s, 1H), 4.49 (d, J = 2.7 Hz, | | | |
| | 2H), 4.41-4.31 (m, 2H), 4.28 (d, J = 7.3 Hz, 1H), 3.77 | | | |
| | (d, J = 4.3 Hz, 2H), 2.57 (s, 1H), 2.47-2.32 (m, 3H), | | | |
| | 2.28 (dd, J = 9.4, 5.4 Hz, 1H), 2.16-2.00 (m, 3H), 1.90 | | | |
| | (q, J = 11.3, 8.5 Hz, 1H), 1.68 (d, J = 13.0 Hz, 1H). | | | |
| I-37 | ¹H NMR: (400 MHz, DMSO-d₆) δ 8.55-8.41 (m, 1H), | Calculated | 29.6% | White |
| | 8.32 (dd, J = 4.2, 2.2 Hz, 1H), 7.76 (d, J = 2.8 Hz, 1H), | exact mass = | | solid |
| | 7.53-7.37 (m, 2H), 7.37-7.26 (m, 5H), 7.20 (td, J = | 754.777 | | |
| | 9.8, 2.8 Hz, 2H), 7.07 (s, 1H), 7.03 (dd, J = 8.4, 4.0 Hz, | Found | | |
| | 1H), 6.96 (ddt, J = 11.0, 5.1, 2.6 Hz, 1H), 6.85-6.75 | [M + H]⁺ | | |
| | (m, 2H), 6.68 (q, J = 6.3 Hz, 1H), 4.64-4.49 (m, 2H), | (ESI+) = 754.4 | | |
| | 4.42-4.25 (m, 3H), 3.88-3.65 (m, 3H), 3.61-3.49 | | | |
| | (m, 1H), 3.28-3.17 (m, 1H), 3.03 (ddd, J = 23.5, 15.1, | | | |
| | 4.9 Hz, 1H), 2.85-2.72 (m, 1H), 2.19-1.98 (m, 1H), | | | |
| | 1.86 (ddd, J = 27.0, 13.2, 6.8 Hz, 1H). | | | |
| I-38 | ¹H NMR: (400 MHz, DMSO-d₆) δ 8.28 (d, J = 3.7 Hz, | Calculated | 1.5% | White |
| | 2H), 7.74 (s, 1H), 7.43-7.33 (m, 2H), 7.34-7.28 (m, | exact mass = | | solid |
| | 5H), 7.14 (ddd, J = 11.9, 9.1, 2.6 Hz, 1H), 7.11-7.04 | 738.778 | | |
| | (m, 2H), 7.01 (d, J = 8.4 Hz, 1H), 6.95 (td, J = 8.5, 2.6 | Found | | |
| | Hz, 1H), 6.81-6.71 (m, 2H), 5.59 (s, 1H), 4.57-4.44 | [M + H]⁺ | | |
| | (m, 2H), 4.34 (d, J = 12.0 Hz, 2H), 3.76 (q, J = 4.8 Hz, | (ESI+) = 738.2 | | |
| | 2H), 3.00-2.91 (m, 2H), 2.80 (d, J = 8.7 Hz, 1H), 2.73 | | | |
| | (d, J = 13.5 Hz, 1H), 2.66 (dt, J = 4.5, 2.3 Hz, 1H), 2.44 | | | |
| | (dd, J = 8.8, 2.9 Hz, 1H), 2.36 (dd, J = 8.7, 2.9 Hz, 1H), | | | |
| | 1.46 (q, J = 2.6 Hz, 2H). | | | |
| I-39 | ¹H NMR: (400 MHz, DMSO-d₆) δ 8.70 (d, J = 6.9 Hz, | Calculated | 27.0% | White |
| | 1H), 8.31 (d, J = 9.1 Hz, 1H), 7.79 (d, J = 18.7 Hz, 1H), | exact mass = | | solid |
| | 7.34 (d, J = 4.3 Hz, 5H), 7.30-7.23 (m, 2H), 7.23 (s, | 752.804 | | |
| | 1H), 7.10-7.06 (m, 1H), 7.04 (d, J = 8.4 Hz, 1H), 6.96 | Found | | |
| | (td, J = 8.5, 2.6 Hz, 1H), 6.82 (d, J = 2.4 Hz, 1H), 6.79 | [M + H]⁺ | | |
| | (dd, J = 8.4, 2.4 Hz, 1H), 4.52 (d, J = 2.7 Hz, 2H), 4.40- | (ESI+) = 752.2 | | |
| | 4.28 (m, 3H), 3.99-3.93 (m, 1H), 3.77 (d, J = 5.5 Hz, | | | |
| | 2H), 3.51-3.40 (m, 3H), 2.85 (dt, J = 13.3, 6.8 Hz, 2H), | | | |
| | 2.35-2.19 (m, 3H), 2.03 (p, J = 7.7 Hz, 1H), 1.89 (dd, J | | | |
| | = 12.7, 9.1 Hz, 1H). | | | |
| I-40 | ¹H NMR: (400 MHz, DMSO-d₆) δ 8.44 (t, J = 5.8 Hz, | Calculated | 12.5% | White |
| | 1H), 8.29 (s, 1H), 7.74 (s, 1H), 7.57 (d, J = 8.1 Hz, 1H), | exact mass = | | solid |
| | 7.44-7.32 (m, 3H), 7.31-7.18 (m, 4H), 7.10 (t, J = 7.4 | 670.7 | | |
| | Hz, 1H), 7.07-7.00 (m, 4H), 6.96 (td, J = 8.4, 2.6 Hz, | Found | | |
| | 1H), 4.61-4.51 (m, 2H), 3.94 (t, J = 7.7 Hz, 1H), 3.54 | [M + H]⁺ | | |
| | (t, J = 7.8 Hz, 1H), 3.11 (d, J = 46.7 Hz, 2H), 2.31 (s, 5H), | (ESI+) = 670.2 | | |
| | 2.01-1.92 (m, 1H). | | | |
| I-41 | ¹H NMR: (400 MHz, DMSO-d₆) δ 8.37 (t, J = 5.8 Hz, | Calculated | 21.6% | White |
| | 1H), 8.26 (s, 1H), 7.90-7.84 (m, 2H), 7.71 (d, J = 2.6 | exact mass = | | solid |
| | Hz, 2H), 7.63-7.52 (m, 4H), 7.41 (d, J = 7.1 Hz, 2H), | 679.71 | | |
| | 7.32-7.12 (m, 4H), 7.03 (s, 1H), 6.97-6.90 (m, 1H), | Found | | |
| | 6.87 (d, J = 3.3 Hz, 1H), 4.52 (d, J = 2.4 Hz, 2H), 3.88 (t, | [M + H]⁺ | | |
| | J = 7.7 Hz, 1H), 3.47 (t, J = 7.9 Hz, 1H), 3.12 (dd, J = | (ESI+) = 679.2 | | |
| | 13.3, 6.4 Hz, 1H), 2.99 (s, 1H), 2.23 (h, J = 7.1, 6.3 Hz, | | | |
| | 2H), 1.96-1.79 (m, 1H). | | | |
| I-42 | ¹H NMR: (400 MHz, DMSO-d₆) δ 8.31 (s, 1H), 8.25 | Calculated | 2.1% | White |
| | (d, J = 7.8 Hz, 1H), 7.81 (s, 1H), 7.50-7.42 (m, 1H), 7.34 (d, | exact mass = | | solid |
| | J = 4.3 Hz, 5H), 7.23-7.16 (m, 2H), 7.11-7.06 (m, | 798.83 | | |
| | 1H), 7.03 (dd, J = 8.6, 6.5 Hz, 2H), 6.83-6.76 (m, 2H), | Found | | |
| | 6.27 (s, 1H), 4.59 (s, 2H), 4.47 (d, J = 11.6 Hz, 1H), 4.36 | [M + H]⁺ | | |
| | (d, J = 12.0 Hz, 2H), 4.28 (d, J = 11.5 Hz, 1H), 3.77 | (ESI+) = 798.2 | | |
| | (q, J = 4.3 Hz, 2H), 3.60 (dd, J = 7.7, 4.0 Hz, 1H), 3.13 (d, J = | | | |
| | 10.2 Hz, 2H), 2.73-2.63 (m, 2H), 2.09 (ddd, J = 16.4, | | | |
| | 12.9, 10.1 Hz, 2H), 1.65 (d, J = 11.0 Hz, 2H), 1.44 (d, J = | | | |
| | 12.6 Hz, 2H). | | | |
| I-43 | ¹H NMR: (400 MHz, DMSO-d₆) δ 8.51 (t, J = 5.8 Hz, | Calculated | 24.5% | White |
| | 1H), 8.30 (s, 1H), 7.74 (s, 1H), 7.39-7.19 (m, 9H), 7.12 | exact mass = | | solid |
| | (s, 1H), 7.08 (td, J = 6.4, 5.7, 2.9 Hz, 1H), 6.99-6.93 | 695.753 | | |
| | (m, 1H), 6.69 (dd, J = 7.9, 2.0 Hz, 1H), 6.53 (d, J = 2.0 | Found | | |
| | Hz, 1H), 4.62-4.51 (m, 2H), 3.95 (t, J = 7.7 Hz, 1H), | [M + H]⁺ | | |
| | 3.68 (s, 1H), 3.64-3.52 (m, 2H), 3.13 (dd, J = 38.2, | (ESI+) = 695.2 | | |
| | 25.0 Hz, 2H), 2.90 (dt, J = 11.3, 6.4 Hz, 2H), 2.37-2.25 | | | |
| | (m, 2H), 2.05 (q, J = 6.1 Hz, 2H), 1.97 (d, J = 3.8 Hz, | | | |
| | 1H). | | | |
| I-44 | ¹H NMR: (400 MHz, DMSO-d₆) δ 8.44 (s, 1H), 8.36- | Calculated | 4.9% | White |
| | 8.26 (m, 1H), 7.78 (dd, J = 13.1, 3.5 Hz, 1H), 7.41- | exact mass = | | solid |
| | 7.21 (m, 9H), 7.10-7.01 (m, 2H), 6.97 (d, J = 8.0 Hz, | 766.831 | | |
| | 1H), 6.83-6.76 (m, 2H), 4.55-4.47 (m, 1H), 4.32 (dd, | Found | | |
| | J = 33.9, 10.8 Hz, 4H), 4.10-3.94 (m, 1H), 3.77 (s, 2H), | [M + H]⁺ | | |
| | 3.55-3.44 (m, 1H), 2.71-2.58 (m, 2H), 2.33 (td, J = | (ESI+) = 766.2 | | |
| | 10.5, 10.1, 5.6 Hz, 3H), 2.22 (d, J = 12.1 Hz, 2H), 2.08- | | | |
| | 1.92 (m, 2H), 1.70 (d, J = 16.2 Hz, 2H). | | | |
| I-45 | ¹H NMR: (400 MHz, DMSO-d₆) δ 8.44 (s, 1H), 8.36- | Calculated | 10.8% | White |
| | 8.27 (m, 1H), 7.79 (dd, J = 13.1, 3.6 Hz, 1H), 7.41- | exact mass = | | solid |
| | 7.20 (m, 9H), 7.10-7.01 (m, 2H), 6.97 (d, J = 8.2 Hz, | 766.831 | | |
| | 1H), 6.83-6.76 (m, 2H), 4.52 (d, J = 2.5 Hz, 1H), 4.32 | Found | | |
| | (dd, J = 33.8, 11.0 Hz, 3H), 4.02 (d, J = 29.3 Hz, 1H), | [M + H]⁺ | | |
| | 3.82-3.73 (m, 2H), 3.51 (d, J = 6.9 Hz, 1H), 2.71- | (ESI+) = 766.2 | | |
| | 2.60 (m, 2H), 2.39-2.17 (m, 6H), 2.11-1.87 (m, 2H), | | | |
| | 1.70 (d, J = 19.7 Hz, 1H). | | | |
| I-46 | ¹H NMR: (400 MHz, Methanol-d₄) δ 8.29 (s, 1H), 7.90 | Calculated | 2.6% | White |
| | (s, 1H), 7.62-7.50 (m, 3H), 7.33-7.26 (m, 7H), 7.09- | exact mass = | | solid |
| | 6.96 (m, 4H), 6.78 (d, J = 2.4 Hz, 1H), 6.72 (dd, J = 8.4, | 846.915 | | |
| | 2.4 Hz, 1H), 4.69 (s, 2H), 4.48 (s, 2H), 4.38 (t, J = 4.4 | Found | | |
| | Hz, 2H), 3.76 (d, J = 4.2 Hz, 2H), 3.63 (s, 2H), 3.13 (s, | [M + H]⁺ | | |
| | 4H), 2.00 (d, J = 22.2 Hz, 2H), 1.85 (s, 2H). | (ESI+) = 846.2 | | |
| I-48 | ¹H NMR: (400 MHz, DMSO-d₆) δ 8.29 (s, 1H), 8.25 | Calculated | 20.5% | White |
| | (d, J = 7.8 Hz, 1H), 7.78 (s, 1H), 7.42-7.37 (m, 1H), 7.34 (d, | exact mass = | | solid |
| | J = 4.2 Hz, 5H), 7.24-7.18 (m, 2H), 7.08 (p, J = 4.3 Hz, | 812.856 | | |
| | 1H), 7.04 (d, J = 8.3 Hz, 1H), 6.98 (td, J = 8.6, 2.6 Hz, | Found | | |
| | 1H), 6.83-6.76 (m, 2H), 5.95 (s, 1H), 4.51 (d, J = 2.1 | [M + H]⁺ | | |
| | Hz, 2H), 4.36 (d, J = 12.0 Hz, 2H), 4.04-3.97 (m, 1H), | (ESI+) = 812.2 | | |
| | 3.87 (dt, J = 11.0, 7.0 Hz, 1H), 3.77 (d, J = 4.0 Hz, 2H), | | | |
| | 3.68-3.57 (m, 1H), 3.02-2.87 (m, 2H), 2.71 (s, 2H), | | | |
| | 2.44-2.34 (m, 1H), 2.16-2.08 (m, 2H), 2.00 (dt, J = | | | |
| | 13.4, 6.2 Hz, 1H), 1.70 (d, J = 11.8 Hz, 2H), 1.48 (d, J = | | | |
| | 12.4 Hz, 2H). | | | |
| I-50 | ¹H NMR: (400 MHz, DMSO-d₆) δ 8.28 (s, 1H), 8.23 (dd, | Calculated | 17.5% | White |
| | J = 7.9, 4.9 Hz, 1H), 7.73 (s, 1H), 7.44-7.37 (m, 1H), | exact mass = | | solid |
| | 7.37-7.26 (m, 5H), 7.21 (s, 1H), 7.14 (ddd, J = 11.9, | 754.82 | | |
| | 9.1, 2.6 Hz, 1H), 7.10-7.05 (m, 1H), 7.03 (dd, J = 8.3, | Found | | |
| | 1.5 Hz, 1H), 6.93 (t, J = 7.3 Hz, 1H), 6.82-6.75 (m, | [M + H]⁺ | | |
| | 2H), 5.63 (s, 1H), 4.60-4.46 (m, 2H), 4.35 (d, J = 15.1 | (ESI+) = 754.2 | | |
| | Hz, 2H), 3.89-3.67 (m, 3H), 3.04 (dd, J = 14.3, 5.9 Hz, | | | |
| | 1H), 2.85 (dd, J = 14.0, 2.9 Hz, 1H), 2.69-2.57 (m, 4H), | | | |
| | 1.70 (s, 2H), 1.51 (d, J = 9.7 Hz, 3H), 1.39 (d, J = 12.8 | | | |
| | Hz, 1H). | | | |
| I-53 | ¹H NMR: (400 MHz, DMSO-d₆) δ 8.28 (s, 1H), 7.72 (s, | Calculated | 28.6% | White |
| | 1H), 7.51 (s, 1H), 7.41-7.31 (m, 6H), 7.14 (t, J = 10.5 | exact mass = | | solid |
| | Hz, 1H), 7.09-7.05 (m, 1H), 7.03 (d, J = 8.4 Hz, 1H), | 754.82 | | |
| | 6.94 (td, J = 8.5, 2.6 Hz, 1H), 6.82 (d, J = 2.4 Hz, 1H), | Found | | |
| | 6.78 (dd, J = 8.4, 2.4 Hz, 1H), 5.59 (s, 1H), 4.59-4.47 | [M + H]⁺ | | |
| | (m, 2H), 4.36 (d, J = 14.6 Hz, 2H), 3.83-3.71 (m, 2H), | (ESI+) = 754.2 | | |
| | 2.83 (d, J = 13.8 Hz, 1H), 2.67 (d, J = 13.9 Hz, 1H), 2.43 | | | |
| | (s, 1H), 2.34 (s, 3H), 2.18-1.95 (m, 2H), 1.44 (s, 2H), | | | |
| | 1.25 (s, 3H). | | | |
| I-64 | ¹H NMR: (400 MHz, Methanol-d₄) δ 8.32 (s, 1H), 7.77 | Calculated | 8.4% | White |
| | (s, 1H), 7.49 (td, J = 8.9, 6.6 Hz, 1H), 7.37-7.30 (m, 4H), | exact mass = | | solid |
| | 7.33-7.20 (m, 2H), 7.11 (s, 1H), 7.09-7.08 (m, 1h), | 768.847 | | |
| | 7.05 (d, J = 8.4 Hz, 1H), 6.93 (ddd, J = 11.6, 8.8, 2.5 Hz, | Found | | |
| | 1H), 6.86 (td, J = 8.4, 2.6 Hz, 1H), 6.77 (d, J = 2.4 Hz, | [M + H]⁺ | | |
| | 1H), 6.72 (dd, J = 8.4, 2.4 Hz, 1H), 4.57 (d, J = 14.2 Hz, | (ESI+) = 768.2 | | |
| | 1H), 4.52 (d, J = 14.2 Hz, 1H), 4.40-4.33 (m, 2H), 3.79 | | | |
| | (s, dH), 2.95-2.72 (m, 3H), 2.49 (m, 1H), 2.46 (m, 1H), | | | |
| | 2.33 (m, 1H), 1.91-1.82 (m, 2H), 1.70-1.58 (m, 4H), | | | |
| | 1.45-1.38 (m, 2H). | | | |
| I-67 | ¹H NMR: (400 MHz, Methanol-d₄) δ 8.34 (s, 1H), 7.73 | Calculated | 4.2% | White |
| | (s, 1H), 7.46 (td, J = 8.9, 6.6 Hz, 1H), 7.33 (dd, J = 8.6, | exact mass = | | solid |
| | 7.1 Hz, 2H), 7.30-7.25 (m, 2H), 7.12-7.05 (m, 1H), | 740.837 | | |
| | 6.99 (d, J = 8.4 Hz, 1H), 6.91 (ddd, J = 11.6, 8.8, 2.5 Hz, | Found | | |
| | 1H), 6.82 (td, J = 8.4, 2.6 Hz, 1H), 6.70 (d, J = 2.4 Hz, | [M + H]⁺ | | |
| | 1H), 6.66 (dd, J = 8.4, 2.4 Hz, 1H), 6.27 (s, 1H), 4.65 (d, | (ESI+) = 740.6 | | |
| | J = 14.2 Hz, 1H), 4.54 (d, J = 14.2 Hz, 1H), 4.40-4.33 | | | |
| | (m, 2H), 3.75 (d, J = 5.3 Hz, 4H), 2.95 (dd, J = 13.7, 1.6 | | | |
| | Hz, 1H), 2.73 (dd, J = 16.9, 12.6 Hz, 2H), 2.46 (d, J = | | | |
| | 11.6 Hz, 1H), 2.36 (d, J = 6.6 Hz, 2H), 2.29 (td, J = 11.6, | | | |
| | 2.6 Hz, 1H), 2.05 (td, J = 11.7, 2.6 Hz, 1H), 1.62 (d, J = | | | |
| | 12.9 Hz, 1H), 1.48 (d, J = 12.9 Hz, 1H), 1.35 (ddd, J = | | | |
| | 14.9, 6.6, 3.1 Hz, 1H), 1.23-1.14 (m, 1H), 1.09-0.98 | | | |
| | (m, 1H). | | | |
| I-69 | ¹H NMR: (400 MHz, DMSO-d₆) δ 8.45 (d, J = 7.0 Hz, | Calculated | 29.6% | White |
| | 1H), 8.27 (s, 1H), 7.75 (s, 1H), 7.43-7.30 (m, 6H), 7.26 | exact mass = | | solid |
| | (s, 1H), 7.17 (s, 1H), 7.08 (q, J = 4.2 Hz, 1H), 7.03 (d, J = | 740.794 | | |
| | 8.4 Hz, 1H), 6.93 (s, 1H), 6.84-6.77 (m, 2H), 6.68 (s, | Found | | |
| | 1H), 4.49 (s, 2H), 4.31 (dd, J = 35.9, 9.5 Hz, 3H), 3.82- | [M + H]⁺ | | |
| | 3.66 (m, 2H), 2.67 (p, J = 1.9 Hz, 2H), 2.45-2.38 (m, | (ESI+) = 740.2 | | |
| | 1H), 2.27 (dd, J = 9.4, 5.3 Hz, 2H), 2.17-2.00 (m, 3H), | | | |
| | 1.95-1.84 (m, 1H), 1.67 (d, J = 6.6 Hz, 1H). | | | |
| I-70 | ¹H NMR: (400 MHz, DMSO-d₆) δ 8.44 (d, J = 7.0 Hz, | Calculated | 32.1% | White |
| | 1H), 8.24 (d, J = 6.5 Hz, 1H), 7.74 (s, 1H), 7.45-7.30 | exact mass = | | solid |
| | (m, 6H), 7.25 (s, 1H), 7.18 (ddd, J = 11.9, 9.1, 2.6 Hz, | 740.794 | | |
| | 1H), 7.08 (q, J = 4.1 Hz, 1H), 7.03 (d, J = 8.4 Hz, 1H), | Found | | |
| | 7.00-6.95 (m, 1H), 6.82 (d, J = 2.4 Hz, 1H), 6.78 (dd, J = | [M + H]⁺ | | |
| | 8.4, 2.4 Hz, 1H), 6.71 (s, 1H), 4.48 (s, 2H), 4.36 (d, J = | (ESI+) = 740.2 | | |
| | 10.8 Hz, 2H), 4.32-4.22 (m, 1H), 3.77 (d, J = 4.3 Hz, | | | |
| | 2H), 2.45 (s, 1H), 2.37 (dd, J = 9.5, 5.6 Hz, 2H), 2.17- | | | |
| | 2.00 (m, 3H), 1.95-1.83 (m, 1H), 1.68 (dd, J = 13.2, | | | |
| | 6.6 Hz, 1H). | | | |
| I-83 | ¹H NMR: (400 MHz, DMSO-d₆) δ 8.49 (t, J = 5.8 Hz, | Calculated | 18.7% | White |
| | 1H), 8.31 (s, 1H), 7.75 (s, 1H), 7.32 (d, J = 4.3 Hz, 5H), | exact mass = | | solid |
| | 7.28-7.22 (m, 2H), 7.13 (s, 1H), 7.08 (q, J = 4.4 Hz, | 697.726 | | |
| | 1H), 7.03 (d, J = 8.4 Hz, 1H), 6.96 (td, J = 8.5, 2.6 Hz, | Found | | |
| | 1H), 6.82 (d, J = 2.4 Hz, 1H), 6.78 (dd, J = 8.5, 2.4 Hz, | [M + H]⁺ | | |
| | 1H), 4.63-4.50 (m, 2H), 4.36 (d, J = 13.5 Hz, 2H), 3.95 | (ESI+) = 697.2 | | |
| | (t, J = 7.7 Hz, 1H), 3.82-3.72 (m, 2H), 3.55 (t, J = 7.9- | | | |
| | Hz, 1H), 3.26-2.98 (m, 2H), 2.37-2.22 (m, 2H), 2.03 | | | |
| | 1.92 (m, 1H). | | | |
| I-85 | ¹H NMR: (400 MHz, DMSO-d₆) δ 10.04 (s, 1H), 8.48 (d, | Calculated | 15.6% | White |
| | J = 0.8 Hz, 2H), 7.57-7.52 (m, 2H), 7.45 (s, 1H), 7.38- | exact mass = | | solid |
| | 7.28 (m, 5H), 7.09-6.98 (m, 4H), 6.86 (d, J = 2.4 Hz, | 676.735 | | |
| | 1H), 6.83 (dd, J = 8.4, 2.4 Hz, 1H), 4.37 (d, J = 10.8 Hz, | Found | | |
| | 2H), 3.84 (t, J = 5.0 Hz, 4H), 3.78 (q, J = 4.4 Hz, 2H), | [M + H]⁺ | | |
| | 3.20 (t, J = 5.1 Hz, 4H). | (ESI+) = 676.2 | | |
| I-86 | ¹H NMR: (400 MHz, DMSO-d₆) δ 8.94 (d, J = 7.1 Hz, | Calculated | 14.3% | White |
| | 1H), 7.34 (d, J = 4.3 Hz, 6H), 7.22 (s, 1H), 7.08 (q, J = | exact mass = | | solid |
| | 4.3 Hz, 1H), 7.04 (d, J = 8.4 Hz, 1H), 6.83 (d, J = 2.4 Hz, | 735.949 | | |
| | 1H), 6.79 (dd, J = 8.4, 2.4 Hz, 1H), 5.41-5.19 (m, 6H), | Found | | |
| | 4.69-4.54 (m, 1H), 4.36 (d, J = 9.8 Hz, 3H), 4.09 (t, J = | [M + H]⁺ | | |
| | 9.0 Hz, 1H), 3.98 (dd, J = 8.8, 5.3 Hz, 1H), 3.78 (dq, J = | (ESI+) = 735.5 | | |
| | 9.5, 4.9 Hz, 3H), 2.76 (t, J = 5.9 Hz, 4H), 2.08-1.93 (m, | | | |
| | 6H), 1.44 (t, J = 7.2 Hz, 2H), 1.26 (dd, J = 18.1, 5.5 Hz, | | | |
| | 9H), 0.92 (t, J = 7.5 Hz, 3H). | | | |
| I-87 | ¹H NMR: (400 MHz, DMSO-d₆) δ 8.44 (s, 2H), 8.25 | Calculated | 27.3% | White |
| | (d, J = 7.9 Hz, 1H), 7.32 (d, J = 4.3 Hz, 6H), 7.20 (s, 1H), 7.08 | exact mass = | | solid |
| | (q, J = 4.4 Hz, 1H), 7.02 (d, J = 8.4 Hz, 1H), 6.84-6.75 | 599.651 | | |
| | (m, 2H), 4.55 (d, J = 13.1 Hz, 2H), 4.34 (d, J = 14.6 Hz, | Found | | |
| | 2H), 4.13-3.97 (m, 1H), 3.75 (q, J = 4.9 Hz, 2H), 2.99 | [M + H]⁺ | | |
| | (t, J = 12.6 Hz, 2H), 1.83 (d, J = 11.7 Hz, 2H), 1.42 (d, J = | (ESI+) = 599.2 | | |
| | 12.6 Hz, 2H). | | | |
| I-88 | ¹H NMR: (400 MHz, DMSO-d₆) δ 9.49 (s, 1H), 8.59 (dd, | Calculated | 1.4% | White |
| | J = 17.5, 5.9 Hz, 1H), 7.95-7.85 (m, 1H), 7.59-7.26 | exact mass = | | solid |
| | (m, 6H), 7.15 (d, J = 12.3 Hz, 1H), 7.11-7.07 (m, 1H), | 807.07 | | |
| | 7.04 (d, J = 8.4 Hz, 1H), 6.85 (d, J = 2.4 Hz, 1H), 6.77 (d, | Found | | |
| | J = 8.5 Hz, 1H), 5.44-5.18 (m, 5H), 4.48-4.33 (m, | [M + H]⁺ | | |
| | 3H), 3.93-3.73 (m, 4H), 3.16-3.02 (m, 6H), 2.96- | (ESI+) = 806.3 | | |
| | 2.87 (m, 1H), 2.76 (t, J = 6.1 Hz, 3H), 2.41-2.17 (m, | | | |
| | 2H), 2.03 (tdd, J = 12.4, 7.6, 4.3 Hz, 7H), 1.78-1.68 | | | |
| | (m, 2H), 1.48 (d, J = 10.1 Hz, 3H), 1.24 (s, 9H), 0.92 | | | |
| | (t, J = 7.5 Hz, 3H). | | | |
| I-89 | ¹H NMR: (400 MHz, DMSO-d₆) δ 10.15 (s, 1H), 8.43 (d, | Calculated | 38.6% | White |
| | J = 0.8 Hz, 2H), 7.68-7.59 (m, 2H), 7.49 (s, 1H), 7.38- | exact mass = | | solid |
| | 7.25 (m, 7H), 7.06 (dd, J = 7.7, 3.2 Hz, 2H), 6.87 (d, J = | 690.761 | | |
| | 2.4 Hz, 1H), 6.83 (dd, J = 8.4, 2.4 Hz, 1H), 4.44-4.30 | Found | | |
| | (m, 2H), 3.82-3.73 (m, 2H), 3.67 (t, J = 5.0 Hz, 4H), | [M + H]⁺ | | |
| | 3.46 (s, 2H), 2.41 (t, J = 5.1 Hz, 4H). | (ESI+) = 690.2 | | |
| I-90 | ¹H NMR: (400 MHz, DMSO-d₆) δ 7.35 (dd, J = 8.5, 7.1 | Calculated | 9.3% | White |
| | Hz, 3H), 7.31-7.18 (m, 3H), 7.09 (t, J = 7.3 Hz, 1H), | exact mass = | | solid |
| | 7.00 (d, J = 8.4 Hz, 1H), 6.81 (d, J = 2.4 Hz, 1H), 6.78 | 707.939 | | |
| | (dd, J = 8.4, 2.4 Hz, 1H), 6.53 (s, 1H), 5.17 (d, J = 6.4 | Found | | |
| | Hz, 1H), 5.11-4.99 (m, 2H), 4.35 (d, J = 5.1 Hz, 2H), | [M + H]⁺ | | |
| | 4.03 (d, J = 13.0 Hz, 2H), 3.75 (t, J = 4.4 Hz, 2H), 3.16 | (ESI+) = 707.3 | | |
| | (d, J = 6.6 Hz, 2H), 3.06 (d, J = 14.9 Hz, 2H), 2.70 (d, J = | | | |
| | 4.1 Hz, 1H), 1.98 (ddt, J = 31.9, 15.5, 7.4 Hz, 9H), 1.80 | | | |
| | (dd, J = 13.6, 3.8 Hz, 2H), 1.65-1.57 (m, 6H), 1.55 (d, J = | | | |
| | 5.6 Hz, 6H), 1.18 (d, J = 10.7 Hz, 2H). | | | |
| I-91 | ¹H NMR: (400 MHz, DMSO-d₆) δ 7.60-7.21 (m, 6H), | Calculated | 13.1% | White |
| | 7.12-7.06 (m, 1H), 7.00 (d, J = 8.4 Hz, 1H), 6.84- | exact mass = | | solid |
| | 6.68 (m, 3H), 5.16 (s, 1H), 5.04 (d, J = 7.2 Hz, 2H), 4.34 | 693.913 | | |
| | (d, J = 4.4 Hz, 2H), 3.76 (t, J = 4.4 Hz, 2H), 3.60 (d, J = | Found | | |
| | 49.5 Hz, 3H), 3.13 (s, 2H), 2.18-1.79 (m, 10H), 1.73- | [M + H]⁺ | | |
| | 1.48 (m, 12H). | (ESI+) = 693.1 | | |
| I-142 | ¹H NMR: (400 MHz, DMSO-d₆) δ 7.36 (dd, J = 8.5, 7.2 | Calculated | 17.6% | White |
| | Hz, 2H), 7.31-7.26 (m, 2H), 7.10 (d, J = 7.6 Hz, 1H), | exact mass = | | solid |
| | 7.02 (d, J = 8.4 Hz, 1H), 6.86 (s, 1H), 6.84 (d, J = 2.4 Hz, | 705.801 | | |
| | 1H), 6.77 (dd, J = 8.5, 2.4 Hz, 1H), 4.36 (t, J = 4.3 Hz, | Found | | |
| | 2H), 3.30 (d, , J = 8.8, 2H). | [M + H]⁺ | | |
| | | (ESI+) = 705.2 | | |
| I-144 | ¹H NMR: (400 MHz, DMSO-d₆) δ 9.35 (s, 1H), 8.55 (s, | Calculated | 8.0% | White |
| | 1H), 8.37 (s, 1H), 8.23 (s, 1H), 7.88 (s, 2H), 7.67-7.50 | exact mass = | | solid |
| | (m, 1H), 7.45-7.18 (m, 8H), 7.15-6.94 (m, 3H), 6.85- | 705.801 | | |
| | 6.73 (m, 2H), 4.69-4.51 (m, 2H), 4.36 (d, J = 13.7 | Found | | |
| | Hz, 2H), 4.09-3.85 (m, 1H), 3.77 (s, 2H), 3.61 (d, J = | [M + H]⁺ | | |
| | 13.6 Hz, 1H), 2.97 (s, 1H), 2.74-2.56 (m, 1H), 2.04 (d, | (ESI+) = 705.2 | | |
| | J = 38.9 Hz, 1H), 1.81 (d, J = 41.4 Hz, 3H), 1.48 (d, J = | | | |
| | 62.9 Hz, 1H). | | | |
| I-153 | Free base: ¹H NMR: (400 MHz, DMSO-d₆) δ 8.15 (d, J = | Calculated | 2.8% | Pale |
| | 7.9 Hz, 1H), 7.44-7.25 (m, 8H), 7.21-7.11 (m, 2H), | exact mass = | | yellow |
| | 7.10-7.05 (m, 1H), 7.03 (d, J = 8.4 Hz, 1H), 6.94 (td, J = | 739.805 | | solid |
| | 8.6, 2.6 Hz, 1H), 6.84 (d, J = 1.3 Hz, 1H), 6.81-6.74 | Found | | |
| | (m, 2H), 6.71 (s, 1H), 5.61 (s, 1H), 4.29 (dt, J = 29.8, | [M + H]⁺ | | |
| | 14.8 Hz, 4H), 3.77 (s, 2H), 3.59 (d, J = 8.6 Hz, 1H), 2.85 | (ESI+) = 739.3 | | |
| | (dd, J = 28.4, 12.5 Hz, 2H), 2.68-2.57 (m, 2H), 2.15 (q, | | | |
| | J = 11.1 Hz, 2H), 1.56 (s, 2H), 1.35 (s, 2H). | | | |
| | HCl salt : 1H NMR: (400 MHz, DMSO-d6) δ 10.12 (s, | | | |
| | 1H), 8.90 (s, 1H), 8.45 (s, 1H), 7.51 (s, 2H), 7.41-7.16 | | | |
| | (m, 11H), 7.16-6.97 (m, 4H), 6.87-6.69 (m, 2H), | | | |
| | 4.85 (d, J = 13.8 Hz, 1H), 4.57 (d, J = 14.2 Hz, 1H), 4.37 | | | |
| | (s, 2H), 3.83 (d, J = 48.0 Hz, 6H), 1.80 (d, J = 37.0 Hz, | | | |
| | 5H). | | | |
| I-154 | Free base : ¹H NMR: (400 MHz, DMSO-d₆) δ 8.19 (d, J = | Calculated | 5.0% | Pale |
| | 8.4 Hz, 2H), 7.83 (s, 1H), 7.43 (dt, J = 6.5, 1.3 Hz, 2H), | exact mass = | | yellow |
| | 7.38-7.31 (m, 5H), 7.31-7.24 (m, 3H), 7.21 (d, J = | 704.813 | | solid |
| | 8.1 Hz, 2H), 7.08 (dq, J = 5.4, 2.6 Hz, 1H), 7.04 (d, J = | Found | | |
| | 8.4 Hz, 1H), 6.81 (d, J = 2.4 Hz, 1H), 6.78 (dd, J = 8.4, | [M + H]⁺ | | |
| | 2.4 Hz, 1H), 5.32 (s, 1H), 4.58 (q, J = 14.1 Hz, 2H), 4.36 | (ESI+) = 704.4 | | |
| | (d, J = 11.4 Hz, 2H), 3.78 (s, 2H), 3.65-3.53 (m, 1H), | | | |
| | 2.82 (d, J = 11.3 Hz, 1H), 2.68 (d, J = 13.7 Hz, 1H), 2.56 | | | |
| | (s, 1H), 2.44 (d, J = 11.1 Hz, 1H), 2.17-2.05 (m, 2H), | | | |
| | 1.64-1.37 (m, 4H). | | | |
| | HCl salt: ^{1H NMR:} (400 MHz, DMSO-d₆) δ 8.42-8.15 (m, | | | |
| | 2H), 7.90 (d, J = 63.7 Hz, 1H), 7.45 (dt, J = 27.2, 9.1 Hz, | | | |
| | 3H), 7.37-7.24 (m, 7H), 7.22 (s, 2H), 7.10-6.98 (m, | | | |
| | 2H), 6.84-6.72 (m, 2H), 5.32 (s, 1H), 4.58 (q, J = 14.0 | | | |
| | Hz, 2H), 4.37 (s, 2H), 3.78 (s, 3H), 3.63 (d, J = 14.9 Hz, | | | |
| | 1H), 3.04 (s, 1H), 2.80 (s, 1H), 2.72-2.61 (m, 1H), 2.11 | | | |
| | (s, 1H), 1.91 (s, 1H), 1.83-1.36 (m, 4H). | | | |
| I-160 | ¹H NMR: (400 MHz, DMSO-d₆) δ 14.38 (s, 1H), 9.79 (d, | Calculated | 93.2% | White |
| | J = 212.0 Hz, 1H), 8.78 (d, J = 18.5 Hz, 1H), 8.50-8.26 | exact mass = | | solid |
| | (m, 1H), 7.57-7.45 (m, 3H), 7.44-7.30 (m, 8H), 7.30- | 703.825 | | |
| | 7.16 (m, 3H), 7.10 (s, 2H), 7.02 (d, J = 8.4 Hz, 1H), | Found | | |
| | 6.88-6.72 (m, 2H), 4.93 (d, J = 14.0 Hz, 1H), 4.60 | [M + H]⁺ | | |
| | (t, J = 12.6 Hz, 1H), 4.37 (s, 2H), 4.00-3.53 (m, 6H), 3.29- | (ESI+) = 703.3 | | |
| | 3.11 (m, 2H), 2.89 (d, J = 10.6 Hz, 1H), 2.13-1.90 (m, | | | |
| | 2H), 1.77 (d, J = 15.8 Hz, 2H). | | | |
| I-163 | ¹H NMR: (400 MHz, DMSO-d₆) δ 8.57 (t, J = 5.7 Hz, | Calculated | 11.0% | White |
| | 1H), 8.45 (s, 1H), 7.96 (s, 1H), 7.40-7.26 (m, 5H), 7.13- | exact mass = | | solid |
| | 7.05 (m, 2H), 7.02 (d, J = 8.4 Hz, 1H), 6.80 (d, J = 2.4 | 515.559 | | |
| | Hz, 1H), 6.75 (dd, J = 8.4, 2.4 Hz, 1H), 4.43-4.26 (m, | Found | | |
| | 4H), 3.81-3.69 (m, 2H), 3.63-3.51 (m, 2H). | [M + H]⁺ | | |
| | | (ESI+) = 515.0 | | |
| I-164 | ¹H NMR: (400 MHz, DMSO-d₆) δ 8.96 (t, J = 5.9 Hz, | Calculated | 9.2% | White |
| | 1H), 8.36 (s, 1H), 7.44-7.30 (m, 5H), 7.25 (s, 1H), 7.07 | exact mass = | | solid |
| | (dt, J = 6.1, 3.4 Hz, 1H), 7.02 (d, J = 8.4 Hz, 1H), 6.81 (d, | 515.559 | | |
| | J = 2.4 Hz, 1H), 6.77 (dd, J = 8.4, 2.4 Hz, 1H), 4.45- | Found | | |
| | 4.28 (m, 4H), 3.80 (s, 3H), 3.76 (d, J = 4.6 Hz, 2H). | [M + H]⁺ | | |
| | | (ESI+) = 515.0 | | |
| I-167 | ¹H NMR: (400 MHz, DMSO-d₆) δ 8.47 (d, J = 6.9 Hz, | Calculated | 1.7% | White |
| | 1H), 8.37 (s, 1H), 7.34 (d, J = 4.4 Hz, 5H), 7.26 (s, 1H), | exact mass = | | solid |
| | 7.08 (q, J = 4.2 Hz, 1H), 7.04 (d, J = 8.4 Hz, 1H), 6.83- | 584.664 | | |
| | 6.76 (m, 2H), 4.33 (dt, J = 21.8, 6.6 Hz, 3H), 3.76 (t, J = | Found | | |
| | 4.7 Hz, 4H), 3.63 (s, 3H), 2.78 (s, 1H), 2.61 (s, 1H), 2.40 | [M + H]⁺ | | |
| | (dd, J = 9.4, 5.6 Hz, 1H), 2.11 (s, 1H), 1.73 (s, 1H). | (ESI+) = 584.2 | | |
| I-168 | ¹H NMR: (400 MHz, DMSO-d₆) δ 8.57 (t, J = 5.7 Hz, | Calculated | 24.3% | White |
| | 1H), 8.32 (s, 1H), 7.60-7.22 (m, 6H), 7.14 (s, 1H), 7.10- | exact mass = | | solid |
| | 7.06 (m, 1H), 7.03 (d, J = 8.4 Hz, 1H), 6.82 (d, J = 2.4 | 529.586 | | |
| | Hz, 1H), 6.77 (dd, J = 8.4, 2.4 Hz, 1H), 4.36 (d, J = 11.4 | Found | | |
| | Hz, 2H), 3.78 (s, 5H), 3.47 (d, J = 6.6 Hz, 2H), 2.80 (t, J = | [M + H]⁺ | | |
| | 7.6 Hz, 2H). | (ESI+) = 529.2 | | |
| I-169 | ¹H NMR: (400 MHz, DMSO-d₆) δ 8.66 (t, J = 5.8 Hz, | Calculated | 10.0% | White |
| | 1H), 7.78 (s, 1H), 7.49-7.25 (m, 6H), 7.15-7.06 (m, | exact mass = | | solid |
| | 2H), 7.03 (d, J = 8.4 Hz, 1H), 6.82 (d, J = 2.4 Hz, 1H), | 529.586 | | |
| | 6.77 (dd, J = 8.4, 2.4 Hz, 1H), 4.49-4.20 (m, 2H), 3.84- | Found | | |
| | 3.70 (m, 5H), 3.50 (q, J = 6.8 Hz, 2H), 2.96 (t, J = 7.2 | [M + H]⁺ | | |
| | Hz, 2H). | (ESI+) = 529.1 | | |
| I-170 | ¹H NMR: (400 MHz, DMSO-d₆) δ 8.35 (s, 1H), 8.23 | Calculated | 4.4% | White |
| | (d, J = 7.9 Hz, 1H), 7.93 (s, 1H), 7.38-7.29 (m, 5H), 7.24 (s, | exact mass = | | solid |
| | 1H), 7.09 (dt, J = 5.1, 3.2 Hz, 1H), 7.04 (d, J = 8.3 Hz, | 642.743 | | |
| | 1H), 6.83-6.77 (m, 2H), 4.69 (s, 1H), 4.35 (d, J = 11.9 | Found | | |
| | Hz, 2H), 4.16 (d, J = 1.9 Hz, 2H), 3.77 (q, J = 4.4 Hz, 2H), | [M + H]⁺ | | |
| | 3.73-3.62 (m, 1H), 3.08 (d, J = 11.4 Hz, 1H), 2.96- | (ESI+) = 642.2 | | |
| | 2.87 (m, 1H), 2.32-2.14 (m, 4H), 1.69 (t, J = 7.5 Hz, | | | |
| | 2H), 1.58 (s, 2H), 0.98 (s, 3H). | | | |

### Synthesis of ethyl-3-amino-7-((1-(2-(2,4-difluorophenyl)-2-hydroxy-3-(1H-1,2,4-triazol-1-yl)propyl)piperidin-4-yl)carbamoyl)-2-oxo-1-(4-phenyl-3,4-dihydro-2H-benzo[b][1,4]oxazin-6-yl)-1,2-dihydrothieno[2,3-b]pyrazine-6-carboxylate—I-58)

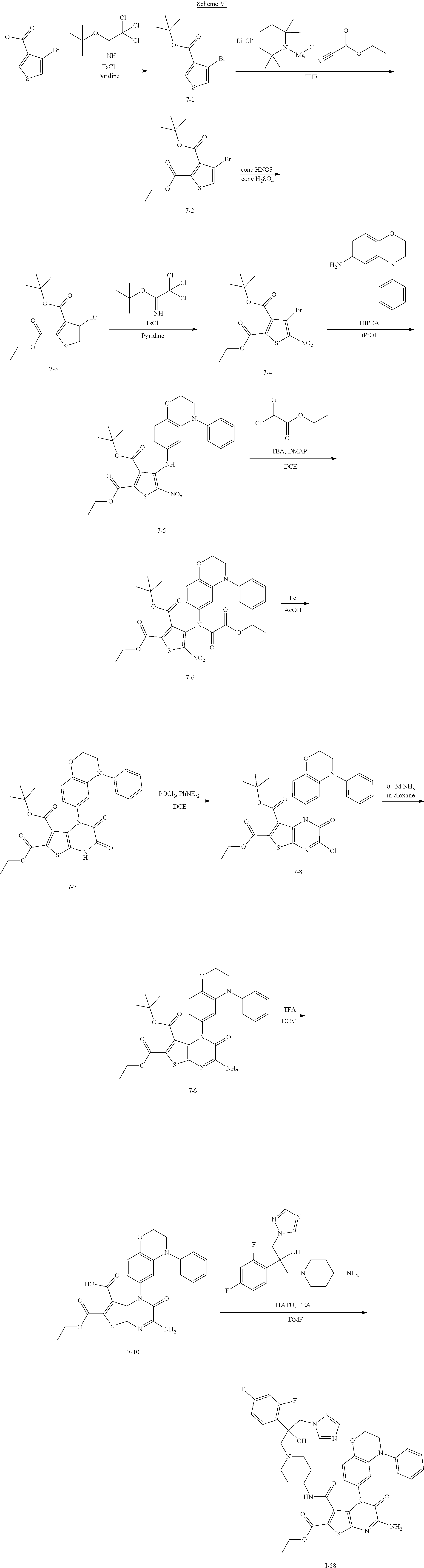

### Tert-butyl-4-bromothiophene-3-carboxylate (7-1)

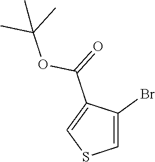

A solution of 4-bromothiophene-3-carboxylic acid (33 g, 160 mmol) in pyridine (300 mL) was cooled to 0° C. and treated with 4-methylbenzenesulfonyl chloride (59.4 g, 320 mmol) and t-BuOH (30.7 mL, 320 mmol). The reaction was stirred for 2 hours at 0° C., then for 16 hours at room temperature. The reaction mixture was poured into 500 mL of saturated NaHCO₃ aqueous solution. The aqueous phase was extracted with EtOAc (3×500 mL), and the organic layers were combined, dried over Na₂SO₄, filtered and concentrated. The crude product was purified by column chromatography on silica gel (PE/EA=10:1) to give tert-butyl 4-bromothiophene-3-carboxylate 7-1 as a pale yellow solid (35 g, 83% yield).

¹H NMR (400 MHz, DMSO) δ 8.32 (d, J=3.5 Hz, 1H), 7.77 (d, J=3.5 Hz, 1H), 1.53 (s, 9H).

### 3-(tert-butyl)-2-ethyl 4-bromothiophene-2,3-dicarboxylate (7-2)

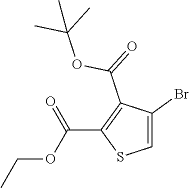 [SMILES: CCOC(=O)c1scc(Br)c1C(=O)OC(C)(C)C]

Tert-butyl-4-bromothiophene-3-carboxylate 7-1 (30 g, 110 mmol) was dissolved in 300 mL dry THF and the solution was cooled to −30° C., lithium magnesium 2,2,6,6-tetramethylpiperidin-1-ide dichloride (136.8 mL, 140 mmol) was added dropwise and stirred at −30° C. for 30 minutes. Ethyl carbonocyanidate (17.1 mL, 170 mmol) was added dropwise and stirred at 0° C. for 30 minutes under N₂ atmosphere. The reaction mixture was poured into 500 mL of saturated NH₄Cl aqueous solution. The aqueous phase was extracted with EtOAc (3×500 mL), and the organic layers were combined, dried over Na₂SO₄, filtered and concentrated. The crude product was purified by column chromatography on silica gel (PE/EA=10:1) to give 3-(tert-butyl)-2-ethyl-4-bromothiophene-2,3-dicarboxylate 7-2 as a pale yellow oil (35 g, 95% yield).

¹H NMR (400 MHz, DMSO) δ 8.13 (s, 1H), 4.31 (q, J=7.1 Hz, 2H), 1.55 (s, 1OH), 1.27 (t, J=7.1 Hz, 4H).

### 4-bromo-2-(ethoxycarbonyl)-5-nitrothiophene-3-carboxylic acid (7-3)

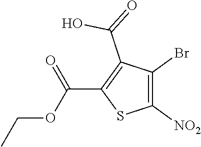 [SMILES: CCOC(=O)c1sc([N+](=O)[O-])c(Br)c1C(=O)O]

3-(tert-butyl)-2-ethyl-4-bromothiophene-2,3-dicarboxylate 7-2 (40 g, 120 mmol) was dissolved in 80 mL H₂SO₄ and the solution was cooled to −10° C. A mixture of 20 mL HNO₃ and 16 mL H₂SO₄ was added dropwise and stirred at 0° C. for 30 minutes. The reaction mixture was poured into 500 mL ice water. The solid was collected by filtration and washed with H₂O (500 mL) and dried by vacuum to give 4-bromo-2-(ethoxycarbonyl)-5-nitrothiophene-3-carboxylic acid 7-3 as a pale yellow solid (35 g, 90% yield).

¹H NMR (400 MHz, DMSO) b 4.35 (t, J=7.1 Hz, 1H), 1.30 (t, J=7.1 Hz, 2H).

### 3-(tert-butyl)-2-ethyl-4-bromo-5-nitrothiophene-2,3-dicarboxylate (7-4)

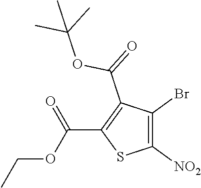 [SMILES: CCOC(=O)c1sc([N+](=O)[O-])c(Br)c1C(=O)OC(C)(C)C]

4-bromo-2-(ethoxycarbonyl)-5-nitrothiophene-3-carboxylic acid 7-3 (35 g, 110 mmol) was dissolved in 400 mL dry DCM and the solution was cooled to 0° C., tert-butyl 2,2,2-trichloroacetimidate (117 g, 550 mmol) was added and stirred at room temperature for 10 minutes. The reaction mixture was poured into 500 mL H₂O. The aqueous phase was extracted with DCM (3×500 mL), and the organic layers were combined, dried over Na₂SO₄, filtered and concentrated. The crude product was purified by column chromatography on silica gel (PE/EA=10:1) to 3-(tert-butyl)-2-ethyl-4-bromo-5-nitrothiophene-2,3-dicarboxylate 7-4 as a pale yellow solid (38 g, 91% yield).

¹H NMR (400 MHz, DMSO) b 4.38 (q, J=7.1 Hz, 1H), 1.57 (s, 4H), 1.30 (t, J=7.1 Hz, 2H).

### 3-(tert-butyl)-2-ethyl-5-nitro-4-((4-phenyl-3,4-dihydro-2H-benzo[b][1,4]oxazin-6-yl)amino)thiophene-2,3-dicarboxylate (7-5)

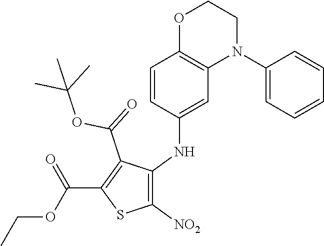 [SMILES: CCOC(=O)c1sc([N+](=O)[O-])c(Nc2ccc3c(c2)N(c2ccccc2)CCO3)c1C(=O)OC(C)(C)C]

3-(tert-butyl)-2-ethyl-5-nitro-4-((4-phenyl-3,4-dihydro-2H-benzo[b][1,4]oxazin-6-yl)amino)thiophene-2,3-dicarboxylate 7-5 was synthesized following the same procedure used for methyl-5-nitro-4-((4-phenyl-3,4-dihydro-2H-benzo[b][1,4]oxazin-6-yl)amino)thiophene-2-carboxylate 1-6 to obtain a yellow solid in 76% yield.

LCMS: Calculated Exact Mass=525.2, Found [M+H]⁺ (ESI+)=526.5.

### 3-(tert-butyl)-2-ethyl-4-(2-ethoxy-2-oxo-N-(4-phenyl-3,4-dihydro-2H-benzo[b][1,4]oxazin-6-yl) acetamido)-5-nitrothiophene-2,3-dicarboxylate (7-6)

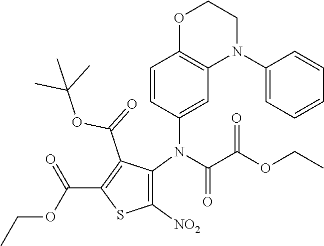 [SMILES: CCOC(=O)C(=O)N(c1ccc2c(c1)N(c1ccccc1)CCO2)c1c([N+](=O)[O-])sc(C(=O)OCC)c1C(=O)OC(C)(C)C]

3-(tert-butyl)-2-ethyl-4-(2-ethoxy-2-oxo-N-(4-phenyl-3,4-dihydro-2H-benzo[b][1,4]oxazin-6-yl)acetamido)-5-nitrothiophene-2,3-dicarboxylate 7-6 was synthesized following the same procedure used for methyl-4-(2-ethoxy-2-oxo-N-(4-phenyl-3,4-dihydro-2H-benzo[b][1,4]oxazin-6-yl)acetamido)-5-nitrothiophene-2-carboxylate 1-7 to obtain a yellow solid in 63% yield.

LCMS: Calculated Exact Mass=625.2, Found [M+H]⁺ (ESI+)=626.1.

### 7-(tert-butyl)-6-ethyl-2,3-dioxo-1-(4-phenyl-3,4-dihydro-2H-benzo[b][1,4]oxazin-6-yl)-1,2,3,4-tetrahydrothieno[2,3-b]pyrazine-6,7-dicarboxylate (7-7)

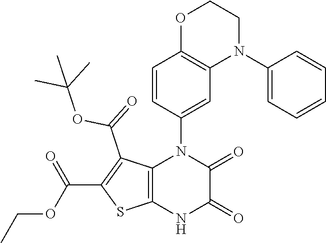 [SMILES: CCOC(=O)c1sc2[nH]c(=O)c(=O)n(-c3ccc4c(c3)N(c3ccccc3)CCO4)c2c1C(=O)OC(C)(C)C]

7-(tert-butyl)-6-ethyl-2,3-dioxo-1-(4-phenyl-3,4-dihydro-2H-benzo[b][1,4]oxazin-6-yl)-1,2,3,4-tetrahydrothieno[2,3-b]pyrazine-6,7-dicarboxylate 7-7 was synthesized following the same procedure used for methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate 1-8 to obtain a crude brown solid.

LCMS: Calculated Exact Mass=549.2, Found [M+H]⁺ (ESI+)=550.0.

7-(tert-butyl)-6-ethyl-3-chloro-2-oxo-1-(4-phenyl-3,4-dihydro-2H-benzo[b][1,4]oxazin-6-yl)-1,2-dihydrothieno[2,3-b]pyrazine-6,7-dicarboxylate (7-8)

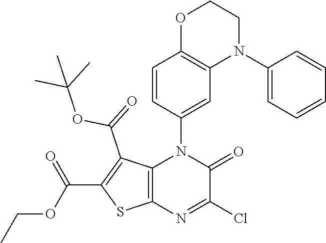 [SMILES: CCOC(=O)c1sc2nc(Cl)c(=O)n(-c3ccc4c(c3)N(c3ccccc3)CCO4)c2c1C(=O)OC(C)(C)C]

7-(tert-butyl)-6-ethyl-3-chloro-2-oxo-1-(4-phenyl-3,4-dihydro-2H-benzo[b][1,4]oxazin-6-yl)-1,2-dihydrothieno[2,3-b]pyrazine-6,7-dicarboxylate 7-8 was synthesized following the same procedure used for methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate 1-9 to obtain a yellow solid in 17% yield for two steps.

LCMS: Calculated Exact Mass=567.1, Found [M+H]⁺ (ESI+)=568.1.

### 7-(tert-butyl)-6-ethyl-3-amino-2-oxo-1-(4-phenyl-3,4-dihydro-2H-benzo[b][1,4]oxazin-6-yl)-1,2-dihydrothieno[2,3-b]pyrazine-6,7-dicarboxylate (7-9)

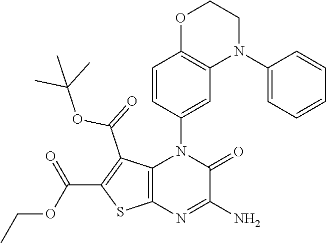 [SMILES: CCOC(=O)c1sc2nc(N)c(=O)n(-c3ccc4c(c3)N(c3ccccc3)CCO4)c2c1C(=O)OC(C)(C)C]

7-(tert-butyl)-6-ethyl-3-amino-2-oxo-1-(4-phenyl-3,4-dihydro-2H-benzo[b][1,4]oxazin-6-yl)-1,2-dihydrothieno[2,3-b]pyrazine-6,7-dicarboxylate 7-9 was synthesized following the same procedure used for methyl-3-amino-2-oxo-1-(4-phenyl-3,4-dihydro-2H-benzo[b][1,4]oxazin-6-yl)-1,2-dihydrothieno[2,3-b]pyrazine-6-carboxylate 1-10 to obtain a yellow solid in 88% yield.

LCMS: Calculated Exact Mass=548.1, Found [M+H]⁺ (ESI+)=549.2.

### 3-amino-6-(ethoxycarbonyl)-2-oxo-1-(4-phenyl-3,4-dihydro-2H-benzo[b][1,4]oxazin-6-yl)-1,2-dihydrothieno[2,3-b]pyrazine-7-carboxylic acid (7-10)

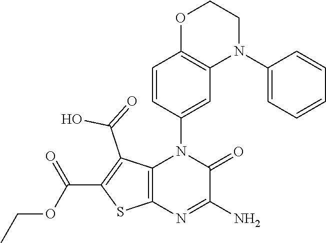 [SMILES: CCOC(=O)c1sc2nc(N)c(=O)n(-c3ccc4c(c3)N(c3ccccc3)CCO4)c2c1C(=O)O]

7-(tert-butyl)-6-ethyl-3-amino-2-oxo-1-(4-phenyl-3,4-dihydro-2H-benzo[b][1,4]oxazin-6-yl)-1,2-dihydrothieno[2,3-b]pyrazine-6,7-dicarboxylate 7-9 (850 mg, 1.55 mmol) was dissolved in 10 mL DCM and TFA (2 mL) was added. The reaction was stirred at room temperature for 1 hour. The mixture was concentrated. The crude was purified by Prep-HPLC (ACN/H₂O with 0.05% NH₃ as the mobile phase) to afford 3-amino-6-(ethoxycarbonyl)-2-oxo-1-(4-phenyl-3,4-dihydro-2H-benzo[b][1,4]oxazin-6-yl)-1,2-dihydrothieno[2,3-b]pyrazine-7-carboxylic acid 7-10 as a pale yellow solid (600 mg, 79%).

LCMS: Calculated Exact Mass=492.1, Found [M+H]⁺ (ESI+)=493.2.

¹H NMR (400 MHz, DMSO) δ 13.03 (s, 1H), 8.04-7.17 (m, 6H), 7.09-6.95 (m, 1H), 6.81 (s, 1H), 6.75 (d, J=2.3 Hz, 1H), 6.61 (s, 1H), 4.37 (dt, J=10.2, 3.5 Hz, 1H), 4.32-4.07 (m, 3H), 3.81-3.66 (m, 2H), 1.22 (t, J=7.1 Hz, 3H).

### Ethyl-3-amino-7-((1-(2-(2,4-difluorophenyl)-2-hydroxy-3-(1H-1,2,4-triazol-1-yl)propyl)piperidin-4-yl)carbamoyl)-2-oxo-1-(4-phenyl-3,4-dihydro-2H-benzo[b][1,4]oxazin-6-yl)-1,2-dihydrothieno[2,3-b]pyrazine-6-carboxylate—I-58)

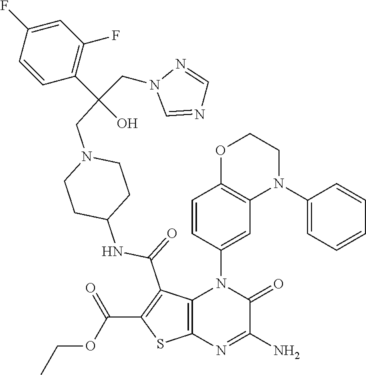 [SMILES: CCOC(=O)c1sc2nc(N)c(=O)n(-c3ccc4c(c3)N(c3ccccc3)CCO4)c2c1C(=O)NC1CCN(CC(O)(Cn2cncn2)c2ccc(F)cc2F)CC1]

3-amino-6-(ethoxycarbonyl)-2-oxo-1-(4-phenyl-3,4-dihydro-2H-benzo[b][1,4]oxazin-6-yl)-1,2-dihydrothieno[2,3-b]pyrazine-7-carboxylic acid 7-10 (450 mg, 0.91 mmol) was dissolved in 2 mL DMF and treated with HATU (521 mg, 1.37 mmol), Et₃N (460 mg, 4.5 mmol) at 0° C. and stirred for 5 minutes. 1-(4-aminopiperidin-1-yl)-2-(2,4-difluorophenyl)-3-(1H-1,2,4-triazol-1-yl)propan-2-ol (613 mg, 1.73 mmol) was added. The mixture was stirred at room temperature for 1 hour. The reaction mixture was poured into 50 mL H₂O. The aqueous phase was extracted with EtOAc (3×50 mL) and the organic layers were combined, dried over Na₂SO₄, filtered and concentrated. The crude product was purified by column chromatography on silica gel (DCM/MeOH=5:1) to afford ethyl-3-amino-7-((1-(2-(2,4-difluorophenyl)-2-hydroxy-3-(1H-1,2,4-triazol-1-yl) propyl) piperid in-4-yl)carbamoyl)-2-oxo-1-(4-phenyl-3,4-dihydro-2H-benzo[b][1,4]oxazin-6-yl)-1,2-dihydrothieno[2,3-b]pyrazine-6-carboxylate I-58 as a white solid (330 mg, 45% yield).

LCMS: Calculated Exact Mass=811.3, Found [M+H] (ESI+)=812.2.

¹H NMR (400 MHz, DMSO) δ 8.28 (s, 1H), 7.82 (d, J=4.7 Hz, 1H), 7.74 (d, J=1.2 Hz, 1H), 7.36 (dt, J=21.3, 8.0 Hz, 6H), 7.14 (ddd, J=15.4, 9.0, 4.4 Hz, 1H), 7.05 (d, J=7.8 Hz, 1H), 6.94 (td, J=8.6, 1.2 Hz, 1H), 6.78 (d, J=8.5 Hz, 1H), 6.74 (d, J=2.3 Hz, 1H), 6.51 (d, J=8.0 Hz, 1H), 5.61 (s, 1H), 4.60-4.47 (m, 2H), 4.35-4.32 (, 1H), 4.23-24.09 (i, 3H), 3.83 3.72 (i, 1H), 3.7.5-3.63 (i, 1H), 2.97-2.88 (, 1H), 2.84 (d, J=13.9 Hz, 1H), 2.72-2.56 (m, 3H), 2.12-2.07 (m, 2H), 1.61-1.42 (m, 2H), 1.19 (t, J=7.1 Hz, 3H), 1.09-1.06 (in, 2H).

In a similar manner, using the above procedure, the following amide compounds were synthesized.

| Compound | | | Yield | |
|---|---|---|---|---|
| ID# | ¹H NMR | LCMS | % | Appearance |
| I-59 | ¹H NMR: (400 MHz, DMSO-d₆) δ 8.27 (s, 1H), 8.17 (d, | Calculated | 9.9% | White |
| | J = 6.3 Hz, 1H), 7.75 (d, J = 1.1 Hz, 1H), 7.49-7.21 (m, | exact mass = | | solid |
| | 7H), 7.14 (q, J = 9.5 Hz, 1H), 7.06 (t, J = 7.4 Hz, 1H), | 783.818 | | |
| | 6.95 (t, J = 8.5 Hz, 1H), 6.79 (d, J = 8.3 Hz, 2H), 6.54 | Found | | |
| | (dd, J = 8.4, 2.4 Hz, 1H), 5.61 (s, 1H), 4.58-4.45 (m, | [M + H]⁺ | | |
| | 2H), 4.27 (d, J = 26.5 Hz, 2H), 3.77 (d, J = 13.0 Hz, 1H), | (ESI+) = | | |
| | 3.61 (s, 1H), 2.91-2.79 (m, 2H), 2.75-2.59 (m, 4H), | 783.2 | | |
| | 2.08 (q, J = 11.0 Hz, 2H), 1.52 (s, 2H), 1.05 (t, J = 7.0 | | | |
| | Hz, 2H). | | | |
| I-60 | ¹H NMR: (400 MHz, DMSO-d₆) δ 8.27 (s, 1H), 8.18- | Calculated | 7.9% | White |
| | 8.08 (m, 1H), 7.74 (s, 1H), 7.58 (s, 1H), 7.43-7.30 (m, | exact mass = | | solid |
| | 4H), 7.27 (s, 2H), 7.18-7.10 (m, 1H), 7.06 (t, J = 7.3 | 811.871 | | |
| | Hz, 1H), 6.94 (td, J = 8.5, 2.6 Hz, 1H), 6.78 (d, J = 8.5 | Found | | |
| | Hz, 2H), 6.53 (dd, J = 8.4, 2.4 Hz, 1H), 5.60 (d, J = 4.7 | [M + H]⁺ | | |
| | Hz, 1H), 4.53 (d, J = 2.3 Hz, 2H), 4.27 (d, J = 20.5 Hz, | (ESI+) = | | |
| | 2H), 3.78 (dd, J = 12.7, 4.9 Hz, 1H), 3.60 (d, J = 12.4 Hz, | 811.2 | | |
| | 1H), 3.16 (ddd, J = 7.6, 5.8, 1.9 Hz, 2H), 2.90-2.78 (m, | | | |
| | 2H), 2.68-2.53 (m, 3H), 2.09 (q, J = 10.5, 10.0 Hz, 2H), | | | |
| | 1.50 (d, J = 39.6 Hz, 2H), 1.14-1.03 (m, 2H), 0.99 (t, | | | |
| | J = 7.1 Hz, 3H). | | | |
| I-61 | ¹H NMR: (400 MHz, DMSO-d₆) δ 8.28 (s, 1H), 7.91 (s, | Calculated | 9.3% | White |
| | 1H), 7.74 (s, 1H), 7.46-7.26 (m, 6H), 7.20-7.09 (m, | exact mass = | | solid |
| | 1H), 7.03 (p, J = 4.2 Hz, 1H), 6.94 (td, J = 8.5, 2.6 Hz, | 784.803 | | |
| | 1H), 6.77 (dd, J = 8.5, 1.2 Hz, 1H), 6.72 (d, J = 2.4 Hz, | Found | | |
| | 1H), 6.50 (dd, J = 8.4, 2.4 Hz, 1H), 4.52 (d, J = 2.3 Hz, | [M + H]⁺ | | |
| | 2H), 4.32 (d, J = 11.2 Hz, 1H), 4.15 (t, J = 8.4 Hz, 1H), | (ESI+) = | | |
| | 3.82-3.72 (m, 1H), 3.62 (d, J = 12.8 Hz, 1H), 2.95 (s, | 784.3 | | |
| | 1H), 2.83 (d, J = 13.8 Hz, 1H), 2.69-2.55 (m, 3H), 2.04 | | | |
| | (ddd, J = 31.2, 13.0, 7.7 Hz, 3H), 1.48 (d, J = 17.5 Hz, | | | |
| | 2H), 1.18-1.03 (m, 2H). | | | |

### Synthesis of 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-7-carboxamide—I-18

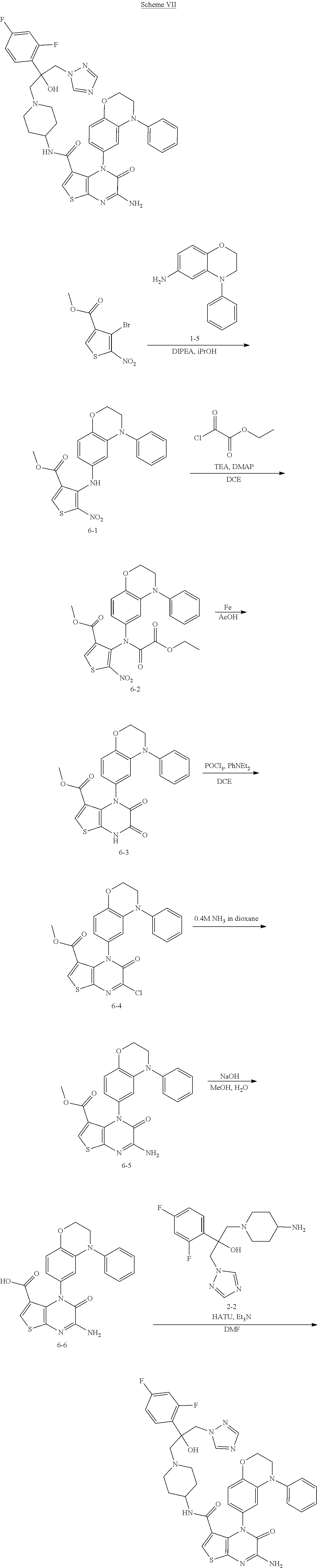

### Methyl-5-nitro-4-((4-phenyl-3,4-dihydro-2H-benzo[b][1,4]oxazin-6-yl)amino)thiophene-3-carboxylate (6-1)

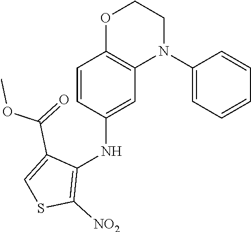 [SMILES: COC(=O)c1csc([N+](=O)[O-])c1Nc1ccc2c(c1)N(c1ccccc1)CCO2]

Methyl-5-nitro-4-((4-phenyl-3,4-dihydro-2H-benzo[b][1,4]oxazin-6-yl)amino)thiophene-3-carboxylate 6-1 was synthesized following the same procedure used for methyl-5-nitro-4-((4-phenyl-3,4-dihydro-2H-benzo[b][1,4]oxazin-6-yl)amino)thiophene-2-carboxylate 1-6 to obtain a brown solid in 65% yield.

LCMS: Calculated Exact Mass=411.1, Found [M+H]⁺ (ESI+)=412.2.

### Methyl-4-(2-ethoxy-2-oxo-N-(4-phenyl-3,4-dihydro-2H-benzo[b][1,4]oxazin-6-yl)acetamido)-5-nitrothiophene-3-carboxylate (6-2)

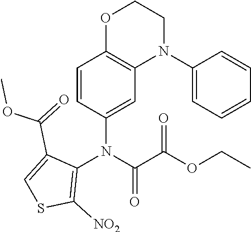 [SMILES: CCOC(=O)C(=O)N(c1ccc2c(c1)N(c1ccccc1)CCO2)c1c(C(=O)OC)csc1[N+](=O)[O-]]

Methyl-4-(2-ethoxy-2-oxo-N-(4-phenyl-3,4-dihydro-2H-benzo[b][1,4]oxazin-6-yl)acetamido)-5-nitrothiophene-3-carboxylate 6-2 was synthesized following the same procedure used for methyl-4-(2-ethoxy-2-oxo-N-(4-phenyl-3,4-dihydro-2H-benzo[b][1,4]oxazin-6-yl)acetamido)-5-nitrothiophene-2-carboxylate 1-7 to obtain a brown solid in 85% yield.

LCMS: Calculated Exact Mass=511.1, Found [M+H]⁺ (ESI+)=512.2.

### Methyl-2,3-dioxo-1-(4-phenyl-3,4-dihydro-2H-benzo[b][1,4]oxazin-6-yl)-1,2,3,4-tetrahydrothieno[2,3-b]pyrazine-7-carboxylate (6-3)

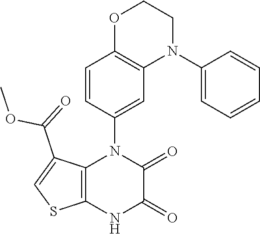 [SMILES: COC(=O)c1csc2[nH]c(=O)c(=O)n(-c3ccc4c(c3)N(c3ccccc3)CCO4)c12]

Methyl-2,3-dioxo-1-(4-phenyl-3,4-dihydro-2H-benzo[b][1,4]oxazin-6-yl)-1,2,3,4-tetrahydrothieno[2,3-b]pyrazine-7-carboxylate 6-3 was synthesized following the same procedure used for methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate 1-8 to obtain a brown solid in 46% yield.

LCMS: Calculated Exact Mass=435.1, Found [M+H]⁺ (ESI+)=436.3.

### Methyl-3-chloro-2-oxo-1-(4-phenyl-3,4-dihydro-2H-benzo[b][1,4]oxazin-6-yl)-1,2-dihydrothieno[2,3-b]pyrazine-7-carboxylate (6-4)

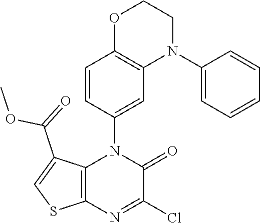 [SMILES: COC(=O)c1csc2nc(Cl)c(=O)n(-c3ccc4c(c3)N(c3ccccc3)CCO4)c12]

Methyl-3-chloro-2-oxo-1-(4-phenyl-3,4-dihydro-2H-benzo[b][1,4]oxazin-6-yl)-1,2-dihydrothieno[2,3-b]pyrazine-7-carboxylate 6-4 was synthesized following the same procedure used for methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate 1-9 to obtain a yellow solid in 25% yield.

LCMS: Calculated Exact Mass=453.1, Found [M+H]⁺ (ESI+)=454.1.

### Methyl-3-amino-2-oxo-1-(4-phenyl-3,4-dihydro-2H-benzo[b][1,4]oxazin-6-yl)-1,2-dihydrothieno[2,3-b]pyrazine-7-carboxylate (6-5)

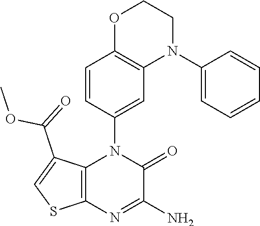 [SMILES: COC(=O)c1csc2nc(N)c(=O)n(-c3ccc4c(c3)N(c3ccccc3)CCO4)c12]

Methyl-3-amino-2-oxo-1-(4-phenyl-3,4-dihydro-2H-benzo[b][1,4]oxazin-6-yl)-1,2-dihydrothieno[2,3-b]pyrazine-7-carboxylate 6-5 was synthesized following the same procedure used for methyl-3-amino-2-oxo-1-(4-phenyl-3,4-dihydro-2H-benzo[b][1,4]oxazin-6-yl)-1,2-dihydrothieno[2,3-b]pyrazine-6-carboxylate 1-10 to obtain a yellow solid in 50% yield.

LCMS: Calculated Exact Mass=434.1, Found [M+H]⁺ (ESI+)=435.2.

### 3-amino-2-oxo-1-(4-phenyl-3,4-dihydro-2H-benzo[b][1,4]oxazin-6-yl)-1,2-dihydrothieno[2,3-b]pyrazine-7-carboxylic acid (6-6)

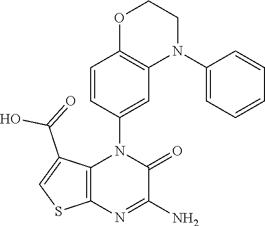 [SMILES: Nc1nc2scc(C(=O)O)c2n(-c2ccc3c(c2)N(c2ccccc2)CCO3)c1=O]

To a solution of methyl-3-amino-2-oxo-1-(4-phenyl-3,4-dihydro-2H-benzo[b][1,4]oxazin-6-yl)-1,2-dihydrothieno[2,3-b]pyrazine-7-carboxylate 6-5 (600 mg, 1.38 mmol) in MeOH (20 mL) was added a solution of NaOH (570 mg, 14.2 mmol) in H₂O (100 mL). The mixture was stirred at 100° C. for 3 hours. The reaction mixture was cooled down and the pH was adjusted to 5 and extracted with EtOAc (50 mL×3), dried over Na₂SO₄, filtered and concentrated to give crude 3-amino-2-oxo-1-(4-phenyl-3,4-dihydro-2H-benzo[b][1,4]oxazin-6-yl)-1,2-dihydrothieno[2,3-b]pyrazine-7-carboxylic acid 6-6 as a brown solid (510 mg, 88% yield).

LCMS: Calculated Exact Mass=420.1, Found [M+H]⁺ (ESI+)=421.1.

### 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-7-carboxamide (I-18

3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-7-carboxamide I-18 was synthesized following the same procedure used for 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-6-carboxamide 1-4 to obtain a pale yellow solid in 18% yield.

LCMS: Calculated Exact Mass=739.3, Found [M+H]⁺ (ESI+)=740.2.

¹H NMR (400 MHz, DMSO) δ 8.28 (s, 1H), 7.83 (dd, J=6.6, 2.8 Hz, 1H), 7.75 (d, J=0.6 Hz, 1H), 7.40 (qd, J=9.0, 2.0 Hz, 1H), 7.36-7.26 (m, 4H), 7.19-7.11 (m, 1H), 7.10 (s, 1H), 7.05 (t, J=6.5 Hz, 3H), 6.95 (td, J=8.5, 2.5 Hz, 1H), 6.80-6.73 (m, 2H), 6.50 (dd, J=8.5, 2.4 Hz, 1H), 5.62 (d, J=6.0 Hz, 1H), 4.58-4.46 (m, 2H), 4.35-4.23 (m, 1H), 4.19-4.07 (m, 1H), 3.80-3.68 (m, 1H), 3.59 (ddd, J=12.3, 5.2, 2.3 Hz, 1H), 3.07-2.93 (m, 1H), 2.86 (d, J=13.9 Hz, 1H), 2.71-2.56 (m, 3H), 2.14-2.02 (m, 2H), 1.50-1.37 (m, 2H), 1.19-1.01 (m, 2H).

In a similar manner, using the above procedure, the following amide compounds were synthesized.

| Compound | | | Yield | |
|---|---|---|---|---|
| ID# | ¹H NMR | LCMS | % | Appearance |
| I-17 | ¹H NMR: (400 MHz, DMSO-d₆) δ 8.32-8.23 (m, 1H), | Calculated | 16.7% | White |
| | 8.06 (dt, J = 13.2, 6.5 Hz, 1H), 7.76-7.71 (m, 1H), 7.46- | exact mass = | | solid |
| | 7.38 (m, 1H), 7.37-7.28 (m, 4H), 7.14 (ddd, J = 13.6, | 726.767 | | |
| | 11.2, 6.6 Hz, 3H), 7.08-6.88 (m, 4H), 6.80-6.62 (m, | Found | | |
| | 2H), 6.54-6.45 (m, 1H), 5.57 (d, J = 7.1 Hz, 1H), 4.61- | [M + H]⁺ | | |
| | 4.46 (m, 2H), 4.31 (dd, J = 10.8, 4.1 Hz, 1H), 4.24- | (ESI+) = | | |
| | 4.16 (m, 1H), 3.80-3.71 (m, 1H), 3.69-3.57 (m, 2H), | 726.2 | | |
| | 2.99-2.78 (m, 2H), 2.44-2.32 (m, 1H), 2.18 (ddd, J = | | | |
| | 45.8, 9.2, 4.2 Hz, 1H), 1.81 (td, J = 11.8, 5.7 Hz, 1H), | | | |
| | 1.43-1.22 (m, 1H). | | | |
| I-19 | ¹H NMR: (400 MHz, DMSO-d₆) δ 8.28 (d, J = 4.0 Hz, | Calculated | 15.9% | White |
| | 2H), 7.75 (d, J = 4.2 Hz, 1H), 7.45-7.37 (m, 1H), 7.35- | exact mass = | | solid |
| | 7.27 (m, 4H), 7.17 (d, J = 3.0 Hz, 1H), 7.09-6.96 (m, | 712.74 | | |
| | 4H), 6.77-6.65 (m, 2H), 6.48 (ddd, J = 8.5, 6.0, 2.4 Hz, | Found | | |
| | 1H), 5.57 (d, J = 2.9 Hz, 1H), 4.49 (s, 2H), 4.32 (dt, J = | [M + H]⁺ | | |
| | 8.2, 2.9 Hz, 1H), 4.22-4.12 (m, 1H), 3.80-3.62 (m, | (ESI+) = | | |
| | 3H), 3.39-3.34 (m, 1H), 3.03 (d, J = 18.3 Hz, 1H), 2.90- | 712.2 | | |
| | 2.83 (m, 1H), 2.77 (dd, J = 17.6, 10.5 Hz, 1H), 2.72- | | | |
| | 2.59 (m, 2H). | | | |
| I-34 | ¹H NMR: (400 MHz, DMSO-d₆) δ 8.28 (s, 1H), 7.83 (dd, | Calculated | 38.0% | White |
| | J = 6.8, 2.8 Hz, 1H), 7.75 (d, J = 1.0 Hz, 1H), 7.43-7.37 | exact mass = | | solid |
| | (m, 1H), 7.35-7.24 (m, 4H), 7.14 (dt, J = 9.2, 2.7 Hz, | 740.794 | | |
| | 1H), 7.10 (d, J = 0.5 Hz, 1H), 7.09-6.99 (m, 3H), 6.95 | Found | | |
| | (td, J = 8.5, 2.6 Hz, 1H), 6.80-6.73 (m, 2H), 6.50 (dd, | [M + H]⁺ | | |
| | J = 8.5, 2.4 Hz, 1H), 5.62 (d, J = 5.9 Hz, 1H), 4.53 (s, 2H), | (ESI+) = | | |
| | 4.34-4.25 (m, 1H), 4.14 (dd, J = 9.7, 5.7 Hz, 1H), 3.78- | 740.2 | | |
| | 3.71 (m, 1H), 3.63-3.54 (m, 1H), 3.01 (s, 1H), 2.85 | | | |
| | (d, J = 13.8 Hz, 1H), 2.72-2.57 (m, 3H), 2.08 (s, 2H), | | | |
| | 1.44 (d, J = 12.3 Hz, 2H), 1.17-1.02 (m, 2H). | | | |
| I-35 | ¹H NMR: (400 MHz, DMSO-d₆) δ 8.28 (s, 1H), 7.83 (dd, | Calculated | 29.8% | White |
| | J = 6.8, 2.9 Hz, 1H), 7.75 (d, J = 1.0 Hz, 1H), 7.43-7.37 | exact mass = | | solid |
| | (m, 1H), 7.36-7.26 (m, 4H), 7.19-7.12 (m, 1H), 7.10 | 740.794 | | |
| | (s, 1H), 7.07-6.98 (m, 3H), 6.98-6.93 (m, 1H), 6.80- | Found | | |
| | 6.74 (m, 2H), 6.50 (dd, J = 8.4, 2.4 Hz, 1H), 5.62 (d, J = | [M + H]⁺ | | |
| | 6.0 Hz, 1H), 4.53 (s, 2H), 4.34-4.26 (m, 1H), 4.18- | (ESI+) = | | |
| | 4.09 (m, 1H), 3.75 (dd, J = 12.5, 6.0 Hz, 1H), 3.63- | 740.2 | | |
| | 3.54 (m, 1H), 3.01 (s, 1H), 2.85 (d, J = 13.8 Hz, 1H), | | | |
| | 2.72-2.56 (m, 3H), 2.07 (s, 2H), 1.44 (d, J = 11.9 Hz, | | | |
| | 2H), 1.19-1.03 (m, 2H). | | | |
| I-49 | ¹H NMR: (400 MHz, Methanol-d₄) δ 8.40 (d, J = 13.1 | Calculated | 1.7% | White |
| | Hz, 1H), 7.87 (d, J = 2.0 Hz, 1H), 7.57-7.49 (m, 1H), | exact mass = | | solid |
| | 7.37-7.29 (m, 4H), 7.10-7.04 (m, 1H), 6.94 (tt, J = | 657.662 | | |
| | 11.0, 8.2 Hz, 2H), 6.86-6.73 (m, 3H), 6.52 (ddd, J = | Found | | |
| | 8.5, 4.9, 2.5 Hz, 1H), 4.60 (dd, J = 5.3, 2.8 Hz, 2H), 4.33 | [M + H]⁺ | | |
| | (dd, J = 5.1, 3.4 Hz, 2H), 3.79-3.68 (m, 2H), 3.59- | (ESI+) = | | |
| | 3.42 (m, 2H), 3.26-3.12 (m, 2H). | 657.2 | | |

### Synthesis of 3-amino-N-(1-(2-(2,4-difluorophenyl)-2-hydroxy-3-(1H-1,2,4-triazol-1-yl)propyl)piperidin-4-yl)-1-(4-methyl-3-phenoxyphenyl)-2-oxo-1,2-dihydrothieno[2,3-b]pyrazine-6-carboxamide—I-12

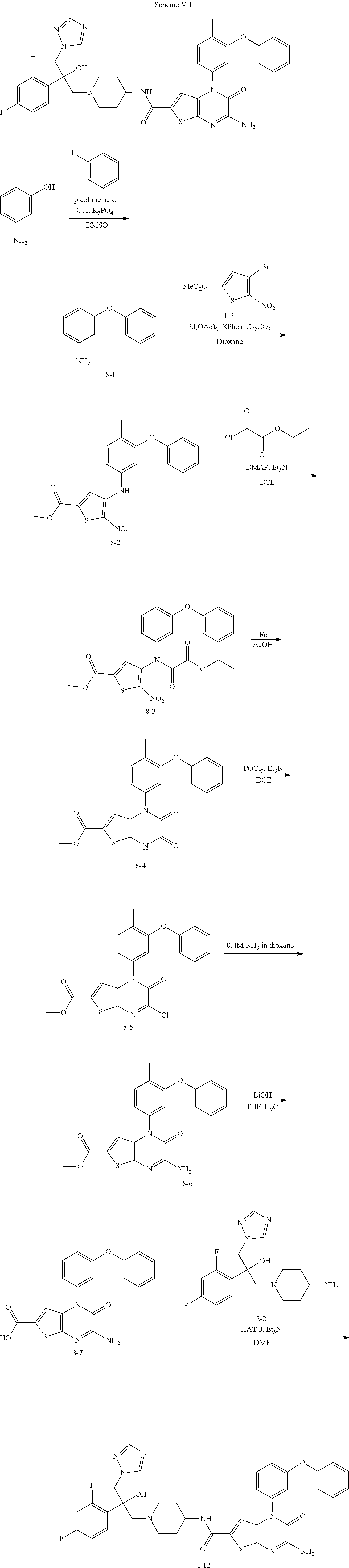

### 4-ethyl-3-phenoxyaniline (8-1)

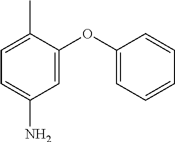

A solution of copper(I) iodide (1.9 g, 10 mmol), 2-picolinic acid (2.5 g, 20 mmol), iodobenzene (41 g, 201 mmol), 5-amino-2-methylphenol (25 g, 201 mmol) and K₃PO₄ (81.2 g, 402 mmol) in dimethylsulfoxide (400 mL) was purged with argon. The mixture was stirred at 80° C. overnight. The reaction mixture was cooled to room temperature. Ethyl acetate (500 mL) and H₂O (400 mL) were added and the mixture was stirred. The organic layer was separated and the aqueous layer was extracted with ethyl acetate (500 mL×3). Combined organic layer was dried over Na₂SO₄ and filtered. The filtrate was concentrated and the resulting residue was purified by column chromatography on silica gel (petroleum ether/EtOAc=4:1) to give 4-ethyl-3-phenoxyaniline 8-1 as a brown solid (21.9 g, 54% yield).

LCMS: Calculated Exact Mass=199.1, Found [M+H]⁺ (ESI+)=200.2.

### Methyl 4-((4-methyl-3-phenoxyphenyl)amino)-5-nitrothiophene-2-carboxylate (8-2)

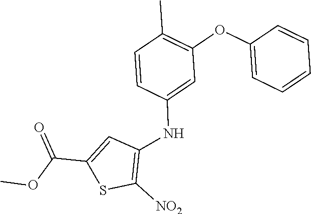 [SMILES: COC(=O)c1cc(Nc2ccc(C)c(Oc3ccccc3)c2)c([N+](=O)[O-])s1]

4-ethyl-3-phenoxyaniline 8-1 (4.0 g, 20 mmol), methyl 4-bromo-5-nitrothiophene-2-carboxylate 1-5 (6.4 g, 24 mmol) and Cs₂CO₃ (13 g, 40 mmol) were dissolved in dioxane (100 mL), and the reaction vessel was purged with N₂. Pd(OAc)₂ (450 mg, 2.0 mmol) and XPhos (1.9 g, 4.0 mmol) were added to the mixture, and the reaction was heated at reflux overnight. The reaction mixture was cooled to room temperature and filtered to remove solids. The mixture was diluted with H₂O (100 mL) and extracted with EtOAc (100 mL×2), and the organic layers were combined, dried over Na₂SO₄, filtered and concentrated. The crude product was purified by column chromatography on silica gel (petroleum ether/EtOAc=4:1) to give methyl 4-((4-methyl-3-phenoxyphenyl)amino)-5-nitrothiophene-2-carboxylate 8-2 as a yellow solid (3.7 g, 48% yield).

LCMS: Calculated Exact Mass=384.1, Found [M+H]⁺ (ESI+)=385.2.

### Methyl-4-(2-ethoxy-N-(4-methyl-3-phenoxyphenyl)-2-oxoacetamido)-5-nitrothiophene-2-carboxylate (8-3)

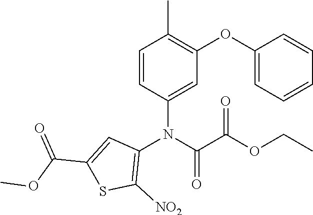 [SMILES: CCOC(=O)C(=O)N(c1ccc(C)c(Oc2ccccc2)c1)c1cc(C(=O)OC)sc1[N+](=O)[O-]]

Methyl-4-(2-ethoxy-N-(4-methyl-3-phenoxyphenyl)-2-oxoacetamido)-5-nitrothiophene-2-carboxylate 8-3 was synthesized following the same procedure used for methyl-4-(2-ethoxy-2-oxo-N-(4-phenyl-3,4-dihydro-2H-benzo[b][1,4]oxazin-6-yl)acetamido)-5-nitrothiophene-2-carboxylate 1-7 to obtain a brown solid in 80% yield.

LCMS: Calculated Exact Mass=484.1, Found [M+H]⁺ (ESI+)=485.2.

### Methyl-1-(4-methyl-3-phenoxyphenyl)-2,3-dioxo-1,2,3,4-tetrahydrothieno[2,3-b]pyrazine-6-carboxylate (8-4)

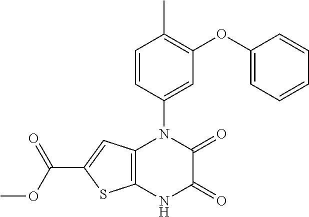 [SMILES: COC(=O)c1cc2c([nH]c(=O)c(=O)n2-c2ccc(C)c(Oc3ccccc3)c2)s1]

Methyl-1-(4-methyl-3-phenoxyphenyl)-2,3-dioxo-1,2,3,4-tetrahydrothieno[2,3-b]pyrazine-6-carboxylate 8-4 was synthesized following the same procedure used for methyl-2,3-dioxo-1-(4-phenyl-3,4-dihydro-2H-benzo[b][1,4]oxazin-6-yl)-1,2,3,4-tetrahydrothieno[2,3-b]pyrazine-6-carboxylate 1-8 to obtain a brown solid in 21% yield.

LCMS: Calculated Exact Mass=408.1, Found [M+H]⁺ (ESI+)=409.2.

### Methyl-3-chloro-1-(4-methyl-3-phenoxyphenyl)-2-oxo-1,2-dihydrothieno[2,3-b]pyrazine-6-carboxylate (8-5)

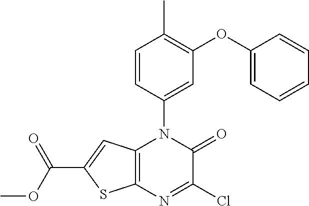 [SMILES: COC(=O)c1cc2c(nc(Cl)c(=O)n2-c2ccc(C)c(Oc3ccccc3)c2)s1]

Methyl-3-chloro-1-(4-methyl-3-phenoxyphenyl)-2-oxo-1,2-dihydrothieno[2,3-b]pyrazine-6-carboxylate 8-5 was synthesized following the same procedure used for methyl-3-chloro-2-oxo-1-(4-phenyl-3,4-dihydro-2H-benzo[b][1,4]oxazin-6-yl)-1,2-dihydrothieno[2,3-b]pyrazine-6-carboxylate 1-9 to obtain a yellow solid in 67% yield.

LCMS: Calculated Exact Mass=426.0, Found [M+H]⁺ (ESI+)=427.1.

### Methyl-3-amino-1-(4-methyl-3-phenoxyphenyl)-2-oxo-1,2-dihydrothieno[2,3-b]pyrazine-6-carboxylate (8-6)

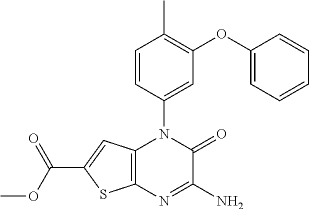 [SMILES: COC(=O)c1cc2c(nc(N)c(=O)n2-c2ccc(C)c(Oc3ccccc3)c2)s1]

Methyl-3-amino-1-(4-methyl-3-phenoxyphenyl)-2-oxo-1,2-dihydrothieno[2,3-b]pyrazine-6-carboxylate 8-6 was synthesized following the same procedure used for methyl-3-amino-2-oxo-1-(4-phenyl-3,4-dihydro-2H-benzo[b][1,4]oxazin-6-yl)-1,2-dihydrothieno[2,3-b]pyrazine-6-carboxylate 1-10 to obtain a yellow solid in 86% yield.

LCMS: Calculated Exact Mass=407.1, Found [M+H]⁺ (ESI+)=408.2.

### 3-amino-1-(4-methyl-3-phenoxyphenyl)-2-oxo-1,2-dihydrothieno[2,3-b]pyrazine-6-carboxylic acid (8-7)

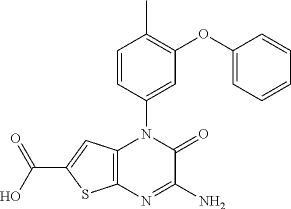 [SMILES: Cc1ccc(-n2c(=O)c(N)nc3sc(C(=O)O)cc32)cc1Oc1ccccc1]

3-amino-1-(4-methyl-3-phenoxyphenyl)-2-oxo-1,2-dihydrothieno[2,3-b]pyrazine-6-carboxylic acid 8-7 was synthesized following the same procedure used for 3-amino-2-oxo-1-(4-phenyl-3,4-dihydro-2H-benzo[b][1,4]oxazin-6-yl)-1,2-dihydrothieno[2,3-b]pyrazine-6-carboxylic acid 1 to obtain a yellow solid in 82% yield.

LCMS: Calculated Exact Mass=393.1, Found [M+H]⁺ (ESI+)=394.2.

### 3-amino-N-(1-(2-(2,4-difluorophenyl)-2-hydroxy-3-(1H-1,2,4-triazol-1-yl)propyl)piperidin-4-yl)-1-(4-methyl-3-phenoxyphenyl)-2-oxo-1,2-dihydrothieno[2,3-b]pyrazine-6-carboxamide (I-12)

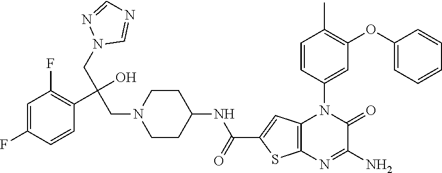 [SMILES: Cc1ccc(-n2c(=O)c(N)nc3sc(C(=O)NC4CCN(CC(O)(Cn5cncn5)c5ccc(F)cc5F)CC4)cc32)cc1Oc1ccccc1]

3-amino-N-(1-(2-(2,4-difluorophenyl)-2-hydroxy-3-(1H-1,2,4-triazol-1-yl)propyl)piperidin-4-yl)-1-(4-methyl-3-phenoxyphenyl)-2-oxo-1,2-dihydrothieno[2,3-b]pyrazine-6-carboxamide I-12 was synthesized following the same procedure used for 3-amino-N-(1-(2-(2,4-difluorophenyl)-2-hydroxy-3-(1H-1,2,4-triazol-1-yl)propyl)piperidin-4-yl)-2-oxo-1-(4-phenyl-3,4-dihydro-2H-benzo[b][1,4]oxazin-6-yl)-1,2-dihydrothieno[2,3-b]pyrazine-6-carboxamide I-4 to obtain a pale yellow solid in 10% yield.

LCMS: Calculated Exact Mass=712.2, Found [M+H]⁺ (ESI+)=713.2.

¹H NMR (400 MHz, DMSO) δ 8.28 (s, 1H), 7.83 (dd, J=6.6, 2.8 Hz, 1H), 7.75 (d, J=0.6 Hz, 1H), 7.40 (qd, J=9.0, 2.0 Hz, 1H), 7.36-7.26 (m, 4H), 7.19-7.11 (m, 1H), 7.10 (s, 1H), 7.05 (t, J=6.5 Hz, 3H), 6.95 (td, J=8.5, 2.5 Hz, 1H), 6.80-6.73 (m, 2H), 6.50 (dd, J=8.5, 2.4 Hz, 1H), 5.62 (d, J=6.0 Hz, 1H), 4.58-4.46 (m, 2H), 4.35-4.23 (m, 1H), 4.19-4.07 (m, 1H), 3.80-3.68 (m, 1H), 3.59 (ddd, J=12.3, 5.2, 2.3 Hz, 1H), 3.07-2.93 (i, 1H), 2.86 (d, J=13.9 Hz, 1H), 2.71-2.56 (, 3H), 2.14-2.02 (m, 2H), 1.50-1.37 (m, 2H), 1.19-1.01 (m, 2H).

In a similar manner, using the above procedure, the following amide compounds were synthesized

| Compound | | | Yield | |
|---|---|---|---|---|
| ID# | ¹H NMR | LCMS | % | Appearance |
| I-24 | ¹H NMR: (400 MHz, Methanol-d₄) δ 8.33 (s, 1H), 7.75 | Calculated | 16.7% | White |
| | (s, 1H), 7.55-7.51 (m, 1H), 7.47 (td, J = 9.0, 6.6 Hz, | exact mass = | | solid |
| | 1H), 7.36-7.31 (m, 2H), 7.12 (dd, J = 8.0, 2.1 Hz, 1H), | 713.768 | | |
| | 7.10-7.06 (m, 1H), 7.05 (s, 1H), 7.03-6.99 (m, 2H), | Found | | |
| | 6.96-6.89 (m, 2H), 6.84 (td, J = 8.5, 2.6 Hz, 1H), 4.67 | [M + H]⁺ | | |
| | (d, J = 14.3 Hz, 1H), 4.60 (d, J = 14.3 Hz, 1H), 3.69 | (ESI+) = | | |
| | (ddd, J = 11.3, 7.1, 4.2 Hz, 1H), 3.01 (dd, J = 13.8, 1.6 | 713.2 | | |
| | Hz, 1H), 2.79 (dd, J = 13.0, 8.4 Hz, 2H), 2.58 (d, J = | | | |
| | 11.9 Hz, 1H), 2.45-2.36 (m, 1H), 2.22 (td, J = 11.9, | | | |
| | 2.5 Hz, 1H), 1.77 (d, J = 12.6 Hz, 1H), 1.66 (d, J = 12.7 | | | |
| | Hz, 1H), 1.48 (dqd, J = 39.5, 11.9, 3.9 Hz, 2H). | | | |
| I-25 | ¹H NMR: (400 MHz, Methanol-d₄) δ 8.32 (s, 1H), 7.75 | Calculated | 13.3% | White |
| | (s, 1H), 7.53 (dd, J = 8.0, 0.9 Hz, 1H), 7.47 (td, J = 8.9, | exact mass = | | solid |
| | 6.6 Hz, 1H), 7.36-7.29 (m, 2H), 7.12 (dd, J = 8.0, 2.1 | 713.768 | | |
| | Hz, 1H), 7.10-7.04 (m, 2H), 7.03-6.98 (m, 2H), 6.96- | Found | | |
| | 6.87 (m, 2H), 6.84 (td, J = 8.5, 2.6 Hz, 1H), 4.67 (d, | [M + H]⁺ | | |
| | J = 14.3 Hz, 1H), 4.59(d, J = 14.3 Hz, 1H), 3.73-3.62 | (ESI+) = | | |
| | (m, 1H), 3.00 (dd, J = 13.8, 1.6 Hz, 1H), 2.78 (dd, J = | 713.2 | | |
| | 12.8, 7.4 Hz, 2H), 2.57 (d, J = 11.8 Hz, 1H), 2.40 (dd, | | | |
| | J = 12.0, 2.6 Hz, 1H), 2.21 (td, J = 11.9, 2.6 Hz, 1H), 1.76 | | | |
| | (d, J = 12.5 Hz, 1H), 1.65 (d, J = 12.9 Hz, 1H), 1.58- | | | |
| | 1.48 (m, 1H), 1.47-1.37 (m, 1H). | | | |
| I-40 | ¹H NMR: (400 MHz, DMSO-d₆) δ 8.44 (t, J = 5.8 Hz, | Calculated | 13.4% | White |
| | 1H), 8.29 (s, 1H), 7.74 (s, 1H), 7.57 (d, J = 8.1 Hz, 1H), | exact mass = | | solid |
| | 7.44-7.32 (m, 3H), 7.31-7.18 (m, 4H), 7.10 (t, J = | 670.7 | | |
| | 7.4 Hz, 1H), 7.07-7.00 (m, 4H), 6.96 (td, J = 8.4, 2.6 | Found | | |
| | Hz, 1H), 4.61-4.51 (m, 2H), 3.94 (t, J = 7.7 Hz, 1H), | [M + H]⁺ | | |
| | 3.54 (t, J = 7.8 Hz, 1H), 3.11 (d, J = 46.7 Hz, 2H), 2.31 | (ESI+) = | | |
| | (s, 5H), 2.01-1.92 (m, 1H). | 670.2 | | |

### Synthesis of 3-amino-6-(((1-(2-(2,4-difluorophenyl)-2-hydroxy-3-(1H-1,2,4-triazol-1-yl)propyl)piperidin-4-yl)amino)methyl)-1-(4-phenyl-3,4-dihydro-2H-benzo[b][1,4]oxazin-6-yl)thieno[2,3-b]pyrazin-2(1H)-one—I-47

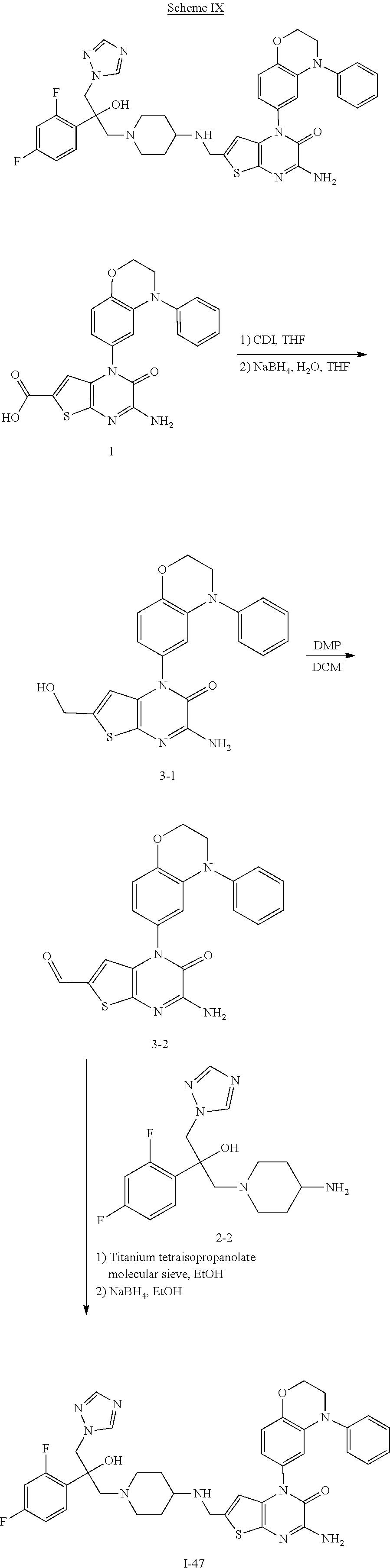

### 3-amino-6-(hydroxymethyl)-1-(4-phenyl-3,4-dihydro-2H-benzo[b][1,4]oxazin-6-yl)thieno[2,3-b]pyrazin-2(1H)-one (3-1)

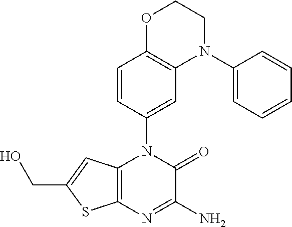 [SMILES: Nc1nc2sc(CO)cc2n(-c2ccc3c(c2)N(c2ccccc2)CCO3)c1=O]

To a solution of 3-amino-2-oxo-1-(4-phenyl-3,4-dihydro-2H-benzo[b][1,4]oxazin-6-yl)-1,2-dihydrothieno[2,3-b] pyrazine-6-carboxylic acid 1 (24 g, 57 mmol) in THF (800 mL) was added CDI (10.2 g, 63 mmol) at room temperature. The mixture was stirred at 50° C. for 2 hours. The reaction mixture was cooled down and a solution of NaBH₄ (6.5 g, 171 mmol) in H₂O (200 mL) was added at 0° C. The reaction was stirred for 1 hour at room temperature. The reaction mixture was diluted with saturated KHSO₄ solution (100 mL) and stirred for another 0.5 hour. The reaction mixture was neutralized with saturated Na₂CO₃ solution. The aqueous phase was extracted with EtOAc (500 mL×3). The combined organic layers were dried over anhydrous Na₂SO₄, filtered and concentrated. The crude product was washed with EtOAc (500 mL) and dried under reduced pressure to give 3-amino-6-(hydroxymethyl)-1-(4-phenyl-3,4-dihydro-2H-benzo[b][1,4]oxazin-6-yl)thieno[2,3-b]pyrazin-2(1H)-one 3-1 as a pale yellow solid (14.6 g, 63% yield).

LCMS: Calculated Exact Mass=406.1, Found [M+H]⁺ (ESI+)=407.2.

¹H NMR (400 MHz, DMSO) δ 7.38-7.35 (m, 2H), 7.30-7.28 (m, 2H), 7.09 (t, J=7.3 Hz, 1H), 7.00 (d, J=8.2 Hz, 1H), 6.91 (s, 2H), 6.75-6.71 (m, 2H), 6.24 (s, 1H), 5.41 (t, J=5.3 Hz, 1H), 4.49 (d, J=4.5 Hz, 2H), 4.35-4.34 (m, 2H), 3.80-3.72 (m, 2H).

### 3-amino-2-oxo-1-(4-phenyl-3,4-dihydro-2H-benzo[b][1,4]oxazin-6-yl)-1,2-dihydrothieno[2,3-b]pyrazine-6-carbaldehyde (3-2)

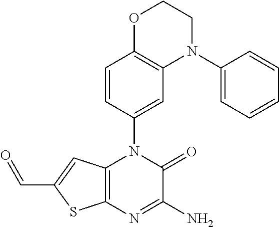 [SMILES: Nc1nc2sc(C=O)cc2n(-c2ccc3c(c2)N(c2ccccc2)CCO3)c1=O]

A solution of 3-amino-6-(hydroxymethyl)-1-(4-phenyl-3,4-dihydro-2H-benzo[b][1,4]oxazin-6-yl)thieno[2,3-b]pyrazin-2(1H)-one 3-1 in DCM (600 mL) was cooled to 0° C. and Dess-Martin Oxidizer (18.3 g, 43.2 mmol) was added in batches over 15 minutes. The reaction was stirred for 2 hours at room temperature. Saturated Na₂SO₃ solution and saturated Na₂CO₃ solution were poured into the reaction mixture. The solution was extracted with DCM (200 mL×3). The combined organic layers were dried over anhydrous Na₂SO₄, filtered and concentrated. The crude product was purified by column chromatography on silica gel (DCM/MeOH=95:5) to afford 3-amino-2-oxo-1-(4-phenyl-3,4-dihydro-2H-benzo[b][1,4]oxazin-6-yl)-1,2-dihydrothieno[2,3-b]pyrazine-6-carbaldehyde 3-2 as a brown solid (6.8 g, 47% yield).

LCMS: Calculated Exact Mass=404.1, Found [M+H]⁺ (ESI+)=405.2.

¹H NMR (400 MHz, DMSO) 59.71 (s, 1H), 7.48-7.28 (m, 6H), 7.27 (s, 1H), 7.11-7.07 (m, 1H), 7.03 (d, J=8.4 Hz, 1H), 6.88 (d, J=2.4 Hz, 1H), 6.80 (dd, J=8.4, 2.4 Hz, 1H), 4.35 (s, 2H), 3.77-3.76 (m, 2H).

### 3-amino-6-(((1-(2-(2,4-difluorophenyl)-2-hydroxy-3-(1H-1,2,4-triazol-1-yl)propyl)piperidin-4-yl)amino)methyl)-1-(4-phenyl-3,4-dihydro-2H-benzo[b][1,4]oxazin-6-yl)thieno[2,3-b]pyrazin-2(1H)-one (1-47)

3-amino-2-oxo-1-(4-phenyl-3,4-dihydro-2H-benzo[b][1,4]oxazin-6-yl)-1,2-dihydrothieno[2,3-b]pyrazine-6-carbaldehyde 3-2 (100 mg, 0.25 mmol) and 1-(4-aminopiperidin-1-yl)-2-(2,4-difluorophenyl)-3-(1H-1,2,4-triazol-1-yl)propan-2-ol 2-2 (169 mg, 0.5 mmol) were dissolved in EtOH (12 mL) and treated with titanium tetraisopropanolate (71 mg, 0.25 mmol) and molecular sieves (10 mg). The mixture was heated in a microwave reactor at 90° C. for 2 hours. NaBH₄ (29 mg, 0.75 mmol) was added to the solution. The reaction mixture was stirred for 30 minutes at room temperature. When the reaction was complete, the solvent was evaporated. The crude was purified by Prep-HPLC (ACN/H₂O with 0.05% NH₃ as the mobile phase) to give 3-amino-6-(((1-(2-(2,4-difluorophenyl)-2-hydroxy-3-(1H-1,2,4-triazol-1-yl)propyl)piperidin-4-yl)amino)methyl)-1-(4-phenyl-3,4-dihydro-2H-benzo[b][1,4]oxazin-6-yl)thieno[2,3-b]pyrazin-2(1H)-one I-47 as a pale yellow solid. (40 mg, 22% yield).

LCMS: Calculated Exact Mass=725.3, Found [M+H]⁺ (ESI+)=726.9.

¹H NMR (400 MHz, DMSO) δ 8.29 (s, 1H), 7.73 (s, 1H), 7.42-7.37 (m, 1H), 7.34 (d, J=7.5 Hz, 2H), 7.28 (d, J=7.8 Hz, 2H), 7.17-7.11 (m, 1H), 7.08 (t, J=7.2 Hz, 1H), 7.00-6.93 (m, 2H), 6.84 (s, 2H), 6.71-6.70 (m, 2H), 6.20 (s, 1H), 5.59 (s, 1H), 4.61-4.45 (m, 2H), 4.34 (s, 2H), 3.75-3.72 (m, 4H), 2.81 (d, J=14.1 Hz, 1H), 2.66 (d, J=7.9 Hz, 2H), 2.58-2.55 (m, 1H), 2.28-2.23 (m, 1H), 2.09-2.02 (m, 3H), 1.64-1.56 (m, 2H), 1.14-1.05 (m, 2H).

In a similar manner, using the above procedure, the following amide compounds were synthesized:

| Compound | | | Yield | |
|---|---|---|---|---|
| ID# | ¹H NMR | LCMS | % | Appearance |
| I-62 | ¹H NMR: (400 MHz, DMSO-d₆) δ 8.29 (s, 1H), 7.73 (s, | Calculated | 49.6% | White |
| | 1H), 7.42-7.31 (m, 3H), 7.30-7.25 (m, 2H), 7.14 | exact mass = | | solid |
| | (ddd, J = 11.9, 9.1, 2.6 Hz, 1H), 7.08 (tt, J = 7.2, 1.3 | 726.81 | | |
| | Hz, 1H), 7.01-6.92 (m, 2H), 6.84 (s, 2H), 6.74-6.68 | Found | | |
| | (m, 2H), 6.21 (s, 1H), 5.59 (s, 1H), 4.53 (d, J = 2.7 Hz, | [M + H]⁺ | | |
| | 2H), 4.34 (d, J = 5.0 Hz, 2H), 3.74 (dd, J = 9.9, 5.5 Hz, | (ESI+) = | | |
| | 4H), 2.82 (d, J = 13.9 Hz, 1H), 2.70-2.61 (m, 3H), | 726.5 | | |
| | 2.27 (s, 1H), 2.05 (q, J = 10.3 Hz, 3H), 1.60 (s, 2H), | | | |
| | 1.09 (dd, J = 18.7, 9.1 Hz, 2H). | | | |
| I-63 | ¹H NMR: (400 MHz, DMSO-d₆) δ 8.29 (s, 1H), 7.73 (s, | Calculated | 47.7% | White |
| | 1H), 7.41-7.32 (m, 3H), 7.30-7.25 (m, 2H), 7.14 | exact mass = | | solid |
| | (ddd, J = 11.9, 9.1, 2.6 Hz, 1H), 7.08 (tt, J = 7.2, 1.3 | 726.81 | | |
| | Hz, 1H), 7.00-6.92 (m, 2H), 6.88-6.78 (m, 2H), | Found | | |
| | 6.73-6.67 (m, 2H), 6.20 (s, 1H), 5.59 (s, 1H), 4.53 | [M + H]⁺ | | |
| | (d, J = 2.9 Hz, 2H), 4.38-4.28 (m, 2H), 3.79-3.67 | (ESI+) = | | |
| | (m, 4H), 2.81 (dd, J = 13.8, 1.8 Hz, 1H), 2.69-2.61 | 726.5 | | |
| | (m, 3H), 2.26 (s, 1H), 2.06 (q, J = 10.1 Hz, 3H), 1.60 | | | |
| | (s, 2H), 1.17-1.05 (m, 2H). | | | |
| I-66 | ¹H NMR: (400 MHz, DMSO-d₆) δ 8.26 (s, 1H), 7.73 (s, | Calculated | 5.8% | White |
| | 1H), 7.39-7.33 (m, 3H), 7.31-7.26 (m, 2H), 7.11- | exact mass = | | solid |
| | 7.04 (m, 2H), 6.99 (d, J = 8.3 Hz, 1H), 6.86 (s, 2H), | 698.757 | | |
| | 6.69 (d, J = 8.1 Hz, 2H), 6.19 (s, 1H), 4.47 (s, 2H), | Found | | |
| | 4.35 (s, 2H), 3.76 (t, J = 4.3 Hz, 2H), 3.60 (d, J = 6.0 | [M + H]⁺ | | |
| | Hz, 2H), 3.17 (q, J = 4.3, 2.8 Hz, 1H), 3.01 (s, 1H), | (ESI+) = | | |
| | 2.85 (dd, J = 12.8, 2.0 Hz, 1H), 2.73-2.65 (m, 2H), | 698.6 | | |
| | 2.58 (s, 1H). | | | |
| I-67 | ¹H NMR: (400 MHz, Methanol-d₄) δ 8.34 (s, 1H), | Calculated | 4.2% | White |
| | 7.73 (s, 1H), 7.46 (td, J = 8.9, 6.6 Hz, 1H), 7.33 (dd, | exact mass = | | solid |
| | J = 8.6, 7.1 Hz, 2H), 7.30-7.25 (m, 2H), 7.12-7.05 | 740.837 | | |
| | (m, 1H), 6.99 (d, J = 8.4 Hz, 1H), 6.91 (ddd, J = 11.6, | Found | | |
| | 8.8, 2.5 Hz, 1H), 6.82 (td, J = 8.4, 2.6 Hz, 1H), 6.70 (d, | [M + H]⁺ | | |
| | J = 2.4 Hz, 1H), 6.66 (dd, J = 8.4, 2.4 Hz, 1H), 6.27 (s, | (ESI+) = | | |
| | 1H), 4.65 (d, J = 14.2 Hz, 1H), 4.54 (d, J = 14.2 Hz, | 740.6 | | |
| | 1H), 4.40-4.33 (m, 2H), 3.75 (d, J = 5.3 Hz, 4H), | | | |
| | 2.95 (dd, J = 13.7, 1.6 Hz, 1H), 2.73 (dd, J = 16.9, | | | |
| | 12.6 Hz, 2H), 2.46 (d, J = 11.6 Hz, 1H), 2.36 (d, J = 6.6 | | | |
| | Hz, 2H), 2.29 (td, J = 11.6, 2.6 Hz, 1H), 2.05 (td, J = | | | |
| | 11.7, 2.6 Hz, 1H), 1.62 (d, J = 12.9 Hz, 1H), 1.48 (d, | | | |
| | J = 12.9 Hz, 1H), 1.35 (ddd, J = 14.9, 6.6, 3.1 Hz, 1H), | | | |
| | 1.23-1.14 (m, 1H), 1.09-0.98 (m, 1H). | | | |
| I-68 | ¹H NMR: (400 MHz, Methanol-d₄) δ 8.32 (s, 1H), | Calculated | 2.0% | White |
| | 7.74 (s, 1H), 7.48-7.42 (m, 1H), 7.35-7.26 (m, 4H), | exact mass = | | solid |
| | 7.09 (t, J = 7.2 Hz, 1H), 7.01-6.97 (m, 1H), 6.93- | 712.783 | | |
| | 6.87 (m, 1H), 6.85-6.80 (m, 1H), 6.70-6.63 (m, | Found | | |
| | 2H), 6.23 (s, 1H), 4.69-4.55 (m, 3H), 4.37 (dd, J = | [M + H]⁺ | | |
| | 5.3, 3.5 Hz, 2H), 3.79-3.73 (m, 2H), 3.53 (s, 2H), | (ESI+) = | | |
| | 2.98 (dd, J = 13.9, 1.6 Hz, 1H), 2.78 (d, J = 13.8 Hz, | 712.6 | | |
| | 1H), 2.46 (s, 4H), 2.36 (d, J = 9.3 Hz, 4H). | | | |
| I-71 | ¹H NMR: (400 MHz, DMSO-d₆) δ 8.27 (s, 1H), 7.73 (d, | Calculated | 13.1% | White |
| | J = 0.8 Hz, 1H), 7.36 (dt, J = 8.5, 7.1 Hz, 3H), 7.28 | exact mass = | | solid |
| | (ddd, J = 8.6, 2.3, 1.3 Hz, 2H), 7.15-7.05 (m, 2H), | 712.783 | | |
| | 7.01-6.97 (m, 1H), 6.87 (d, J = 16.4 Hz, 3H), 6.74- | Found | | |
| | 6.69 (m, 2H), 6.19 (d, J = 4.4 Hz, 1H), 5.57 (d, J = 6.8 | [M + H]⁺ | | |
| | Hz, 1H), 4.57-4.45 (m, 2H), 4.34 (s, 2H), 3.75 (s, | (ESI+) = | | |
| | 2H), 3.60 (d, J = 10.6 Hz, 2H), 3.02 (s, 1H), 2.95 (dd, | 712.6 | | |
| | J = 13.4, 9.2 Hz, 1H), 2.80 (dd, J = 13.4, 10.9 Hz, 1H), | | | |
| | 2.42-2.36 (m, 1H), 2.34-2.28 (m, 1H), 2.19 (dd, J = | | | |
| | 9.3, 4.7 Hz, 1H), 1.78 (dd, J = 8.1, 5.1 Hz, 1H), 1.37 | | | |
| | (d, J = 5.5 Hz, 1H). | | | |
| I-72 | ¹H NMR: (400 MHz, DMSO-d₆) δ 8.28 (s, 1H), 7.74 (s, | Calculated | 9.6% | White |
| | 1H), 7.43 (td, J = 9.0, 6.8 Hz, 1H), 7.35 (dd, J = 8.5, | exact mass = | | solid |
| | 7.2 Hz, 2H), 7.31-7.26 (m, 2H), 7.18 (ddd, J = 11.8, | 740.837 | | |
| | 9.1, 2.6 Hz, 1H), 7.08 (tt, J = 7.2, 1.3 Hz, 1H), 7.01- | Found | | |
| | 6.95 (m, 2H), 6.91-6.79 (m, 2H), 6.74-6.69 (m, | [M + H]⁺ | | |
| | 2H), 6.22 (s, 1H), 4.48 (s, 2H), 4.34 (dd, J = 5.6, 3.2 | (ESI+) = | | |
| | Hz, 2H), 3.75 (d, J = 3.2 Hz, 4H), 2.77 (d, J = 11.1 Hz, | 740.3 | | |
| | 1H), 2.43 (d, J = 11.2 Hz, 1H), 2.33 (p, J = 1.9 Hz, 1H), | | | |
| | 2.22-2.15 (m, 2H), 2.03 (d, J = 14.6 Hz, 1H), 1.90 | | | |
| | (dd, J = 14.7, 8.2 Hz, 2H), 1.74 (d, J = 13.7 Hz, 3H), | | | |
| | 1.16 (dd, J = 21.0, 10.9 Hz, 2H). | | | |
| I-73 | ¹H NMR: (400 MHz, Methanol-d₄) δ 8.34 (s, 1H), | Calculated | 7.2% | White |
| | 7.75 (s, 1H), 7.50 (td, J = 9.0, 6.7 Hz, 1H), 7.35-7.24 | exact mass = | | solid |
| | (m, 4H), 7.11-7.04 (m, 1H), 6.98 (d, J = 8.4 Hz, 1H), | 754.863 | | |
| | 6.91 (ddd, J = 11.7, 8.9, 2.6 Hz, 1H), 6.87-6.82 (m, | Found | | |
| | 1H), 6.71 (d, J = 2.4 Hz, 1H), 6.66 (dd, J = 8.5, 2.4 Hz, | [M + H]⁺ | | |
| | 1H), 6.30 (s, 1H), 4.59-4.45 (m, 2H), 4.36 (dt, J = | (ESI+) = | | |
| | 5.1, 2.3 Hz, 2H), 3.87-3.79 (m, 2H), 3.75 (dd, J = | 754.3 | | |
| | 5.2, 3.6 Hz, 2H), 2.81-2.73 (m, 1H), 2.61 (d, J = 11.5 | | | |
| | Hz, 1H), 2.49 (ddt, J = 14.7, 10.7, 6.4 Hz, 2H), 2.32- | | | |
| | 2.15 (m, 2H), 2.02 (td, J = 11.8, 2.6 Hz, 1H), 1.82 (dt, | | | |
| | J = 38.0, 11.4 Hz, 3H), 1.65 (td, J = 8.9, 4.3 Hz, 1H), | | | |
| | 1.39 (tt, J = 19.8, 6.7 Hz, 4H). | | | |
| I-74 | ¹H NMR: (400 MHz, DMSO-d₆) δ 8.27 (s, 1H), 7.75 (s, | Calculated | 9.2% | White |
| | 1H), 7.34 (t, J = 7.7 Hz, 3H), 7.30-7.24 (m, 2H), 7.09 | exact mass = | | solid |
| | (t, J = 7.3 Hz, 2H), 6.99 (d, J = 8.6 Hz, 2H), 6.91 (s, | 657.705 | | |
| | 2H), 6.79 (s, 1H), 6.69 (s, 2H), 6.17 (s, 1H), 4.45 (s, | Found | | |
| | 2H), 4.37-4.26 (m, 2H), 3.75 (t, J = 4.4 Hz, 2H), 3.64 | [M + H]⁺ | | |
| | (d, J = 14.5 Hz, 1H), 3.52 (d, J = 14.5 Hz, 1H), 2.43 (t, | (ESI+) = | | |
| | J = 6.2 Hz, 1H), 2.35-2.28 (m, 1H), 2.14-2.04 (m, | 657.5 | | |
| | 1H), 1.86-1.77 (m, 1H). | | | |
| I-75 | ¹H NMR: (400 MHz, DMSO-d₆) δ 8.28 (d, J = 1.9 Hz, | Calculated | 8.8% | White |
| | 1H), 7.73 (d, J = 3.5 Hz, 1H), 7.35 (qd, J = 8.0, 7.3, 5.7 | exact mass = | | solid |
| | Hz, 3H), 7.30-7.24 (m, 2H), 7.16-7.02 (m, 2H), | 726.81 | | |
| | 6.99 (dd, J = 8.3, 2.5 Hz, 1H), 6.94-6.78 (m, 3H), | Found | | |
| | 6.77-6.69 (m, 2H), 6.17 (d, J = 6.2 Hz, 1H), 5.63 (s, | [M + H]⁺ | | |
| | 1H), 4.60-4.45 (m, 2H), 4.34 (s, 2H), 3.75 (s, 2H), | (ESI+) = | | |
| | 3.60 (s, 2H), 2.87-2.75 (m, 1H), 2.69-2.57 (m, 2H), | 726.6 | | |
| | 2.37-2.31 (m, 1H), 2.11 (t, J = 16.6 Hz, 2H), 1.97 (s, | | | |
| | 1H), 1.51 (d, J = 37.3 Hz, 2H), 1.24 (s, 1H), 1.00 (s, 1H). | | | |
| I-77 | ¹H NMR: (400 MHz, Methanol-d₄) δ 8.33 (d, J = 3.1 | Calculated | 7.2% | White |
| | Hz, 1H), 7.73 (d, J = 1.1 Hz, 1H), 7.49-7.41 (m, 1H), | exact mass = | | solid |
| | 7.38-7.32 (m, 2H), 7.29 (dt, J = 8.7, 1.3 Hz, 2H), | 712.783 | | |
| | 7.14-7.07 (m, 1H), 7.01 (dd, J = 8.4, 4.2 Hz, 1H), | Found | | |
| | 6.88 (ddd, J = 11.7, 8.8, 2.5 Hz, 1H), 6.78 (s, 1H), | [M + H]⁺ | | |
| | 6.71-6.65 (m, 2H), 6.29-6.18 (m, 1H), 4.64 (dd, J = | (ESI+) = | | |
| | 14.3, 5.9 Hz, 1H), 4.55 (d, J = 14.0 Hz, 1H), 4.37 (t, J = | 712.7 | | |
| | 4.4 Hz, 2H), 3.77 (t, J = 4.4 Hz, 2H), 3.69 (s, 1H), 3.59 | | | |
| | (s, 1H), 3.15-3.08 (m, 1H), 3.03 (d, J = 14.1 Hz, 1H), | | | |
| | 2.92 (dd, J = 13.4, 4.6 Hz, 1H), 2.75 (s, 1H), 2.51 (dd, | | | |
| | J = 8.5, 4.9 Hz, 1H), 2.46-2.19 (m, 3H), 1.93 (ddd, J = | | | |
| | 13.4, 8.9, 4.7 Hz, 1H), 1.46 (ddd, J = 12.9, 8.5, 4.8 | | | |
| | Hz, 1H). | | | |
| I-78 | ¹H NMR: (400 MHz, DMSO-d₆) δ 8.33 (d, J = 2.7 Hz, | Calculated | 6.9% | White |
| | 1H), 7.76 (s, 1H), 7.44-7.31 (m, 3H), 7.31-7.25 (m, | exact mass = | | solid |
| | 2H), 7.16 (ddt, J = 11.7, 9.2, 2.4 Hz, 1H), 7.09 (ddd, | 754.82 | | |
| | J = 8.7, 6.6, 1.5 Hz, 1H), 7.02-6.91 (m, 2H), 6.85 (s, | Found | | |
| | 2H), 6.72 (d, J = 7.3 Hz, 2H), 6.63 (s, 1H), 6.24 (d, J = | [M + H]⁺ | | |
| | 1.8 Hz, 1H), 4.63-4.51 (m, 2H), 4.35 (dd, J = 5.6, 3.2 | (ESI+) = | | |
| | Hz, 2H), 3.99 (t, J = 13.5 Hz, 1H), 3.76 (d, J = 5.6 Hz, | 754.5 | | |
| | 5H), 3.22 (dd, J = 15.8, 10.0 Hz, 1H), 2.99 (q, J = 11.0 | | | |
| | Hz, 1H), 2.77-2.67 (m, 1H), 2.67-2.54 (m, 2H), | | | |
| | 2.29-2.17 (m, 1H), 1.69 (dt, J = 33.3, 15.7 Hz, 2H), | | | |
| | 1.24-0.76 (m, 2H). | | | |
| I-79 | ¹H NMR: (400 MHz, DMSO-d₆) δ 8.27 (d, J = 2.0 Hz, | Calculated | 12.1% | White |
| | 1H), 7.73 (d, J = 3.4 Hz, 1H), 7.42-7.31 (m, 3H), | exact mass = | | solid |
| | 7.28 (d, J = 7.9 Hz, 2H), 7.16-7.03 (m, 2H), 6.99 (dd, | 726.81 | | |
| | J = 8.3, 2.6 Hz, 1H), 6.83 (d, J = 15.1 Hz, 3H), 6.76- | Found | | |
| | 6.69 (m, 2H), 6.16 (d, J = 6.3 Hz, 1H), 5.56 (d, J = 15.5 | [M + H]⁺ | | |
| | Hz, 1H), 4.58-4.45 (m, 2H), 4.34 (s, 2H), 3.75 (s, | (ESI+) = | | |
| | 2H), 3.59 (d, J = 9.6 Hz, 2H), 2.81 (t, J = 13.9 Hz, 1H), | 726.7 | | |
| | 2.68-2.57 (m, 1H), 2.33 (p, J = 1.9 Hz, 2H), 2.24- | | | |
| | 2.08 (m, 2H), 1.97 (s, 1H), 1.50 (d, J = 36.7 Hz, 2H), | | | |
| | 1.24 (d, J = 5.8 Hz, 1H), 0.97 (d, J = 26.9 Hz, 1H). | | | |
| I-80 | ¹H NMR: (400 MHz, Methanol-d₄) δ 7.89 (d, J = 1.1 | Calculated | 3.7% | White |
| | Hz, 1H), 7.56 (d, J = 1.1 Hz, 1H), 7.45 (td, J = 9.0, 6.6 | exact mass = | | solid |
| | Hz, 1H), 7.34 (dd, J = 8.5, 7.2 Hz, 2H), 7.30-7.25 (m, | 726.81 | | |
| | 2H), 7.13-7.06 (m, 1H), 6.99 (d, J = 8.4 Hz, 1H), | Found | | |
| | 6.91 (ddd, J = 11.7, 8.9, 2.5 Hz, 1H), 6.81 (td, J = 8.3, | [M + H]⁺ | | |
| | 2.3 Hz, 1H), 6.70 (d, J = 2.4 Hz, 1H), 6.66 (dd, J = 8.4, | (ESI+) = | | |
| | 2.4 Hz, 1H), 6.27 (s, 1H), 4.91 (d, J = 14.0 Hz, 1H), | 726.7 | | |
| | 4.71 (d, J = 14.0 Hz, 1H), 4.37 (dd, J = 5.8, 2.9 Hz, | | | |
| | 2H), 3.82-3.72 (m, 4H), 2.98 (dd, J = 13.8, 1.6 Hz, | | | |
| | 1H), 2.77-2.69 (m, 2H), 2.47 (d, J = 11.6 Hz, 1H), | | | |
| | 2.43-2.35 (m, 1H), 2.28 (t, J = 11.1 Hz, 1H), 2.12- | | | |
| | 2.04 (m, 1H), 1.76 (d, J = 13.0 Hz, 1H), 1.63 (d, J = | | | |
| | 12.8 Hz, 1H), 1.35 (dd, J = 11.8, 3.8 Hz, 1H), 1.22 (dd, | | | |
| | J = 11.6, 3.8 Hz, 1H). | | | |
| I-81 | ¹H NMR: (400 MHz, Chloroform-d) δ 8.11 (s, 1H), | Calculated | 7.5% | White |
| | 7.78 (s, 1H), 7.52 (s, 1H), 7.37-7.31 (m, 2H), 7.24 (t, | exact mass = | | solid |
| | J = 1.1 Hz, 2H), 7.13-7.06 (m, 1H), 7.01 (d, J = 8.4 | 724.794 | | |
| | Hz, 1H), 6.84 (s, 3H), 6.66 (dd, J = 8.4, 2.4 Hz, 1H), | Found | | |
| | 6.23 (s, 1H), 5.47 (s, 2H), 4.48 (s, 2H), 4.38 (s, 2H), | [M + H]⁺ | | |
| | 3.78 (d, J = 14.8 Hz, 4H), 3.00 (s, 1H), 2.82 (s, 1H), | (ESI+) = | | |
| | 2.66 (s, 1H), 2.42 (s, 1H), 2.28 (s, 1H), 2.14 (s, 1H), | 724.4 | | |
| | 1.39 (s, 1H). | | | |
| I-82 | ¹H NMR: (400 MHz, Methanol-d₄) δ 8.27 (s, 1H), | Calculated | 9.4% | White |
| | 7.71 (s, 1H), 7.43 (td, J = 9.1, 6.6 Hz, 1H), 7.36-7.26 | exact mass = | | solid |
| | (m, 4H), 7.08 (tt, J = 7.0, 1.4 Hz, 1H), 7.01 (d, J = 8.4 | 643.678 | | |
| | Hz, 1H), 6.81 (dd, J = 9.4, 6.9 Hz, 2H), 6.73-6.64 (m, | Found | | |
| | 2H), 6.16 (s, 1H), 4.67-4.58 (m, 2H), 4.41-4.35 (m, | [M + H]⁺ | | |
| | 2H), 3.75 (ddd, J = 12.5, 7.5, 1.6 Hz, 4H), 3.09 (dd, J = | (ESI+) = | | |
| | 12.7, 1.3 Hz, 1H), 2.91 (d, J = 12.7 Hz, 1H). | 643.5 | | |
| I-116 | ¹H NMR: (400 MHz, Methanol-d₄) δ 8.33 (s, 1H), | Calculated | 11.0% | White |
| | 7.75 (s, 1H), 7.48 (td, J = 9.0, 6.6 Hz, 1H), 7.37-7.31 | exact mass = | | solid |
| | (m, 2H), 7.29-7.25 (m, 2H), 7.09 (tt, J = 7.3, 1.4 Hz, | 752.847 | | |
| | 1H), 6.99 (d, J = 8.4 Hz, 1H), 6.90 (ddd, J = 11.6, 8.8, | Found | | |
| | 2.6 Hz, 1H), 6.86-6.81 (m, 1H), 6.70 (d, J = 2.4 Hz, | [M + H]⁺ | | |
| | 1H), 6.65 (dd, J = 8.4, 2.4 Hz, 1H), 6.26 (d, J = 0.8 Hz, | (ESI+) = | | |
| | 1H), 4.65-4.51 (m, 2H), 4.36 (dd, J = 5.2, 3.5 Hz, | 752.3 | | |
| | 2H), 3.81-3.72 (m, 4H), 3.06 (dd, J = 13.7, 2.2 Hz, | | | |
| | 1H), 2.81 (d, J = 10.7 Hz, 1H), 2.72-2.55 (m, 3H), | | | |
| | 2.29 (dd, J = 10.9, 3.6 Hz, 1H), 1.99 (dd, J = 10.8, 3.7 | | | |
| | Hz, 1H), 1.88 (d, J = 5.6 Hz, 1H), 1.76 (s, 1H), 1.57- | | | |
| | 1.43 (m, 4H). | | | |
| I-117 | ¹H NMR: (400 MHz, Methanol-d₄) δ 8.34 (s, 1H), | Calculated | 2.7% | White |
| | 7.73 (s, 1H), 7.47 (td, J = 9.0, 6.6 Hz, 1H), 7.37-7.30 | exact mass = | | solid |
| | (m, 2H), 7.30-7.26 (m, 2H), 7.09 (tt, J = 7.1, 1.3 Hz, | 740.837 | | |
| | 1H), 6.99 (d, J = 8.4 Hz, 1H), 6.91 (ddd, J = 11.7, 8.9, | Found | | |
| | 2.6 Hz, 1H), 6.82 (tt, J = 8.6, 1.5 Hz, 1H), 6.70 (d, J = | [M + H]⁺ | | |
| | 2.4 Hz, 1H), 6.66 (dd, J = 8.4, 2.4 Hz, 1H), 6.27 (d, J = | (ESI+) = | | |
| | 0.9 Hz, 1H), 4.68-4.53 (m, 3H), 4.41-4.34 (m, 2H), | 740.2 | | |
| | 3.76 (dd, J = 5.1, 3.6 Hz, 2H), 3.74-3.68 (m, 2H), | | | |
| | 2.99 (dd, J = 13.7, 1.6 Hz, 1H), 2.78 (d, J = 13.8 Hz, | | | |
| | 1H), 2.58-2.48 (m, 2H), 2.31 (td, J = 8.0, 3.8 Hz, | | | |
| | 2H), 1.50 (dd, J = 7.7, 3.7 Hz, 2H), 1.47-1.40 (m, | | | |
| | 2H), 1.06 (s, 3H). | | | |
| I-118 | ¹H NMR: (400 MHz, DMSO-d₆) δ 8.26 (s, 1H), 7.74 (s, | Calculated | 11.8% | White |
| | 1H), 7.39-7.33 (m, 3H), 7.31-7.26 (m, 2H), 7.18- | exact mass = | | solid |
| | 7.12 (m, 1H), 7.09 (dt, J = 7.3, 1.3 Hz, 1H), 7.01- | 782.916 | | |
| | 6.98 (m, 1H), 6.97-6.92 (m, 1H), 6.86 (s, 2H), 6.71 | Found | | |
| | (d, J = 7.5 Hz, 2H), 6.24 (s, 1H), 5.66 (s, 1H), 4.48 (q, | [M + H]⁺ | | |
| | J = 14.1 Hz, 2H), 4.34 (dd, J = 5.5, 3.1 Hz, 2H), 3.81- | (ESI+) = | | |
| | 3.70 (m, 4H), 2.79-2.66 (m, 2H), 2.33 (p, J = 1.9 Hz, | 782.8 | | |
| | 1H), 2.16 (s, 2H), 2.00 (t, J = 12.5 Hz, 1H), 1.82 (s, | | | |
| | 1H), 1.73 (d, J = 12.3 Hz, 2H), 1.61 (td, J = 13.6, 12.8, | | | |
| | 4.3 Hz, 1H), 1.37-1.09 (m, 8H), 0.82 (q, J = 11.1 Hz, 1H). | | | |
| I-119 | ¹H NMR: (400 MHz, Methanol-d₄) δ 8.35 (s, 1H), | Calculated | 2.4% | White |
| | 7.74 (s, 1H), 7.48 (td, J = 9.0, 6.6 Hz, 1H), 7.34 (t, J = | exact mass = | | solid |
| | 7.8 Hz, 2H), 7.31-7.24 (m, 2H), 7.09 (t, J = 7.3 Hz, | 740.837 | | |
| | 1H), 6.99 (d, J = 8.4 Hz, 1H), 6.94-6.88 (m, 1H), | Found | | |
| | 6.84 (td, J = 8.5, 2.6 Hz, 1H), 6.71-6.65 (m, 2H), | [M + H]⁺ | | |
| | 6.23 (s, 1H), 4.70-4.53 (m, 3H), 4.37 (t, J = 4.4 Hz, | (ESI+) = | | |
| | 2H), 3.80-3.72 (m, 2H), 3.66 (s, 2H), 2.99-2.91 (m, | 740.7 | | |
| | 1H), 2.78 (s, 2H), 2.55 (d, J = 11.8 Hz, 1H), 2.30 (dt, | | | |
| | J = 21.2, 10.6 Hz, 2H), 2.18 (s, 3H), 2.06 (t, J = 11.4 Hz, | | | |
| | 1H), 1.71 (dd, J = 14.1, 6.8 Hz, 1H), 1.62-1.53 (m, | | | |
| | 2H), 1.47-1.38 (m, 2H). | | | |
| I-120 | ¹H NMR: (400 MHz, Methanol-d₄) δ 8.33 (d, J = 3.0 | Calculated | 20.9% | White |
| | Hz, 1H), 7.77 (s, 1H), 7.53 (q, J = 8.3 Hz, 1H), 7.39- | exact mass = | | solid |
| | 7.32 (m, 2H), 7.31-7.26 (m, 2H), 7.13-7.05 (m, | 726.81 | | |
| | 1H), 7.00 (dd, J = 8.4, 0.9 Hz, 1H), 6.97-6.83 (m, | Found | | |
| | 2H), 6.72 (d, J = 2.4 Hz, 1H), 6.68 (dd, J = 8.4, 2.4 Hz, | [M + H]⁺ | | |
| | 1H), 6.31 (d, J = 2.4 Hz, 1H), 4.57 (d, J = 2.8 Hz, 2H), | (ESI+) = | | |
| | 4.40-4.33 (m, 2H), 3.81-3.69 (m, 4H), 2.76-2.46 | 726.2 | | |
| | (m, 4H), 2.40-2.16 (m, 4H), 1.99 (qd, J = 16.0, 14.3, | | | |
| | 6.2 Hz, 2H), 1.54 (d, J = 12.4 Hz, 1H). | | | |
| I-121 | HCl salt ¹H NMR: (400 MHz, DMSO-d₆) δ 9.61 (s, 2H), | Calculated | 82.6% | Pale |
| | 8.49 (s, 1H), 7.84 (s, 1H), 7.46 (s, 1H), 7.37 (t, J = 7.8 | exact mass = | | yellow |
| | Hz, 2H), 7.30 (d, J = 7.8 Hz, 4H), 7.10 (t, J = 7.2 Hz, | 726.81 | | solid |
| | 1H), 7.01 (d, J = 8.4 Hz, 2H), 6.78 (d, J = 2.5 Hz, 1H), | Found | | |
| | 6.73 (dd, J = 8.4, 2.4 Hz, 1H), 6.66 (s, 1H), 4.71 (d, J = | [M + H]⁺ | | |
| | 23.2 Hz, 2H), 4.30 (d, J = 37.2 Hz, 4H), 2.98 (s, 2H), | (ESI+) = | | |
| | 1.87 (s, 3H), 1.52 (s, 1H). | 726.9 | | |
| I-122 | HCl salt ¹H NMR: (400 MHz, DMSO-d₆) δ 9.54 (s, 1H), | Calculated | 96.0% | Pale |
| | 9.14 (s, 2H), 8.43 (d, J = 15.5 Hz, 1H), 7.82 (d, J = 9.6 | exact mass = | | yellow |
| | Hz, 1H), 7.49 (td, J = 9.1, 6.5 Hz, 1H), 7.36 (tdd, J = | 740.837 | | solid |
| | 8.1, 5.1, 2.6 Hz, 3H), 7.31-7.26 (m, 3H), 7.17 (s, | Found | | |
| | 1H), 7.12-7.07 (m, 2H), 7.02 (dd, J = 8.4, 3.2 Hz, | [M + H]⁺ | | |
| | 1H), 6.78 (d, J = 2.4 Hz, 1H), 6.73 (dt, J = 6.0, 2.8 Hz, | (ESI+) = | | |
| | 1H), 6.63 (d, J = 9.2 Hz, 1H), 4.80-4.59 (m, 2H), | 740.9 | | |
| | 4.35 (s, 2H), 4.21 (t, J = 5.9 Hz, 2H), 3.76 (s, 2H), 3.17- | | | |
| | 3.07 (m, 2H), 2.88 (s, 2H), 2.76-2.66 (m, 3H), 1.88 | | | |
| | (d, J = 14.1 Hz, 2H), 1.76 (d, J = 13.6 Hz, 2H), 1.61 (d, | | | |
| | J = 13.1 Hz, 1H), 1.47 (d, J = 12.8 Hz, 1H). | | | |
| I-123 | HCl salt ¹H NMR: (400 MHz, DMSO-d₆) δ 11.05 (d, J = | Calculated | 95.0% | Pale |
| | 165.4 Hz, 1H), 9.79 (d, J = 102.9 Hz, 2H), 8.41 (d, J = | exact mass = | | yellow |
| | 7.4 Hz, 1H), 7.82 (d, J = 3.8 Hz, 1H), 7.37 (dtd, J = | 726.81 | | solid |
| | 11.8, 6.5, 5.2, 3.4 Hz, 3H), 7.33-7.29 (m, 2H), 7.28- | Found | | |
| | 7.22 (m, 1H), 7.12-7.07 (m, 1H), 7.01 (dd, J = 8.9, | [M + H]⁺ | | |
| | 5.8 Hz, 2H), 6.79 (d, J = 2.4 Hz, 1H), 6.73 (dd, J = 8.5, | (ESI+) = | | |
| | 2.4 Hz, 1H), 6.65 (s, 1H), 6.35 (s, 1H), 4.59 (s, 2H), | 726.2 | | |
| | 4.35 (s, 4H), 3.57 (s, 1H), 3.26 (s, 2H), 3.04 (s, 1H), | | | |
| | 2.76 (s, 1H), 2.45-2.32 (m, 2H), 2.24-2.12 (m, 2H). | | | |
| I-124 | HCl salt ¹H NMR: (400 MHz, DMSO-d₆) δ 10.97 (d, J = | Calculated | 95.0% | Pale |
| | 179.0 Hz, 1H), 9.65 (d, J = 46.7 Hz, 2H), 8.39 (d, J = | exact mass = | | yellow |
| | 2.9 Hz, 1H), 7.81 (s, 1H), 7.37 (ddt, J = 8.3, 5.5, 2.5 | 726.81 | | solid |
| | Hz, 3H), 7.33-7.28 (m, 2H), 7.25 (td, J = 6.3, 3.1 Hz, | Found | | |
| | 1H), 7.10 (tt, J = 7.2, 1.3 Hz, 1H), 7.04-6.96 (m, 2H), | [M + H]⁺ | | |
| | 6.79 (d, J = 2.4 Hz, 1H), 6.73 (dd, J = 8.4, 2.4 Hz, 1H), | (ESI+) = | | |
| | 6.65 (d, J = 4.2 Hz, 1H), 6.34 (s, 1H), 4.59 (s, 2H), | 726.2 | | |
| | 4.34 (s, 4H), 3.32 (d, J = 12.1 Hz, 1H), 3.18 (d, J = 7.1 | | | |
| | Hz, 2H), 2.97 (s, 1H), 2.79 (s, 1H), 2.45-2.30 (m, | | | |
| | 2H), 2.26-2.10 (m, 2H). | | | |
| I-125 | HCl salt ¹H NMR: (400 MHz, DMSO-d₆) δ 10.09- | Calculated | 83.3% | Pale |
| | 8.94 (m, 2H), 8.50-8.09 (m, 1H), 7.82 (d, J = 37.7 | exact mass = | | yellow |
| | Hz, 1H), 7.44 (d, J = 37.2 Hz, 1H), 7.39-7.34 (m, | 724.794 | | solid |
| | 2H), 7.32-7.27 (m, 2H), 7.15 (s, 2H), 7.11-7.08 (m, | Found | | |
| | 1H), 7.03 (d, J = 8.4 Hz, 1H), 6.79 (d, J = 2.4 Hz, 1H), | [M + H]⁺ | | |
| | 6.72 (dd, J = 8.5, 2.5 Hz, 1H), 6.57 (s, 1H), 4.69-4.43 | (ESI+) = | | |
| | (m, 2H), 4.35 (s, 2H), 4.23 (s, 2H), 3.77 (s, 4H), 3.08 | 724.7 | | |
| | (t, J = 31.3 Hz, 2H), 2.85 (s, 1H), 2.67 (p, J = 1.9 Hz, | | | |
| | 1H), 2.39-2.15 (m, 3H), 1.73 (s, 1H), 1.24 (s, 1H). | | | |
| I-126 | ¹H NMR: (400 MHz, Methanol-d₄) δ 7.39-7.33 (m, | Calculated | 24.1% | White |
| | 2H), 7.32-7.27 (m, 2H), 7.10 (tt, J = 7.1, 1.4 Hz, 1H), | exact mass = | | solid |
| | 7.00 (d, J = 8.4 Hz, 1H), 6.71 (d, J = 2.4 Hz, 1H), 6.68 | 489.605 | | |
| | (dd, J = 8.4, 2.4 Hz, 1H), 6.31 (d, J = 1.0 Hz, 1H), 4.37 | Found | | |
| | (t, J = 4.4 Hz, 2H), 3.85 (d, J = 1.0 Hz, 2H), 3.77 (dd, | [M + H]⁺ | | |
| | J = 5.0, 3.8 Hz, 2H), 3.15-3.10 (m, 2H), 2.70-2.61 | (ESI+) = | | |
| | (m, 3H), 1.91 (d, J = 13.4 Hz, 2H), 1.40-1.28 (m, 2H). | 489.5 | | |
| I-127 | ¹H NMR: (400 MHz, Methanol-d₄) δ 8.26 (s, 1H), | Calculated | 92.1% | Pale |
| | 7.73 (s, 1H), 7.39-7.28 (m, 5H), 7.12-7.07 (m, 1H), | exact mass = | | yellow |
| | 7.01 (d, J = 8.5 Hz, 1H), 6.77 (s, 1H), 6.75 (d, J = 2.4 | 724.794 | | solid |
| | Hz, 1H), 6.70-6.67 (m, 1H), 6.27 (s, 1H), 4.60-4.56 | Found | | |
| | (m, 1H), 4.48 (s, 1H), 4.37 (t, J = 4.4 Hz, 2H), 3.77 (t, | [M + H]⁺ | | |
| | J = 4.4 Hz, 2H), 3.73 (d, J = 6.8 Hz, 2H), 3.00 (d, J = | (ESI+) = | | |
| | 13.7 Hz, 1H), 2.86 (d, J = 8.8 Hz, 1H), 2.76 (d, J = 13.6 | 724.9 | | |
| | Hz, 1H), 2.62 (s, 1H), 2.48 (dd, J = 8.5, 3.0 Hz, 1H), | | | |
| | 2.28 (d, J = 8.7 Hz, 1H), 2.10 (d, J = 2.2 Hz, 1H), 1.29 | | | |
| | (q, J = 2.6 Hz, 2H). | | | |
| | HCl salt 1H NMR: (400 MHz, DMSO-d6) δ 9.37 (d, J = | | | |
| | 270.2 Hz, 2H), 8.35 (d, J = 62.8 Hz, 1H), 7.81 (d, J = | | | |
| | 31.8 Hz, 1H), 7.49 (s, 1H), 7.40-7.33 (m, 3H), 7.32- | | | |
| | 7.24 (m, 3H), 7.11 (q, J = 9.3, 7.1 Hz, 4H), 7.05-6.96 | | | |
| | (m, 2H), 6.79 (d, J = 2.5 Hz, 1H), 6.72 (dt, J = 8.3, 2.3 | | | |
| | Hz, 1H), 6.58 (d, J = 6.7 Hz, 1H), 4.75-4.42 (m, 2H), | | | |
| | 4.35 (s, 2H), 4.18 (d, J = 40.5 Hz, 2H), 3.76 (s, 4H), | | | |
| | 3.04 (d, J = 24.7 Hz, 2H), 2.89 (s, 1H), 2.67 (p, J = 1.9 | | | |
| | Hz, 1H), 2.42-2.19 (m, 3H), 1.74 (d, J = 8.4 Hz, 1H). | | | |
| I-128 | ¹H NMR: (400 MHz, DMSO-d₆) δ 9.23 (s, 2H), 8.44 (d, | Calculated | 36.2% | White |
| | J = 9.8 Hz, 1H), 7.83 (d, J = 4.9 Hz, 1H), 7.42-7.33 | exact mass = | | solid |
| | (m, 3H), 7.33-7.27 (m, 2H), 7.16 (ddd, J = 11.9, 9.1, | 754.82 | | |
| | 2.6 Hz, 1H), 7.09 (t, J = 7.2 Hz, 1H), 7.03 (d, J = 8.4 | Found | | |
| | Hz, 1H), 6.95 (dd, J = 8.9, 5.6 Hz, 1H), 6.79 (d, J = 2.4 | [M + H]⁺ | | |
| | Hz, 1H), 6.73 (dd, J = 8.4, 2.4 Hz, 1H), 6.66 (d, J = 1.5 | (ESI+) = | | |
| | Hz, 1H), 4.68-4.56 (m, 2H), 4.31 (d, J = 31.2 Hz, | 754.8 | | |
| | 8H), 4.02 (d, J = 38.9 Hz, 9H), 3.76 (s, 2H), 3.16 (t, J = | | | |
| | 15.0 Hz, 2H), 2.96 (q, J = 13.7 Hz, 1H), 2.85 (dd, J = | | | |
| | 15.6, 6.2 Hz, 1H), 2.10-1.92 (m, 2H), 1.62-1.15 | | | |
| | (m, 2H). | | | |
| I-129 | HCl salt ¹H NMR: (400 MHz, DMSO-d₆) δ 8.94 (s, 2H), | Calculated | 95.6% | Pale |
| | 8.35 (s, 1H), 7.79 (d, J = 7.0 Hz, 1H), 7.45-7.33 (m, | exact mass = | | yellow |
| | 3H), 7.29 (d, J = 7.9 Hz, 2H), 7.17 (d, J = 9.9 Hz, 2H), | 754.82 | | solid |
| | 7.10 (t, J = 7.3 Hz, 1H), 7.03 (dd, J = 8.4, 1.2 Hz, 1H), | Found | | |
| | 6.97 (d, J = 12.3 Hz, 1H), 6.81-6.77 (m, 1H), 6.73 | [M + H]⁺ | | |
| | (dd, J = 8.4, 2.4 Hz, 1H), 6.64 (s, 1H), 6.41 (s, 1H), | (ESI+) = | | |
| | 4.58 (dd, J = 14.9, 9.4 Hz, 2H), 4.32 (d, J = 27.9 Hz, | 754.8 | | |
| | 5H), 3.94 (d, J = 14.3 Hz, 2H), 3.76 (s, 5H), 3.27- | | | |
| | 3.11 (m, 3H), 2.97 (q, J = 13.9 Hz, 1H), 2.83 (d, J = | | | |
| | 15.6 Hz, 1H), 1.99 (d, J = 19.6 Hz, 2H), 1.51 (s, 1H), | | | |
| | 1.40-1.10 (m, 2H). | | | |
| I-130 | HCl salt ¹H NMR: (400 MHz, DMSO-d₆) δ 9.77 (d, J = | Calculated | 91.1% | Pale |
| | 121.8 Hz, 1H), 8.89 (s, 1H), 8.37 (s, 1H), 7.75 (s, 1H), | exact mass = | | yellow |
| | 7.37 (dd, J = 8.5, 7.2 Hz, 3H), 7.31-7.26 (m, 2H), | 726.81 | | solid |
| | 7.25-7.07 (m, 4H), 7.02 (d, J = 8.5 Hz, 1H), 6.92 (s, | Found | | |
| | 1H), 6.79 (d, J = 2.4 Hz, 1H), 6.73 (dd, J = 8.4, 2.4 Hz, | [M + H]⁺ | | |
| | 1H), 6.62 (s, 1H), 5.75 (s, 1H), 4.59 (s, 2H), 4.35 (s, | (ESI+) = | | |
| | 2H), 4.19 (d, J = 23.4 Hz, 2H), 3.76 (s, 2H), 3.01 (s, | 726.8 | | |
| | 1H), 2.84 (s, 3H), 1.73 (d, J = 38.3 Hz, 2H), 1.51- | | | |
| | 1.29 (m, 2H). | | | |
| I-131 | HCl salt ¹H NMR: (400 MHz, DMSO-d₆) δ 9.16 (d, J = | Calculated | 94.0% | Pale |
| | 158.2 Hz, 1H), 8.70 (s, 1H), 8.35 (d, J = 51.9 Hz, 1H), | exact mass = | | yellow |
| | 7.80 (d, J = 43.4 Hz, 1H), 7.48 (s, 1H), 7.36 (t, J = 7.8 | 740.837 | | solid |
| | Hz, 3H), 7.28 (d, J = 7.9 Hz, 2H), 7.16 (d, J = 9.6 Hz, | Found | | |
| | 3H), 7.09 (t, J = 7.1 Hz, 2H), 7.02 (d, J = 8.4 Hz, 1H), | [M + H]⁺ | | |
| | 6.96 (t, J = 8.6 Hz, 1H), 6.78 (d, J = 2.4 Hz, 1H), 6.72 | (ESI+) = | | |
| | (dd, J = 8.5, 2.4 Hz, 1H), 6.60 (s, 1H), 5.62 (s, 1H), | 740.8 | | |
| | 4.61 (d, J = 61.5 Hz, 2H), 4.35 (s, 2H), 4.21 (d, J = 8.2 | | | |
| | Hz, 2H), 3.76 (s, 3H), 3.11 (s, 1H), 2.84 (d, J = 14.1 | | | |
| | Hz, 2H), 2.67 (s, 3H), 2.04 (s, 2H), 1.79 (d, J = 46.3 | | | |
| | Hz, 2H), 1.51 (s, 3H), 1.23 (s, 2H), 1.09-0.94 (m, 2H). | | | |
| I-132 | HCl salt ¹H NMR: (400 MHz, DMSO-d₆) δ 10.32 (s, | Calculated | 94.2% | Pale |
| | 1H), 9.33 (s, 2H), 8.35 (s, 1H), 7.79 (s, 1H), 7.36 (qt, | exact mass = | | yellow |
| | J = 5.9, 3.3 Hz, 3H), 7.29 (dt, J = 9.1, 2.0 Hz, 2H), 7.27- | 740.837 | | solid |
| | 7.21 (m, 1H), 7.17 (s, 1H), 7.09 (tt, J = 7.1, 1.2 Hz, | Found | | |
| | 1H), 7.03 (dq, J = 8.5, 2.9 Hz, 2H), 6.79 (d, J = 2.4 Hz, | [M + H]⁺ | | |
| | 1H), 6.72 (dd, J = 8.4, 2.4 Hz, 1H), 6.65 (s, 1H), 6.24 | (ESI+) = | | |
| | (s, 1H), 4.57 (s, 2H), 4.31 (d, J = 26.8 Hz, 4H), 3.76 (s, | 740.8 | | |
| | 2H), 3.47 (d, J = 11.9 Hz, 2H), 3.18 (s, 1H), 3.03 (s, | | | |
| | 1H), 2.94-2.80 (m, 2H), 2.18 (d, J = 12.4 Hz, 3H), | | | |
| | 1.91 (s, 2H). | | | |
| I-133 | HCl salt ¹H NMR: (400 MHz, DMSO-d₆) δ 10.43 (s, | Calculated | 77.4% | Pale |
| | 1H), 9.38 (s, 2H), 8.36 (t, J = 1.4 Hz, 1H), 7.79 (d, J = | exact mass = | | yellow |
| | 1.2 Hz, 1H), 7.40-7.33 (m, 3H), 7.32-7.28 (m, 2H), | 740.837 | | solid |
| | 7.28-7.22 (m, 1H), 7.17 (s, 1H), 7.09 (ddd, J = 7.2, | Found | | |
| | 5.9, 1.3 Hz, 1H), 7.05-6.99 (m, 2H), 6.78 (d, J = 2.5 | [M + H]⁺ | | |
| | Hz, 1H), 6.72 (dd, J = 8.4, 2.4 Hz, 1H), 6.66 (s, 1H), | (ESI+) = | | |
| | 6.26 (d, J = 4.5 Hz, 1H), 4.58 (s, 2H), 4.31 (d, J = 28.0 | 740.8 | | |
| | Hz, 4H), 3.76 (s, 2H), 3.47 (d, J = 11.9 Hz, 2H), 3.25- | | | |
| | 2.98 (m, 3H), 2.87 (t, J = 12.5 Hz, 2H), 2.19 (d, J = | | | |
| | 13.5 Hz, 3H), 1.92 (d, J = 12.4 Hz, 2H). | | | |
| I-134 | HCl salt ¹H NMR: (400 MHz, DMSO-d₆) δ 10.86 (s, | Calculated | 93.3% | Pale |
| | 1H), 9.20 (s, 1H), 8.82 (s, 1H), 8.42 (d, J = 3.4 Hz, 1H), | exact mass = | | yellow |
| | 7.82 (s, 1H), 7.46-7.39 (m, 1H), 7.36 (dd, J = 6.8, | 726.81 | | solid |
| | 1.8 Hz, 2H), 7.33-7.29 (m, 2H), 7.26 (td, J = 9.2, 4.5 | Found | | |
| | Hz, 1H), 7.14-7.04 (m, 3H), 7.02 (d, J = 8.4 Hz, 1H), | [M + H]⁺ | | |
| | 6.79 (d, J = 2.4 Hz, 1H), 6.75 (dd, J = 8.4, 2.4 Hz, 1H), | (ESI+) = | | |
| | 6.68 (s, 1H), 4.81-4.62 (m, 2H), 4.36 (d, J = 4.2 Hz, | 726.8 | | |
| | 4H), 3.42 (q, J = 7.1, 5.0 Hz, 3H), 3.28 (s, 1H), 2.86 (d, | | | |
| | J = 11.6 Hz, 2H), 2.26 (t, J = 15.4 Hz, 2H), 1.98 (d, J = | | | |
| | 13.0 Hz, 2H). | | | |
| I-135 | HCl salt ¹H NMR: (400 MHz, DMSO-d₆) δ 9.13 (d, J = | Calculated | 99.3% | Pale |
| | 117.6 Hz, 2H), 8.31 (d, J = 12.9 Hz, 1H), 7.70 (s, 1H), | exact mass = | | yellow |
| | 7.36 (t, J = 7.7 Hz, 4H), 7.29 (d, J = 7.8 Hz, 2H), 7.21- | 740.794 | | solid |
| | 7.07 (m, 4H), 7.02 (d, J = 8.4 Hz, 1H), 6.94 (td, J = | Found | | |
| | 8.4, 2.7 Hz, 1H), 6.77 (d, J = 2.4 Hz, 1H), 6.71 (dd, J = | [M + H]⁺ | | |
| | 8.4, 2.4 Hz, 1H), 6.63 (d, J = 14.3 Hz, 1H), 4.73 (dd, | (ESI+) = | | |
| | J = 11.1, 4.0 Hz, 2H), 4.47 (t, J = 12.4 Hz, 1H), 4.35 (q, | 740.8 | | |
| | J = 4.6 Hz, 2H), 4.27-4.16 (m, 2H), 3.90 (d, J = 13.4 | | | |
| | Hz, 1H), 3.10 (d, J = 14.5 Hz, 1H), 2.81 (t, J = 13.0 Hz, | | | |
| | 1H), 2.70-2.59 (m, 1H), 2.39 (t, J = 12.4 Hz, 1H), | | | |
| | 2.11-1.99 (m, 1H), 1.85-1.47 (m, 2H), 1.34-1.21 | | | |
| | (m, 1H), 0.44 (d, J = 12.1 Hz, 1H). | | | |
| I-136 | HCl salt ¹H NMR: (400 MHz, DMSO-d₆) δ 10.62 (d, J = | Calculated | 76.9% | Pale |
| | 109.3 Hz, 1H), 9.55 (s, 2H), 8.40 (s, 1H), 7.83 (s, 1H), | exact mass = | | yellow |
| | 7.45-7.40 (m, 1H), 7.37 (dd, J = 8.6, 7.2 Hz, 2H), | 798.873 | | solid |
| | 7.33-7.28 (m, 2H), 7.24 (ddd, J = 11.9, 8.9, 2.5 Hz, | Found | | |
| | 2H), 7.12-7.07 (m, 1H), 7.05-6.99 (m, 2H), 6.79 | [M + H]⁺ | | |
| | (d, J = 2.4 Hz, 1H), 6.73 (dd, J = 8.5, 2.4 Hz, 1H), 6.68 | (ESI+) = | | |
| | (s, 1H), 6.22 (s, 1H), 4.63-4.49 (m, 2H), 4.39-4.25 | 799.1 | | |
| | (m, 4H), 4.20-4.12 (m, 1H), 3.97 (dd, J = 25.3, 10.2 | | | |
| | Hz, 3H), 3.76 (s, 2H), 3.23 (s, 2H), 3.04 (s, 2H), 2.40 | | | |
| | (dt, J = 14.3, 7.2 Hz, 2H), 2.24 (d, J = 12.9 Hz, 2H), | | | |
| | 2.11 (dt, J = 14.0, 7.1 Hz, 2H), 2.00 (d, J = 13.5 Hz, 2H). | | | |
| I-137 | HCl salt ¹H NMR: (400 MHz, DMSO-d₆) δ 9.80 (s, 1H), | Calculated | 95.7% | Pale |
| | 9.51 (s, 1H), 8.50 (d, J = 2.9 Hz, 1H), 7.90 (d, J = 1.1 | exact mass = | | yellow |
| | Hz, 1H), 7.45-7.38 (m, 1H), 7.36 (dd, J = 8.5, 7.2 Hz, | 762.791 | | solid |
| | 2H), 7.31-7.25 (m, 2H), 7.17 (dddd, J = 11.9, 9.3, | Found | | |
| | 6.8, 2.6 Hz, 1H), 7.09 (dd, J = 8.0, 6.5 Hz, 1H), 7.04- | [M + H]⁺ | | |
| | 6.95 (m, 2H), 6.78 (d, J = 2.4 Hz, 1H), 6.73 (s, 1H), | (ESI+) = | | |
| | 6.68 (s, 1H), 6.11 (s, 1H), 4.57 (dt, J = 10.1, 5.0 Hz, | 762.9 | | |
| | 2H), 4.32 (d, J = 21.1 Hz, 4H), 3.76 (s, 3H), 3.44 (p, J = | | | |
| | 7.0 Hz, 1H), 3.20 (t, J = 13.5 Hz, 1H), 3.06 (dd, J = | | | |
| | 30.7, 14.0 Hz, 1H), 2.82 (s, 1H), 2.78-2.66 (m, 1H), | | | |
| | 2.33 (p, J = 1.9 Hz, 1H), 2.22 (s, 1H), 2.14-1.99 (m, | | | |
| | 1H), 1.62-1.39 (m, 1H). | | | |
| I-138 | HCl salt ¹H NMR: (400 MHz, DMSO-d₆) δ 9.56 (d, J = | Calculated | 98.6% | Pale |
| | 66.4 Hz, 3H), 8.59-8.41 (m, 1H), 7.91-7.79 (m, | exact mass = | | yellow |
| | 1H), 7.60-7.41 (m, 2H), 7.40-7.33 (m, 3H), 7.32- | 740.837 | | solid |
| | 7.27 (m, 2H), 7.26-7.05 (m, 4H), 7.01 (dd, J = 8.5, | Found | | |
| | 6.3 Hz, 1H), 6.79 (dt, J = 5.8, 2.7 Hz, 1H), 6.72 (dtd, | [M + H]⁺ | | |
| | J = 13.3, 7.2, 6.7, 3.6 Hz, 1H), 6.65 (d, J = 6.1 Hz, 1H), | (ESI+) = | | |
| | 4.92-4.74 (m, 1H), 4.64 (dd, J = 20.4, 12.9 Hz, 1H), | 740.8 | | |
| | 4.35 (s, 2H), 4.31-4.18 (m, 2H), 3.92 (d, J = 14.1 Hz, | | | |
| | 1H), 3.76 (s, 2H), 3.07 (dq, J = 29.7, 13.5 Hz, 2H), | | | |
| | 2.80 (dd, J = 43.9, 8.2 Hz, 1H), 2.26-2.06 (m, 2H), | | | |
| | 2.06-1.68 (m, 3H), 1.32 (d, J = 6.3 Hz, 1H), 1.21 (q, J = | | | |
| | 7.3, 5.8 Hz, 1H), 1.00-0.80 (m, 1H). | | | |
| I-139 | HCl salt ¹H NMR: (400 MHz, DMSO-d₆) δ 9.37 (s, 1H), | Calculated | 91.2% | Pale |
| | 8.90 (s, 1H), 7.50 (d, J = 2.8 Hz, 1H), 7.42-7.33 (m, | exact mass = | | yellow |
| | 4H), 7.30 (d, J = 8.3 Hz, 3H), 7.16 (s, 1H), 7.12-7.04 | 725.822 | | solid |
| | (m, 2H), 7.01 (dd, J = 8.4, 1.5 Hz, 1H), 6.78 (t, J = 2.0 | Found | | |
| | Hz, 1H), 6.72 (dd, J = 8.4, 2.4 Hz, 1H), 6.65 (s, 1H), | [M + H]⁺ | | |
| | 4.74 (s, 1H), 4.54 (d, J = 13.7 Hz, 1H), 4.35 (s, 2H), | (ESI+) = | | |
| | 4.22 (s, 2H), 3.76 (s, 2H), 3.03 (s, 3H), 2.04 (d, J = | 725.2 | | |
| | 39.0 Hz, 3H). | | | |
| I-141 | HCl salt ¹H NMR: (400 MHz, DMSO-d₆) δ 9.02 (s, 2H), | Calculated | 87.7% | Pale |
| | 8.32 (d, J = 10.7 Hz, 1H), 7.75 (d, J = 4.0 Hz, 1H), 7.37 | exact mass = | | yellow |
| | (t, J = 7.6 Hz, 3H), 7.33-7.26 (m, 2H), 7.18 (ddd, J = | 740.794 | | solid |
| | 11.9, 9.0, 2.6 Hz, 2H), 7.10 (t, J = 7.3 Hz, 1H), 7.03 (d, | Found | | |
| | J = 8.5 Hz, 1H), 6.96 (dd, J = 9.7, 6.8 Hz, 1H), 6.79 (d, | [M + H]⁺ | | |
| | J = 2.4 Hz, 1H), 6.74-6.70 (m, 1H), 6.64 (d, J = 1.7 | (ESI+) = | | |
| | Hz, 1H), 6.26 (d, J = 41.1 Hz, 1H), 4.69 (dd, J = 14.4, | 740.2 | | |
| | 6.1 Hz, 1H), 4.32 (d, J = 23.4 Hz, 4H), 4.04 (d, J = 14.0 | | | |
| | Hz, 1H), 3.88 (d, J = 14.2 Hz, 1H), 3.76 (s, 2H), 3.26 | | | |
| | (d, J = 12.1 Hz, 1H), 2.66 (d, J = 44.6 Hz, 2H), 2.12 (s, | | | |
| | 1H), 1.68 (s, 1H). | | | |
| I-143 | HCl salt ¹H NMR: (400 MHz, DMSO-d₆) δ 8.52 (s, 2H), | Calculated | 88.3% | Pale |
| | 8.27 (s, 1H), 7.73 (s, 1H), 7.36 (dd, J = 8.5, 7.2 Hz, | exact mass = | | yellow |
| | 2H), 7.31-7.26 (m, 3H), 7.16 (s, 1H), 7.10 (t, J = 7.4 | 740.837 | | solid |
| | Hz, 1H), 7.06-7.00 (m, 2H), 6.88 (d, J = 9.0 Hz, 1H), | Found | | |
| | 6.77 (d, J = 2.4 Hz, 1H), 6.71 (dd, J = 8.4, 2.4 Hz, 1H), | [M + H]⁺ | | |
| | 6.55 (s, 1H), 5.59 (s, 1H), 4.55 (s, 2H), 4.35 (s, 2H), | (ESI+) = | | |
| | 4.16 (s, 2H), 3.76 (s, 2H), 2.63 (s, 2H), 1.93-1.78 (m, | 740.4 | | |
| | 3H), 1.64 (s, 2H), 1.15 (s, 2H), 0.99 (d, J = 6.7 Hz, 3H). | | | |
| I-145 | HCl salt ¹H NMR: (400 MHz, DMSO-d₆) δ 9.60 (d, J = | Calculated | 90.8% | Pale |
| | 105.8 Hz, 3H), 8.26 (d, J = 29.3 Hz, 1H), 7.95 (d, J = | exact mass = | | yellow |
| | 15.8 Hz, 1H), 7.49 (d, J = 7.6 Hz, 2H), 7.44-7.33 (m, | 690.829 | | solid |
| | 5H), 7.32-7.26 (m, 2H), 7.17 (s, 1H), 7.09 (t, J = 7.2 | Found | | |
| | Hz, 1H), 7.01 (dd, J = 8.4, 3.8 Hz, 1H), 6.87 (d, J = | [M + H]⁺ | | |
| | 16.3 Hz, 1H), 6.77 (d, J = 2.5 Hz, 1H), 6.71 (dt, J = 8.5, | (ESI+) = | | |
| | 4.1 Hz, 1H), 6.65 (s, 1H), 4.68 (d, J = 14.5 Hz, 1H), | 690.4 | | |
| | 4.53 (d, J = 14.5 Hz, 1H), 4.35 (s, 2H), 4.22 (s, 2H), | | | |
| | 3.76 (s, 3H), 3.62 (d, J = 14.5 Hz, 1H), 3.50 (s, 1H), | | | |
| | 3.15 (d, J = 17.5 Hz, 2H), 2.98 (s, 1H), 2.75 (s, 1H), | | | |
| | 2.09 (d, J = 36.8 Hz, 3H), 1.92 (d, J = 28.1 Hz, 1H). | | | |
| I-146 | HCl salt ¹H NMR: (400 MHz, DMSO-d₆) δ 10.30 (d, J = | Calculated | 90.8% | Pale |
| | 34.5 Hz, 1H), 9.59 (d, J = 49.0 Hz, 2H), 8.96 (d, J = | exact mass = | | yellow |
| | 14.4 Hz, 1H), 8.90-8.81 (m, 1H), 8.57 (s, 2H), 7.99 | 691.817 | | solid |
| | (dd, J = 8.2, 5.6 Hz, 1H), 7.87 (s, 1H), 7.68 (s, 1H), | Found | | |
| | 7.37 (t, J = 7.8 Hz, 2H), 7.33-7.26 (m, 2H), 7.10 (tt, J = | [M + H]⁺ | | |
| | 7.2, 1.3 Hz, 1H), 7.02 (d, J = 8.4 Hz, 1H), 6.78 (d, J = | (ESI+) = | | |
| | 2.4 Hz, 1H), 6.73-6.64 (m, 2H), 4.89 (d, J = 14.4 Hz, | 691.9 | | |
| | 2H), 4.75 (d, J = 14.4 Hz, 2H), 4.39-4.32 (m, 2H), | | | |
| | 4.23 (s, 2H), 4.00 (d, J = 14.1 Hz, 1H), 3.75 (s, 4H), | | | |
| | 3.33 (s, 3H), 2.12 (d, J = 26.9 Hz, 4H), 1.19 (d, J = | | | |
| | 36.3 Hz, 1H). | | | |
| I-147 | HCl salt ¹H NMR: (400 MHz, DMSO-d₆) δ 9.74 (s, 1H), | Calculated | 96.7% | Pale |
| | 9.44 (s, 2H), 8.55 (d, J = 4.9 Hz, 1H), 8.31 (s, 1H), | exact mass = | | yellow |
| | 7.95-7.83 (m, 2H), 7.65 (d, J = 8.0 Hz, 1H), 7.42- | 691.817 | | solid |
| | 7.33 (m, 3H), 7.30 (d, J = 8.2 Hz, 2H), 7.21-7.15 (m, | Found | | |
| | 1H), 7.09 (dd, J = 15.3, 8.1 Hz, 2H), 7.01 (d, J = 8.4 | [M + H]⁺ | | |
| | Hz, 1H), 6.78 (d, J = 2.4 Hz, 1H), 6.72 (dd, J = 8.4, 2.5 | (ESI+) = | | |
| | Hz, 1H), 6.66 (s, 1H), 4.68-4.54 (m, 2H), 4.35 (s, | 691.3 | | |
| | 2H), 4.23 (d, J = 6.6 Hz, 2H), 3.98 (d, J = 13.7 Hz, 1H), | | | |
| | 3.79-3.73 (m, 2H), 3.32-3.06 (m, 4H), 2.92 (s, 1H), | | | |
| | 2.09 (d, J = 11.3 Hz, 3H), 1.99-1.88 (m, 1H). | | | |
| I-148 | HCl salt ¹H NMR: (400 MHz, DMSO-d₆) δ 10.07 (s, | Calculated | 98.5% | Pale |
| | 1H), 9.76 (s, 2H), 8.51 (s, 1H), 8.12 (s, 1H), 7.43 (s, | exact mass = | | yellow |
| | 1H), 7.39-7.34 (m, 2H), 7.32-7.27 (m, 2H), 7.13- | 740.837 | | solid |
| | 7.06 (m, 2H), 7.03 (d, J = 8.4 Hz, 2H), 6.80 (d, J = 2.4 | Found | | |
| | Hz, 1H), 6.76-6.70 (m, 2H), 5.32 (s, 1H), 4.96 (d, J = | [M + H]⁺ | | |
| | 14.8 Hz, 1H), 4.41-4.22 (m, 4H), 3.97 (s, 1H), 3.81- | (ESI+) = | | |
| | 3.70 (m, 3H), 3.31 (s, 1H), 2.36-2.13 (m, 3H), 1.15 | 740.4 | | |
| | (d, J = 74.9 Hz, 3H). | | | |
| I-149 | HCl salt ¹H NMR: (400 MHz, DMSO-d₆) δ 9.62 (d, J = | Calculated | 95.6% | Pale |
| | 108.7 Hz, 2H), 8.48 (s, 1H), 7.69 (s, 1H), 7.37 (dd, J = | exact mass = | | yellow |
| | 8.5, 7.1 Hz, 3H), 7.32-7.28 (m, 3H), 7.23 (s, 1H), | 740.837 | | solid |
| | 7.10 (td, J = 7.1, 1.3 Hz, 1H), 7.01 (d, J = 8.4 Hz, 1H), | Found | | |
| | 6.94 (d, J = 9.1 Hz, 1H), 6.77 (d, J = 2.4 Hz, 1H), 6.71 | [M + H]⁺ | | |
| | (dd, J = 8.4, 2.4 Hz, 1H), 6.64 (s, 1H), 4.77 (s, 2H), | (ESI+) = | | |
| | 4.41-4.28 (m, 2H), 4.19 (s, 2H), 3.49 (s, 2H), 3.17 (s, | 740.3 | | |
| | 2H), 2.74 (d, J = 5.0 Hz, 1H), 2.08 (d, J = 2.5 Hz, 3H), | | | |
| | 1.69 (s, 2H). | | | |
| I-150 | HCl salt ¹H NMR: (400 MHz, DMSO-d₆) δ 10.14- | Calculated | 68.4% | Pale |
| | 9.11 (m, 3H), 7.42-7.32 (m, 4H), 7.31-7.27 (m, | exact mass = | | yellow |
| | 2H), 7.15 (d, J = 20.9 Hz, 2H), 7.12-7.08 (m, 1H), | 727.798 | | solid |
| | 7.07-6.99 (m, 2H), 6.78 (d, J = 2.4 Hz, 1H), 6.72 (dd, | Found | | |
| | J = 8.4, 2.4 Hz, 1H), 6.65 (s, 1H), 4.97 (s, 2H), 4.30 (d, | [M + H]⁺ | | |
| | J = 40.7 Hz, 4H), 3.76 (s, 3H), 3.60 (d, J = 18.2 Hz, | (ESI+) = | | |
| | 2H), 3.08 (s, 4H), 2.03 (d, J = 31.4 Hz, 4H), 1.24 (d, J = | 727.8 | | |
| | 2.2 Hz, 1H). | | | |
| I-151 | HCl salt ¹H NMR: (400 MHz, DMSO-d₆) δ 14.48 (s, | Calculated | 13.7% | Pale |
| | 1H), 9.07 (d, J = 175.0 Hz, 2H), 7.50 (s, 1H), 7.36 (td, | exact mass = | | yellow |
| | J = 7.1, 2.5 Hz, 4H), 7.31-7.26 (m, 2H), 7.22-7.12 | 725.822 | | solid |
| | (m, 2H), 7.10 (t, J = 7.3 Hz, 1H), 7.02 (d, J = 8.4 Hz, | Found | | |
| | 2H), 6.78 (d, J = 2.4 Hz, 1H), 6.72 (dd, J = 8.5, 2.4 Hz, | [M + H]⁺ | | |
| | 1H), 6.62 (s, 1H), 4.54 (s, 2H), 4.35 (s, 2H), 4.23 (s, | (ESI+) = | | |
| | 2H), 3.76 (s, 3H), 2.90 (s, 2H), 2.09 (s, 2H), 1.84 (s, | 725.3 | | |
| | 1H), 1.35 (s, 1H). | | | |
| I-152 | HCl salt ¹H NMR: (400 MHz, DMSO-d₆) δ 14.59 (s, | Calculated | 7.3% | Pale |
| | 1H), 9.86 (d, J = 287.8 Hz, 2H), 8.96 (s, 1H), 7.62 (s, | exact mass = | | yellow |
| | 1H), 7.53 (t, J = 1.7 Hz, 1H), 7.46-7.33 (m, 5H), 7.33- | 725.822 | | solid |
| | 7.28 (m, 2H), 7.17 (s, 2H), 7.09 (t, J = 7.2 Hz, 2H), | Found | | |
| | 7.01 (d, J = 8.4 Hz, 1H), 6.78 (d, J = 2.4 Hz, 1H), 6.72 | [M + H]⁺ | | |
| | (dd, J = 8.5, 2.4 Hz, 1H), 6.66 (s, 1H), 4.87 (s, 1H), | (ESI+) = | | |
| | 4.57 (d, J = 14.1 Hz, 1H), 4.41-4.30 (m, 2H), 4.24 (s, | 725.3 | | |
| | 2H), 3.76 (s, 3H), 3.17 (s, 3H), 2.30-1.86 (m, 5H), | | | |
| | 1.24 (s, 1H). | | | |
| I-155 | HCl salt ¹H NMR: (400 MHz, DMSO-d₆) δ 9.82 (s, 1H), | Calculated | 79.0% | Pale |
| | 9.58 (s, 2H), 8.34 (d, J = 30.0 Hz, 1H), 7.97 (d, J = | exact mass = | | yellow |
| | 18.8 Hz, 1H), 7.54-7.45 (m, 2H), 7.43-7.33 (m, | 690.829 | | solid |
| | 5H), 7.32-7.27 (m, 2H), 7.09 (t, J = 7.2 Hz, 1H), 7.01 | Found | | |
| | (dd, J = 8.5, 3.5 Hz, 1H), 6.91 (s, 1H), 6.77 (d, J = 2.4 | [M + H]⁺ | | |
| | Hz, 1H), 6.75-6.68 (m, 1H), 6.66 (s, 1H), 4.71 (d, J = | (ESI+) = | | |
| | 14.4 Hz, 1H), 4.56 (d, J = 14.4 Hz, 1H), 4.42-4.31 | 690.4 | | |
| | (m, 2H), 4.30-4.17 (m, 2H), 3.62 (d, J = 13.7 Hz, | | | |
| | 1H), 3.51 (d, J = 12.0 Hz, 1H), 3.07 (d, J = 51.9 Hz, | | | |
| | 3H), 2.76 (d, J = 10.7 Hz, 1H), 2.24-1.83 (m, 4H). | | | |
| I-156 | HCl salt ¹H NMR: (400 MHz, DMSO-d₆) δ 9.83 (s, 1H), | Calculated | 78.8% | Pale |
| | 9.58 (s, 2H), 8.37 (d, J = 28.9 Hz, 1H), 7.99 (d, J = | exact mass = | | yellow |
| | 18.2 Hz, 1H), 7.56-7.45 (m, 2H), 7.43-7.33 (m, | 690.829 | | solid |
| | 5H), 7.32-7.28 (m, 2H), 7.09 (t, J = 7.2 Hz, 1H), 7.01 | Found | | |
| | (dd, J = 8.4, 3.5 Hz, 1H), 6.92 (s, 1H), 6.77 (d, J = 2.4 | [M + H]⁺ | | |
| | Hz, 1H), 6.74-6.68 (m, 1H), 6.66 (s, 1H), 4.72 (dd, J = | (ESI+) = | | |
| | 14.2, 4.0 Hz, 1H), 4.56 (d, J = 14.4 Hz, 1H), 4.35 (s, | 690.3 | | |
| | 2H), 4.29-4.18 (m, 2H), 3.74 (d, J = 18.5 Hz, 3H), | | | |
| | 3.62 (d, J = 13.8 Hz, 1H), 3.51 (d, J = 12.0 Hz, 1H), | | | |
| | 3.35-2.98 (m, 3H), 2.82-2.71 (m, 1H), 2.25-1.87 | | | |
| | (m, 4H). | | | |

### Synthesis of 3-amino-6-(1-(1-(2-(2,4-difluorophenyl)-2-hydroxy-3-(1H-1,2,4-triazol-1-yl)propyl)piperidin-4-yl)-1H-1,2,3-triazol-4-yl)-1-(4-phenyl-3,4-dihydro-2H-benzo[b][1,4]oxazin-6-yl)thieno[2,3-b]pyrazin-2(1H)-one—I-51

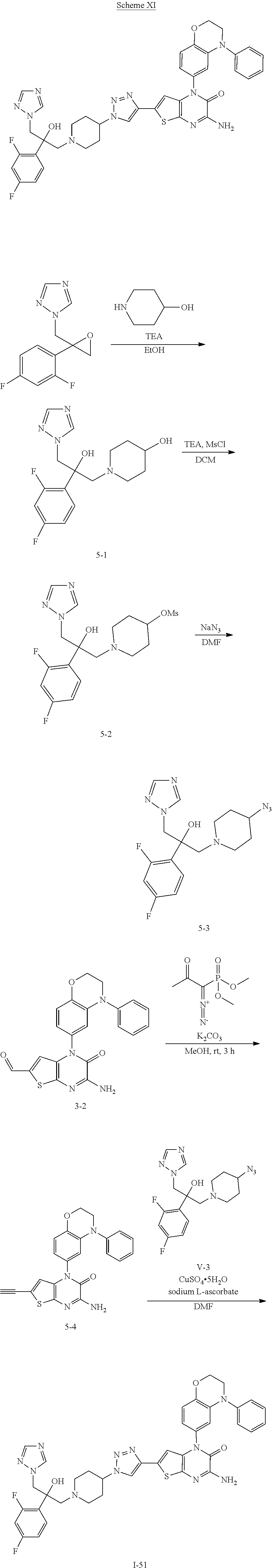

### 1-(2-(2,4-difluorophenyl)-2-hydroxy-3-(1H-1,2,4-triazol-1-yl)propyl)piperidin-4-ol (5-1)

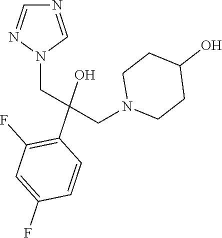 [SMILES: OC1CCN(CC(O)(Cn2cncn2)c2ccc(F)cc2F)CC1]

1-((2-(2,4-difluorophenyl)oxiran-2-yl)methyl)-1H-1,2,4-triazole (2.0 g, 8.4 mmol) was dissolved in EtOH (15 mL) and treated with 4-hydroxypiperidine (1.3 g, 12.7 mmol) and triethylamine (2.5 g, 25.2 mmol). The mixture was heated in a microwave reactor at 80° C. for 2 hours. When the reaction was complete, the solvent was evaporated and the product was purified by column chromatography on silica gel (DCM/MeOH=10:1) to give 1-(2-(2,4-difluorophenyl)-2-hydroxy-3-(1H-1,2,4-triazol-1-yl)propyl)piperidin-4-ol 5-1 as a pale yellow solid (2.6 g, 91% yield).

LCMS: Calculated Exact Mass=338.2, Found [M+H]⁺ (ESI+)=339.2.

¹H NMR (400 MHz, DMSO) δ 8.29 (s, 1H), 7.74 (s, 1H), 7.39 (td, J=9.0, 7.0 Hz, 1H), 7.15 (ddd, J=11.9, 9.2, 2.5 Hz, 1H), 6.95 (td, J=8.5, 2.5 Hz, 1H), 5.60 (s, 1H), 4.55 (t, J=8.5 Hz, 2H), 4.46 (d, J=4.2 Hz, 1H), 3.39-3.31 (m, 1H), 2.82 (dd, J=13.8, 1.5 Hz, 1H), 2.65 (d, J=13.8 Hz, 1H), 2.56-2.55 (m, 1H), 2.17-2.11 (m, 2H), 1.57-1.54 (m, 2H), 1.32-1.19 (m, 2H).

### 1-(2-(2,4-difluorophenyl)-2-hydroxy-3-(1H-1,2,4-triazol-1-yl)propyl)piperidin-4-ylmethanesulfonate (5-2)

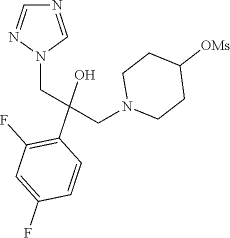 [SMILES: CS(=O)(=O)OC1CCN(CC(O)(Cn2cncn2)c2ccc(F)cc2F)CC1]

Methanesulfonyl chloride (374 mg, 3.2 mmol) was added dowise to a solution of 1-(2-(2,4-difluorophenyl)-2-hydroxy-3-(1H-1,2,4-triazol-1-yl)propyl)piperidin-4-ol 5-1 (1.0 g, 2.9 mmol) and triethylamine (586 mg, 5.8 mmol) were dissolved in anhydrous DCM (20 mL) at 0° C. The reaction mixture was stirred for 1.5 hours at this temperature. Then, water (50 mL) was added to quench the reaction and the mixture was extracted with DCM (100 mL). After drying and evaporation, the crude product 1-(2-(2,4-difluorophenyl)-2-hydroxy-3-(1H-1,2,4-triazol-1-yl)propyl)piperidin-4-ylmethanesulfonate 5-2 was used in the next step without further purification.

### 1-(4-azidopiperidin-1-yl)-2-(2,4-difluorophenyl)-3-(1H-1,2,4-triazol-1-yl)propan-2-ol (5-3)

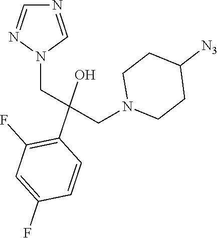 [SMILES: [N-]=[N+]=NC1CCN(CC(O)(Cn2cncn2)c2ccc(F)cc2F)CC1]

(2-(2,4-difluorophenyl)-2-hydroxy-3-(1H-1,2,4-triazol-1-yl)propyl)piperidin-4-yl methanesulfonate 5-2 (1.3 g, 3.1 mmol) was dissolved in DMF (10 mL) and sodium azide (604 mg, 9.3 mmol)was added. The reaction mixture was stirred for 6 hours at 40° C. Then, water (100 mL) was added and the mixture was extracted with EtOAc (100 mL×3). The combined organic layers were dried over anhydrous Na₂SO₄, filtered and concentrated. The crude product was purified by column chromatography on silica gel (PE/EtOAc=1:2) to afford 1-(4-azidopiperidin-1-yl)-2-(2,4-difluorophenyl)-3-(1H-1,2,4-triazol-1-yl)propan-2-ol 5-3 as a yellow oil (480 mg, 43.6% yield for two steps).

LCMS: Calculated Exact Mass=363.1, Found [M+H]⁺ (ESI+)=364.2.

¹H NMR (400 MHz, DMSO) δ 8.29 (s, 1H), 7.74 (s, 1H), 7.40 (td, J=9.0, 7.0 Hz, 1H), 7.15 (ddd, J=11.9, 9.2, 2.6 Hz, 1H), 6.96 (td, J=8.5, 2.4 Hz, 1H), 5.62 (s, 1H), 4.55 (s, 2H), 3.48-3.39 (m, 1H), 2.85-2.82 (m, 1H), 2.69-2.62 (m, 2H), 2.62-2.56 (m, 1H), 2.28-2.19 (m, 2H), 1.74-1.65 (m, 2H), 1.42-1.34 (m, 2H).

### 2-amino-6-ethynyl-1-(4-phenyl-3,4-dihydro-2H-benzo[b][1,4]oxazin-6-yl)thieno[2,3-b]pyrazin-2(1H)-one (5-4)

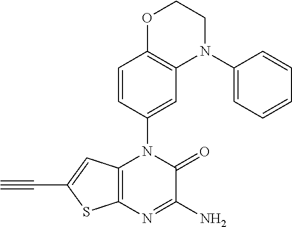 [SMILES: C#Cc1cc2c(nc(N)c(=O)n2-c2ccc3c(c2)N(c2ccccc2)CCO3)s1]

To a solution of dimethyl 1-diazo-2-oxopropyiphosphonate (142 mg, 0.73 mmol) in MeOH (5 mL) were added the 3-amino-2-oxo-1-(4-phenyl-3,4-dihydro-2H-benzo[b][1,4]oxazin-6-yl)-1,2-dihydrothieno[2,3-b]pyrazine-6-carbaldehyde 3-2 (100 mg, 0.25 mmol) and K₂CO₃ (136 mg, 0.98 mmol). The mixture was heated in a microwave reactor at 50° C. for 3 hours. After removing the volatiles without any heating, the crude product was purified by column chromatography on silica gel (DCM/MeOH=50:1) to afford 2-amino-6-ethynyl-1-(4-phenyl-3,4-dihydro-2H-benzo[b][1,4]oxazin-6-yl)thieno[2,3-b]pyrazin-2(1H)-one 5-4 as a yellow oil (40 mg, 39% yield).

LCMS: Calculated Exact Mass=400.1, Found [M+H]⁺ (ESI+)=401.2.

¹H NMR (400 MHz, DMSO) δ 7.43-7.28 (m, 6H), 7.09 (t, J=7.2 Hz, 1H), 6.99 (d, J=8.4 Hz, 1H), 6.79 (d, J=2.2 Hz, 1H), 6.74 (dd, J=8.4, 2.2 Hz, 1H), 6.49 (s, 1H), 4.56 (s, 1H), 4.36-4.33 (m, 2H), 3.77-3.74 (m, 2H).

### 3-amino-6-(1-(1-(2-(2,4-difluorophenyl)-2-hydroxy-3-(1H-1,2,4-triazol-1-yl)propyl)piperidin-4-yl)-1H-1,2,3-triazol-4-yl)-1-(4-phenyl-3,4-dihydro-2H-benzo[b][1,4]oxazin-6-yl)thieno[2,3-b]pyrazin-2(1H)-one (1-51)

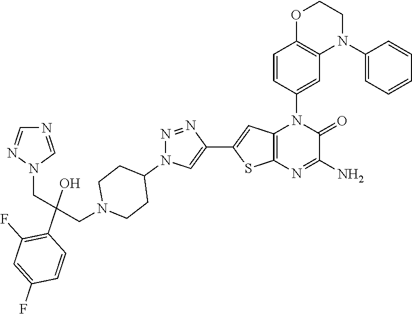 [SMILES: Nc1nc2sc(-c3cn(C4CCN(CC(O)(Cn5cncn5)c5ccc(F)cc5F)CC4)nn3)cc2n(-c2ccc3c(c2)N(c2ccccc2)CCO3)c1=O]

To a solution of the 2-amino-6-ethynyl-1-(4-phenyl-3,4-dihydro-2H-benzo[b][1,4]oxazin-6-yl) thieno[2,3-b]pyrazin-2(1H)-one 5-4 (40 mg, 0.1 mmol) and the 1-(4-azidopiperidin-1-yl)-2-(2,4-difluorophenyl)-3-(1H-1,2,4-triazol-1-yl)propan-2-ol 5-3 (47 mg, 0.13 mmol) in DMF (2 mL) were added sodium L-ascorbate (20 mg, 0.1 mmol) and CuSO₄·5H₂O (25 mg, 0.1 mmol). The reaction mixture was stirred for 6 hours at room temperature. Water (20 mL) was added and the mixture was extracted with EtOA (50 mL). The organic layer was dried over anhydrous Na₂SO₄, filtered and concentrated. The crude product was purified by Prep-HPLC (ACN/H₂O with 0.5% NH₃ as the mobile phase) to afford 3-amino-6-(1-(1-(2-(2,4-difluorophenyl)-2-hydroxy-3-(1H-1,2,4-triazol-1-yl)propyl)piperidin-4-yl)-1H-1,2,3-triazol-4-yl)-1-(4-phenyl-3,4-dihydro-2H-benzo[b][1,4]oxazin-6-yl)thieno[2,3-b]pyrazin-2(1H)-one 1-51 as a pale yellow solid (2.0 mg, 2.6%).

LCMS: Calculated Exact Mass=763.3, Found [M+H]⁺ (ESI+)=764.2.

¹H NMR (400 MHz, MeOD) δ 8.34 (s, 1H), 8.23 (s, 1H), 7.75 (s, 1H), 7.53-7.46 (m, 1H), 7.35-7.28 (m, 4H), 7.09-7.04 (m, 1H), 7.01 (d, J=8.4 Hz, 1H), 6.93 (ddd, J=11.7, 8.9, 2.5 Hz, 1H), 6.85 (dt, J=8.4, 4.2 Hz, 1H), 6.75-6.68 (m, 3H), 4.67 (d, J=17.3 Hz, 2H), 4.46-4.40 (m, 1H), 4.38 (t, J=4.3 Hz, 2H), 3.78-3.74 (m, 2H), 3.04 (d, J=14.8 Hz, 1H), 2.91 (d, J=11.5 Hz, 1H), 2.85 (d, J=13.9 Hz, 1H), 2.73 (d, J=12.0 Hz, 1H), 2.55-2.49 (m, 1H), 2.40-2.33 (m, 1H), 2.11-2.05 (m, 2H), 2.01-1.97 (m, 2H).

### Synthesis of 2,2-difluoropropyl 2-amino-4-(2,4-dichloro-6-(3-(2,4-difluorophenyl)-3-hydroxy-4-(1H-1,2,4-triazol-1-yl)butoxy)phenyl)-5,7-dihydro-6H-pyrrolo[3,4-d]pyrimidine-6-carboxylate—I-115

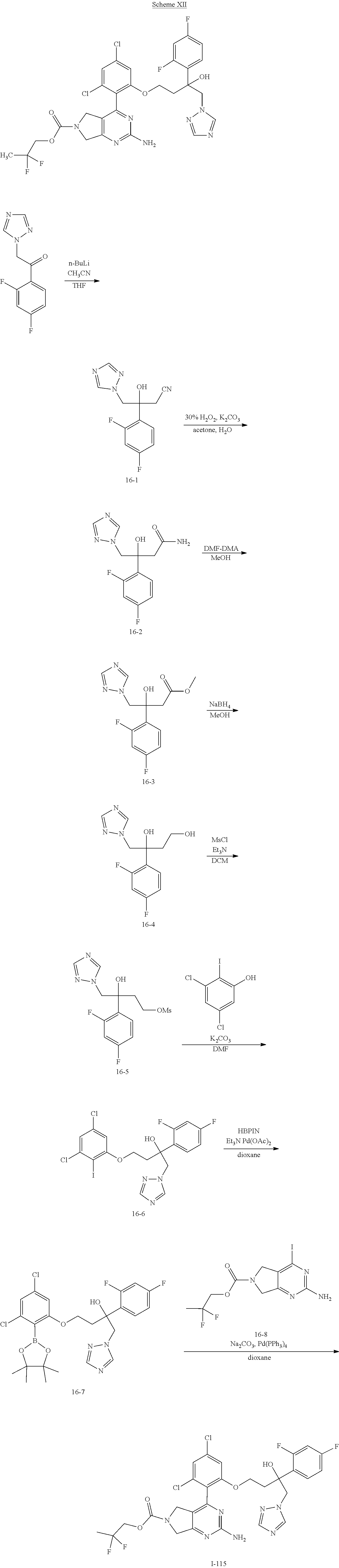

### 3-(2,4-difluorophenyl)-3-hydroxy-4-(1H-1,2,4-triazol-1-yl)butanenitrile (16-1)

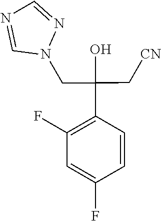 [SMILES: N#CCC(O)(Cn1cncn1)c1ccc(F)cc1F]

n-BuLi (1.6 M, 119 mL, 190 mmol) was added to a solution of CH₃ CN (6.8 g, 167 mmol) in 200 mL THF at −65° C., and the reaction vessel was purged with N₂. The mixture was stirred for 1 hour. 1-(2,4-difluorophenyl)-2-(1H-1,2,4-triazol-1-yl) ethan-1-one (25 g, 111 mmol) was dissolved in 200 mL THF, and it was added to the above solution at −65° C. The mixture was stirred for 1 h at −65° C. When the reaction was complete, ice water (200 mL) was added to the reaction mixture. The aqueous phase was extracted with EtOAc (3×200 mL), and the organic layers were combined, dried over Na₂SO₄, filtered and concentrated. The crude product was purified by column chromatography on silica gel (petroleum ether/EtOAc=1:1) to give 3-(2,4-difluorophenyl)-3-hydroxy-4-(1H-1,2,4-triazol-1-yl) butanenitrile 16-1 as a yellow solid (15 g, 51% yield). LCMS: Calculated Exact Mass=264.1, Found [m+H]⁺ (ESI+)=265.2

### 3-(2,4-difluorophenyI)-3-hydroxy-4-(1H-1,2,4-triazol-1-yl) butanamide (16-2)

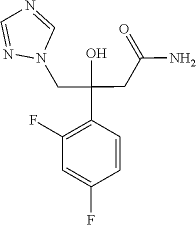 [SMILES: NC(=O)CC(O)(Cn1cncn1)c1ccc(F)cc1F]

30% H₂O₂(36.1 mL, 339 mmol) was added drop-wise to a mixture of 3-(2,4-difluorophenyl)-3-hydroxy-4-(1H-1,2,4-triazol-1-yl)butanenitrile 16-1 (18 g, 68 mmol) and Na₂CO₃ (21 g, 198 mmol) in 100 mL acetone and 200 mL H₂O. The mixture was stirred at room temperature overnight. When the reaction was complete, Na₂SO₃ (20 g, 158 mmol) was added to the mixture. The aqueous phase was extracted with EtOAc (3×100 mL), and the organic layers were combined, dried over Na₂SO₄, filtered and concentrated. The crude product was purified by column chromatography on silica gel (DCM/MeOH=10:1) to give 3-(2,4-difluorophenyl)-3-hydroxy-4-(1H-1,2,4-triazol-1-yl)butanamide 16-2 as a white solid (15 g, 78% yield).

LCMS: Calculated Exact Mass=282.1, Found [M+H]⁺ (ESI+)=283.2.

### Methyl 3-(2,4-difluorophenyl)-3-hydroxy-4-(1H-1,2,4-triazol-1-yl)butanoate (16-3)

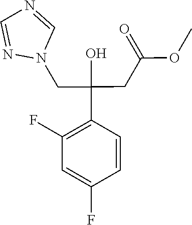 [SMILES: COC(=O)CC(O)(Cn1cncn1)c1ccc(F)cc1F]

DMF-DMA (25.5 mL, 190 mmol) was added to a solution of 3-(2,4-difluorophenyl)-3-hydroxy-4-(1H-1,2,4-triazol-1-yl)butanamide 16-2 (18 g, 63 mmol) in 150 mL MeOH. The mixture was stirred at room temperature overnight. The mixture was concentrated and the residue was diluted with EtOAc (500 mL). The organic layer was washed with H₂O (200 mL) and brine (200 mL), dried over Na₂SO₄, filtered and concentrated. The crude product was purified by column chromatography on silica gel (DCM/MeOH=10:1) to give methyl 3-(2,4-difluorophenyl)-3-hydroxy-4-(1H-1,2,4-triazol-1-yl)butanoate 16-3 as a white solid (11 g, 61% yield).

LCMS: Calculated Exact Mass=297.1, Found [M+H]⁺ (ESI+)=298.2.

### 3-(2,4-difluorophenyl)-4-(1H-1,2,4-triazol-1-yl)butane-1,3-diol (16-4)

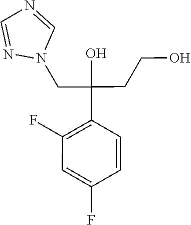 [SMILES: OCCC(O)(Cn1cncn1)c1ccc(F)cc1F]

To a solution of methyl 3-(2,4-difluorophenyl)-3-hydroxy-4-(1H-1,2,4-triazol-1-yl)butanoate 16-3 (11.5 g, 38.5 mmol) in 100 mL MeOH was added NaBH₄ (14.7 g, 386 mmol) at 0° C. The mixture was stirred at room temperature overnight. The mixture was quenched with H₂O (100 mL), extracted with EtOAc (3×100 mL). The organic layers were combined, dried over Na₂SO₄, filtered and concentrated to give crude 3-(2,4-difluorophenyl)-4-(1H-1,2,4-triazol-1-yl)butane-1,3-diol 16-4 as a white solid (10 g, 97% yield).

LCMS: Calculated Exact Mass=269.1, Found [M+H]⁺ (ESI+)=270.0.

### 3-(2,4-difluorophenyl)-3-hydroxy-4-(1H-1,2,4-triazol-1-yl)butyl methanesulfonate (16-5)

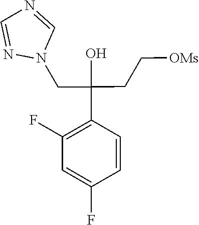 [SMILES: CS(=O)(=O)OCCC(O)(Cn1cncn1)c1ccc(F)cc1F]

To a solution of 3-(2,4-difluorophenyl)-4-(1H-1,2,4-triazol-1-yl)butane-1,3-diol 16-4 (6.0 g, 22 mmol) and Et₃N (3.36 g, 33 mmol) in 60 mL DCM was added methanesulfonyl chloride (3.0 g, 26 mmol) at 0° C. The mixture was stirred at room temperature for 4 hours. The mixture was directly used in the next reaction.

LCMS: Calculated Exact Mass=347.1, Found [M+H]⁺ (ESI+)=348.0.

### 4-(3,5-dichloro-2-iodophenoxy)-2-(2,4-difluorophenyl)-1-(1H-1,2,4-triazol-1-yl)butan-2-ol (16-6)

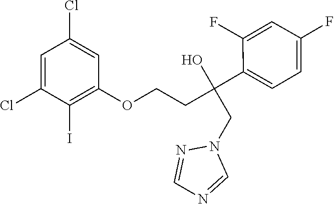 [SMILES: OC(CCOc1cc(Cl)cc(Cl)c1I)(Cn1cncn1)c1ccc(F)cc1F]

To a solution of 3,5-dichloro-2-iodophenol (9.5 g, 33 mmol) and K₂CO₃ (18.5 g, 134 mmol) in 50 mL DMF was added the mixture of 3-(2,4-difluorophenyl)-3-hydroxy-4-(1H-1,2,4-triazol-1-yl)butyl methanesulfonate 16-5 in DCM (60 mL). The mixture was stirred at 90° C. for 4 hours. The reaction was cooled down, the mixture was quenched with H₂O (200 mL) and extracted with EtOAc (3×200 mL). The organic layers were combined, dried over Na₂SO₄, filtered and concentrated. The crude product was purified by column chromatography on silica gel (PE/EA=2:1) to give 4-(3,5-dichloro-2-iodophenoxy)-2-(2,4-difluorophenyl)-1-(1H-1,2,4-triazol-1-yl) butan-2-ol 16-6 as a yellow solid (8 g, 67% yield).

LCMS: Calculated Exact Mass=538.9, Found [M+H]⁺ (ESI+)=540.1.

¹H NMR (400 MHz, DMSO) δ 8.29 (s, 1H), 7.76 (s, 1H), 7.37 (td, J=9.0, 6.9 Hz, 1H), 7.31 (d, J=2.1 Hz, 1H), 7.17 (ddd, J=11.9, 9.2, 2.5 Hz, 1H), 6.96 (dd, J=8.4, 2.4 Hz, 1H), 6.92 (dd, J=5.5, 2.2 Hz, 1H), 6.01 (s, 1H), 4.63 (q, J=14.4 Hz, 2H), 4.20 (dt, J=9.9, 6.7 Hz, 1H), 4.02-3.94 (m, 1H), 2.49-2.43 (m, 1H), 2.38-2.30 (m, 1H).

### 4-(3,5-dichloro-2-(4,4,5,5-tetramethyl-1,3,2-dioxaborolan-2-yl)phenoxy)-2-(2,4-difluorophenyl)-1-(1H-1,2,4-triazol-1-yl)butan-2-ol (16-7)

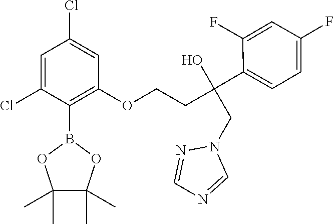 [SMILES: CC1(C)OB(c2c(Cl)cc(Cl)cc2OCCC(O)(Cn2cncn2)c2ccc(F)cc2F)OC1(C)C]

A solution of 4-(3,5-dichloro-2-iodophenoxy)-2-(2,4-difluorophenyl)-1-(1H-1,2,4-triazol-1-yl) butan-2-ol 16-6 (500 mg, 0.9 mmol), HBPin (1.7 g, 9.2 mmol), Et₃N (140 mg, 1.4 mmol) and Pd(PPh₃)₄(535 mg, 0.46 mmol) in 50 mL dioxane was stirred at 120° C. for 6 hours in microwave under N₂ atmosphere. When the reaction was complete, the mixture was concentrated. The crude product was purified by Prep-HPLC (ACN/H₂O with 0.01% TFA as the mobile phase) to give 4-(3,5-dichloro-2-(4,4,5,5-tetramethyl-1,3,2-dioxaborolan-2-yl)phenoxy)-2-(2,4-difluorophenyl)-1-(1H-1,2,4-triazol-1-yl)butan-2-ol 16-7 as a yellow solid (100 mg, 20% yield).

LCMS: Calculated Exact Mass=538.9, Found [M+H]⁺ (ESI+)=540.1.

### Synthesis of compound 2,2-difluoropropyl 2-amino-4-iodo-5,7-dihydro-6H-pyrrolo[3,4-d]pyrimidine-6-carboxylate (16-8)

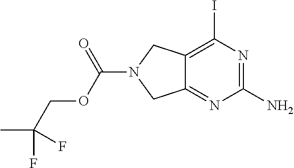 [SMILES: CC(F)(F)COC(=O)N1Cc2nc(N)nc(I)c2C1]

To a solution of 2,2-difluoropropan-1-ol (334 mg, 3.4 mmol) in THF (5 mL) was added CDI (826 mg, 5.1 mmol). The mixture was stirred at 60° C. for 2 hours. The mixture was cooled down and added to a solution of 4-iodo-6,7-dihydro-5H-pyrrolo[3,4-d]pyrimidin-2-amine (synthesized according to patent US2010/4168) (600 mg, 2.3 mmol) and Et₃N (1.0 g, 10 mmol) in DMF (6 mL). The mixture was stirred at 60° C. for 3 hours under N₂ atmosphere. The reaction mixture was diluted with EtOAc (50 mL) and washed with H₂O (20 mL) and brine (30 mL), dried over Na₂SO₄, filtered and concentrated. The crude was purified by column chromatography on silica gel (petroleum ether/EtOAc=1:1) to give 2,2-difluoropropyl 2-amino-4-iodo-5,7-dihydro-6H-pyrrolo [3,4-d]pyrimidine-6-carboxylate 16-8 as a white solid (200 mg, 22%).

LCMS: Calculated Exact Mass=384.0, Found [M+H]⁺ (ESI+)=385.2.

¹H NMR (400 MHz, d₆-DMSO) δ 7.04 (s, 2H), 6.87 (t, J=6.0 Hz, 1H), 4.47 (s, 2H), 4.32 (s, 2H), 3.50 (td, J=13.8, 6.2 Hz, 2H), 1.57 (t, J=19.0 Hz, 3H).

### 2,2-difluoropropyl-2-amino-4-(2,4-dichloro-6-(3-(2,4-difluorophenyl)-3-hydroxy-4-(1H-1,2,4-triazol-1-yl)butoxy)phenyl)-5,7-dihydro-6H-pyrrolo[3,4-d]pyrimidine-6-carboxylate (1-115)

A solution of 4-(3,5-dichloro-2-(4,4,5,5-tetramethyl-1,3,2-dioxaborolan-2-yl)phenoxy)-2-(2,4-difluorophenyl)-1-(1H-1,2,4-triazol-1-yl)butan-2-ol 16-7 (60 mg, 0.11 mmol), 2,2-difluoropropyl 2-amino-4-iodo-5,7-dihydro-6H-pyrrolo[3,4-d]pyrimidine-6-carboxylate 16-8 (64 mg, 0.16 mmol), Na₂CO₃ (17.7 mg, 0.16 mmol) and Pd(PPh₃)₄(64 mg, 0.05 mmol) in 2 mL dioxane and 0.5 mL H₂O was purged with N₂ three times and stirred at 90° C. for 2 hours in microwave. The mixture was filtered and concentrated. The crude product was purified by Prep-HPLC (ACN/H₂O with 0.05% NH₃ as the mobile phase) to give 2,2-difluoropropyl 2-amino-4-(2,4-dichloro-6-(3-(2,4-difluorophenyl)-3-hydroxy-4-(1H-1,2,4-triazol-1-yl)butoxy)phenyl)-5,7-dihydro-6H-pyrrolo[3,4-d]pyrimidine-6-carboxylate I-115 as a white solid (20 mg, 27% yield).

LCMS: Calculated Exact Mass=669.1, Found [M+H]⁺ (ESI+)=670.1.

¹H NMR (400 MHz, MeOD) δ 8.20 (d, J=11.1 Hz, 1H), 7.71 (s, 1H), 7.39-7.27 (m, 1H), 7.20 (s, 1H), 7.07-7.03 (m, 1H), 6.85-6.74 (m, 2H), 4.63 (d, J=18.7 Hz, 2H), 4.49-4.42 (m, 2H), 4.41-4.35 (m, 2H), 4.34-4.27 (m, 2H), 4.16-4.05 (m, 2H), 2.41-2.24 (m, 2H), 1.76-1.56 (m, 3H).

In a similar manner, using the above procedure, the following amide compounds were synthesized

| Compound | | | Yield | |
|---|---|---|---|---|
| ID# | ¹H NMR | LCMS | % | Appearance |
| I-113 | ¹H NMR: (400 MHz, Chloroform-d) δ 7.30-7.27 (m, | Calculated | 6.8% | White |
| | 1H), 7.14 (dd, J = 6.8, 1.7 Hz, 1H), 6.92 (dd, J = 13.7, | exact mass = | | solid |
| | 4.7 Hz, 1H), 6.84 (dd, J = 7.0, 1.8 Hz, 1H), 5.23 (s, 2H), | 532.315 | | |
| | 4.72-4.62 (m, 2H), 4.40-4.19 (m, 8H), 4.03 (d, J = | Found | | |
| | 6.9 Hz, 1H), 1.74-1.59 (m, 3H). | [M + H]⁺ | | |
| | | (ESI+) = 532.2 | | |
| I-114 | ¹H NMR: (400 MHz, Methanol-d₄) δ 8.32 (d, J = 6.7 Hz, | Calculated | 14.4% | White |
| | 1H), 7.86 (d, J = 3.6 Hz, 1H), 7.30 (t, J = 7.9 Hz, 1H), | exact mass = | | solid |
| | 7.21 (dt, J = 2.7, 1.4 Hz, 1H), 7.13 (dd, J = 7.5, 1.8 Hz, | 657.416 | | |
| | 1H), 6.85-6.72 (m, 2H), 4.63-4.42 (m, 6H), 4.39- | Found | | |
| | 4.30 (m, 2H), 4.28-4.21 (m, 1H), 4.19-4.06 (m, 2H), | [M + H]⁺ | | |
| | 3.78 (dd, J = 30.6, 14.1 Hz, 1H), 1.75-1.56 (m, 3H). | (ESI+) = 656.3 | | |
| I-157 | ¹H NMR: (400 MHz, DMSO-d₆) δ 8.15 (d, J = 25.5 Hz, | Calculated | 3.8% | White |
| | 1H), 7.67 (d, J = 15.3 Hz, 1H), 7.32 (d, J = 1.8 Hz, 1H), | exact mass = | | solid |
| | 7.31-7.21 (m, 1H), 7.15 (dd, J = 11.1, 1.8 Hz, 1H), | 670.457 | | |
| | 7.02 (dddd, J = 14.3, 11.8, 9.0, 2.6 Hz, 1H), 6.90 (ddt, | Found | | |
| | J = 13.3, 8.6, 4.2 Hz, 1H), 6.82 (d, J = 18.9 Hz, 2H), 6.71 | [M + H]⁺ | | |
| | (t, J = 6.3 Hz, 1H), 5.90 (d, J = 13.1 Hz, 1H), 4.49-4.33 | (ESI+) = 669.2 | | |
| | (m, 3H), 4.26-4.03 (m, 4H), 3.99-3.83 (m, 1H), 3.48 | | | |
| | (tt, J = 13.7, 6.8 Hz, 2H), 2.26-2.12 (m, 2H), 1.55 (td, | | | |
| | J = 19.0, 3.7 Hz, 3H). | | | |
| I-158 | ¹H NMR: (400 MHz, Methanol-d₄) δ 8.10 (dd, J = 11.2, | Calculated | 26.7% | White |
| | 1.2 Hz, 1H), 7.61 (d, J = 1.2 Hz, 1H), 7.29-7.18 (m, | exact mass = | | solid |
| | 1H), 7.13-7.09 (m, 1H), 6.95 (dd, J = 4.7, 1.6 Hz, 1H), | 671.442 | | |
| | 6.75-6.63 (m, 2H), 4.57-4.50 (m, 2H), 4.40-4.30 | Found | | |
| | (m, 3H), 4.28-4.19 (m, 3H), 4.19-4.10 (m, 1H), 4.08- | [M + H]⁺ | | |
| | 3.95 (m, 2H), 2.24 (ddq, J = 26.3, 13.7, 6.9, 5.8 Hz, | (ESI+) = 670.2 | | |
| | 2H), 1.66-1.46 (m, 3H). | | | |
| I-159 | ¹H NMR: (400 MHz, Methanol-d₄) δ 8.20 (dd, J = 11.0, | Calculated | 25% | White |
| | 1.2 Hz, 1H), 7.71 (d, J = 1.1 Hz, 1H), 7.39-7.27 (m, | exact mass = | | solid |
| | 1H), 7.20 (dt, J = 2.9, 1.4 Hz, 1H), 7.07-7.04 (m, 1H), | 671.442 | | |
| | 6.85-6.74 (m, 2H), 4.67-4.58 (m, 2H), 4.51-4.39 | Found | | |
| | (m, 3H), 4.38-4.30 (m, 3H), 4.29-4.21 (m, 1H), 4.19- | [M + H]⁺ | | |
| | 4.04 (m, 2H), 2.41-2.24 (m, 2H), 1.76-1.57 (m, 3H). | (ESI+) = 670.3 | | |
| I-161 | ¹H NMR: (400 MHz, DMSO-d₆) δ 8.14 (d, J = 25.5 Hz, | Calculated | 11.3% | White |
| | 1H), 7.67 (d, J = 15.3 Hz, 1H), 7.32 (d, J = 1.7 Hz, 1H), | exact mass = | | solid |
| | 7.31-7.22 (m, 1H), 7.15 (dd, J = 11.1, 1.8 Hz, 1H), | 670.457 | | |
| | 7.07-6.96 (m, 1H), 6.90 (ddt, J = 13.4, 8.5, 4.1 Hz, | Found | | |
| | 1H), 6.85-6.76 (m, 2H), 6.71 (t, J = 6.3 Hz, 1H), 5.89 | [M + H]⁺ | | |
| | (d, J = 13.1 Hz, 1H), 4.48-4.32 (m, 3H), 4.25-4.02 | (ESI+) = 669.4 | | |
| | (m, 4H), 3.99-3.85 (m, 1H), 3.46 (dt, J = 13.5, 6.7 Hz, | | | |
| | 2H), 2.25-2.11 (m, 2H), 1.55 (td, J = 19.0, 3.7 Hz, 3H). | | | |
| I-162 | ¹H NMR: (400 MHz, DMSO-d₆) δ 8.15 (d, J = 25.5 Hz, | Calculated | 8.3% | White |
| | 1H), 7.67 (d, J = 15.3 Hz, 1H), 7.32 (d, J = 1.7 Hz, 1H), | exact mass = | | solid |
| | 7.30-7.21 (m, 1H), 7.15 (dd, J = 11.1, 1.9 Hz, 1H), | 670.457 | | |
| | 7.02 (dddd, J = 14.4, 11.9, 9.0, 2.6 Hz, 1H), 6.90 (ddd, | Found | | |
| | J = 12.3, 8.0, 2.7 Hz, 1H), 6.86-6.77 (m, 2H), 6.78- | [M + H]⁺ | | |
| | 6.67 (m, 1H), 5.89 (d, J = 13.1 Hz, 1H), 4.49-4.33 (m, | (ESI+) = 669.2 | | |
| | 3H), 4.26-4.04 (m, 4H), 3.99-3.86 (m, 1H), 3.48 (tt, | | | |
| | J = 13.8, 6.8 Hz, 2H), 2.27-2.11 (m, 2H), 1.55 (td, J = | | | |
| | 19.0, 3.7 Hz, 3H). | | | |

### B: Biological Assays

### (a) Assay

Surface Plasmon Resonance (SPR) Experiments. SPR experiments were performed on a Biacore S200 instrument at 25° C. Biotinylated Hsp90 Nuclear Binding Domain (NBD) was diluted to 40 μg/mL and immobilized on a streptavidin chip (Sensor Chip SA, GE Healthcare) at a density of 2000-2500 response units (RU) on the biosensor surface.

Recombinant Hsp90 NBDs were expressed and purified as previously described (Whitesell et al., Nat Commun 10, 402 (2019)) with the following modification; Hsp90 NBD expression constructs were modified to encode a C-terminal AviTag for site-specific on-column biotinylation with a BirA biotin-ligase kit (Avidity LLC; BirA-500). Stock protein solutions in 50% glycerol were stored at −20° C. until dilution into relevant buffers. Binding experiments were done in HBS-P (0.01 M HEPES, pH 7.4, 0.15 M NaCl, 0.005% v/v surfactant P20, GE Healthcare) with 2% DMSO at flow rate of 40 μL/min. Test compounds (dilution series) were injected with a 60 s association time and 600 s dissociation time. Resulting sensorgrams were analyzed with a fit to a 1:1 binding model, using BIA evaluation software.

TABLE 3
SPR experiments results

| | |
|---|---|
| Compound | SPR KD uM |

| ID # | C. albicans | A. fumigatus |
|---|---|---|
| I-4 | 0.0280 | 0.1130 |
| I-7 | 0.0228 | 0.0764 |
| I-9 | 0.1360 | 3.8700 |
| I-11 | 0.0008 | 0.0248 |
| I-12 | 0.1460 | 0.4310 |
| I-13 | 0.0504 | 0.0751 |
| I-14 | 0.0234 | 0.1460 |
| I-15 | 0.0304 | 0.1880 |
| I-16 | 0.0160 | 0.1400 |
| I-17 | 2.6700 | 2.7000 |
| I-18 | 0.4590 | 0.7410 |
| I-21 | 0.0067 | 0.0373 |
| I-23 | 0.0286 | 0.0460 |
| I-25 | 0.1710 | 0.2990 |
| I-27 | 0.0092 | 0.0134 |
| I-28 | 0.0133 | 0.1590 |
| I-31 | 0.0003 | 0.0023 |
| I-32 | 0.0036 | 0.0505 |
| I-35 | 1.9500 | 1.8400 |
| I-36 | 0.0021 | 0.0037 |
| I-47 | 0.2890 | 0.5500 |
| I-50 | 0.0427 | NB |
| I-51 | 0.0030 | 0.0146 |
| I-53 | 0.0013 | 0.0236 |
| I-54 | 0.0735 | 0.3390 |
| I-55 | 0.3180 | 0.3140 |
| I-56 | 0.0646 | 0.2290 |
| I-57 | 0.0321 | 0.0469 |
| I-62 | 0.2340 | 0.4230 |
| I-63 | 0.3770 | 0.5910 |
| I-64 | 0.0009 | 0.0193 |
| I-66 | 1.0600 | 1.3800 |
| I-67 | 0.4020 | 0.5030 |
| I-68 | 0.4170 | 0.5450 |
| I-70 | 0.0003 | 0.0029 |
| I-71 | 0.8650 | 1.2600 |
| I-72 | 0.1210 | 0.1970 |
| I-74 | 0.4290 | 0.8530 |
| I-75 | 0.2330 | 0.3400 |
| I-77 | 0.1870 | 0.3730 |
| I-78 | 0.3000 | 0.3660 |
| I-79 | 0.8770 | 1.0200 |
| I-81 | 0.8250 | 1.0400 |
| I-82 | 0.8260 | 1.3500 |
| I-115 | 1.2000 | 1.1700 |
| I-116 | 1.3500 | NB |
| I-117 | 0.4610 | 0.2980 |
| I-118 | 0.1910 | 0.4060 |
| I-120 | 0.1790 | 0.2650 |
| I-122 | 1.6300 | 1.9000 |
| I-123 | 0.1450 | 0.2380 |
| I-125 | 0.5750 | 0.8860 |
| I-127 | 0.6990 | 1.1900 |
| I-129 | 0.5460 | 0.6980 |
| I-130 | 1.5800 | 2.1900 |
| I-133 | 0.3000 | 0.6200 |
| I-134 | 0.6650 | 1.0500 |
| I-136 | 0.2710 | 0.5290 |
| I-137 | 0.0541 | 0.1270 |
| I-138 | 0.4450 | 0.6740 |
| I-139 | 0.1510 | 0.2460 |
| I-140 | 0.7460 | 0.4340 |
| I-141 | 0.3420 | 0.6200 |
| I-142 | 0.0024 | 0.0341 |
| I-145 | 0.1140 | 0.1610 |

### (b) Assay

Antifungal Sensitivity Testing. Minimum inhibitory concentrations (MICs) were determined in flat bottom, 96-well plate format using a modified broth microdilution protocol as previously described (Singh et al., PLoS Pathog. 5, e1000532 (2009); LaFayette et al., PLoS Pathog. 6, 79-80 (2010)). Compounds were formulated in dimethyl sulfoxide (DMSO, Sigma-Aldrich Co.); fluconazole was dissolved in sterile ddH₂O. Each compound was tested in duplicate in at least two independent experiments. To test for fungicidal activity, cultures from MIC plates were spotted on YPD agar plates using a spotter (Frogger, V&P Scientific, Inc.). Plates were photographed after 24 h of incubation at 30° C.

TABLE 4
Antifungal Sensitivity Testing Results

| Compound ID # | CaCl₂ MIC50 uM |
|---|---|
| I-21 | 0.085 |
| I-12 | 0.18 |
| I-23 | 0.35 |
| I-13 | 0.37 |
| I-25 | 0.5 |
| I-16 | 0.6 |
| I-54 | 0.6 |
| I-4 | 0.75 |
| I-7 | 0.75 |
| I-8 | 0.75 |
| I-68 | 0.75 |
| I-137 | 0.78 |
| I-46 | 0.85 |
| I-31 | 1 |
| I-47 | 1 |
| I-51 | 1 |
| I-139 | 1.2 |
| I-131 | 1.25 |
| I-53 | 1.5 |
| I-82 | 1.6 |
| I-130 | 1.75 |
| I-118 | 2 |
| I-133 | 2.13 |
| I-67 | 2.25 |
| I-138 | 2.3 |
| I-11 | 2.5 |
| I-27 | 2.5 |
| I-32 | 2.5 |
| I-81 | 2.5 |
| I-15 | 2.75 |
| I-56 | 3 |
| I-62 | 3 |
| I-79 | 3 |
| I-116 | 3 |
| I-117 | 3 |
| I-64 | 3.125 |
| I-134 | 3.13 |
| I-50 | 3.25 |
| I-75 | 4 |
| I-70 | 5.75 |
| I-125 | 5.75 |
| I-28 | 6 |
| I-71 | 6.25 |
| I-78 | 6.25 |
| I-127 | 6.25 |
| I-136 | 6.25 |
| I-140 | 6.3 |
| I-141 | 6.3 |
| I-115 | 6.5 |
| I-119 | 6.5 |
| I-122 | 6.5 |
| I-129 | 7 |
| I-72 | 7.5 |
| I-36 | 8 |
| I-55 | 9 |
| I-63 | 9 |
| I-9 | 10 |
| I-123 | 11.5 |
| I-74 | 12 |
| I-77 | 12.5 |
| I-145 | 12.5 |
| I-120 | 17.5 |
| I-18 | 20 |
| I-57 | 20 |
| I-66 | 22.5 |
| I-14 | 25 |
| I-35 | 25 |
| I-142 | 25 |
| I-17 | 45 |

### (c) Assay

Mammalian Cell Toxicity. HepG2 cells were seeded overnight at 500 cells/well in 384-well plate, then incubated with compounds (10-point concentration titration) for 48 h. Cell Titer-Glo® reagent (ThermoFisher Scientific) was added to each well and after 10-minute incubation at room temperature the luminescent signal was measured using an Envision plate reader (Perkin Elmer).

TABLE 5
Mammalian Cell Toxicity Results

| Compound ID # | HepG2 MIC50 uM |
|---|---|
| I-21 | >50 |
| I-12 | >10 |
| I-23 | >50 |
| I-54 | >10 |
| I-4 | >10 |
| I-31 | 4.6 |
| I-47 | 4.4 |
| I-139 | 3.9 |
| I-131 | 9 |
| I-53 | 4 |
| I-130 | >20 |
| I-118 | 3 |

### (d) Assay

Microsomal Stability Studies. Compounds (1 μM) were incubated at 37° C. for 45 min in a final volume of 270 μL of 100 mM potassium phosphate buffer (pH 7.4) containing pooled liver microsomes (0.5 mg/mL protein) and 2 mM NADPH. Reactions were initiated with the addition of NADPH following a 10 min preincubation. Aliquots of incubation samples were protein precipitated with cold methanol and centrifuged, and supernatants were analyzed by LCMS/MS. All incubations were performed in duplicate. T_{1/2} was calculated from the following equation T_{1/2}=0.693/K (K is the rate constant from a plot of In [concentration] vs. incubation time).

TABLE 6
Mammalian Cell Toxicity Results

| Compound ID # | HepG2 MIC50 uM | CaCl2 MIC50 uM |
|---|---|---|
| I-21 | >50 | 0.09 |
| I-12 | >10 | 0.18 |
| I-23 | >50 | 0.35 |
| I-54 | >10 | 0.60 |
| I-4 | >10 | 0.75 |
| I-31 | 4.6 | 1.00 |
| I-47 | 4.4 | 1.00 |
| I-139 | 3.9 | 1.20 |
| I-131 | 9 | 1.25 |
| I-53 | 4 | 1.50 |
| I-130 | >20 | 1.75 |
| I-118 | 3 | 2.00 |
| I-133 | 16.8 | 2.13 |
| I-67 | 4.7 | 2.25 |
| I-138 | 7.9 | 2.30 |
| I-11 | 2.4 | 2.50 |
| I-27 | >10 | 2.50 |
| I-32 | 8.2 | 2.50 |
| I-56 | >10 | 3.00 |
| I-62 | 10.6 | 3.00 |
| I-64 | 2 | 3.13 |
| I-134 | 15 | 3.13 |
| I-70 | 1.5 | 5.75 |
| I-136 | 19.4 | 6.25 |
| I-115 | >20 | 6.50 |
| I-122 | 14.3 | 6.50 |
| I-72 | 4.6 | 7.50 |
| I-36 | 1.9 | 8.00 |
| I-55 | >10 | 9.00 |
| I-18 | >50 | 20.00 |

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A conjugate compound of Formula (I) or an enantiomer thereof, or a pharmaceutically acceptable salt, solvate and/or prodrug thereof,
A-L¹-B (I)
wherein:
A is a moiety that increases fungal cell uptake and/or fungal cell permeability;
B is a HSP90 inhibiting moiety; and
L¹ is a linker comprising at least one complimentary functional group to covalently bind with A and at least one complimentary functional group to react with B, to form the conjugate of Formula (I).

2. The conjugate compound of claim 1, wherein A is an antifungal moiety.

3. The conjugate compound of claim 1, wherein A comprises at least one of: an azole moiety, a polyamine moiety, a fatty acid ester moiety, a fatty acid amide moiety, a fatty alcohol moiety, a flucytosine moiety, and a triphenylphosphonium moiety compound.

4. The conjugate compound of claim 3, wherein the azole moiety is selected from an imidazole moiety, a triazole moiety, a tetrazole moiety and a thiazole moiety.

5. The conjugate of claim 1, wherein A is selected from fluconazole, itraconazole, clotrimazole, ketoconazole, voriconazole, posaconazole, isavuconazonium, miconazole, flucytosine, olorofim, manogepix, ibrexafungerp, caspofungin, micafungin, anidulafungin, rezafungin, amphotericin B and VT-1161.

6. The conjugate compound of claim 1, wherein B is a substituted C₄₋₂₀heteroaryl moiety.

7. The conjugate compound of claim 6, wherein B comprises at least one of: a pyrazinone moiety, an indazole moiety and a pyrimidine moiety.

8. The conjugate compound of claim 6, wherein B comprises a pyrrolopyrazinone moiety, a thienopyrizanone moiety, a tetrahydroindazole moiety and/or a pyrrolopyrimidine moiety.

9. The conjugate of claim 1, wherein B comprises a compound listed in Table 1.

10. The conjugate of claim 1, wherein B is selected from: 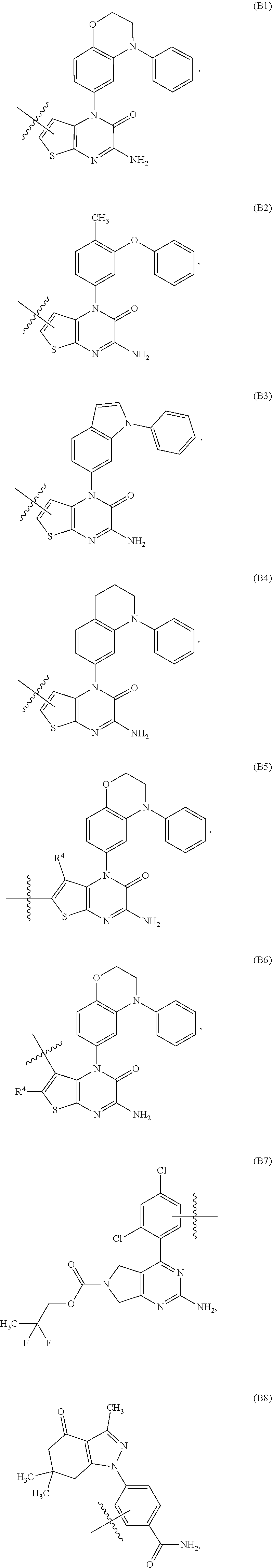 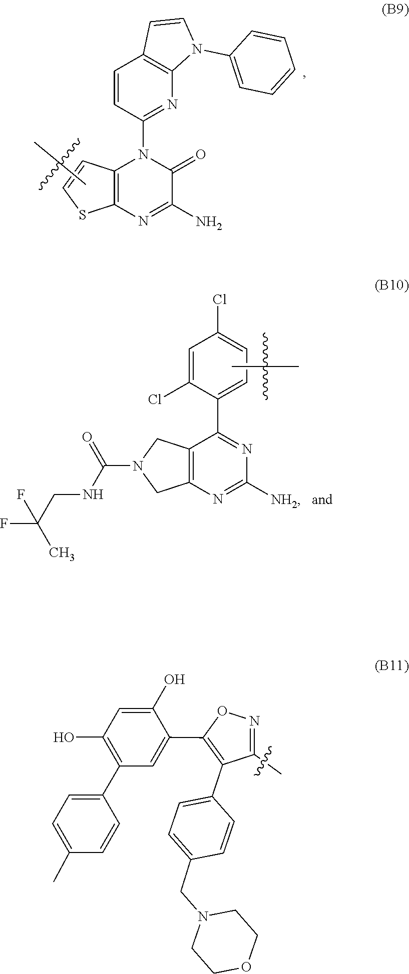
wherein R⁴ is selected from C(O)OC₁₋₆alkyl, C(S)OC₁₋₆alkyl, C(O)OH, C(O)NH₂, C(O)NHC₁₋₆alkyl, C(S)NHC₁₋₆alkyl and CH(OH)C₁₋₆alkyl, and each alkyl group is optionally fluorosubstituted; and [symbol] represents the point of attachment to the remainder of the conjugate, wherein the point of attachment is at the 6- or 7-position in B1, B2, B3, B4, and B9, or the ortho or meta position in B7, B8 and A10.

11. The conjugate compound of claim 1, wherein L¹ comprises one or more of C(O),C(S), O, S, S(O), SO₂, NR², C₁₋₁₂alkylene, C₆₋₂₀arylene, C₃₋₁₀cycloalkylene, C₂₋₂₀heteroarylene and C₃₋₁₀heterocycloalkylene, wherein each of the alkylene, arylene, cycloalkylene, heteroarylene and heterocycloalkylene is optionally substituted with one or more R¹, and each R¹ is independently selected from C₁₋₁₂alkyl, NR²C(O)R³, NR²C(S)R³, NR²R³, NR²C(S)NR²R³, halo, C(O)NR²R³, C(S)NR²R³, SR² and OR²; and R² and R³ are independently selected from H, C₁₋₁₂alkyl and C₁₋₁₂fluoroalkyl.

12. The conjugate compound of claim 1, wherein L¹ is selected from C₁₋₁₂alkylene-NR²—C(O), C₁₋₆alkylene-C₆₋₁₀arylene-NR²—C(O), C₁₋₆alkylene-NR²—C(O)—C₂₋₁₀heteroarylene, C₁₋₆alkylene-C₂₋₁₀heteroarylene-NR²—C(O), C₂₋₁₀heteroarylene-NR²—C(O), C₁₋₆alkylene-C₃₋₁₀heterocycloalkylene-NR²—C(O)—C₂₋₁₀heteroarylene, C₁₋₆alkylene-C₃₋₁₀heterocycloalkylene-C₂₋₁₀heteroarylene, C₁₋₆alkylene-C₃₋₁₀heterocycloalkylene-NR²—C(O), C₁₋₆alkylene-C₃₋₁₀heterocycloalkylene-C₁₋₆alkylene-NR²—C(O), C₁₋₆alkylene-C₃₋₁₀heterocycloalkylene-C₆₋₁₀arylene-NR²—C(O), C₁₋₆alkylene-C₃₋₁₀heterocycloalkylene-C₁₋₆alkylene-C₆₋₁₀arylene-NR²—C(O), C₁₋₆alkylene-C(O)—C₃₋₁₀heterocycloalkylene-NR²—C(O), C₁₋₆alkylene-C(O)—C₃₋₁₀heterocycloalkylene-NR²—C₁₋₆alkylene, C₁₋₆alkylene-O—C(O)—C₁₋₆alkylene-C₃₋₁₀heterocycloalkylene-NR²—C(O), C₁₋₆alkylene-C₃₋₁₀heterocycloalkylene-NR²—C₁₋₆alkylene, C₁₋₆alkylene-C₃₋₁₀heterocycloalkylene-C₁₋₆alkylene-NR²—C₁₋₆alkylene, C₁₋₆alkylene-C₃₋₁₀heterocycloalkylene-C₁₋₆alkylene, C₁₋₆alkylene-NR²—C₁₋₆alkylene, O—C₁₋₁₂alkylene, C₁₋₆alkylene-C₃₋₁₀heterocycloalkylene-O—C₁₋₆alkylene-C₆₋₁₀arylene-NR²—C(O), C₁₋₆alkylene-C₃₋₁₀heterocycloalkylene-O—C₁₋₆alkylene-O—C₆₋₁₀arylene-NR²—C(O), and NR²—C₁₋₆alkylene-O—C₁₋₆alkylene-O—C₁₋₆alkylene-NR², each L¹ is optionally substituted with one or more R¹ and A and B are covalently bonded to either end of L¹.

13. The conjugate of claim 11, wherein each R¹ is independently selected from C₁₋₆alkyl, NR²C(O)R³, NR²R³, F, C₁, C(O)NR²R³, and OR²;
and R² and R³ are independently selected from H, C₁₋₆alkyl and C₁₋₆fluoroalkyl.

14. The conjugate of claim 13, wherein each R¹ is independently selected from methyl, ethyl, propyl, F, Cl, NH₂, NHC(O)C₁₋₆alkyl, C(O)NH₂ and OH.

15. The conjugate of claim 13, wherein R² and R³ are independently selected from H, methyl, ethyl, propyl, CF₃, CHF₂ and CH₂F.

16. The conjugate compound of claim 1, wherein the compound of Formula (I) is selected from the compounds listed in Table 2 or a salt, and/or solvate thereof.

17. A method of treating or preventing a fungal-related disease, disorder or condition comprising administering a therapeutically effective amount of one or more conjugate compounds of claim 1 to a subject in need thereof.

18. A method of inhibiting or preventing fungal growth comprising administering a therapeutically effective amount of one or more conjugate compounds of claim 1 to a subject in need thereof.

19. A method of inhibiting fungal HSP90 activity comprising administering a therapeutically effective amount of one or more conjugate compounds of claim 1 to a subject in need thereof.

20. (canceled)

21. (canceled)

22. (canceled)

23. A pharmaceutical composition comprising one or more conjugate compounds of claim 1, or a pharmaceutically acceptable salt, and/or solvate thereof, and a pharmaceutically acceptable carrier and/or diluent.

24. (canceled)

25. (canceled)
